SEQUENCE LISTING (63 sequences)

SEQ ID NO: 1 - protein, 214 aa, synthetic construct
  source 1..214: note = Ranibizumab Fab Amino Acid Sequence (Light chain)
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, synthetic construct
  source 1..231: note = Ranibizumab Fab Amino Acid Sequence (Heavy chain)
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 3 - protein, 214 aa, synthetic construct
  source 1..214: note = Bevacizumab, Hyperglycosylated Bevacizumab Fab Amino Acid Sequence (Light chain)
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 231 aa, synthetic construct
  source 1..231: note = Bevacizumab, Hyperglycosylated Bevacizumab Fab Amino Acid Sequence (Heavy chain)
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 26 aa, synthetic construct
  source 1..26: note = VEGF-A signal peptide
MNFLLSWVHWSLALLLYLHHAKWSQA

SEQ ID NO: 6 - protein, 29 aa, synthetic construct
  source 1..29: note = Fibulin-1 signal peptide
MERAAPSRRVPLPLLLLGGLALLAAGVDA

SEQ ID NO: 7 - protein, 19 aa, synthetic construct
  source 1..19: note = Vitronectin signal peptide
MAPLRPLLILALLAWVALA

SEQ ID NO: 8 - protein, 18 aa, synthetic construct
  source 1..18: note = Complement Factor H signal peptide
MRLLAKIICLMLWAICVA

SEQ ID NO: 9 - protein, 19 aa, synthetic construct
  source 1..19: note = Opticin signal peptide
MRLLAFLSLLALVLQETGT

SEQ ID NO: 10 - DNA, 728 bp, synthetic construct
  source 1..728: mol_type = other DNA; note = Bevacizumab cDNA (Light chain)
gctagcgccaccatgggctggtcctgcatcatcctgttcctggtggccaccgccaccggcgtgcactccgacatccagatgacccagtccccctcctccctgtccgcctccgtgggcgaccgggtgaccatcacctgctccgcctcccaggacatctccaactacctgaactggtaccagcagaagcccggcaaggcccccaaggtgctgatctacttcacctcctccctgcactccggcgtgccctcccggttctccggctccggctccggcaccgacttcaccctgaccatctcctccctgcagcccgaggacttcgccacctactactgccagcagtactccaccgtgccctggaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgctgagcggccgcctcgag

SEQ ID NO: 11 - DNA, 1440 bp, synthetic construct
  source 1..1440: mol_type = other DNA; note = Bevacizumab cDNA (Heavy chain)
gctagcgccaccatgggctggtcctgcatcatcctgttcctggtggccaccgccaccggcgtgcactccgaggtgcagctggtggagtccggcggcggcctggtgcagcccggcggctccctgcggctgtcctgcgccgcctccggctacaccttcaccaactacggcatgaactgggtgcggcaggcccccggcaagggcctggagtgggtgggctggatcaacacctacaccggcgagcccacctacgccgccgacttcaagcggcggttcaccttctccctggacacctccaagtccaccgcctacctgcagatgaactccctgcgggccgaggacaccgccgtgtactactgcgccaagtacccccactactacggctcctcccactggtacttcgacgtgtggggccagggcaccctggtgaccgtgtcctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccggacccccgaggtgacctgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaagtgagcggccgcc

SEQ ID NO: 12 - DNA, 733 bp, synthetic construct
  source 1..733: mol_type = other DNA; note = Ranibizumab cDNA (Light chain comprising a signal sequence)
gagctccatggagtttttcaaaaagacggcacttgccgcactggttatgggttttagtggtgcagcattggccgatatccagctgacccagagcccgagcagcctgagcgcaagcgttggtgatcgtgttaccattacctgtagcgcaagccaggatattagcaattatctgaattggtatcagcagaaaccgggtaaagcaccgaaagttctgatttattttaccagcagcctgcatagcggtgttccgagccgttttagcggtagcggtagtggcaccgattttaccctgaccattagcagcctgcagccggaagattttgcaacctattattgtcagcagtatagcaccgttccgtggacctttggtcagggcaccaaagttgaaattaaacgtaccgttgcagcaccgagcgtttttatttttccgcctagtgatgaacagctgaaaagcggcaccgcaagcgttgtttgtctgctgaataatttttatccgcgtgaagcaaaagtgcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagttttaatcgtggcgaatgctaatagaagcttggtacc

SEQ ID NO: 13 - DNA, 779 bp, synthetic construct
  source 1..779: mol_type = other DNA; note = Ranibizumab cDNA (Heavy chain comprising a signal sequence)
gagctcatatgaaatacctgctgccgaccgctgctgctggtctgctgctcctcgctgcccagccggcgatggccgaagttcagctggttgaaagcggtggtggtctggttcagcctggtggtagcctgcgtctgagctgtgcagcaagcggttatgattttacccattatggtatgaattgggttcgtcaggcaccgggtaaaggtctggaatgggttggttggattaatacctataccggtgaaccgacctatgcagcagattttaaacgtcgttttacctttagcctggataccagcaaaagcaccgcatatctgcagatgaatagcctgcgtgcagaagataccgcagtttattattgtgccaaatatccgtattactatggcaccagccactggtatttcgatgtttggggtcagggcaccctggttaccgttagcagcgcaagcaccaaaggtccgagcgtttttccgctggcaccgagcagcaaaagtaccagcggtggcacagcagcactgggttgtctggttaaagattattttccggaaccggttaccgtgagctggaatagcggtgcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaaaaagttgagccgaaaagctgcgataaaacccatctgtaatagggtacc

SEQ ID NO: 14 - protein, 11 aa, synthetic construct
  source 1..11: note = Bevacizumab and Ranibizumab Light Chain CDR1
SASQDISNYLN

SEQ ID NO: 15 - protein, 7 aa, synthetic construct
  source 1..7: note = Bevacizumab and Ranibizumab Light Chain CDR2
FTSSLHS

SEQ ID NO: 16 - protein, 9 aa, synthetic construct
  source 1..9: note = Bevacizumab and Ranibizumab Light Chain CDR3
QQYSTVPWT

SEQ ID NO: 17 - protein, 10 aa, synthetic construct
  source 1..10: note = Bevacizumab Heavy Chain CDR1
GYTFTNYGMN

SEQ ID NO: 18 - protein, 17 aa, synthetic construct
  source 1..17: note = Bevacizumab and Ranibizumab Heavy Chain CDR2
WINTYTGEPTYAADFKR

SEQ ID NO: 19 - protein, 14 aa, synthetic construct
  source 1..14: note = Bevacizumab Heavy Chain CDR3
YPHYYGSSHWYFDV

SEQ ID NO: 20 - protein, 10 aa, synthetic construct
  source 1..10: note = Ranibizumab Heavy Chain CDR1
GYDFTHYGMN

SEQ ID NO: 21 - protein, 14 aa, synthetic construct
  source 1..14: note = Ranibizumab Heavy Chain CDR3
YPYYYGTSHWYFDV

SEQ ID NO: 22 - protein, 18 aa, synthetic construct
  source 1..18: note = Albumin signal peptide
MKWVTFISLLFLFSSAYS

SEQ ID NO: 23 - protein, 18 aa, synthetic construct
  source 1..18: note = Chymotrypsinogen signal peptide
MAFLWLLSCWALLGTTFG

SEQ ID NO: 24 - protein, 20 aa, synthetic construct
  source 1..20: note = Interleukin-2 signal peptide
MYRMQLLSCIALILALVTNS

SEQ ID NO: 25 - protein, 15 aa, synthetic construct
  source 1..15: note = Trypsinogen-2 signal peptide
MNLLLILTFVAAAVA

SEQ ID NO: 26 - protein, 19 aa, synthetic construct
  source 1..19: note = F2A site
LLNFDLLKLAGDVESNPGP

SEQ ID NO: 27 - protein, 21 aa, synthetic construct
  source 1..21: note = T2A site
GSGEGRGSLLTCGDVEENPGP

SEQ ID NO: 28 - protein, 22 aa, synthetic construct
  source 1..22: note = P2A site
GSGATNFSLLKQAGDVEENPGP

SEQ ID NO: 29 - protein, 23 aa, synthetic construct
  source 1..23: note = E2A site
GSGQCTNYALLKLAGDVESNPGP

SEQ ID NO: 30 - protein, 25 aa, synthetic construct
  source 1..25: note = F2A site
GSGVKQTLNFDLLKLAGDVESNPGP

SEQ ID NO: 31 - protein, 4 aa, synthetic construct
  source 1..4: note = Furin linker
RKRR

SEQ ID NO: 32 - protein, 4 aa, synthetic construct
  source 1..4: note = Furin linker
RRRR

SEQ ID NO: 33 - protein, 4 aa, synthetic construct
  source 1..4: note = Furin linker
RRKR

SEQ ID NO: 34 - protein, 4 aa, synthetic construct
  source 1..4: note = Furin linker
RKKR

SEQ ID NO: 35-37 - skipped (3 entries)

SEQ ID NO: 38 - protein, 215 aa, synthetic construct
  source 1..215: note = Ranibizumab Fab amino acid sequence (Light chain)
MDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 236 aa, synthetic construct
  source 1..236: note = Ranibizumab Fab amino acid sequence (Heavy chain)
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLRKRR

SEQ ID NO: 40 - protein, 232 aa, synthetic construct
  source 1..232: note = Ranibizumab Fab amino acid sequence (Heavy chain)
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 41 - protein, 736 aa, synthetic construct
  source 1..736: note = AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 735 aa, synthetic construct [sequence omitted]

SEQ ID NO: 43 - protein, 736 aa, synthetic construct
  source 1..736: note = AAV3-3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 734 aa, synthetic construct
  source 1..734: note = AAV4-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 45 - protein, 724 aa, synthetic construct
  source 1..724: note = AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, synthetic construct
  source 1..736: note = AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 737 aa, synthetic construct
  source 1..737: note = AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 48 - protein, 738 aa, synthetic construct
  source 1..738: note = AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 736 aa, synthetic construct
  source 1..736: note = hu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 50 - protein, 736 aa, synthetic construct
  source 1..736: note = hu32
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, synthetic construct
  source 1..736: note = AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52-54 - skipped (3 entries)

SEQ ID NO: 55 - protein, 20 aa, synthetic construct
  source 1..20: note = Mutant Interleukin-2 signal peptide
MYRMQLLLLIALSLALVTNS

SEQ ID NO: 56 - DNA, 3951 bp, synthetic construct
  source 1..3951: mol_type = other DNA; note = ITR.CB7.CI.aVEGRv3.rBG.ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccgtatgcagctgctgctgctgatcgcactgtcactggcactggttaccaactcagaagttcagctggttgaatcaggcggcggcctggttcagcccggcggctcactgcgtctgtcatgtgcagcatcaggctacgatttcacccactacggcatgaactgggttcgtcaggcacccggcaaaggcctggaatgggttggctggatcaacacctacaccggcgaacccacctacgcagcagatttcaaacgtcgtttcaccttctcactggatacctcaaaatcaaccgcatacctgcagatgaactcactgcgtgcagaagataccgcagtttactactgtgcaaaatacccctactactacggcacctcacactggtacttcgatgtttggggccagggcaccctggttaccgtttcatcagcatcaaccaaaggcccctcagttttccccctggcaccctcatcaaaatcaacctcaggcggcaccgcagcactgggctgtctggttaaagattacttccccgaacccgttaccgtttcatggaactcaggcgcactgacctcaggcgttcacaccttccccgcagttctgcagtcatcaggcctgtactcactgtcatcagttgttaccgttccctcatcatcactgggcacccagacctacatctgtaacgttaaccacaaaccctcaaacaccaaagttgataaaaaagttgaacccaaatcatgtgataaaacccacctgcgtaaacgtcgtgcacccgttaaacagaccctgaacttcgatctgctgaaactggcaggcgatgttgaatcaaaccccggccccatgtaccgtatgcagctgctgctgctgatcgcactgtcactggcactggttaccaactcagatatccagctgacccagtcaccctcatcactgtcagcatcagttggcgatcgtgttaccatcacctgttcagcatcacaggatatctcaaactacctgaactggtaccagcagaaacccggcaaagcacccaaagttctgatctacttcacctcatcactgcactcaggcgttccctcacgtttctcaggctcaggctcaggcaccgatttcaccctgaccatctcatcactgcagcccgaagatttcgcaacctactactgtcagcagtactcaaccgttccctggaccttcggccagggcaccaaagttgaaatcaaacgtaccgttgcagcaccctcagttttcatcttccccccctcagatgaacagctgaaatcaggcaccgcatcagttgtttgtctgctgaacaacttctacccccgtgaagcaaaagttcagtggaaagttgataacgcactgcagtcaggcaactcacaggaatcagttaccgaacaggattcaaaagattcaacctactcactgtcatcaaccctgaccctgtcaaaagcagattacgaaaaacacaaagtttacgcatgtgaagttacccaccagggcctgtcatcacccgttaccaaatcattcaaccgtggcgaatgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 57 - protein, 234 aa, synthetic construct
  source 1..234: note = Ranibizumab Fab Amino Acid Sequence (Signal Peptide and Light chain)
MYRMQLLLLIALSLALVTNSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 250 aa, synthetic construct
  source 1..250: note = Ranibizumab Fab Amino Acid Sequence (Signal Peptide and Heavy chain)
MYRMQLLLLIALSLALVTNSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 59 - protein, 7 aa, synthetic construct
  source 1..7: note = Optionally amino acid insertion
LGETTRP

SEQ ID NO: 60 - protein, 9 aa, synthetic construct
  source 1..9: note = Optionally amino acid insertion
LALGETTRP

SEQ ID NO: 61 - protein, 214 aa, synthetic construct
  source 1..214: note = Hyperglycosylated Ranibizumab Fab Amino Acid Sequence (Light chain)
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSNESVTEQDSKDSTYSLSSTLTLSKADYEKHKVYACNVTHQGLSSPVTKSFNRGEC

SEQ ID NO: 62 - protein, 231 aa, synthetic construct
  source 1..231: note = Hyperglycosylated Ranibizumab Fab Amino Acid Sequence (Heavy chain)
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTNVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 63 - protein, 10 aa, synthetic construct
  source 1..10: note = Bevacizumab and Ranibizumab Light Chain CDR3
QQYSTVPWTF

(19) United States
(12) Patent Application Publication
(10) Pub. No.: US 2026/0034250 A1
(43) Pub. Date: Feb. 5, 2026

(54) TREATMENT OF OCULAR DISEASES WITH RECOMBINANT VIRAL VECTORS ENCODING ANTI-VEGF FAB

(71) Applicants: REGENXBIO Inc., Rockville, MD (US); ABBVIE GLOBAL ENTERPRISES LTD., Hamilton (BM)

(72) Inventors: Sherri VAN EVEREN, Menlo Park, CA (US); Saba June KADLEC, Wauwatosa, WI (US); Stephen Joseph PAKOLA, Irvington, NY (US); Anna Meghedi ABOLIAN, San Francisco, CA (US); Francisco LOPEZ, Ladera Ranch, CA (US)

(21) Appl. No.: 19/116,634

(22) PCT Filed: Sep. 29, 2023
(86) PCT No.: PCT/US2023/075520

§ 371 Date: Mar. 28, 2025

Related U.S. Application Data
(60) Provisional application No. 63/412,087, filed on Sep. 30, 2022.
(60) Provisional application No. 63/421,741, filed on Nov. 2, 2022.

(51) Int. Cl. A61K 48/00; A61K 31/573; A61K 31/58; A61K 39/395; A61P 27/02

(52) U.S. Cl. CPC A61K 48/0075; A61K 31/573; A61K 31/58; A61K 39/3955; A61K 48/0066; A61P 27/02

(57) ABSTRACT

Provided herein are methods of treating neovascular age-related macular degeneration (nAMD) and diabetic retinopathy (DR) in a subject in need thereof comprising administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to an eye of the subject; and the steroid treatment comprises administering a therapeutically effective amount of a steroid to the eye of the subject.

### CROSS-REFERENCE TO RELATED APPLICATIONS

This application claims priority to U.S. Provisional Application No. 63/412,087 filed Sep. 30, 2022 and U.S. Provisional Application No. 63/421,741 filed Nov. 2, 2022, the content of each of which is incorporated by reference in its entirety herein, and to which priority is claimed.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “12656-177-228_SEQ_LISTING.xml”, was created on Sep. 20, 2023, and is 80,588 bytes in size.

### 1. FIELD

Provided herein are methods of treating neovascular age-related macular degeneration (nAMD) and diabetic retinopathy (DR) in a subject in need thereof comprising administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to an eye of the subject; and the steroid treatment comprises administering a therapeutically effective amount of a steroid to the eye of the subject.

### 2. BACKGROUND

The human eye is a highly intricate and highly developed sensory organ, which is prone to a host of diseases and disorders. About 285 million people in the world are visually impaired, of whom 39 million are blind and 246 million have moderate to severe visual impairment (World Health Organization, 2012, “Global Data On Visual Impairments 2010,” Geneva: World Health Organization). Some of the leading causes of blindness are cataract (47%), glaucoma (12%), age-related macular degeneration (AMD) (9%), and diabetic retinopathy (5%) (World Health Organization, 2007, “Global Initiative For The Elimination Of Avoidable Blindness: Action Plan 2006-2011,” Geneva: World Health Organization).

An extensive number of ocular diseases and diseases with pathological manifestations in the eye can be traced to genetic alterations or protein dysregulations (Stone et al., 2017, Ophthalmology 124(9): 1314-1331). Recent advances in genomics and proteomics have made a huge impact in our understanding of disease mechanisms and/or genetic basis underlying such ocular diseases or manifestations. Gene therapy has been employed in treating certain eye diseases (see, e.g. International Patent Application No. PCT/US2017/027650 (International Publication No. WO 2017/181021 A1)).

There is a significant unmet medical need for therapies that specifically address the underlying genetic anomalies to treat ocular pathologies.

### 3. SUMMARY

Provided herein is a method of treating neovascular age-related macular degeneration (nAMD) in a subject in need thereof, wherein the method comprises administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to an eye of the subject; and the steroid treatment comprises administering a therapeutically effective amount of a steroid to the eye of the subject.

Also provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to the suprachoroidal space of an eye of the subject; and the steroid treatment comprises administering a therapeutically effective amount of a steroid to the eye of the subject.

Also provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to an eye of the subject; and the steroid treatment comprises administering a therapeutically effective amount of triamcinolone acetonide to the eye of the subject. Also provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to an eye of the subject; and the steroid treatment comprises administering a therapeutically effective amount of difluprednate to the eye of the subject.

In certain embodiments of the methods provided herein, the method is a method of treating neovascular age-related macular degeneration (nAMD). In certain embodiments, the method is a method of treating diabetic retinopathy (DR).

In certain embodiments of the methods provided herein, the recombinant viral vector is administered to the suprachoroidal space of the eye of the subject. In certain embodiments, the recombinant viral vector is administered by injection into the suprachoroidal space of the eye using a suprachoroidal drug delivery device. In certain embodiments, the suprachoroidal drug delivery device is a microinjector. In other embodiments, the recombinant viral vector is administered to the subretinal space of the eye of the subject. In certain embodiments, wherein the method does not comprise performing a vitrectomy on the eye of the subject. In certain embodiments, the subretinal administration comprises performing a vitrectomy on the eye of said subject. In certain embodiments, the vitrectomy is a partial vitrectomy. In other embodiments, the recombinant viral vector is administered to the subretinal space via the suprachoroidal space of the eye of the subject. In certain embodiments, the recombinant viral vector is administered with a subretinal drug delivery device comprising a catheter that can be inserted and tunneled through the suprachoroidal space toward the posterior pole, where a small needle injects into the subretinal space. In certain embodiments, the anti-hVEGF treatment comprises inserting and tunneling the catheter of the subretinal drug delivery device through the suprachoroidal space to administer the recombinant viral vector.

In certain embodiments, the steroid treatment comprises administering a therapeutically effective amount of a corticosteroid. In certain embodiments of the methods provided herein, the corticosteroid is triamcinolone acetonide. In other embodiments, the corticosteroid is difluprednate. In certain embodiments of the methods provided herein, the steroid is triamcinolone acetonide. In other embodiments, the steroid is difluprednate.

In certain embodiments of the methods provided herein the anti-hVEGF treatment comprises administering a recombinant viral vector comprising a nucleotide sequence encoding an anti-hVEGF antigen-binding fragment to the suprachoroidal space of an eye of the subject; and the steroid treatment comprises administering triamcinolone acetonide to the eye of the subject. In certain embodiments, wherein the triamcinolone acetonide is administered after administering the recombinant viral vector. In other embodiments, the triamcinolone acetonide is administered before administering the recombinant viral vector. In certain embodiments, the triamcinolone acetonide is administered to the eye of the subject within about 24 hours, about 20 hours, about 16 hours, about 12 hours, about 8 hours, about 4 hours, about 3 hours, about 2 hours, about 1 hour, about 50 minutes, about 40 minutes, about 30 minutes, about 20 minutes, about 10 minutes, about 9 minutes, about 8 minutes, about 7 minutes, about 6 minutes, about 5 minutes, about 4 minutes, about 3 minutes, about 2 minutes, or about 1 minute of administering the recombinant viral vector. In certain embodiments, the triamcinolone acetonide is administered by injection into the eye of the subject. In certain embodiments, the triamcinolone acetonide is administered by a single injection into the eye of the subject. In certain embodiments, the steroid treatment consists of a single injection of triamcinolone acetonide into the eye of the subject. In certain embodiments, the triamcinolone acetonide is administered in a different quadrant of the eye than is the recombinant viral vector. In certain embodiments, the triamcinolone acetonide is administered to the subtenon of the eye. In certain embodiments, the triamcinolone acetonide is administered at a dose of about 40 mg. In certain embodiments, the triamcinolone acetonide is administered in a volume of about 1 mL.

In certain embodiments of the methods provided herein, the anti-hVEGF treatment comprises administering a recombinant viral vector comprising a nucleotide sequence encoding an anti-hVEGF antigen-binding fragment to the suprachoroidal space of an eye of the subject; and the steroid treatment comprises administering difluprednate to the eye of the subject. In certain embodiments, the difluprednate is administered daily to the eye of the subject. In certain embodiments, the steroid treatment comprises administering difluprednate four times daily. In certain embodiments, the difluprednate is administered four times daily for at least one week, at least two weeks, at least three weeks, or at least four weeks. In certain embodiments, the difluprednate is administered four times daily for about four weeks. In certain embodiments, the steroid treatment comprises administering difluprednate three times daily. In certain embodiments, the difluprednate is administered three times daily for at least one day, at least two days, at least three days, at least four days, at least five days, at least six days, or at least one week. In certain embodiments, the difluprednate is administered three times daily for about one week. In certain embodiments, the steroid treatment comprises administering difluprednate two times daily. In certain embodiments, the difluprednate is administered two times daily for at least one day, at least two days, at least three days, at least four days, at least five days, at least six days, or at least one week. In certain embodiments, the difluprednate is administered two times daily for about one week. In certain embodiments, the steroid treatment comprises administering difluprednate one time daily. In certain embodiments, the difluprednate is administered one time daily for at least one day, at least two days, at least three days, at least four days, at least five days, at least six days, or at least one week. In certain embodiments, the difluprednate is administered one time daily for about one week. In certain embodiments, the difluprednate is administered to the eye of the subject for a period of at least one week, at least two weeks, at least three weeks, at least four weeks, at least five weeks, at least six weeks, or at least seven weeks. In certain embodiments, the difluprednate is administered to the eye of the subject for a period of about seven weeks. In certain embodiments, the steroid treatment comprises administering difluprednate once on the first day of the steroid treatment, followed by four times daily for about four weeks, followed by three times daily for about one week, followed by two times daily for about one week, followed by one time daily for about one week. In certain embodiments, the steroid treatment consists of administering difluprednate once on the first day of the steroid treatment, followed by four times daily for about four weeks, followed by three times daily for about one week, followed by two times daily for about one week, followed by one time daily for about one week. In certain embodiments, the difluprednate is administered in the form of a ophthalmic emulsion. In certain embodiments, the ophthalmic emulsion comprises 0.5 mg/mL (0.05%) difluprednate. In certain embodiments, each administration of difluprednate comprises instilling one drop of the ophthalmic emulsion in the eye of the subject. In certain embodiments, each administration of difluprednate consists of instilling one drop of the ophthalmic emulsion in the eye of the subject. In certain embodiments, difluprednate is first administered to the eye of the subject within about seven days, about six days, about five days, about four days, about three days, about two days, or about one day of administering the recombinant viral vector. In certain embodiments, difluprednate is first administered to the eye of the subject on the same day as the recombinant viral vector is administered. In certain embodiments, the first administration of difluprednate occurs after the first administration of the recombinant viral vector.

In certain embodiments of the methods provided herein, the anti-hVEGF antigen-binding fragment is a Fab, F(ab′)₂, or single chain variable fragment (scFv).

In certain embodiments, the anti-hVEGF antigen-binding fragment comprises a heavy chain comprising the amino acid sequence of SEQ ID NO:2 or SEQ ID NO. 4, and a light chain comprising the amino acid sequence of SEQ ID NO:1, or SEQ ID NO:3. In certain embodiments, the anti-hVEGF antigen-binding fragment comprises (a) a heavy chain comprising heavy chain CDRs 1-3 of the amino acid sequence of SEQ ID NO: 2, and (b) a light chain comprising light chain CDRs 1-3 of the amino acid sequence of SEQ ID NO: 1. In certain embodiments, the anti-hVEGF antigen-binding fragment comprises (a) a heavy chain comprising heavy chain CDRs 1-3 of the amino acid sequence of SEQ ID NO: 4, and (b) a light chain comprising light chain CDRs 1-3 of the amino acid sequence of SEQ ID NO: 3. In certain embodiments, the anti-hVEGF antigen-binding fragment comprises light chain CDRs 1-3 of SEQ ID NOs: 14-16 or SEQ ID NOs: 14, 15 and 63 and heavy chain CDRs 1-3 of SEQ ID NOs: 17-19 or SEQ ID NOs: 20, 18, and 21. In certain embodiments, administration of the recombinant viral vector delivers a therapeutically effective amount of the anti-hVEGF antigen-binding fragment to the retina of said human subject. In certain embodiments, the therapeutically effective amount of the anti-hVEGF antigen-binding fragment is produced by retinal cells of the subject. In certain embodiments, the recombinant viral vector is an rAAV vector. In certain embodiments, the recombinant viral vector is an rAAV8 vector.

In certain embodiments, the recombinant viral vector comprises an expression cassette encoding an anti-hVEGF antigen-binding fragment, wherein the expression cassette is flanked by AAV2 inverted terminal repeats (ITRs), and wherein the expression cassette comprises:
a CB7 promotor consisting of a chicken 3-actin promoter and a CMV enhancer;
a chicken β-actin intron;
a nucleotide sequence encoding:
   an IL-2 signal peptide;
   a heavy chain of the anti-hVEGF antigen-binding fragment comprising the amino acid sequence of SEQ ID NO: 2;
   a self-cleaving furin (F)/F2A linker;
   a second IL-2 signal peptide; and
   a light chain of the anti-hVEGF antigen-binding fragment comprising the amino acid sequence of SEQ ID NO: 1; and
a rabbit β-globin poly A signal.

In certain embodiments, the recombinant viral vector comprises the nucleotide sequence of SEQ ID NO: 56.

In certain embodiments, the recombinant viral vector is administered at a dose about 2.5×10¹¹ genome copies per eye. In certain embodiments, the recombinant viral vector is administered at a dose about 5.0×10¹¹ genome copies per eye. In certain embodiments, the recombinant viral vector is administered at a dose about 1.0×10¹² genome copies per eye.

Also provided herein is a kit for use in a method of treating neovascular age-related macular degeneration (nAMD) provided herein comprising a recombinant viral vector comprising a nucleotide sequence encoding an anti-hVEGF antigen-binding fragment; and a steroid.

Also provided herein is a kit for use in a method of treating diabetic retinopathy (DR) provided herein comprising a recombinant viral vector comprising a nucleotide sequence encoding an anti-hVEGF antigen-binding fragment; and a steroid.

In certain embodiments of the methods provided herein, the recombinant viral vector is formulated to be suitable for administration to the suprachoroidal space of the eye of the subject. In certain embodiments, the recombinant viral vector is formulated to be suitable for administration to the subretinal space of the eye of the subject. In certain embodiments, the steroid is triamcinolone acetonide. In certain embodiments, the steroid is difluprednate.

Also provided herein is use of a recombinant viral vector comprising a nucleotide sequence encoding an anti-hVEGF antigen-binding fragment and a steroid in the manufacture of a medicament for the treatment of neovascular age-related macular degeneration (nAMD) as provided herein. Also provided herein is use of a recombinant viral vector comprising a nucleotide sequence encoding an anti-hVEGF antigen-binding fragment and a steroid in the manufacture of a medicament for the treatment of diabetic retinopathy (DR) as provided herein.

In certain embodiments of the methods provided herein, the recombinant viral vector is formulated to be suitable for administration to the suprachoroidal space of the eye of the subject. In certain embodiments, the recombinant viral vector is formulated to be suitable for administration to the subretinal space of the eye of the subject. In certain embodiments, the steroid is triamcinolone acetonide. In certain embodiments, the steroid is difluprednate.

### 3.1 Illustrative Embodiments

1. A method of treating neovascular age-related macular degeneration (nAMD) in a subject in need thereof, wherein the method comprises administering an anti-hVEGF treatment and a steroid treatment; wherein
a. the anti-hVEGF treatment comprises administering a therapeutically effective amount of a recombinant viral vector comprising a nucleotide sequence encoding an anti-hVEGF antigen-binding fragment to an eye of the subject; and
b. the steroid treatment comprises administering a therapeutically effective amount of a steroid to the eye of the subject.

2. A method of treating neovascular age-related macular degeneration (nAMD) or diabetic retinopathy (DR) in a subject in need thereof, wherein the method comprises administering an anti-hVEGF treatment and a steroid treatment; wherein
a. the anti-hVEGF treatment comprises administering a therapeutically effective amount of a recombinant viral vector comprising a nucleotide sequence encoding an anti-hVEGF antigen-binding fragment to the suprachoroidal space of an eye of the subject; and
b. the steroid treatment comprises administering a therapeutically effective amount of a steroid to the eye of the subject.

3. A method of treating neovascular age-related macular degeneration (nAMD) or diabetic retinopathy (DR) in a subject in need thereof, wherein the method comprises administering an anti-hVEGF treatment and a steroid treatment; wherein
a. the anti-hVEGF treatment comprises administering a therapeutically effective amount of a recombinant viral vector comprising a nucleotide sequence encoding an anti-hVEGF antigen-binding fragment to an eye of the subject; and
b. the steroid treatment comprises administering a therapeutically effective amount of triamcinolone acetonide to the eye of the subject.

4. A method of treating neovascular age-related macular degeneration (nAMD) or diabetic retinopathy (DR) in a subject in need thereof, wherein the method comprises administering an anti-hVEGF treatment and a steroid treatment; wherein
a. the anti-hVEGF treatment comprises administering a therapeutically effective amount of a recombinant viral vector comprising a nucleotide sequence encoding an anti-hVEGF antigen-binding fragment to an eye of the subject; and
b. the steroid treatment comprises administering a therapeutically effective amount of difluprednate to the eye of the subject.

5. The method of any one of embodiments 2-4, wherein the method is a method of treating neovascular age-related macular degeneration (nAMD).

6. The method of any one of embodiments 2-4, wherein the method is a method of treating diabetic retinopathy (DR).

7. The method of any one of embodiments 1 or 3-6, wherein the recombinant viral vector is administered to the suprachoroidal space of the eye of the subject.

8. The method of any one of embodiments 1-7, wherein the recombinant viral vector is administered by injection into the suprachoroidal space of the eye using a suprachoroidal drug delivery device.

9. The method of embodiment 8, wherein the suprachoroidal drug delivery device is a microinjector.

10. The method of any one of embodiments 1 or 3-6, wherein the recombinant viral vector is administered to the subretinal space of the eye of the subject.

11. The method of embodiment 10, wherein the method does not comprise performing a vitrectomy on the eye of the subject.

12. The method of embodiment 10, wherein the subretinal administration comprises performing a vitrectomy on the eye of the subject.

13. The method of embodiment 12, wherein the vitrectomy is a partial vitrectomy.

14. The method of embodiment 10 or embodiment 11, wherein the recombinant viral vector is administered to the subretinal space via the suprachoroidal space of the eye of the subject.

15. The method of embodiment 14, wherein the recombinant viral vector is administered with a subretinal drug delivery device comprising a catheter that can be inserted and tunneled through the suprachoroidal space toward the posterior pole, where a small needle injects into the subretinal space.

16. The method of embodiment 15, wherein the anti-hVEGF treatment comprises inserting and tunneling the catheter of the subretinal drug delivery device through the suprachoroidal space to administer the recombinant viral vector.

17. The method of any one of embodiments 1-16, wherein the steroid treatment ameliorates or prevents intraocular inflammation.

18. The method of any one of embodiments 1-17, wherein the steroid treatment ameliorates or prevents intraocular inflammation associated with the dose of the recombinant viral vector, the number of suprachoroidal injections, and/or the location of suprachoroidal injections.

19. The method of any one of embodiments 1, 2, and 4-18, wherein the steroid is topically administered.

20. The method of any one of embodiments 1, 2, and 5-19, wherein the steroid is a corticosteroid.

21. The method of any one of embodiments 1, 2, and 5-20, wherein the steroid is triamcinolone acetonide.

22. The method of any one of embodiments 1, 2, and 5-20, wherein the steroid is difluprednate.

23. The method of any one of embodiments 1-3, 5-9, and 17-21, wherein
a. the anti-hVEGF treatment comprises administering a recombinant viral vector comprising a nucleotide sequence encoding an anti-hVEGF antigen-binding fragment to the suprachoroidal space of an eye of the subject; and
b. the steroid treatment comprises administering triamcinolone acetonide to the eye of the subject.

24. The method of any one of embodiments 3, 5-21, and 23, wherein the triamcinolone acetonide is administered after administering the recombinant viral vector.

25. The method of any one of embodiments 3, 5-21, and 23, wherein the triamcinolone acetonide is administered before administering the recombinant viral vector.

26. The method of any one of embodiments 3, 5-21, and 23-25, wherein the triamcinolone acetonide is administered to the eye of the subject within about 24 hours, about 20 hours, about 16 hours, about 12 hours, about 8 hours, about 4 hours, about 3 hours, about 2 hours, about 1 hour, about 50 minutes, about 40 minutes, about 30 minutes, about 20 minutes, about 10 minutes, about 9 minutes, about 8 minutes, about 7 minutes, about 6 minutes, about 5 minutes, about 4 minutes, about 3 minutes, about 2 minutes, or about 1 minute of administering the recombinant viral vector.

27. The method of any one of embodiments 3, 5-18, 20, 21, and 23-26, wherein the triamcinolone acetonide is administered by injection into the eye of the subject.

28. The method of embodiment 27, wherein the triamcinolone acetonide is administered by a single injection into the eye of the subject.

29. The method of any one of embodiments 3, 5-18, 20, 21, and 23-28, wherein the steroid treatment consists of a single injection of triamcinolone acetonide into the eye of the subject.

30. The method of any one of embodiments 3, 5-18, 20, 21, and 23-29, wherein the triamcinolone acetonide is administered in a different quadrant of the eye than is the recombinant viral vector.

31. The method of any one of embodiments 3, 5-18, 20, 21, and 23-30, wherein the triamcinolone acetonide is administered to the subtenon of the eye.

32. The method of any one of embodiments 3, 5-18, 20, 21, and 23-31, wherein the triamcinolone acetonide is administered at a dose of about 40 mg.

33. The method of any one of embodiments 3, 5-18, 20, 21, and 23-32, wherein the triamcinolone acetonide is administered in a volume of about 1 mL.

34. The method of any one of embodiments 1, 2, 4-9, 17-20, and 22, wherein
a. the anti-hVEGF treatment comprises administering a recombinant viral vector comprising a nucleotide sequence encoding an anti-hVEGF antigen-binding fragment to the suprachoroidal space of an eye of the subject; and
b. the steroid treatment comprises administering difluprednate to the eye of the subject.

35. The method of any one of embodiments 4-20, 22, and 34, wherein the difluprednate is administered daily to the eye of the subject.

36. The method of embodiment 35, wherein the steroid treatment comprises administering difluprednate four times daily.

37. The method of embodiment 36, wherein the difluprednate is administered four times daily for at least one week, at least two weeks, at least three weeks, or at least four weeks.

38. The method of embodiment 37, wherein the difluprednate is administered four times daily for about four weeks.

39. The method of any one of embodiments 35-38, wherein the steroid treatment comprises administering difluprednate three times daily.

40. The method of embodiment 39, wherein the difluprednate is administered three times daily for at least one day, at least two days, at least three days, at least four days, at least five days, at least six days, or at least one week.

41. The method of embodiment 40, wherein the difluprednate is administered three times daily for about one week.

42. The method of any one of embodiments 35-41, wherein the steroid treatment comprises administering difluprednate two times daily.

43. The method of embodiment 42, wherein the difluprednate is administered two times daily for at least one day, at least two days, at least three days, at least four days, at least five days, at least six days, or at least one week.

44. The method of embodiment 44, wherein the difluprednate is administered two times daily for about one week.

45. The method of any one of embodiments 35-44, wherein the steroid treatment comprises administering difluprednate one time daily.

46. The method of embodiment 45, wherein the difluprednate is administered one time daily for at least one day, at least two days, at least three days, at least four days, at least five days, at least six days, or at least one week.

47. The method of embodiment 46, wherein the difluprednate is administered one time daily for about one week.

48. The method of any one of embodiments 4-20, 22, and 34-47, wherein the difluprednate is administered to the eye of the subject for a period of at least one week, at least two weeks, at least three weeks, at least four weeks, at least five weeks, at least six weeks, or at least seven weeks.

49. The method of embodiment 48, wherein the difluprednate is administered to the eye of the subject for a period of about seven weeks.

50. The method of embodiment 49, wherein the steroid treatment comprises administering difluprednate once on the first day of the steroid treatment, followed by four times daily for about four weeks, followed by three times daily for about one week, followed by two times daily for about one week, followed by one time daily for about one week.

51. The method of embodiment 49, wherein the steroid treatment consists of administering difluprednate once on the first day of the steroid treatment, followed by four times daily for about four weeks, followed by three times daily for about one week, followed by two times daily for about one week, followed by one time daily for about one week.

52. The method of any one of embodiments 4-20, 22, and 34-51, wherein the difluprednate is administered in the form of a ophthalmic emulsion.

53. The method of embodiment 52, wherein the ophthalmic emulsion comprises 0.5 mg/mL (0.05%) difluprednate.

54. The method of embodiment 52 or embodiment 53, wherein each administration of difluprednate comprises instilling one drop of the ophthalmic emulsion in the eye of the subject.

55. The method of embodiment 52 or embodiment 53, wherein each administration of difluprednate consists of instilling one drop of the ophthalmic emulsion in the eye of the subject.

56. The method of any one of embodiments 4-20, 22, and 34-55, wherein difluprednate is first administered to the eye of the subject within about seven days, about six days, about five days, about four days, about three days, about two days, or about one day of administering the recombinant viral vector.

57. The method of embodiment 56, wherein difluprednate is first administered to the eye of the subject on the same day as the recombinant viral vector is administered.

58. The method of embodiment 56 or embodiment 57, wherein the first administration of difluprednate occurs after the first administration of the recombinant viral vector.

59. The method of any one of embodiments 1-58, wherein the anti-hVEGF antigen-binding fragment is a Fab, F(ab′)₂, or single chain variable fragment (scFv).

60. The method of any one of embodiments 1-59, wherein the anti-hVEGF antigen-binding fragment comprises a heavy chain comprising the amino acid sequence of SEQ ID NO:2 or SEQ ID NO:4, and a light chain comprising the amino acid sequence of SEQ ID NO:1 or SEQ ID NO:3.

61. The method of any one of embodiments 1-60, wherein the anti-hVEGF antigen-binding fragment comprises (a) a heavy chain comprising heavy chain CDRs 1-3 of the amino acid sequence of SEQ ID NO: 2, and (b) a light chain comprising light chain CDRs 1-3 of the amino acid sequence of SEQ ID NO: 1.

62. The method of any one of embodiments 1-60, wherein the anti-hVEGF antigen-binding fragment comprises (a) a heavy chain comprising heavy chain CDRs 1-3 of the amino acid sequence of SEQ ID NO: 4, and (b) a light chain comprising light chain CDRs 1-3 of the amino acid sequence of SEQ ID NO: 3.

63. The method of any one of embodiments 1-62, wherein the anti-hVEGF antigen-binding fragment comprises light chain CDRs 1-3 of SEQ ID NOs: 14-16 or SEQ ID NOs: 14, 15 and 63 and heavy chain CDRs 1-3 of SEQ ID NOs: 17-19 or SEQ ID NOs: 20, 18, and 21.

64. The method of any one of embodiments 1-63, wherein administration of the recombinant viral vector delivers a therapeutically effective amount of the anti-hVEGF antigen-binding fragment to the retina of said human subject.

65. The method of embodiment 65, wherein the therapeutically effective amount of the anti-hVEGF antigen-binding fragment is produced by retinal cells of the subject.

66. The method of any one of embodiments 1-65, wherein the recombinant viral vector is an rAAV vector.

67. The method of any one of embodiments 1-66, wherein the recombinant viral vector is an rAAV8 vector.

68. The method of any one of embodiments 1-67, wherein the recombinant viral vector comprises an expression cassette encoding an anti-hVEGF antigen-binding fragment, wherein the expression cassette is flanked by AAV2 inverted terminal repeats (ITRs), and wherein the expression cassette comprises:
a. a CB7 promotor consisting of a chicken β-actin promoter and a CMV enhancer;
b. a chicken β-actin intron;
c. a nucleotide sequence encoding:
   i. an IL-2 signal peptide;
   ii. a heavy chain of the anti-hVEGF antigen-binding fragment comprising the amino acid sequence of SEQ ID NO: 2;
   iii. a self-cleaving furin (F)/F2A linker;
   iv. a second IL-2 signal peptide; and
   v. a light chain of the anti-hVEGF antigen-binding fragment comprising the amino acid sequence of SEQ ID NO: 1; and
d. a rabbit β-globin poly A signal.

69. The method of any one of embodiments 1-68, wherein the recombinant viral vector comprises the nucleotide sequence of SEQ ID NO: 56.

70. The method of any one of embodiments 1-69, wherein the recombinant viral vector is administered at a dose about 2.5×10¹¹ genome copies per eye.

71. The method of any one of embodiments 1-69, wherein the recombinant viral vector is administered at a dose about 5.0×10¹¹ genome copies per eye.

72. The method of any one of embodiments 1-69, wherein the recombinant viral vector is administered at a dose about 1.0×10¹² genome copies per eye.

73. The method of any one of embodiments 1-72, wherein the recombinant viral vector is administered by double suprachoroidal injections.

74. The method of any one of embodiments 1-72, wherein the recombinant viral vector is administered by a single suprachoroidal injection.

75. A kit for use in a method of treating neovascular age-related macular degeneration (nAMD) according to any one of embodiments 1-5 and 7-74 comprising
a. a recombinant viral vector comprising a nucleotide sequence encoding an anti-hVEGF antigen-binding fragment; and
b. a steroid.

76. A kit for use in a method of treating diabetic retinopathy (DR) according to any one of embodiments 2-4 and 6-74 comprising
a. a recombinant viral vector comprising a nucleotide sequence encoding an anti-hVEGF antigen-binding fragment; and
b. a steroid.

77. The kit of embodiment 75 or embodiment 76, wherein the recombinant viral vector is formulated to be suitable for administration to the suprachoroidal space of the eye of the subject.

78. The kit of embodiment 75 or embodiment 76, wherein the recombinant viral vector is formulated to be suitable for administration to the subretinal space of the eye of the subject.

79. The kit of any one of embodiments 75-78, wherein the steroid is triamcinolone acetonide.

80. The kit of any one of embodiments 75-78, wherein the steroid is difluprednate.

81. Use of a recombinant viral vector comprising a nucleotide sequence encoding an anti-hVEGF antigen-binding fragment and a steroid in the manufacture of a medicament for the treatment of neovascular age-related macular degeneration (nAMD) according to any one embodiments 1-5 and 7-74.

82. Use of a recombinant viral vector comprising a nucleotide sequence encoding an anti-hVEGF antigen-binding fragment and a steroid in the manufacture of a medicament for the treatment of diabetic retinopathy (DR) according to any one of embodiments 2-4 and 6-74.

83. The use of embodiment 81 or embodiment 82, wherein the recombinant viral vector is formulated to be suitable for administration to the suprachoroidal space of the eye of the subject.

84. The use of embodiment 81 or embodiment 82, wherein the recombinant viral vector is formulated to be suitable for administration to the subretinal space of the eye of the subject.

85. The use of any one of embodiments 81-84, wherein the steroid is triamcinolone acetonide.

86. The use of any one of embodiments 81-84, wherein the steroid is difluprednate.

### 4. BRIEF DESCRIPTION OF THE DRAWINGS

FIG. 1. The amino acid sequence of ranibizumab (top; SEQ ID NOs: 2 and 1) showing 5 different residues in bevacizumab Fab (below; SEQ ID NOs: 4 and 3). The starts of the variable and constant heavy chains (V_{H} and C_{H}) and light chains (V_{L} and V_{C}) are indicated by arrows (→), and the CDRs are underscored. Non-consensus glycosylation sites (“Gsite”) tyrosine-O-sulfation sites (“Ysite”) are indicated.

FIG. 2. Glycans that can be attached to HuGlyFabVEGFi. (Adapted from Bondt et al., 2014, Mol & Cell Proteomics 13.1: 3029-3039).

FIG. 3. The amino acid sequence of hyperglycosylated variants of ranibizumab (top; SEQ ID NOs: 62 and 61) and bevacizumab Fab (below; SEQ ID NOs: 4 and 3). The starts of the variable and constant heavy chains (V_{H} and C_{H}) and light chains (V_{L} and V_{C}) are indicated by arrows (→), and the CDRs are underscored. Non-consensus glycosylation sites (“Gsite”) and tyrosine-O-sulfation sites (“Ysite”) are indicated. Four hyperglycoslated variants are indicated with an asterisk (*).

FIG. 4. Schematic of AAV8-antiVEGFfab genome

FIG. 5. A subretinal drug delivery device comprising a catheter that can be inserted and tunneled through the suprachoroidal space toward the posterior pole, where a small needle injects into the subretinal space, manufactured by Janssen Pharmaceuticals, Inc.

FIGS. 6A-6D. Illustration of the posterior juxtascleral depot procedure. FIG. 6A depicts that following the creation of a small incision to bare sclera, the cannula tip is inserted. FIGS. 6B, 6C and 6D depict that the curved portion of the cannula shaft is inserted, keeping the cannula tip in direct apposition to the scleral surface.

FIG. 7. Clustal Multiple Sequence Alignment of AAV capsids 1-9 (SEQ ID NOs: 41-51). Amino acid substitutions (shown in bold in the bottom rows) can be made to AAV9 and AAV8 capsids by “recruiting” amino acid residues from the corresponding position of other aligned AAV capsids. Sequence regions designated by “HVR”=hypervariable regions.

FIGS. 8A and 8B. A micro volume injector drug delivery device manufactured by Altaviz. FIG. 8A depicts the micro volume injector drug delivery device, and FIG. 8B depicts the components of the micro volume injector drug delivery device.

FIGS. 9A and 9B. A drug delivery device manufactured by Visionisti OY. Specifically, FIG. 9A depicts the injection adapter, which is able to convert 30 g short hypodermic needles into a suprachoroidal/subretinal needles. The device is able to control the length of the needle tip exposed from the distal tip of the adapter. Adjustments can be made at 10 μL. The device has the ability to adjust for suprachoroidal delivery and/or ab-externo subretinal delivery. FIG. 9B depicts a needle adaptor guide which is able to keep the lids open and hold the needle at the optimal angle and depth for delivery. The needle adapter is locked into the stabilizing device. The needle adapter is an all-in-one tool for standardized and optimized in-office suprachoroidal and/or subretinal injections.

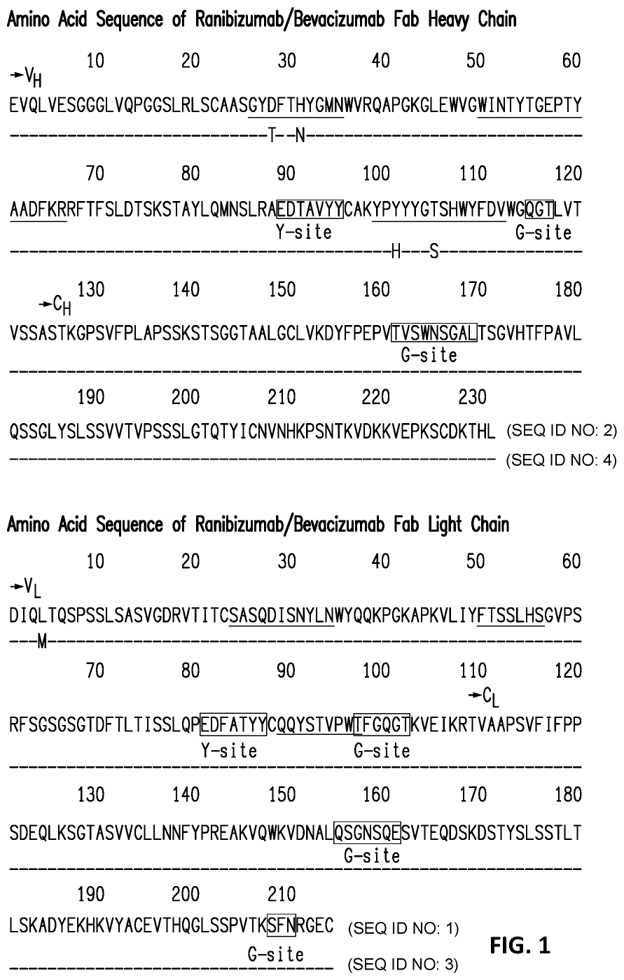

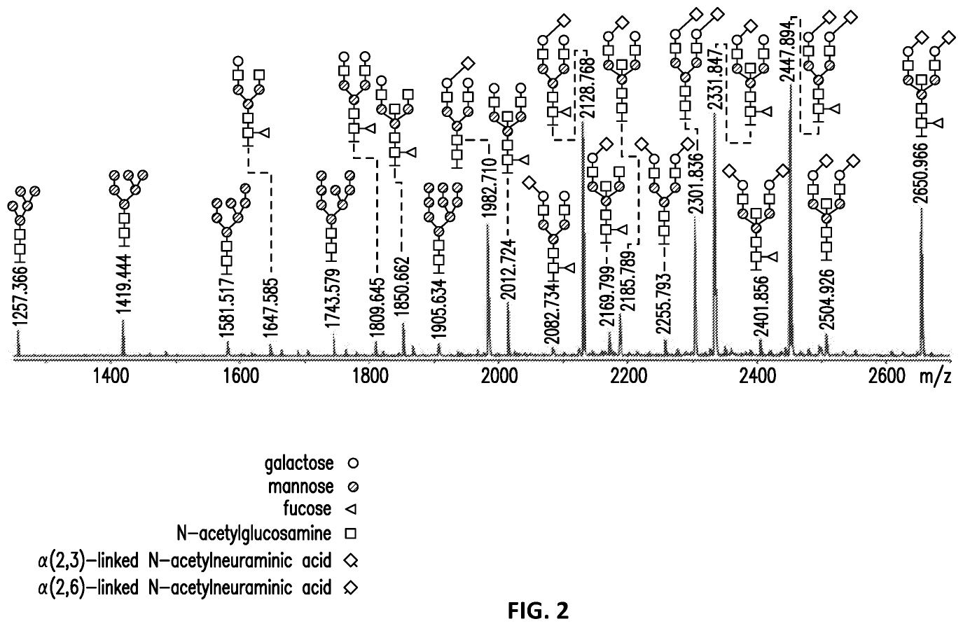

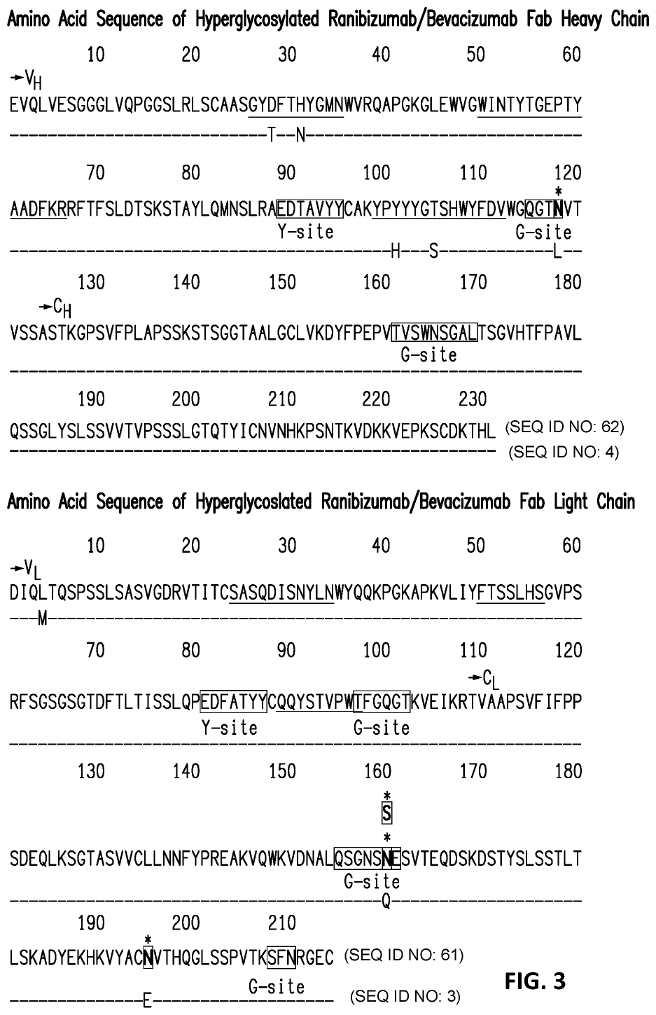

### 5. DETAILED DESCRIPTION

### 5.1 Overview

Provided herein is a method of treating neovascular age-related macular degeneration (nAMD) in a subject in need thereof, wherein the method comprises administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to an eye of the subject; and the steroid treatment comprises administering a therapeutically effective amount of a steroid to the eye of the subject.

Also provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to the suprachoroidal space of an eye of the subject; and the steroid treatment comprises administering a therapeutically effective amount of a steroid to the eye of the subject.

Also provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to an eye of the subject; and the steroid treatment comprises administering a therapeutically effective amount of triamcinolone acetonide to the eye of the subject.

Also provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector comprising a nucleotide sequence encoding an anti-hVEGF antigen-binding fragment to an eye of the subject; and the steroid treatment comprises administering a therapeutically effective amount of difluprednate to the eye of the subject.

In certain embodiments of the methods provided herein, the method is a method of treating neovascular age-related macular degeneration (nAMD). In certain embodiments, the method is a method of treating diabetic retinopathy (DR).

In certain embodiments, the nAMD and DR can be treated using the methods disclosed in Section 5.3 and Section 5.4.

In certain embodiments, the anti-hVEGF treatment provided herein comprises administering a recombinant viral vector as described in Section 5.2. In certain embodiments, the recombinant viral vector is administered as described in Section 5.3. In certain embodiments, the steroid treatment is administered as described in Section 5.4.

In certain embodiments, the anti-hVEGF treatment provided herein comprises delivery of a fully human post-translationally modified (HuPTM) antibody against VEGF to the retina/vitreal humour in the eye(s) of patients (human subjects) diagnosed with neovascular age-related macular degeneration (nAMD) or diabetic retinopathy (DR).

Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, and antigen-binding fragments of full-length antibodies, and fusion proteins of the above. Such antigen-binding fragments include, but are not limited to, single-domain antibodies (variable domain of heavy chain antibodies (VHHs) or nanobodies), Fabs, F(ab′)₂s, and scFvs (single-chain variable fragments) of full-length anti-VEGF antibodies (preferably, full-length anti-VEGF monoclonal antibodies (mAbs)) (collectively referred to herein as “antigen-binding fragments”). In a preferred embodiment, the fully human post-translationally modified antibody against VEGF is a fully human post-translationally modified antigen-binding fragment of a monoclonal antibody (mAb) against VEGF (“HuPTMFabVEGFi”). In a further preferred embodiment, the HuPTMFabVEGFi is a fully human glycosylated antigen-binding fragment of an anti-VEGF mAb (“HuGlyFabVEGFi”). See, also, International Patent Application Publication No. WO/2017/180936 (International Patent Application No. PCT/US2017/027529, filed Apr. 14, 2017), International Patent Application Publication No. WO/2017/181021 (International Patent Application No. PCT/US2017/027650, filed Apr. 14, 2017), International Patent Application Publication No. WO2019/067540 (International Patent Application No. PCT/US2018/052855, filed Sep. 26, 2018), International Patent Application Publication No. WO2020/206098 (International Patent Application No. PCT/US2020/026356, filed Apr. 20, 2020), and International Patent Application Publication No. WO2021/041373 (International Patent Application No. PCT/US2020/047733, filed Aug. 25, 2020), each of which is incorporated by reference herein in its entirety, for compositions and methods that can be used according to the embodiments described herein. In an alternative embodiment, full-length mAbs can be used. Delivery may be accomplished via gene therapy—e.g., by administering a viral vector or other DNA expression construct encoding an anti-VEGF antigen-binding fragment or mAb (or a hyperglycosylated derivative) to the suprachoroidal space, subretinal space (from a transvitreal approach or with a catheter through the suprachoroidal space), intraretinal space, vitreous cavity, and/or outer surface of the sclera (i.e., juxtascleral administration) in the eye(s) of patients (human subjects) diagnosed with neovascular age-related macular degeneration (nAMD) or diabetic retinopathy (DR), to create a permanent depot in the eye that continuously supplies the human PTM, e.g., human-glycosylated, transgene product. See, e.g., administration modes described in Section 5.3.2.

In certain embodiments, the patients have been shown to be responsive to treatment with an anti-VEGF antigen-binding fragment injected intravitreally prior to treatment with gene therapy. In specific embodiments, the patients have previously been treated with LUCENTIS® (ranibizumab), EYLEA® (aflibercept), and/or AVASTIN® (bevacizumab), and have been found to be responsive to one or more of said LUCENTIS® (ranibizumab), EYLEA® (aflibercept), and/or AVASTIN® (bevacizumab).

Subjects to whom such viral vector or other DNA expression construct is delivered should be responsive to the anti-VEGF antigen-binding fragment encoded by the transgene in the viral vector or expression construct. To determine responsiveness, the anti-hVEGF antigen-binding fragment transgene product (e.g., produced in cell culture, bioreactors, etc.) may be administered directly to the subject, such as by intravitreal injection.

The HuPTMFabVEGFi, e.g., HuGlyFabVEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFabVEGFi, e.g., HuGlyFabVEGFi, transgene should be controlled by appropriate expression control elements, for example, the CB7 promoter (a chicken β-actin promoter and CMV enhancer), the RPE65 promoter, or opsin promoter to name a few, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57.

In preferred embodiments, gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant (e.g., rHuGlyFabVEGFi) vector in a formulation buffer comprising a physiologically compatible aqueous buffer, a surfactant and optional excipients. In a specific embodiment, the construct is formulated in Dulbecco's phosphate buffered saline and 0.001% Pluronic F68, pH=7.4.

Therapeutically effective doses of the recombinant vector should be administered subretinally and/or intraretinally (e.g., by subretinal injection via the transvitreal approach (a surgical procedure), or subretinal administration via the suprachoroidal space) in a volume ranging from ≥0.1 mL to 0.5 mL, preferably in 0.1 to 0.30 mL (100-300 μl), and most preferably, in a volume of 0.25 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera in a volume of 500 μl or less, for example, in a volume of 500 μl or less, for example, in a volume of 10-20 μl, 20-50 μl, 50-100 μl, 100-200 μl, 200-300 μl, 300-400 μl, or 400-500 μl. Subretinal injection is a surgical procedure performed by trained retinal surgeons that involves a partial vitrectomy with the subject under local anesthesia, and injection of the gene therapy into the retina. (see, e.g., Campochiaro et al., 2017, Hum Gen Ther 28(1):99-111, which is incorporated by reference herein in its entirety). In a specific embodiment, the subretinal administration is performed via the suprachoroidal space using a subretinal drug delivery device that comprises a catheter which can be inserted and tunneled through the suprachoroidal space to the posterior pole, where a small needle injects into the subretinal space (see, e.g., Baldassarre et al., 2017, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1; each of which is incorporated by reference herein in its entirety). Suprachoroidal administration procedures involve administration of a drug to the suprachoroidal space of the eye, and are normally performed using a suprachoroidal drug delivery device such as a microinjector with a microneedle (see, e.g., Hariprasad, 2016, Retinal Physician 13: 20-23; Goldstein, 2014, Retina Today 9(5): 82-87; each of which is incorporated by reference herein in its entirety). The suprachoroidal drug delivery devices that can be used to deposit the expression vector in the suprachoroidal space according to the embodiments described herein include, but are not limited to, suprachoroidal drug delivery devices manufactured by Clearside® Biomedical, Inc. (see, for example, Hariprasad, 2016, Retinal Physician 13: 20-23). The subretinal drug delivery devices that can be used to deposit the expression vector in the subretinal space via the suprachoroidal space according to the embodiments described herein include, but are not limited to, subretinal drug delivery devices manufactured by Janssen Pharmaceuticals, Inc. (see, for example, International Patent Application Publication No. WO 2016/040635 A1). In a specific embodiment, administration to the outer surface of the sclera is performed by a juxtascleral drug delivery device that comprises a cannula, whose tip can be inserted and kept in direct apposition to the scleral surface. See Section 5.3.2 for more details of the different modes of administration. Suprachoroidal, subretinal, juxtascleral, intravitreal, subconjunctival, and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Müller cells, results in delivery and maintenance of the transgene product in the retina, the vitreous humor, and/or the aqueous humor. In a specific embodiment, doses that maintain a concentration of the transgene product at a Cₘᵢₙ of at least 0.330 μg/mL in the vitreous humour, or 0.110 μg/mL in the aqueous humour (the anterior chamber of the eye) for three months are desired; thereafter, vitreous Cₘᵢₙ concentrations of the transgene product ranging from 1.70 to 6.60 μg/mL, and/or aqueous Cₘᵢₙ concentrations ranging from 0.567 to 2.20 μg/mL should be maintained. However, because the transgene product is continuously produced, maintenance of lower concentrations can be effective. In a specific embodiment, the concentration of the transgene product can be measured in patient samples of the vitreous humour and/or aqueous from the anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

Vector transgenes have the potential to spread to unintended recipients from shedding (release of vectors that did not infect the target cells and were cleared from the body via feces or bodily fluids), mobilization (transgene replication and transfer out of the target cell), or germ line transmission (genetic transmission to offspring through semen). Vector shedding may be determined for example by measuring vector DNA in biological fluids such as tears, serum or urine using quantitative polymerase chain reaction. In some embodiments, no vector gene copies are detectable in a biological fluid (e.g., tears, serum or urine) at any time point after administration of the vector. In some embodiments, less than 1000, less than 500, less than 100, less than 50 or less than 10 vector gene copies/5 μL are detectable by quantitative polymerase chain reaction in a biological fluid (e.g., tears, serum or urine) at any point after administration. In specific embodiments, 210 vector gene copies/5 μL or less are detectable in serum. In some embodiments, less than 1000, less than 500, less than 100, less than 50 or less than 10 vector gene copies/5 μL are detectable by quantitative polymerase chain reaction in a biological fluid (e.g., tears, serum or urine) by 1, 2, 3, 4, 5, 6, 7, 8, 9, 10, 11, 12, 13 or 14 weeks after administration. In specific embodiments, no vector gene copies are detectable in serum by week 14 after administration of the vector.

The embodiments described herein have several advantages over standard of care treatments that involve repeated ocular injections of high dose boluses of the VEGF inhibitor that dissipate over time resulting in peak and trough levels. Sustained expression of the transgene product antibody, as opposed to injecting an antibody repeatedly, allows for a more consistent levels of antibody to be present at the site of action, and is less risky and more convenient for patients, since fewer injections need to be made, resulting in fewer doctor visits. Consistent protein production may leads to better clinical outcomes as edema rebound in the retina is less likely to occur. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

The production of HuPTMFabVEGFi, e.g., HuGlyFabVEGFi, should result in a “biobetter” molecule for the treatment of neovascular age-related macular degeneration (nAMD) and/or diabetic retinopathy (DR) accomplished via gene therapy—e.g., by administering a viral vector or other DNA expression construct encoding HuPTMFabVEGFi, e.g., HuGlyFabVEGFi, to the suprachoroidal space, subretinal space, or outer surface of the sclera in the eye(s) of patients (human subjects) diagnosed with neovascular age-related macular degeneration (nAMD) or diabetic retinopathy (DR), (e.g., by suprachoroidal injection, subretinal injection via the transvitreal approach (a surgical procedure), subretinal administration via the suprachoroidal space, or a posterior juxtascleral depot procedure), to create a permanent depot in the eye that continuously supplies the fully-human post-translationally modified, e.g., human-glycosylated, sulfated transgene product produced by transduced retinal cells. The cDNA construct for the FabVEGFi should include a signal peptide that ensures proper co- and post-translational processing (glycosylation and protein sulfation) by the transduced retinal cells. Such signal sequences used by retinal cells may include but are not limited to:

| | |
|---|---|
| • | (VEGF-A signal peptide) |
| | (SEQ ID NO: 5) |
| | MNFLLSWVHWSLALLLYLHHAKWSQA |
| • | (Fibulin-1 signal peptide) |
| | (SEQ ID NO: 6) |
| | MERAAPSRRVPLPLLLLGGLALLAAGVDA |
| • | (Vitronectin signal peptide) |
| | (SEQ ID NO: 7) |
| | MAPLRPLLILALLAWVALA |
| • | (Complement Factor H signal peptide) |
| | (SEQ ID NO: 8) |
| | MRLLAKIICLMLWAICVA |
| • | (Opticin signal peptide) |
| | (SEQ ID NO: 9) |
| | MRLLAFLSLLALVLQETGT |
| • | (Albumin signal peptide) |
| | (SEQ ID NO: 22) |
| | MKWVTFISLLFLFSSAYS |
| • | (Chymotrypsinogen signal peptide) |
| | (SEQ ID NO: 23) |
| | MAFLWLLSCWALLGTTFG |
| • | (Interleukin-2 signal peptide) |
| | (SEQ ID NO: 24) |
| | MYRMQLLSCIALILALVTNS |
| • | (Trypsinogen-2 signal peptide) |
| | (SEQ ID NO: 25) |
| | MNLLLILTFVAAAVA |

See, e.g., Stern et al., 2007, Trends Cell. Mol. Biol., 2:1-17 and Dalton & Barton, 2014, Protein Sci, 23: 517-525, each of which is incorporated by reference herein in its entirety for the signal peptides that can be used.

As an alternative, or an additional treatment to gene therapy, the HuPTMFabVEGFi product, e.g., HuGlyFabVEGFi glycoprotein, can be produced in human cell lines by recombinant DNA technology, and administered to patients diagnosed with neovascular age-related macular degeneration (nAMD) or diabetic retinopathy (DR) by intravitreal injection. The HuPTMFabVEGFi product, e.g., glycoprotein, may also be administered to patients with neovascular age-related macular degeneration (nAMD) or diabetic retinopathy (DR).

Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. (Early Online, published online Sep. 18, 2015, pp. 1-13) “Human cell lines for biopharmaceutical manufacturing: history, status, and future perspectives” which is incorporated by reference in its entirety for a review of the human cell lines that could be used for the recombinant production of the HuPTMFabVEGFi product, e.g., HuGlyFabVEGFi glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Combinations of delivery of the HuPTMFabVEGFi, e.g., HuGlyFabVEGFi, to the eye/retina accompanied by delivery of other available treatments are encompassed by the methods provided herein. The additional treatments may be administered before, concurrently or subsequent to the gene therapy treatment. Available treatments for neovascular age-related macular degeneration (nAMD) or diabetic retinopathy (DR) that could be combined with the gene therapy provided herein include but are not limited to laser photocoagulation, photodynamic therapy with verteporfin, and intravitreal (IVT) injections with anti-VEGF agents, including but not limited to pegaptanib, ranibizumab, aflibercept, or bevacizumab. Additional treatments with anti-VEGF agents, such as biologics, may be referred to as “rescue” therapy.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (from about 1% to about 10% of the population), including 2,6-sialylation, and sulfation to demonstrate efficacy. The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (SD-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation.

### 5.2 Constructs and Formulations

For use in the methods provided herein are viral vectors or other DNA expression constructs encoding an anti-VEGF antigen-binding fragment or a hyperglycosylated derivative of an anti-VEGF antigen-binding fragment. The viral vectors and other DNA expression constructs provided herein include any suitable method for delivery of a transgene to a target cell (e.g., retinal pigment epithelial cells). The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to retinal pigment epithelial cells.

In some aspects, the disclosure provides for a nucleic acid for use, wherein the nucleic acid encodes a HuPTMFabVEGFi, e.g., HuGlyFabVEGFi operatively linked to a promoter selected from the group consisting of: the CB7 promoter (a chicken j-actin promoter and CMV enhancer), cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter. In a specific embodiment, HuPTMFabVEGFi is operatively linked to the CB7 promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., an anti-VEGF antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

In another specific embodiment, the construct described herein is Construct II, wherein the Construct II comprises the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

In a specific embodiment, the construct comprises an expression cassette encoding an anti-hVEGF antigen-binding fragment, wherein the expression cassette is flanked by AAV2 inverted terminal repeats (ITRs), and wherein the expression cassette comprises:
a CB7 promotor consisting of a chicken j-actin promoter and a CMV enhancer;
a chicken β-actin intron;
a nucleotide sequence encoding:
   an IL-2 signal peptide;
   a heavy chain of the anti-hVEGF antigen-binding fragment comprising the amino acid sequence of SEQ ID NO: 2;
   a self-cleaving furin (F)/F2A linker;
   a second IL-2 signal peptide; and
   a light chain of the anti-hVEGF antigen-binding fragment comprising the amino acid sequence of SEQ ID NO: 1; and
a rabbit β-globin poly A signal.

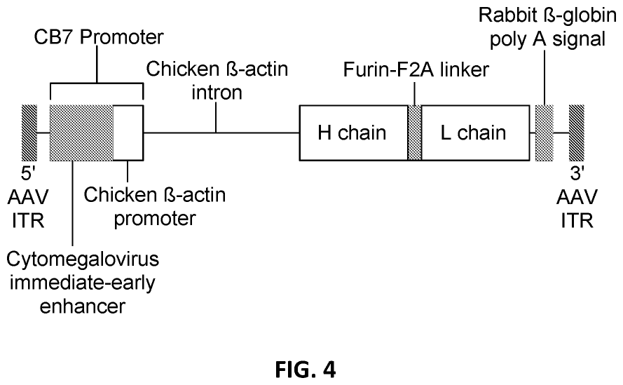

In a specific embodiment, the construct described herein is illustrated in FIG. 4.

5.2.1 mRNA

In certain embodiments, the vectors provided herein are modified mRNA encoding for the gene of interest (e.g., the transgene, for example, an anti-VEGF antigen-binding fragment moiety). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for an anti-VEGF antigen-binding fragment moiety.

### 5.2.2 Viral Vectors

Viral vectors include adenovirus, adeno-associated virus (AAV, e.g., AAV8), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV)- and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

In certain embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV3, AAV4, AAV5, AAV6, AAV7, AAV8, AAV9, AAV10, AAV11, or AAVrh10. In preferred embodiments, AAV based vectors provided herein comprise components from one or more of AAV8, AAV9, AAV10, AAV 11, or AAVrh10 serotypes.

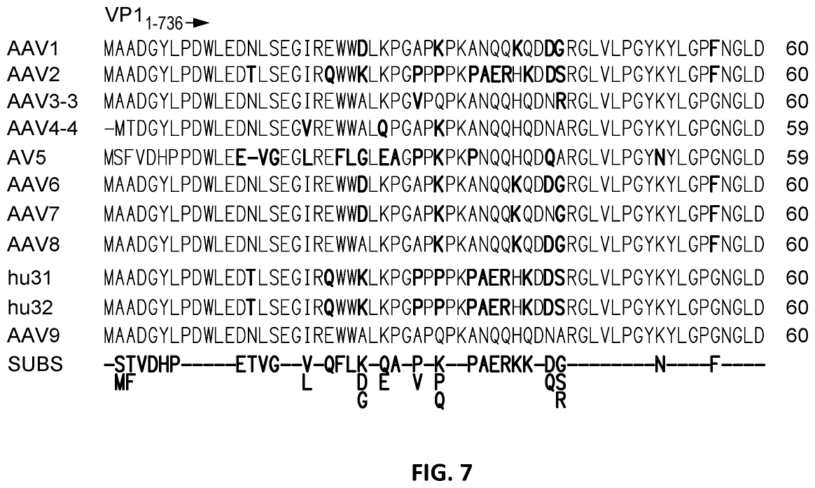

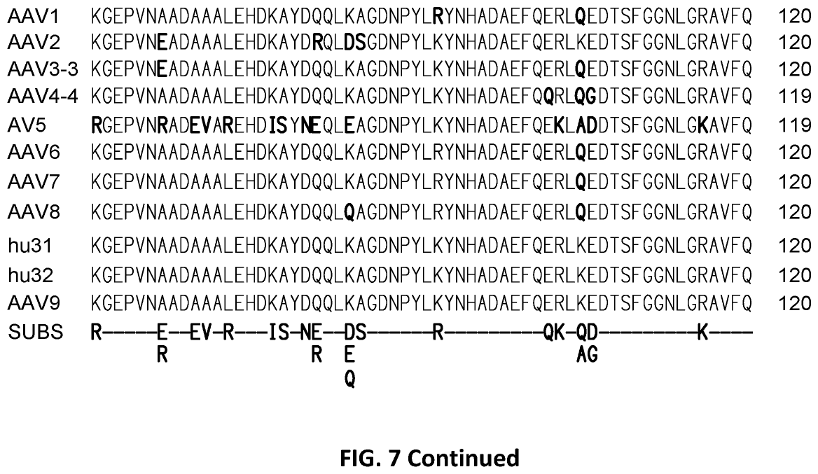

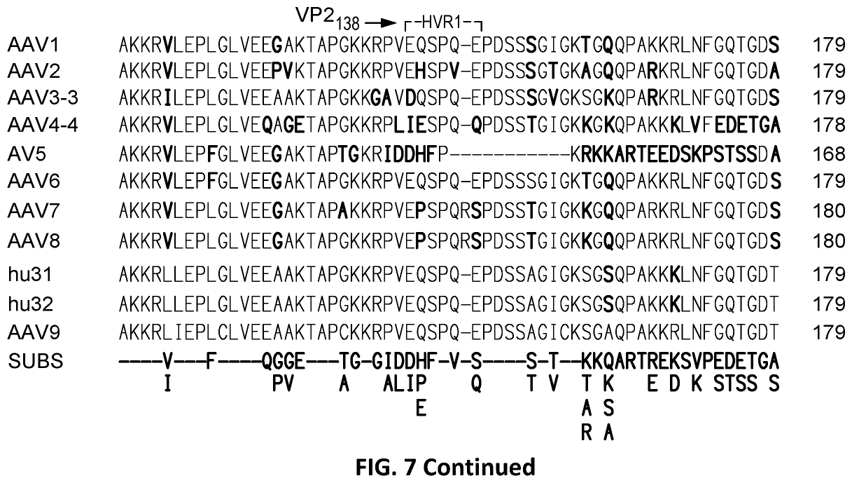

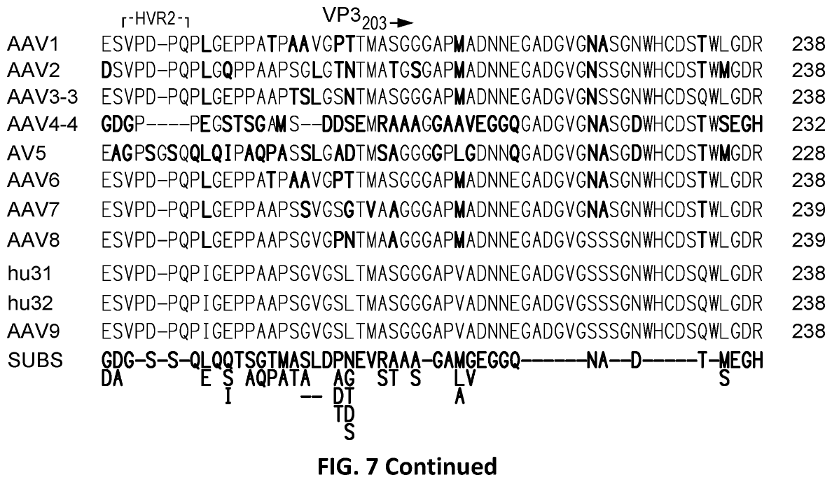

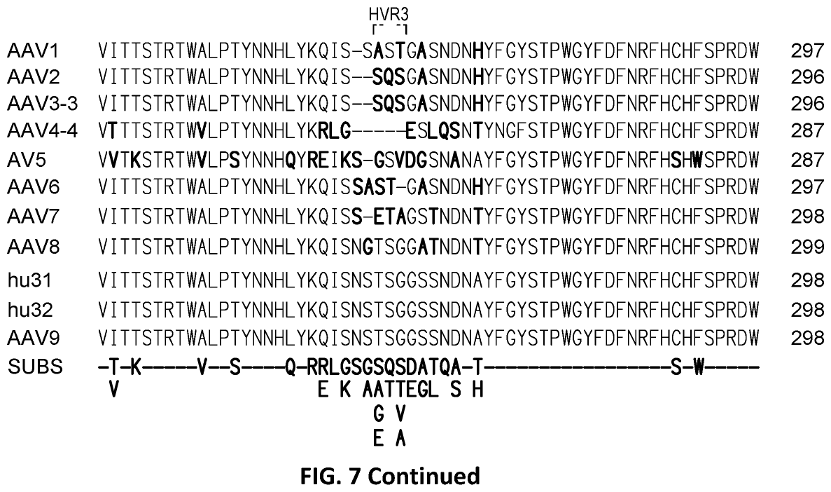

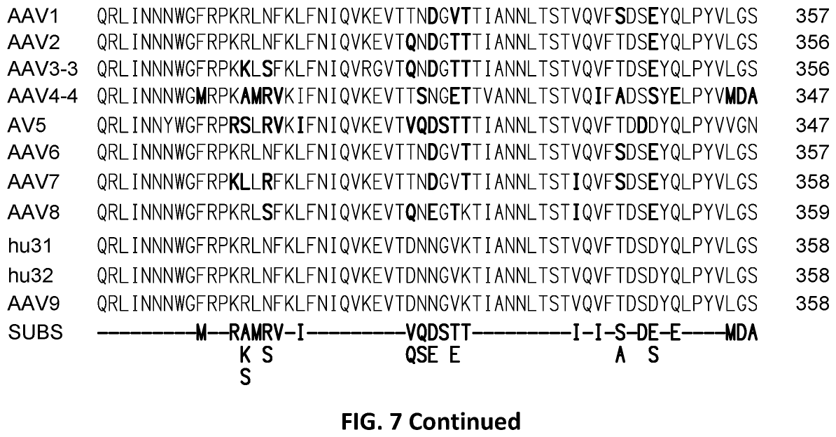

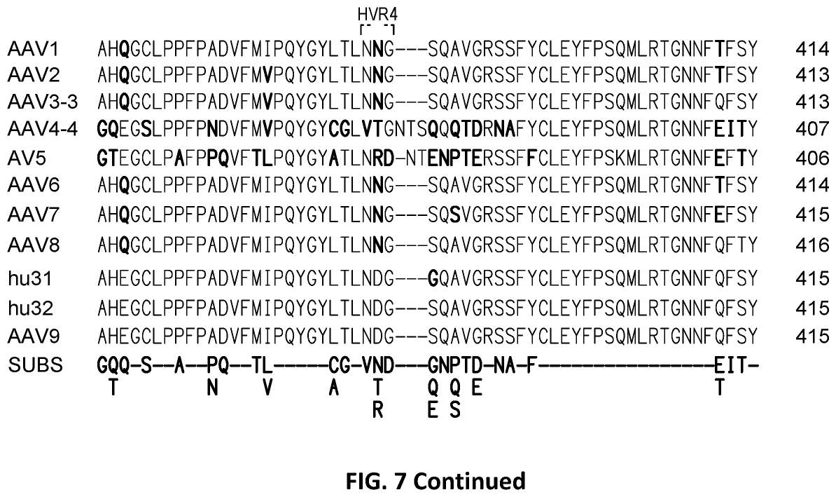

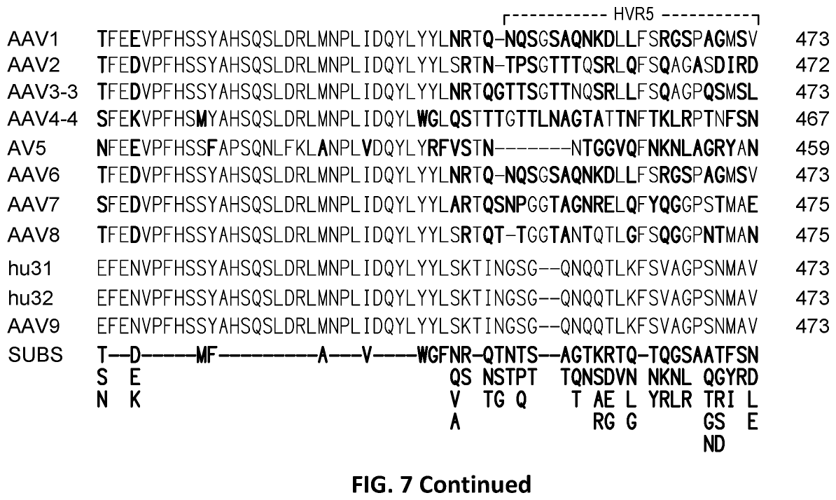

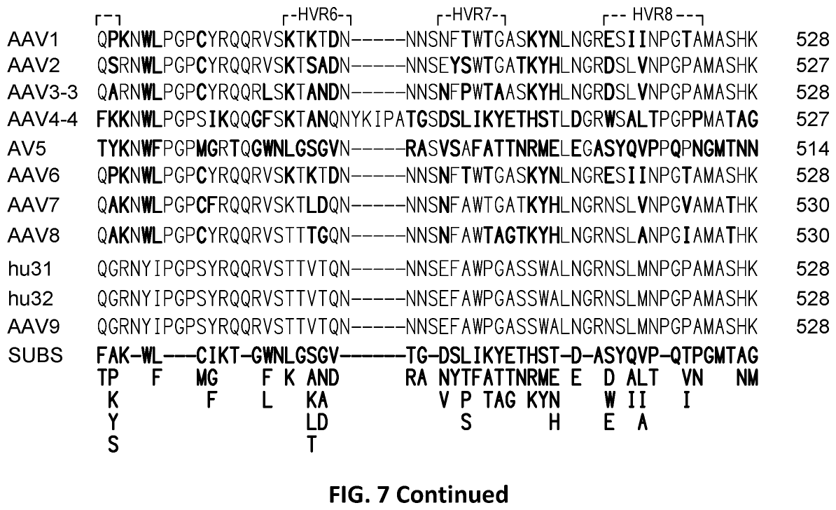

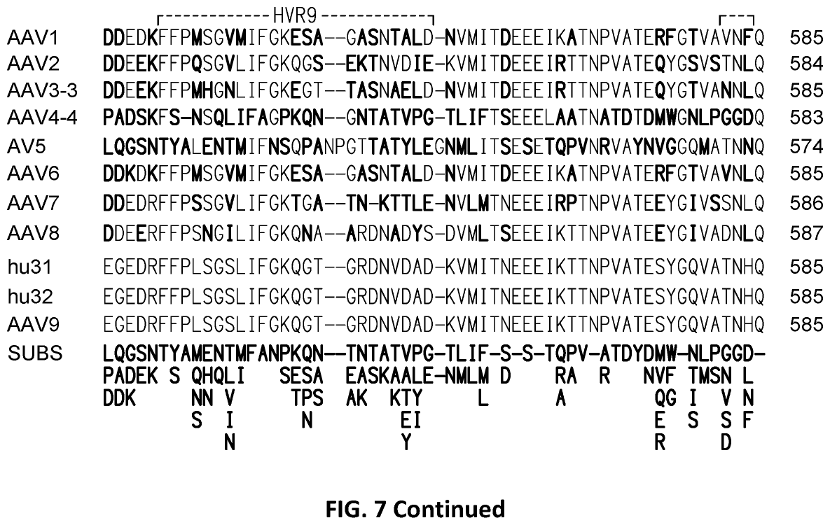

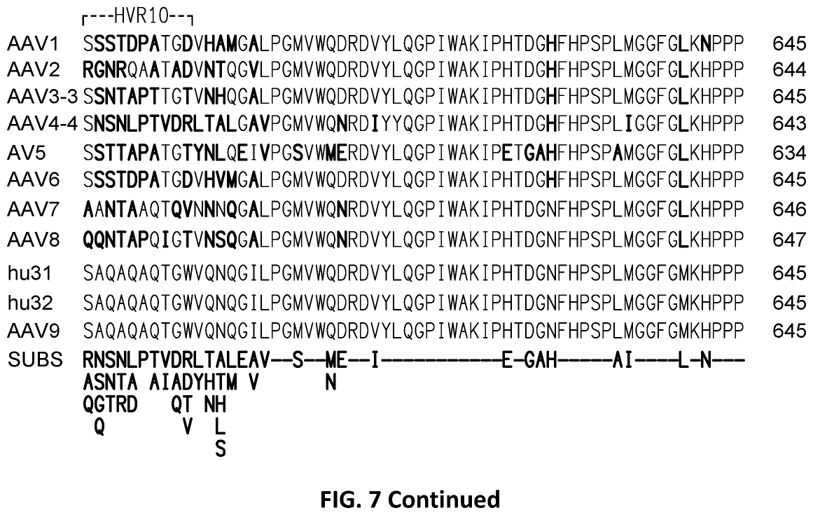

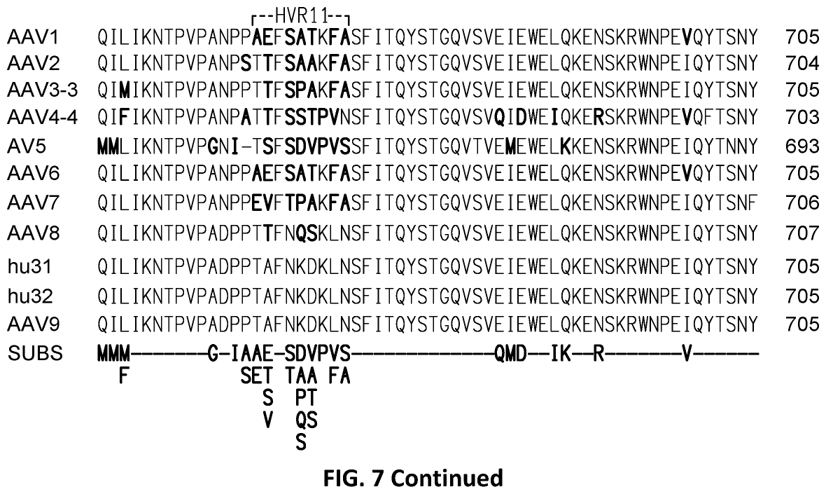

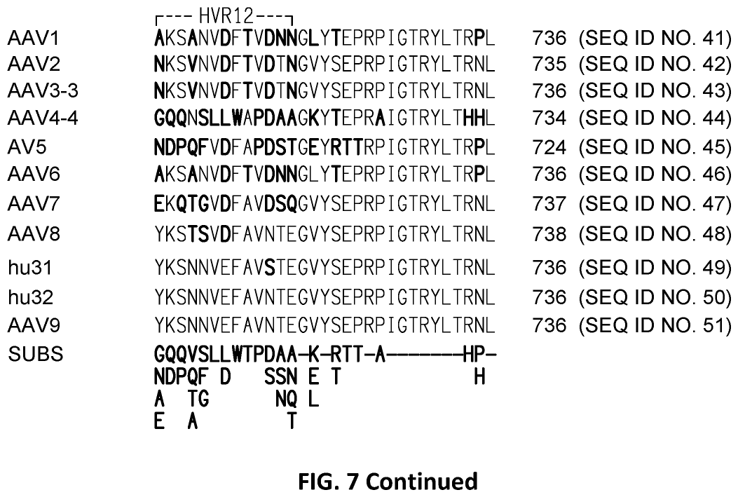

Provided in particular embodiments are AAV8 vectors comprising a viral genome comprising an expression cassette for expression of the transgene, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 48) while retaining the biological function of the AAV8 capsid. In certain embodiments, the encoded AAV8 capsid has the sequence of SEQ ID NO: 48 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8 capsid. FIG. 7 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV8 vector comprises an AAV8 capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions identified in the SUBS row of FIG. 7 that are not present at that position in the native AAV8 sequence.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP (SEQ ID NO: 59) or LALGETTRP (SEQ ID NO: 60), as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV-PHP.B. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8)-based viral vectors encoding a transgene (e.g., an anti-VEGF antigen-binding fragment). In specific embodiments, provided herein are AAV8-based viral vectors encoding an anti-VEGF antigen-binding fragment. In more specific embodiments, provided herein are AAV8-based viral vectors encoding ranibizumab.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the anti-VEGF antigen-binding fragment. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the anti-VEGF antigen-binding fragment. Four plasmids are used to make the construct: Gag/pol sequence containing plasmid, Rev sequence containing plasmids, Envelope protein containing plasmid (i.e. VSV-G), and Cis plasmid with the packaging elements and the anti-VEGF antigen-binding fragment gene.

For lentiviral vector production, the four plasmids are co-transfected into cells (i.e., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an anti-VEGF antigen-binding fragment (e.g., ranibizumab) such that, upon introduction of the vector into a relevant cell (e.g., a retinal cell in vivo or in vitro), a glycosylated and or tyrosine sulfated variant of the anti-VEGF antigen-binding fragment is expressed by the cell. In a specific embodiment, the expressed anti-VEGF antigen-binding fragment comprises a glycosylation and/or tyrosine sulfation pattern.

### 5.2.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters. In certain embodiments, the promoter is a constitutive promoter. In certain embodiments, the promoter is an inducible promoter. Inducible promoters may be preferred so that transgene expression may be turned on and off as desired for therapeutic efficacy. Such promoters include, for example, hypoxia-induced promoters and drug inducible promoters, such as promoters induced by rapamycin and related agents. Hypoxia-inducible promoters include promoters with HIF binding sites, see, for example, Schödel, et al., 2011, Blood 117(23):e207-e217 and Kenneth and Rocha, 2008, Biochem J. 414:19-29, each of which is incorporated by reference for teachings of hypoxia-inducible promoters. In addition, hypoxia-inducible promoters that may be used in the constructs include the erythropoietin promoter and N-WASP promoter (see, Tsuchiya, 1993, J. Biochem. 113:395 for disclosure of the erythropoietin promoter and Salvi, 2017, Biochemistry and Biophysics Reports 9:13-21 for disclosure of N-WASP promoter, both of which are incorporated by reference for the teachings of hypoxia-induced promoters). Alternatively, the constructs may contain drug inducible promoters, for example promoters inducible by administration of rapamycin and related analogs (see, for example, International Patent Application Publication Nos. WO94/18317, WO 96/20951, WO 96/41865, WO 99/10508, WO 99/10510, WO 99/36553, and WO 99/41258, and U.S. Pat. No. 7,067,526 (disclosing rapamycin analogs), which are incorporated by reference herein for their disclosure of drug inducible promoters). In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2α binding site. In certain embodiments, the HIF binding site comprises an RCGTG motif For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety.

In certain embodiments, the promoter is a CB7 promoter (see Dinculescu et al., 2005, Hum Gene Ther 16: 649-663, incorporated by reference herein in its entirety). In some embodiments, the CB7 promoter includes other expression control elements that enhance expression of the transgene driven by the vector. In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs. In certain embodiments, the vectors provided herein comprise one or more tissue specific promoters (e.g., a retinal pigment epithelial cell-specific promoter). In certain embodiments, the viral vectors provided herein comprise a RPE65 promoter. In certain embodiments, the vectors provided herein comprise a VMD2 promoter.

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron or a chimeric intron. In certain embodiments, the viral vectors provided herein comprise a polyadenylation sequence.

### 5.2.4 Signal Peptides

In certain embodiments, the vectors provided herein comprise components that modulate protein delivery. In certain embodiments, the viral vectors provided herein comprise one or more signal peptides. Signal peptides may also be referred to herein as “leader sequences” or “leader peptides”. In certain embodiments, the signal peptides allow for the transgene product (e.g., the anti-VEGF antigen-binding fragment moiety) to achieve the proper packaging (e.g. glycosylation) in the cell. In certain embodiments, the signal peptides allow for the transgene product (e.g., the anti-VEGF antigen-binding fragment moiety) to achieve the proper localization in the cell. In certain embodiments, the signal peptides allow for the transgene product (e.g., the anti-VEGF antigen-binding fragment moiety) to achieve secretion from the cell. Examples of signal peptides to be used in connection with the vectors and transgenes provided herein may be found in Table 1.

TABLE 1
Signal peptides for use with the vectors provided herein.

| SEQ ID NO. | Signal Peptide | Sequence |
|---|---|---|
| 5 | VEGF-A signal peptide | MNFLLSWVHWSLALLLYLHHAKWSQA |
| 6 | Fibulin-1 signal peptide | MERAAPSRRVPLPLLLLGGLALLAAGVDA |
| 7 | Vitronectin signal peptide | MAPLRPLLILALLAWVALA |
| 8 | Complement Factor H signal | MRLLAKIICLMLWAICVA |
| | peptide | |
| 9 | Opticin signal peptide | MRLLAFLSLLALVLQETGT |
| 22 | Albumin signal peptide | MKWVTFISLLFLFSSAYS |
| 23 | Chymotrypsinogen signal | MAFLWLLSCWALLGTTFG |
| | peptide | |
| 24 | Interleukin-2 signal peptide | MYRMQLLSCIALILALVTNS |
| 25 | Trypsinogen-2 signal peptide | MNLLLILTFVAAAVA |

### 5.2.5 Polycistronic Messages—IRES and F2A Linkers

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based vector).

Furin-F2A linkers. In other embodiments, the viral vectors provided herein encode the heavy and light chains separated by a cleavable linker such as the self-cleaving furin/F2A (F/F2A) linkers (Fang et al., 2005, Nature Biotechnology 23: 584-590, and Fang, 2007, Mol Ther 15: 1153-9, each of which is incorporated by reference herein in its entirety).

For example, a furin-F2A linker may be incorporated into an expression cassette to separate the heavy and light chain coding sequences, resulting in a construct with the structure: Leader-Heavy chain-Furin site-F2A site-Leader-Light chain-PolyA.

The F2A site, with the amino acid sequence LLNFDLLKLAGDVESNPGP (SEQ ID NO: 26) is self-processing, resulting in “cleavage” between the final G and P amino acid residues. Additional linkers that could be used include but are not limited to:

T2A:
(SEQ ID NO: 27)
(GSG) E G R G S L L T C G D V E E N P G P;
P2A:
(SEQ ID NO: 28)
(GSG) A T N F S L L K Q A G D V E E N P G P;
E2A:
(SEQ ID NO: 29)
(GSG) Q C T N Y A L L K L A G D V E S N P G P;
F2A:
(SEQ ID NO: 30)
(GSG) V K Q T L N F D L L K L A G D V E S N P G P.

A peptide bond is skipped when the ribosome encounters the F2A sequence in the open reading frame, resulting in the termination of translation, or continued translation of the downstream sequence (the light chain). This self-processing sequence results in a string of additional amino acids at the end of the C-terminus of the heavy chain. However, such additional amino acids are then cleaved by host cell Furin at the furin sites, located immediately prior to the F2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR (SEQ ID NO: 31), RRRR (SEQ ID NO: 32), RRKR (SEQ ID NO: 33), or RKKR (SEQ ID NO: 34). Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR (SEQ ID NO: 31), RRRR (SEQ ID NO: 32), RRKR (SEQ ID NO: 33), or RKKR (SEQ ID NO: 34). In certain embodiments, one the linker is cleaved by an carboxypeptidase, no additional amino acids remain. In certain embodiments, 0.5%, 1%, 2%, 3%, 4%, 5%, 10%, 15%, or 20%, or less but more than 0% of the antibody, e.g., antigen-binding fragment, population produced by the constructs for use in the methods described herein has one, two, three, or four amino acids remaining on the C-terminus of the heavy chain after cleavage. In certain embodiments, 0.5-1%, 0.5%-2%, 0.5%-3%, 0.5%-4%, 0.5%-5%, 0.5%-10%, 0.5%-20%, 1%-2%, 1%-3%, 1%-4%, 1%-5%, 1%-10%, 1%-20%, 2%-3%, 2%-4%, 2%-5%, 2%-10%, 2%-20%, 3%-4%, 3%-5%, 3%-10%, 3%-20%, 4%-5%, 4%-10%, 4%-20%, 5%-10%, 5%-20%, or 10%-20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R (SEQ ID NO: 35), such that the additional amino acids on the C-terminus of the heavy chain are R, RX, RXK, RXR, RXKR (SEQ ID NO: 36), or RXRR (SEQ ID NO: 37), where X is any amino acid, for example, alanine (A). In certain embodiments, no additional amino acids may remain on the C-terminus of the heavy chain.

In certain embodiments, an expression cassette described herein is contained within a viral vector with a restraint on the size of the polynucleotide(s) therein. In certain embodiments, the expression cassette is contained within an AAV virus-based vector (e.g., an AAV8-based vector).

### 5.2.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene. In certain embodiments, the UTRs are optimized for the stability of the mRNA of the transgene. In certain embodiments, the UTRs are optimized for the secondary structure of the mRNA of the transgene.

### 5.2.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety).

### 5.2.8 Transgenes

The HuPTMFabVEGFi, e.g., HuGlyFabVEGFi encoded by the transgene can include, but is not limited to an antigen-binding fragment of an antibody that binds to VEGF, such as bevacizumab; an anti-VEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

In certain embodiments, the vectors provided herein encode an anti-VEGF antigen-binding fragment transgene. In specific embodiments, the anti-VEGF antigen-binding fragment transgene is controlled by appropriate expression control elements for expression in retinal cells. In certain embodiments, the anti-VEGF antigen-binding fragment transgene comprises bevacizumab Fab portion of the light and heavy chain cDNA sequences (SEQ ID NOs. 10 and 11, respectively). In certain embodiments, the anti-VEGF antigen-binding fragment transgene comprises ranibizumab light and heavy chain cDNA sequences (SEQ ID NOs. 12 and 13, respectively). In certain embodiments, the anti-VEGF antigen-binding fragment transgene encodes a bevacizumab Fab, comprising a light chain and a heavy chain of SEQ ID NOs: 3 and 4, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-VEGF antigen-binding fragment transgene encodes an antigen-binding fragment comprising a light chain comprising the amino acid sequence set forth in SEQ ID NO: 3. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes an antigen-binding fragment comprising a heavy chain comprising the amino acid sequence set forth in SEQ ID NO: 4. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes an antigen-binding fragment comprising a light chain comprising the amino acid sequence set forth in SEQ ID NO: 3 and a heavy chain comprising the amino acid sequence set forth in SEQ ID NO: 4. In certain embodiments, the anti-VEGF antigen-binding fragment transgene encodes a hyperglycosylated ranibizumab, comprising a light chain and a heavy chain of SEQ ID NOs: 1 and 2, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-VEGF antigen-binding fragment transgene encodes an antigen-binding fragment comprising a light chain comprising the amino acid sequence set forth in SEQ ID NO: 1. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes an antigen-binding fragment comprising a heavy chain comprising the amino acid sequence set forth in SEQ ID NO: 2. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes an antigen-binding fragment comprising a light chain comprising the amino acid sequence set forth in SEQ ID NO: 1 and a heavy chain comprising the amino acid sequence set forth in SEQ ID NO: 2. In some embodiments, the C-terminal lysine of SEQ ID NO: 2 is removed after translation of the antigen-binding fragment and before the antigen-binding fragment is secreted.

In certain embodiments, the anti-VEGF antigen-binding fragment transgene encodes a hyperglycosylated bevacizumab Fab, comprising a light chain and a heavy chain of SEQ ID NOs: 3 and 4, with one or more of the following mutations: L118N (heavy chain), E195N (light chain), or Q160N or Q160S (light chain). In certain embodiments, the anti-VEGF antigen-binding fragment transgene encodes a hyperglycosylated ranibizumab, comprising a light chain and a heavy chain of SEQ ID NOs: 1 and 2, with one or more of the following mutations: L118N (heavy chain), E195N (light chain), or Q160N or Q160S (light chain). The sequences of the antigen-binding fragment transgene cDNAs may be found, for example, in Table 2. In certain embodiments, the sequence of the antigen-binding fragment transgene cDNAs is obtained by replacing the signal sequence of SEQ ID NOs: 10 and 11 or SEQ ID NOs: 12 and 13 with one or more signal sequences listed in Table 1.

In certain embodiments, the anti-VEGF antigen-binding fragment transgene encodes an antigen-binding fragment and comprises the nucleotide sequences of the six bevacizumab CDRs. In certain embodiments, the anti-VEGF antigen-binding fragment transgene encodes an antigen-binding fragment and comprises the nucleotide sequences of the six ranibizumab CDRs. In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21). In certain embodiments, the anti-VEGF antigen-binding fragment transgene encodes an antigen-binding fragment comprising a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a light chain variable region comprising light chain CDRs 1-3 of ranibizumab (SEQ ID NOs: 14, 15, and 63). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19). In certain embodiments, the anti-VEGF antigen-binding fragment transgene encodes an antigen-binding fragment comprising a light chain variable region comprising light chain CDRs 1-3 of bevacizumab (SEQ ID NOs: 14-16). In certain embodiments, the anti-VEGF antigen-binding fragment transgene encodes an antigen-binding fragment comprising a light chain variable region comprising light chain CDRs 1-3 of bevacizumab (SEQ ID NOs: 14, 15, and 63). In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21) and a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19) and a light chain variable region comprising light chain CDRs 1-3 of bevacizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21) and a light chain variable region comprising light chain CDRs 1-3 of ranibizumab (SEQ ID NOs: 14, 15, and 63). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19) and a light chain variable region comprising light chain CDRs 1-3 of bevacizumab (SEQ ID NOs: 14, 15, and 63).

It will be understood that reference to a heavy chain variable region CDR or CDRs and/or a light chain variable region CDR or CDRs of a specific antibody will encompass all CDR definitions as known to those of skill in the art. Exemplary CDRs according to various numbering systems are shown in the table below.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

In certain embodiments, the anti-VEGF antigen-binding fragment transgene encodes an antigen-binding fragment comprising a heavy chain variable region comprising a heavy chain CDR1, a heavy chain CDR2, a heavy chain CDR3 of the amino acid sequence of SEQ ID NO: 2. In certain embodiments, the anti-VEGF antigen-binding fragment transgene encodes an antigen-binding fragment comprising a heavy chain variable region comprising a heavy chain CDR1, a heavy chain CDR2, a heavy chain CDR3 of the amino acid sequence of SEQ ID NO: 4. In certain embodiments, the anti-VEGF antigen-binding fragment transgene encodes an antigen-binding fragment comprising a light chain variable region comprising a light chain CDR1, a light chain CDR2, a light chain CDR3 of the amino acid sequence of SEQ ID NO: 1. In certain embodiments, the anti-VEGF antigen-binding fragment transgene encodes an antigen-binding fragment comprising a light chain variable region comprising a light chain CDR1, a light chain CDR2, a light chain CDR3 of the amino acid sequence of SEQ ID NO: 3. In certain embodiments, the anti-VEGF antigen-binding fragment transgene encodes an antigen-binding fragment comprising (a) a heavy chain variable region comprising a heavy chain CDR1, a heavy chain CDR2, a heavy chain CDR3 of the amino acid sequence of SEQ ID NO: 2; and (b) a light chain variable region comprising a light chain CDR1, a light chain CDR2, a light chain CDR3 of the amino acid sequence of SEQ ID NO: 1. In certain embodiments, the anti-VEGF antigen-binding fragment transgene encodes an antigen-binding fragment comprising (a) a heavy chain variable region comprising a heavy chain CDR1, a heavy chain CDR2, a heavy chain CDR3 of the amino acid sequence of SEQ ID NO: 4; and (b) a light chain variable region comprising a light chain CDR1, a light chain CDR2, a light chain CDR3 of the amino acid sequence of SEQ ID NO: 3. In certain embodiments, the anti-VEGF antigen-binding fragment transgene encodes an antigen-binding fragment comprising (a) a heavy chain variable region comprising a heavy chain CDR1, a heavy chain CDR2, a heavy chain CDR3 of the amino acid sequence of SEQ ID NO: 2; and (b) a light chain variable region comprising a light chain CDR1, a light chain CDR2, a light chain CDR3 of the amino acid sequence of SEQ ID NO: 3. In certain embodiments, the anti-VEGF antigen-binding fragment transgene encodes an antigen-binding fragment comprising (a) a heavy chain variable region comprising a heavy chain CDR1, a heavy chain CDR2, a heavy chain CDR3 of the amino acid sequence of SEQ ID NO: 4; and (b) a light chain variable region comprising a light chain CDR1, a light chain CDR2, a light chain CDR3 of the amino acid sequence of SEQ ID NO: 1.

In certain embodiments, the anti-VEGF antigen-binding fragment transgene encodes an antigen-binding fragment comprising a heavy chain variable region comprising the amino acid sequences set forth in SEQ ID NO: 20, SEQ ID NO: 18, and SEQ ID NO: 21. In certain embodiments, the anti-VEGF antigen-binding fragment transgene encodes an antigen-binding fragment comprising a heavy chain variable region comprising the amino acid sequences set forth in SEQ ID NO: 17, SEQ ID NO: 18, and SEQ ID NO: 19. In certain embodiments, the anti-VEGF antigen-binding fragment transgene encodes an antigen-binding fragment comprising a light chain variable region comprising the amino acid sequences set forth in SEQ ID NO: 14, SEQ ID NO: 15, and SEQ ID NO: 16. In certain embodiments, the anti-VEGF antigen-binding fragment transgene encodes an antigen-binding fragment comprising a light chain variable region comprising the amino acid sequences set forth in SEQ ID NO: 14, SEQ ID NO: 15, and SEQ ID NO: 63.

In certain embodiments, the anti-VEGF antigen-binding fragment transgene encodes an antigen-binding fragment comprising (a) a heavy chain variable region comprising the amino acid sequences set forth in SEQ ID NO: 20, SEQ ID NO: 18, and SEQ ID NO: 21; and (b) a light chain variable region comprising the amino acid sequences set forth in SEQ ID NO: 14, SEQ ID NO: 15, and SEQ ID NO: 16. In certain embodiments, the anti-VEGF antigen-binding fragment transgene encodes an antigen-binding fragment comprising (a) a heavy chain variable region comprising the amino acid sequences set forth in SEQ ID NO: 20, SEQ ID NO: 18, and SEQ ID NO: 21; and (b) a light chain variable region comprising the amino acid sequences set forth in SEQ ID NO: 14, SEQ ID NO: 15, and SEQ ID NO: 63. In certain embodiments, the anti-VEGF antigen-binding fragment transgene encodes an antigen-binding fragment comprising (a) a heavy chain variable region comprising the amino acid sequences set forth in SEQ ID NO: 17, SEQ ID NO: 18, and SEQ ID NO: 19; and (b) a light chain variable region comprising the amino acid sequences set forth in SEQ ID NO: 14, SEQ ID NO: 15, and SEQ ID NO: 16. In certain embodiments, the anti-VEGF antigen-binding fragment transgene encodes an antigen-binding fragment comprising (a) a heavy chain variable region comprising the amino acid sequences set forth in SEQ ID NO: 17, SEQ ID NO: 18, and SEQ ID NO: 19; and (b) a light chain variable region comprising the amino acid sequences set forth in SEQ ID NO: 14, SEQ ID NO: 15, and SEQ ID NO: 63.

In certain embodiments, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWT (SEQ ID NO: 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWT (SEQ ID NO: 16)) is not acetylated. In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising the amino acid sequences of SEQ ID NOs: 14-16, wherein the second Q in QQYSTVPWT (SEQ ID NO: 16) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising the amino acid sequences of SEQ ID NOs: 14-16, wherein the two Ns in SASQDISNYLN (SEQ ID NO: 14) each carries one or more of the following chemical modifications: oxidation, acetylation, deamidation, and pyroglutamation (pyro Glu), and the second Q in QQYSTVPWT (SEQ ID NO: 16) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising the amino acid sequences of SEQ ID NOs: 14-16, wherein the second Q in QQYSTVPWT (SEQ ID NO: 16) is not acetylated. In a specific embodiment, the anti-VEGF antigen-binding fragment transgene encodes an antigen-binding fragment comprising a light chain variable region comprising the amino acid sequences of SEQ ID NOs: 14-16, wherein the two Ns in SASQDISNYLN (SEQ ID NO: 14) each carries one or more of the following chemical modifications: oxidation, acetylation, deamidation, and pyroglutamation (pyro Glu), and the second Q in QQYSTVPWT (SEQ ID NO: 16) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising light chain CDRs 1-3 of SEQ ID NOs: 14, 15, and 63, wherein the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu). In certain embodiments, the anti-VEGF antigen-binding fragment transgene encodes an antigen-binding fragment comprising a light chain variable region comprising light chain CDRs 1-3 of SEQ ID NOs: 14, 15, and 63, wherein the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14, 15, and 63, wherein the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14, 15, and 63, wherein the second amino acid residue of the light chain CDR3 (i.e., the second Q in QQYSTVPWTF (SEQ ID NO: 63)) is not acetylated. In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising the amino acid sequences of SEQ ID NOs: 14, 15, and 63, wherein the second Q in QQYSTVPWTF (SEQ ID NO: 63) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising the amino acid sequences of SEQ ID NOs: 14, 15, and 63, wherein the two Ns in SASQDISNYLN (SEQ ID NO: 14) each carries one or more of the following chemical modifications: oxidation, acetylation, deamidation, and pyroglutamation (pyro Glu), and the second Q in QQYSTVPWTF (SEQ ID NO: 63) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising the amino acid sequences of SEQ ID NOs: 14, 15, and 63, wherein the second Q in QQYSTVPWTF (SEQ ID NO: 63) is not acetylated. In a specific embodiment, the anti-VEGF antigen-binding fragment transgene encodes an antigen-binding fragment comprising a light chain variable region comprising the amino acid sequences of SEQ ID NOs: 14, 15, and 63, wherein the two Ns in SASQDISNYLN (SEQ ID NO: 14) each carries one or more of the following chemical modifications: oxidation, acetylation, deamidation, and pyroglutamation (pyro Glu), and the second Q in QQYSTVPWTF (SEQ ID NO: 63) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO: 20)) is not acetylated. In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a heavy chain variable region comprising the amino acid sequences of SEQ ID NOs: 20, 18, and 21, wherein the N in GYDFTHYGMN (SEQ ID NO: 20) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising the amino acid sequences of SEQ ID NOs: 20, 18, and 21, wherein the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising the amino acid sequences of SEQ ID NOs: 20, 18, and 21, wherein the N in GYDFTHYGMN (SEQ ID NO: 20) is not acetylated. In a specific embodiment, the anti-VEGF antigen-binding fragment transgene encodes an antigen-binding fragment comprising a heavy chain variable region comprising the amino acid sequences of SEQ ID NOs: 20, 18, and 21, wherein the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWT (SEQ ID NO: 16)) does not carry one or more of the following chemical modifications: oxidation, acetylation, deamidation, and pyroglutamation (pyro Glu), and wherein the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWT (SEQ ID NO: 16)) is not acetylated, and wherein the last amino acid residue of the heavy chain CDR1 (i.e., the N in GYDFTHYGMN (SEQ ID NO: 20)) is not acetylated. In a specific embodiment, the antigen-binding fragment comprises a heavy chain CDR1 of SEQ ID NO: 20, wherein: (1) the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) is not acetylated; and (2) the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising the amino acid sequences of SEQ ID NOs: 14-16 and a heavy chain variable region comprising the amino acid sequences of SEQ ID NOs: 20, 18, and 21, wherein the second Q in QQYSTVPWT (SEQ ID NO: 16) does not carry one or more of the following chemical modifications: oxidation, acetylation, deamidation, and pyroglutamation (pyro Glu), and wherein the N in GYDFTHYGMN (SEQ ID NO: 20) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising the amino acid sequences of SEQ ID NOs: 14-16 and a heavy chain variable region comprising the amino acid sequences of SEQ ID NOs: 20, 18, and 21, wherein: (1) the M in GYDFTHYGMN (SEQ ID NO: 20) carries one or more of the following chemical modifications: acetylation, deamidation, and pyroglutamation (pyro Glu), he N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) does not carry one or more of the following chemical modifications: oxidation, acetylation, deamidation, and pyroglutamation (pyro Glu); and (2) the two Ns in SASQDISNYLN (SEQ ID NO: 14) each carries one or more of the following chemical modifications: oxidation, acetylation, deamidation, and pyroglutamation (pyro Glu), and the second Q in QQYSTVPWT (SEQ ID NO: 16) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising the amino acid sequences of SEQ ID NOs: 14-16 and a heavy chain variable region comprising the amino acid sequences of SEQ ID NOs: 20, 18, and 21, wherein the second Q in QQYSTVPWT (SEQ ID NO: 16) is not acetylated, and wherein the N in GYDFTHYGMN (SEQ ID NO: 20) is not acetylated. In a specific embodiment, the antigen-binding fragment comprises the amino acid sequence of SEQ ID NO: 20, wherein: (1) the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) is not acetylated; and (2) the two Ns in SASQDISNYLN (SEQ ID NO: 14) each carries one or more of the following chemical modifications: oxidation, acetylation, deamidation, and pyroglutamation (pyro Glu), and second Q in QQYSTVPWT (SEQ ID NO: 16) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising light chain CDRs 1-3 of SEQ ID NOs: 14, 15, and 63 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu), and wherein the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14, 15, and 63 and a heavy chain variable region comprising heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14, 15, and 63 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO: 63)) is not acetylated, and wherein the last amino acid residue of the heavy chain CDR1 (i.e., the N in GYDFTHYGMN (SEQ ID NO: 20)) is not acetylated. In a specific embodiment, the antigen-binding fragment comprises a heavy chain CDR1 of SEQ ID NO: 20, wherein: (1) the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) is not acetylated; and (2) the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising the amino acid sequences of SEQ ID NOs: 14, 15, and 63 and a heavy chain variable region comprising the amino acid sequences of SEQ ID NOs: 20, 18, and 21, wherein the second Q in QQYSTVPWTF (SEQ ID NO: 63) does not carry one or more of the following chemical modifications: oxidation, acetylation, deamidation, and pyroglutamation (pyro Glu), and wherein the N in GYDFTHYGMN (SEQ ID NO: 20) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising the amino acid sequences of SEQ ID NOs: 14, 15, and 63 and a heavy chain variable region comprising the amino acid sequences of SEQ ID NOs: 20, 18, and 21, wherein: (1) the M in GYDFTHYGMN (SEQ ID NO: 20) carries one or more of the following chemical modifications: acetylation, deamidation, and pyroglutamation (pyro Glu), he N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) does not carry one or more of the following chemical modifications: oxidation, acetylation, deamidation, and pyroglutamation (pyro Glu); and (2) the two Ns in SASQDISNYLN (SEQ ID NO: 14) each carries one or more of the following chemical modifications: oxidation, acetylation, deamidation, and pyroglutamation (pyro Glu), and the second Q in QQYSTVPWTF (SEQ ID NO: 63) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising the amino acid sequences of SEQ ID NOs: 14, 15, and 63 and a heavy chain variable region comprising the amino acid sequences of SEQ ID NOs: 20, 18, and 21, wherein the second Q in QQYSTVPWTF (SEQ ID NO: 63) is not acetylated, and wherein the N in GYDFTHYGMN (SEQ ID NO: 20) is not acetylated. In a specific embodiment, the antigen-binding fragment comprises the amino acid sequence of SEQ ID NO: 20, wherein: (1) the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) is not acetylated; and (2) the two Ns in SASQDISNYLN (SEQ ID NO: 14) each carries one or more of the following chemical modifications: oxidation, acetylation, deamidation, and pyroglutamation (pyro Glu), and second Q in QQYSTVPWTF (SEQ ID NO: 63) is not acetylated. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the second amino acid residue of the light chain CDR3 (i.e., the second Q in QQYSTVPWT (SEQ ID NO: 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWT (SEQ ID NO: 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising the amino acid sequences of SEQ ID NOs: 14-16 and the amino acid sequences of SEQ ID NOs: 20, 18, and 21, and transgenes encoding such antigen-VEGF antigen-binding fragments, wherein the second Q in QQYSTVPWT (SEQ ID NO: 16) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14-16 and the amino acid sequences of SEQ ID NOs: 20, 18, and 21, wherein the two Ns in SASQDISNYLN (SEQ ID NO: 14) each carries one or more of the following chemical modifications: oxidation, acetylation, deamidation, and pyroglutamation (pyro Glu), and the second Q in QQYSTVPWT (SEQ ID NO: 16) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14-16 and the amino acid sequences of SEQ ID NOs: 20, 18, and 21, wherein the second Q in QQYSTVPWT (SEQ ID NO: 16) is not acetylated. In a specific embodiment, the antigen-binding fragment comprises the amino acid sequences of SEQ ID NOs: 14-16 and the amino acid sequences of SEQ ID NOs: 20, 18, and 21, wherein the two Ns in SASQDISNYLN (SEQ ID NO: 14) each carries one or more of the following chemical modifications: oxidation, acetylation, deamidation, and pyroglutamation (pyro Glu), and the second Q in QQYSTVPWT (SEQ ID NO: 16) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14, 15, and 63 and heavy chain CDRs 1-3 of SEQ ID NOs: 20, 18, and 21, and transgenes encoding such antigen-VEGF antigen-binding fragments, wherein the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein the second amino acid residue of the light chain CDR3 (i.e., the second Q in QQYSTVPWTF (SEQ ID NO: 63)) is not acetylated. In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising the amino acid sequences of SEQ ID NOs: 14, 15, and 63 and the amino acid sequences of SEQ ID NOs: 20, 18, and 21, and transgenes encoding such antigen-VEGF antigen-binding fragments, wherein the second Q in QQYSTVPWTF (SEQ ID NO: 63) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14, 15, and 63 and the amino acid sequences of SEQ ID NOs: 20, 18, and 21, wherein the two Ns in SASQDISNYLN (SEQ ID NO: 14) each carries one or more of the following chemical modifications: oxidation, acetylation, deamidation, and pyroglutamation (pyro Glu), and the second Q in QQYSTVPWTF (SEQ ID NO: 63) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14, 15, and 63 and the amino acid sequences of SEQ ID NOs: 20, 18, and 21, wherein the second Q in QQYSTVPWTF (SEQ ID NO: 63) is not acetylated. In a specific embodiment, the antigen-binding fragment comprises the amino acid sequences of SEQ ID NOs: 14, 15, and 63 and the amino acid sequences of SEQ ID NOs: 20, 18, and 21, wherein the two Ns in SASQDISNYLN (SEQ ID NO: 14) each carries one or more of the following chemical modifications: oxidation, acetylation, deamidation, and pyroglutamation (pyro Glu), and the second Q in QQYSTVPWTF (SEQ ID NO: 63) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO: 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising the amino acid sequences of SEQ ID NOs: 14-16 and the amino acid sequences of SEQ ID NOs: 20, 18, and 21, and transgenes encoding such antigen-VEGF antigen-binding fragments, wherein the N in GYDFTHYGMN (SEQ ID NO: 20) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14-16 and the amino acid sequences of SEQ ID NOs: 20, 18, and 21, wherein the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14-16 and the amino acid sequences of SEQ ID NOs: 20, 18, and 21, wherein the N in GYDFTHYGMN (SEQ ID NO: 20) is not acetylated. In a specific embodiment, the antigen-binding fragment comprises the amino acid sequences of SEQ ID NOs: 14-16 and the amino acid sequences of SEQ ID NOs: 20, 18, and 21, wherein the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14, 15, and 63 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein the last amino acid residue of the heavy chain CDR1 (i.e., the N in GYDFTHYGMN (SEQ ID NO: 20)) is not acetylated. In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising the amino acid sequences of SEQ ID NOs: 14, 15, and 63 and the amino acid sequences of SEQ ID NOs: 20, 18, and 21, and transgenes encoding such antigen-VEGF antigen-binding fragments, wherein the N in GYDFTHYGMN (SEQ ID NO: 20) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14, 15, and 63 and the amino acid sequences of SEQ ID NOs: 20, 18, and 21, wherein the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14, 15, and 63 and the amino acid sequences of SEQ ID NOs: 20, 18, and 21, wherein the N in GYDFTHYGMN (SEQ ID NO: 20) is not acetylated. In a specific embodiment, the antigen-binding fragment comprises the amino acid sequences of SEQ ID NOs: 14, 15, and 63 and the amino acid sequences of SEQ ID NOs: 20, 18, and 21, wherein the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO: 20)) is not acetylated, and the second amino acid residue of the light chain CDR3 (i.e., the second Q in QQYSTVPWT (SEQ ID NO: 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) is not acetylated; and (2) the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWT (SEQ ID NO: 16)) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising the amino acid sequences of SEQ ID NOs: 14-16 and the amino acid sequences of SEQ ID NOs: 20, 18, and 21, and transgenes encoding such antigen-VEGF antigen-binding fragments, wherein the N in GYDFTHYGMN (SEQ ID NO: 20) does not carry one or more of the following chemical modifications: oxidation, acetylation, deamidation, and pyroglutamation (pyro Glu), and the second Q in QQYSTVPWT (SEQ ID NO: 16) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14-16 and the amino acid sequences of SEQ ID NOs: 20, 18, and 21, wherein: (1) the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) does not carry one or more of the following chemical modifications: oxidation, acetylation, deamidation, and pyroglutamation (pyro Glu); and (2) the two Ns in SASQDISNYLN (SEQ ID NO: 14) each carries one or more of the following chemical modifications: oxidation, acetylation, deamidation, and pyroglutamation (pyro Glu), and the second Q in QQYSTVPWT (SEQ ID NO: 16) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14-16 and the amino acid sequences of SEQ ID NOs: 20, 18, and 21, wherein the N in GYDFTHYGMN (SEQ ID NO: 20) is not acetylated, and the second Q in QQYSTVPWT (SEQ ID NO: 16) is not acetylated. In a specific embodiment, the antigen-binding fragment comprises the amino acid sequences of SEQ ID NOs: 14-16 and the amino acid sequences of SEQ ID NOs: 20, 18, and 21, wherein: (1) the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) is not acetylated; and (2) the two Ns in SASQDISNYLN (SEQ ID NO: 14) each carries one or more of the following chemical modifications: oxidation, acetylation, deamidation, and pyroglutamation (pyro Glu), and the second Q in QQYSTVPWT (SEQ ID NO: 16) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14, 15, and 63 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein the last amino acid residue of the heavy chain CDR1 (i.e., the N in GYDFTHYGMN (SEQ ID NO: 20)) is not acetylated, and the second amino acid residue of the light chain CDR3 (i.e., the second Q in QQYSTVPWTF (SEQ ID NO: 63)) is not acetylated. In a specific embodiment, the antigen-binding fragment comprises light chain CDRs 1-3 of SEQ ID NOs: 14, 15, and 63 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO: 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO: 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO: 20)) is not acetylated; and (2) the eighth and eleventh amino acid residues of the light chain CDR1 (i.e., the two Ns in SASQDISNYLN (SEQ ID NO: 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO: 63)) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising the amino acid sequences of SEQ ID NOs: 14, 16, and 63 and the amino acid sequences of SEQ ID NOs: 20, 18, and 21, and transgenes encoding such antigen-VEGF antigen-binding fragments, wherein the N in GYDFTHYGMN (SEQ ID NO: 20) does not carry one or more of the following chemical modifications: oxidation, acetylation, deamidation, and pyroglutamation (pyro Glu), and the second Q in QQYSTVPWTF (SEQ ID NO: 63) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14, 16, and 63 and the amino acid sequences of SEQ ID NOs: 20, 18, and 21, wherein: (1) the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) does not carry one or more of the following chemical modifications: oxidation, acetylation, deamidation, and pyroglutamation (pyro Glu); and (2) the two Ns in SASQDISNYLN (SEQ ID NO: 14) each carries one or more of the following chemical modifications: oxidation, acetylation, deamidation, and pyroglutamation (pyro Glu), and the second Q in QQYSTVPWTF (SEQ ID NO: 63) does not carry one or more of the following chemical modifications: oxidation, acetylation, deamidation, and pyroglutamation (pyro Glu). In a specific embodiment, the antigen-binding fragment comprises the amino acid sequences of SEQ ID NOs: 14, 16, and 63 and the amino acid sequences of SEQ ID NOs: 20, 18, and 21, wherein the N in GYDFTHYGMN (SEQ ID NO: 20) is not acetylated, and the second Q in QQYSTVPWTF (SEQ ID NO: 63) is not acetylated. In a specific embodiment, the antigen-binding fragment comprises the amino acid sequences of SEQ ID NOs: 14, 16, and 63 and the amino acid sequences of SEQ ID NOs: 20, 18, and 21, wherein: (1) the M in GYDFTHYGMN (SEQ ID NO: 20) carries one or more of the following chemical modifications: acetylation, deamidation, and pyroglutamation (pyro Glu), the N in WINTYTGEPTYAADFKR (SEQ ID NO: 18) carries one or more of the following chemical modifications: acetylation, deamidation, and pyroglutamation (pyro Glu), and the N in GYDFTHYGMN (SEQ ID NO: 20) is not acetylated; and (2) the two Ns in SASQDISNYLN (SEQ ID NO: 14) each carries one or more of the following chemical modifications: oxidation, acetylation, deamidation, and pyroglutamation (pyro Glu), and the second Q in QQYSTVPWTF (SEQ ID NO: 63) is not acetylated. The anti-VEGF antigen-binding fragments and transgenes provided herein can be used in any method according to the embodiments described herein. In a preferred embodiment, the chemical modification(s) or lack of chemical modification(s) (as the case may be) described herein is determined by mass spectrometry.

TABLE 2
Exemplary transgene sequences

| SEQ ID | VEGF antigen- | |
|---|---|---|
| NO. | binding fragment | Sequence |
| 1 | ranibizumab Fab | DIQLTQSPSSLSASVGDRVTITCSASQDISNYLNWYQQKP |
| | Amino Acid | GKAPKVLIYFTSSLHSGVPSRFSGSGSGTDFTLTISSLQPED |
| | Sequence (Light | FATYYCQQYSTVPWTFGQGTKVEIKRTVAAPSVFIFPPSD |
| | chain) | EQLKSGTASVVCLLNNFYPREAKVQWKVDNALQSGNSQ |
| | | ESVTEQDSKDSTYSLSSTLTLSKADYEKHKVYACEVTHQ |
| | | GLSSPVTKSFNRGEC |
| 2 | ranibizumab Fab | EVQLVESGGGLVQPGGSLRLSCAASGYDFTHYGMNWVR |
| | Amino Acid | QAPGKGLEWVGWINTYTGEPTYAADFKRRFTFSLDTSKS |
| | Sequence (Heavy | TAYLQMNSLRAEDTAVYYCAKYPYYYGTSHWYFDVWG |
| | chain) | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHL |
| 3 | bevacizumab Fab | DIQMTQSPSSLSASVGDRVTITCSASQDISNYLNWYQQKP |
| | Amino Acid | GKAPKVLIYFTSSLHSGVPSRFSGSGSGTDFTLTISSLQPED |
| | Sequence (Light | FATYYCQQYSTVPWTFGQGTKVEIKRTVAAPSVFIFPPSD |
| | chain) | EQLKSGTASVVCLLNNFYPREAKVQWKVDNALQSGNSQ |
| | | ESVTEQDSKDSTYSLSSTLTLSKADYEKHKVYACEVTHQ |
| | | GLSSPVTKSFNRGEC |
| 4 | bevacizumab Fab | EVQLVESGGGLVQPGGSLRLSCAASGYTFTNYGMNWVR |
| | Amino Acid | QAPGKGLEWVGWINTYTGEPTYAADFKRRFTFSLDTSKS |
| | Sequence (Heavy | TAYLQMNSLRAEDTAVYYCAKYPHYYGSSHWYFDVWG |
| | chain) | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHL |
| 10 | bevacizumab | gctagcgccaccatgggctggtcctgcatcatcctgttcctggtggccaccgccaccggc |
| | cDNA | gtgcactccgacatccagatgacccagtccccctcctccctgtccgcctccgtgggcgacc |
| | (Light chain) | gggtgaccatcacctgctccgcctcccaggacatctccaactacctgaactggtaccagca |
| | | gaagcccggcaaggcccccaaggtgctgatctacttcacctcctccctgcactccggcgt |
| | | gccctcccggttctccggctccggctccggcaccgacttcaccctgaccatctcctccctg |
| | | cagcccgaggacttcgccacctactactgccagcagtactccaccgtgccctggaccttcg |
| | | gccagggcaccaaggtggagatcaagcggaccgtggccgccccctccgtgttcatcttcc |
| | | ccccctccgacgagcagctgaagtccggcaccgcctccgtggtgtgcctgctgaacaact |
| | | tctacccccgggaggccaaggtgcagtggaaggtggacaacgccctgcagtccggcaa |
| | | ctcccaggagtccgtgaccgagcaggactccaaggactccacctactccctgtcctccac |
| | | cctgaccctgtccaaggccgactacgagaagcacaaggtgtacgcctgcgaggtgaccc |
| | | accagggcctgtcctcccccgtgaccaagtccttcaaccggggcgagtgctgagcggcc |
| | | gcctcgag |
| 11 | bevacizumab | gctagcgccaccatgggctggtcctgcatcatcctgttcctggtggccaccgccaccggc |
| | cDNA (Heavy | gtgcactccgaggtgcagctggtggagtccggcggcggcctggtgcagcccggcggct |
| | chain) | ccctgcggctgtcctgcgccgcctccggctacaccttcaccaactacggcatgaactgggt |
| | | gcggcaggcccccggcaagggcctggagtggggggctggatcaacacctacaccgg |
| | | cgagcccacctacgccgccgacttcaagcggcggttcaccttctccctggacacctccaa |
| | | gtccaccgcctacctgcagatgaactccctgcgggccgaggacaccgccgtgtactactg |
| | | cgccaagtacccccactactacggctcctcccactggtacttcgacgtgtggggccaggg |
| | | caccctggtgaccgtgtcctccgcctccaccaagggcccctccgtgttccccctggccccc |
| | | tcctccaagtccacctccggcggcaccgccgccctgggctgcctggtgaaggactacttc |
| | | cccgagcccgtgaccgtgtcctggaactccggcgccctgacctccggcgtgcacaccttc |
| | | cccgccgtgctgcagtcctccggcctgtactccctgtcctccgtggtgaccgtgccctcctc |
| | | ctccctgggcacccagacctacatctgcaacgtgaaccacaagccctccaacaccaaggt |
| | | ggacaagaaggtggagcccaagtcctgcgacaagacccacacctgccccccctgcccc |
| | | gcccccgagctgctgggcggcccctccgtgttcctgttcccccccaagcccaaggacacc |
| | | ctgatgatctcccggacccccgaggtgacctgcgtggtggtggacgtgtcccacgaggac |
| | | cccgaggtgaagttcaactggtacgtggacggcgtggaggtgcacaacgccaagaccaa |
| | | gccccgggaggagcagtacaactccacctaccgggtggtgtccgtgctgaccgtgctgc |
| | | accaggactggctgaacggcaaggagtacaagtgcaaggtgtccaacaaggccctgcc |
| | | cgcccccatcgagaagaccatctccaaggccaagggccagccccgggagccccaggt |
| | | gtacaccctgcccccctcccgggaggagatgaccaagaaccaggtgtccctgacctgcct |
| | | ggtgaagggcttctacccctccgacatcgccgtggagtgggagtccaacggccagcccg |
| | | agaacaactacaagaccaccccccccgtgctggactccgacggctccttcttcctgtactc |
| | | caagctgaccgtggacaagtcccggtggcagcagggcaacgtgttctcctgctccgtgat |
| | | gcacgaggccctgcacaaccactacacccagaagtccctgtccctgtcccccggcaagt |
| | | gagcggccgcc |
| 12 | ranibizumab cDNA | gagctccatggagtttttcaaaaagacggcacttgccgcactggttatgggttttagtggtgc |
| | (Light chain | agcattggccgatatccagctgacccagagcccgagcagcctgagcgcaagcgttggtg |
| | comprising a signal | atcgtgttaccattacctgtagcgcaagccaggatattagcaattatctgaattggtatcagca |
| | sequence) | gaaaccgggtaaagcaccgaaagttctgatttattttaccagcagcctgcatagcggtgttc |
| | | cgagccgttttagcggtagcggtagtggcaccgattttaccctgaccattagcagcctgca |
| | | gccggaagattttgcaacctattattgtcagcagtatagcaccgttccgtggacctttggtca |
| | | gggcaccaaagttgaaattaaacgtaccgttgcagcaccgagcgtttttatttttccgcctag |
| | | tgatgaacagctgaaaagcggcaccgcaagcgttgtttgtctgctgaataatttttatccgcg |
| | | tgaagcaaaagtgcagtggaaagttgataatgcactgcagagcggtaatagccaagaaag |
| | | cgttaccgaacaggatagcaaagatagcacctatagcctgag |
| | | cagcaccctgaccctgagcaaagcagattatgaaaaacacaaagtgtatgcctgcgaagtt |
| | | acccatcagggtctgagcagtccggttaccaaaagttttaatcgtggcgaatgctaatagaa |
| | | gcttggtacc |
| 13 | ranibizumab cDNA | gagctcatatgaaatacctgctgccgaccgctgctgctggtctgctgctcctcgctgcccag |
| | (Heavy chain | ccggcgatggccgaagttcagctggttgaaagcggtggtggtctggttcagcctggtggta |
| | comprising a signal | gcctgcgtctgagctgtgcagcaagcggttatgattttacccattatggtatgaattgggttcg |
| | sequence) | tcaggcaccgggtaaaggtctggaatgggttggttggattaatacctataccggtgaaccg |
| | | acctatgcagcagattttaaacgtcgttttacctttagcctggataccagcaaaagcaccgca |
| | | tatctgcagatgaatagcctgcgtgcagaagataccgcagtttattattgtgccaaatatccg |
| | | tattactatggcaccagccactggtatttcgatgtttggggtcagggcaccctggttaccgtt |
| | | agcagcgcaagcaccaaaggtccgagcgtttttccgctggcaccgagcagcaaaagtac |
| | | cagcggtggcacagcagcactgggttgtctggttaaagattattttccggaaccggttaccg |
| | | tgagctggaatagcggtgcactgaccagcggtgttcatacct |
| | | ttccggcagttctgcagagcagcggtctgtatagcctgagcagcgttgttaccgttccgagc |
| | | agcagcctgggcacccagacctatatttgtaatgttaatcataaaccgagcaataccaaagt |
| | | ggataaaaaagttgagccgaaaagctgcgataaaacccatctgtaatagggtacc |
| 14, 15, | bevacizumab Light | SASQDISNYLN (SEQ ID NO: 14) |
| and 16 | Chain CDRs | FTSSLHS (SEQ ID NO: 15) |
| | | QQYSTVPWT (SEQ ID NO: 16) |
| 17, 18, | bevacizumab | GYTFTNYGMN (SEQ ID NO: 17) |
| and 19 | Heavy Chain CDRs | WINTYTGEPTYAADFKR (SEQ ID NO: 18) |
| | | YPHYYGSSHWYFDV (SEQ ID NO: 19) |
| 14, 15, | ranibizumab Light | SASQDISNYLN (SEQ ID NO: 14) |
| and 16 | Chain CDRs | FTSSLHS (SEQ ID NO: 15) |
| | | QQYSTVPWT (SEQ ID NO: 16) |
| 20, 18, | ranibizumab Heavy | GYDFTHYGMN (SEQ ID NO: 20) |
| and 21 | Chain CDRs | WINTYTGEPTYAADFKR (SEQ ID NO: 18) |
| | | YPYYYGTSHWYFDV (SEQ ID NO: 21) |

### 5.2.9 Constructs

In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety). In certain embodiments, the sequence encoding the transgene comprises multiple ORFs separated by IRES elements. In certain embodiments, the ORFs encode the heavy and light chain domains of the anti-VEGF antigen-binding fragment. In certain embodiments, the sequence encoding the transgene comprises multiple subunits in one ORF separated by F/F2A sequences. In certain embodiments, the sequence comprising the transgene encodes the heavy and light chain domains of the anti-VEGF antigen-binding fragment separated by an F/F2A sequence. In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety), wherein the transgene comprises the signal peptide of VEGF-A (SEQ ID NO: 5), and wherein the transgene encodes a light chain and a heavy chain sequence separated by an IRES element. In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety), wherein the transgene comprises the signal peptide of VEGF-A (SEQ ID NO: 5), and wherein the transgene encodes a light chain and a heavy chain sequence separated by a cleavable F/F2A sequence.

In certain embodiments, the viral vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, 1) a fifth linker sequence, and m) a second ITR sequence.

In certain embodiments, the viral vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, 1) a fifth linker sequence, and m) a second ITR sequence, wherein the transgene comprises the signal peptide of VEGF-A (SEQ ID NO: 5), and wherein the transgene encodes a light chain and a heavy chain sequence separated by a cleavable F/F2A sequence.

In some embodiments, the AAV (AAV viral vectors) provided herein comprise the following elements in the following order: a) a constitutive or a hypoxia-inducible promoter sequence, and b) a sequence encoding the transgene (e.g., an anti-VEGF antigen-binding fragment moiety). In some embodiments, the transgene is a fully human post-translationally modified (HuPTM) antibody against VEGF. In some embodiments, the fully human post-translationally modified antibody against VEGF is a fully human post-translationally modified antigen-binding fragment of a monoclonal antibody (mAb) against VEGF (“HuPTMFabVEGFi”). In some embodiments, the HuPTMFabVEGFi is a fully human glycosylated antigen-binding fragment of an anti-VEGF mAb (“HuGlyFabVEGFi”). In an alternative embodiment, full-length mAbs can be used. In some embodiments, the AAV used for delivering the transgene should have a tropism for human retinal cells or photoreceptor cells. Such AAV can include non-replicating recombinant adeno-associated virus vectors (“rAAV”), particularly those bearing an AAV8 capsid are preferred. In a specific embodiment, the viral vector or other DNA expression construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In some embodiments, the recombinant viral vector comprises the nucleotide sequence of SEQ ID NO: 56. In some embodiments, the viral vector comprises a vector genome comprising SEQ ID NO: 56 (ITR-CB7-CI-aVEGFv3-rBG-ITR). In some embodiments, the vector genome comprises SEQ ID NO: 14 of U.S. Ser. No. 11/197,937 (incorporated herein by reference). In some embodiments, the vector genome comprises any one of the sequences disclosed in U.S. Ser. No. 11/197,937 (incorporated herein by reference). In some embodiments, the vector genome comprises a 5′ AAV-2 inverted terminal repeat, an expression cassette consisting of the contiguous nucleotides 198 to 3733 of SEQ ID NO: 56 (equivalent to SEQ ID NO: 14 of U.S. Ser. No. 11/197,937 (the patent is herein incorporated by reference), and a 3′ AAV-2 inverted terminal repeat. In some embodiments, the viral vector comprises a signal peptide. In some embodiments, the signal peptide is MYRMQLLLLIALSLALVTNS (SEQ ID NO: 55). In some embodiments, the signal peptide is derived from IL-2 signal sequence. In some embodiments, the viral vector comprises a signal peptide from any signal peptide disclosed in Table 1, such as MNFLLSWVHWSLALLLYLHHAKWSQA (VEGF-A signal peptide) (SEQ ID NO: 5); MERAAPSRRVPLPLLLLGGLALLAAGVDA (Fibulin-1 signal peptide) (SEQ ID NO: 6); MAPLRPLLILALLAWVALA (Vitronectin signal peptide) (SEQ ID NO: 7); MRLLAKIICLMLWAICVA (Complement Factor H signal peptide) (SEQ ID NO: 8); MRLLAFLSLLALVLQETGT (Opticin signal peptide) (SEQ ID NO: 9); MKWVTFISLLFLFSSAYS (Albumin signal peptide) (SEQ ID NO: 22); MAFLWLLSCWALLGTTFG (Chymotrypsinogen signal peptide) (SEQ ID NO: 23); MYRMQLLSCIALILALVTNS (Interleukin-2 signal peptide) (SEQ ID NO: 24); MNLLLILTFVAAAVA (Trypsinogen-2 signal peptide) (SEQ ID NO: 25); or MYRMQLLLLIALSLALVTNS (mutant Interleukin-2 signal peptide) (SEQ ID NO: 55). In some embodiments, the recombinant viral vector encodes a polypeptide comprising the amino acid sequence of SEQ ID NO: 57. In some embodiments, the recombinant viral vector encodes a polypeptide comprising the amino acid sequence of SEQ ID NO: 58. In some embodiments, the recombinant viral vector encodes a first polypeptide comprising the amino acid sequence of SEQ ID NO: 57 and a second polypeptide comprising the amino acid sequence of SEQ ID NO: 58. In certain embodiments, the signal peptide is removed from the first and second polypeptide after translation such that the secreted transgene product comprises a light chain comprising the amino acid sequence of SEQ ID NO: 1 and a heavy chain comprising the amino acid sequence of SEQ ID NO: 2. In some embodiments, the C-terminal lysine of SEQ ID NO: 2 is removed from the transgene product after translation and before the transgene product is secreted.

In a specific embodiment, the construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

In another specific embodiment, the construct described herein is Construct II, wherein the Construct II comprises the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In a specific embodiment, the construct comprises an expression cassette encoding an anti-hVEGF antigen-binding fragment, wherein the expression cassette is flanked by AAV2 inverted terminal repeats (ITRs), and wherein the expression cassette comprises:
a CB7 promotor consisting of a chicken j-actin promoter and a CMV enhancer;
a chicken β-actin intron;
a nucleotide sequence encoding:
   an IL-2 signal peptide;
   a heavy chain of the anti-hVEGF antigen-binding fragment comprising the amino acid sequence of SEQ ID NO: 2;
   a self-cleaving furin (F)/F2A linker;
   a second IL-2 signal peptide; and
   a light chain of the anti-hVEGF antigen-binding fragment comprising the amino acid sequence of SEQ ID NO: 1; and
a rabbit β-globin poly A signal.

### 5.2.10 Manufacture and Testing of Vectors

The viral vectors provided herein may be manufactured using host cells. The viral vectors provided herein may be manufactured using mammalian host cells, for example, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (i.e., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

In vitro assays, e.g., cell culture assays, can be used to measure transgene expression from a vector described herein, thus indicating, e.g., potency of the vector. For example, the PER.C6@Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Once expressed, characteristics of the expressed product (i.e., HuGlyFabVEGFi) can be determined, including determination of the glycosylation and tyrosine sulfation patterns associated with the HuGlyFabVEGFi. Glycosylation and tyrosine sulfation patterns, and methods of determining, the same are discussed in PCT/US2017/027650, which is incorporated herein by reference. In addition, benefits resulting from glycosylation/sulfation of the cell-expressed HuGlyFabVEGFi can be determined using assays known in the art, e.g., the methods described in PCT/US2017/027650.

### 5.2.11 Compositions

Compositions are described comprising a vector encoding a transgene described herein and a suitable carrier. A suitable carrier (e.g., for suprachoroidal, subretinal, juxtascleral, intravitreal, subconjunctival, and/or intraretinal administration) would be readily selected by one of skill in the art.

In certain embodiments of the methods provided herein, the recombinant viral vector is formulated to be suitable for administration to the suprachoroidal space of the eye of the subject. In certain embodiments, the recombinant viral vector is formulated to be suitable for administration to the subretinal space of the eye of the subject. In some embodiments, the formulated composition is a formulation and/or pharmaceutical composition described in WO2021/071835, WO2022/076549, WO2022/076591, or WO2022/076595, each of which is incorporated herein by reference. In some embodiments, the formulated composition comprises: (a) the recombinant viral vector (e.g., rHuGlyFabVEGFi), (b) potassium chloride at a concentration of about 0.2 g/L, (c) potassium phosphate monobasic at a concentration of about 0.2 g/L, (d) sodium chloride at a concentration of about 5.84 g/L, € sodium phosphate dibasic anhydrous at a concentration of about 1.15 g/L, (f) sucrose at a concentration of about 4% weight/volume (40 g/L), (g) poloxamer 188, polysorbate 20, or polysorbate 80 at a concentration of about 0.001% weight/volume (0.01 g/L), and (h) water, and wherein pH=about 7.4. In some embodiments, the formulated composition comprises: (a) the recombinant viral vector (e.g., rHuGlyFabVEGFi), (b) potassium chloride at a concentration of about 0.2 g/L, (c) potassium phosphate monobasic at a concentration of about 0.2 g/L, (d) sodium chloride at a concentration of about 5.84 g/L, € sodium phosphate dibasic anhydrous at a concentration of about 1.15 g/L, (f) sucrose at a concentration of about 4% weight/volume (40 g/L), (g) poloxamer 188 at a concentration of about 0.001% weight/volume (0.01 g/L), and (h) water, and wherein pH=about 7.4. In some embodiments, the formulated composition comprises: (a) the recombinant viral vector (e.g., rHuGlyFabVEGFi), (b) potassium chloride at a concentration of about 2.70 mM, (c) potassium phosphate monobasic at a concentration of about 1.47 mM, (d) sodium chloride at a concentration of about 100 mM, (e) sodium phosphate dibasic anhydrous at a concentration of about 8.10 mM, (f) sucrose at a concentration of about 117 mM (400 weight/volume), (g) poloxamer 188 at a concentration of about 0.0010 weight/volume (0.01 g/L), and (h) water, and wherein pH=about 7.4. In one embodiment, the formulated composition is described in the following table:

| | | | | | | Mass | Vendor | | Molecular |
|---|---|---|---|---|---|---|---|---|---|
| | | Quality | Concentration | Concentration | | Fraction | and Part | | Weight |
| Ingredient | Function | Standard | (mg/mL) | (mM or %) | | (g/kg)^{b} | Number | Chemical Formula | (g/mol) |
| Construct II | API | Internal | Varies | — | | — | — | — | — |
| | | | based on | | | | | | |
| | | | dose level | | | | | | |
| Sodium | Buffering | USP, | 5.84 | 100 | mM | 5.736 | Avantor, | NaCl | 58.440 |
| Chloride | Agent | Ph. Eur, | | | | | 3627 | | |
| | | BP, | | | | | | | |
| | | JPE | | | | | | | |
| Potassium | | USP, | 0.201 | 2.70 | mM | 0.198 | Avantor, | KCl | 74.5513 |
| Chloride | | BP, | | | | | 3045 | | |
| | | Ph. Eur, | | | | | | | |
| | | JPE | | | | | | | |
| Sodium | | USP, | 1.15 | 8.10 | mM | 1.129 | Avantor, | Na₂HPO₄ | 141.960 |
| Phosphate | | Ph. Eur, | | | | | 3804 | | |
| Dibasic | | JPE | | | | | | | |
| Anhydrous | | | | | | | | | |
| Potassium | | NF, BP, | 0.200 | 1.47 | mM | 0.196 | Avantor, | KH₂PO₄ | 136.086 |
| Phosphate | | Ph. Eur | | | | | 3248 | | |
| Monobasic | | | | | | | | | |
| Sucrose | Cryopro- | USP, | 40.0 | 117 | mM | 39.26 | Pfanstiehl, | C₁₂H₂₂O₁₁ | 342.3 |

| | | | | | | | | |
|---|---|---|---|---|---|---|---|---|
| | tectant | NF, | | | | S-124-2-MC | | |
| | | Ph. Eur, | | | | | | |
| | | BP, | | | | | | |
| | | JPE | | | | | | |
| Poloxamer | Surfactant^{a} | NF, | 0.010 | 0.001% | 0.1 mL/kg | BASF, | HO(C₃H₆O)ₐ(C₂H₄O)_{b}(C₃H₆O)ₐH | 7680 to 9510 |
| 188 | | Ph. Eur, | | | of 10% | 50424596 | | |
| | | JPE | | | stock | | | |
| Water | Aqueous | WFI | Approximately | Approximately | QS to | Varies | H₂O | 18.0153 |
| | Vehicle | | 971 mg/mL | 54M | 1 kg | | | |
| | | | | | (need | | | |
| | | | | | approx. | | | |
| | | | | | 953 g/kg) | | | |

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant (e.g., rHuGlyFabVEGFi) vector in a formulation buffer comprising a physiologically compatible aqueous buffer, a surfactant and optional excipients. In a specific embodiment, the gene therapy construct is formulated in Dulbecco's phosphate buffered saline and 0.001% Pluronic F68, pH=7.4.

### 5.3 Gene Therapy

Methods are described for the administration of a therapeutically effective amount of a transgene construct to human subjects having an ocular disease, in particular an ocular disease caused by increased neovascularization. More particularly, methods for administration of a therapeutically effective amount of a transgene construct to patients having neovascular age-related macular degeneration (nAMD) or diabetic retinopathy (DR), in particular, for suprachoroidal, subretinal, juxtascleral, intravitreal, subconjunctival, and/or intraretinal administration (e.g., by suprachoroidal injection, subretinal injection via the transvitreal approach (a surgical procedure), subretinal administration via the suprachoroidal space, or a posterior juxtascleral depot procedure), are described.

Methods are described for suprachoroidal, subretinal, juxtascleral, intravitreal, subconjunctival, and/or intraretinal administration of a therapeutically effective amount of a transgene construct to patients diagnosed with neovascular age-related macular degeneration or diabetic retinopathy (e.g., by suprachoroidal injection, subretinal injection via the transvitreal approach (a surgical procedure), or subretinal administration via the suprachoroidal space).

Also provided herein are methods for suprachoroidal, subretinal, juxtascleral, intravitreal, subconjunctival, and/or intraretinal of a therapeutically effective amount of a transgene construct (e.g., by suprachoroidal injection, subretinal injection via the transvitreal approach (a surgical procedure), subretinal administration via the suprachoroidal space, or a posterior juxtascleral depot procedure) and methods of administration of a therapeutically effective amount of a transgene construct to the retinal pigment epithelium.

### 5.3.1 Method for the Delivery of Recombinant Viral Vector

In one aspect, provided herein is a method of subretinal administration without vitrectomy for treating neovascular age-related macular degeneration or diabetic retinopathy, comprising administering to the subretinal space in the eye of a human subject in need of treatment a recombinant viral vector provided herein such that the transgene product is expressed and results in treatment of neovascular age-related macular degeneration or diabetic retinopathy, wherein the method does not comprise performing a vitrectomy on the eye of said human patient. In certain embodiments, the administering step comprises administering to the subretinal space in the eye of said human subject the recombinant viral vector via the suprachoroidal space in the eye of said human subject. In certain embodiments, the administering step is by the use of a subretinal drug delivery device comprising a catheter that can be inserted and tunneled through the suprachoroidal space toward the posterior pole, where a small needle injects into the subretinal space. In certain embodiments, the administering step comprises inserting and tunneling the catheter of the subretinal drug delivery device through the suprachoroidal space.

In another aspect, provided herein is a method for treating neovascular age-related macular degeneration or diabetic retinopathy, comprising administering to the subretinal space in the eye of a human subject in need of treatment a recombinant viral vector provided herein such that the transgene product is expressed and results in treatment of neovascular age-related macular degeneration or diabetic retinopathy, wherein the method does not comprise performing a vitrectomy on the eye of said human patient. In certain embodiments, the administering step comprises administering to the subretinal space in the eye of said human subject the recombinant viral vector via the suprachoroidal space in the eye of said human subject. In certain embodiments, the administering step is by the use of a subretinal drug delivery device comprising a catheter that can be inserted and tunneled through the suprachoroidal space toward the posterior pole, where a small needle injects into the subretinal space. In certain embodiments, the administering step comprises inserting and tunneling the catheter of the subretinal drug delivery device through the suprachoroidal space.

In one aspect, provided herein is a method of subretinal administration with vitrectomy for treating neovascular age-related macular degeneration or diabetic retinopathy, comprising administering to the subretinal space in the eye of a human subject in need of treatment a recombinant viral vector provided herein such that the transgene product is expressed and results in treatment of neovascular age-related macular degeneration or diabetic retinopathy, wherein the method comprises performing a vitrectomy on the eye of said human patient. In certain embodiments, the vitrectomy is a partial vitrectomy.

In another aspect, provided herein is a method for treating neovascular age-related macular degeneration or diabetic retinopathy, comprising administering to the subretinal space in the eye of a human subject in need of treatment a recombinant viral vector provided herein such that the transgene product is expressed and results in treatment of neovascular age-related macular degeneration or diabetic retinopathy, wherein the method comprises performing a vitrectomy on the eye of said human patient. In certain embodiments, the vitrectomy is a partial vitrectomy.

In a preferred embodiment, provided herein is a method of suprachoroidal administration for treating neovascular age-related macular degeneration or diabetic retinopathy, comprising administering to the suprachoroidal space in the eye of a human subject in need of treatment a recombinant viral vector provided herein such that the transgene product is expressed and results in treatment of neovascular age-related macular degeneration or diabetic retinopathy. In certain embodiments, the administering step is by injecting the recombinant viral vector into the suprachoroidal space using a suprachoroidal drug delivery device. In certain embodiments, the suprachoroidal drug delivery device is a microinjector.

In certain embodiments, delivery to the subretinal or suprachoroidal space can be performed using the methods and/or devices described and disclosed in International Publication Nos. WO 2016/042162, WO 2017/046358, WO 2017/158365, and WO 2017/158366, each of which is incorporated by reference in its entirety.

Currently available technologies for suprachoroidal space (SCS) delivery exist. Preclinically, SC injections have been achieved with scleral flap technique, catheters and standard hypodermic needles, as well as with microneedles. A hollow-bore 750 um-long microneedle (Clearside Biomedical, Inc.) can be inserted at the pars, and has shown promise in clinical trials. A microneedle designed with force-sensing technology can be utilized for SC injections, as described by Chitnis, et al. (Chitnis, G. D., et al. A resistance-sensing mechanical injector for the precise delivery of liquids to target tissue. Nat Biomed Eng 3, 621-631 (2019). https://doi.org/10.1038/s41551-019-0350-2). Oxular Limited is developing a delivery system (Oxulumis) that advances an illuminated cannula in the suprachoroidal space. The Orbit device (Gyroscope) is a specially-designed system enabling cannulation of the suprachoroidal space with a flexible cannula. A microneedle inside the cannula is advanced into the subretinal space to enable targeted dose delivery. Ab interno access to the SCS can also be achieved using micro-stents, which serve as minimally-invasive glaucoma surgery (MIGS) devices. Examples include the CyPass® Micro-Stent (Alcon, Fort Worth, Texas, US) and iStent® (Glaukos), which are surgically implanted to provide a conduit from the anterior chamber to the SCS to drain the aqueous humor without forming a filtering bleb. Other devices contemplated for suprachoroidal delivery include those described in UK Patent Publication No. GB 2531910A and U.S. Pat. No. 10,912,883 B2.

In some embodiments, the suprachoroidal drug delivery device is a syringe with a 1 millimeter 30 gauge needle. In some embodiments, the syringe has a larger circumference (e.g., 29 gauge needle). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications.

In certain embodiments, the recombinant viral vector is administered by multiple suprachoroidal injections. In certain embodiments, the recombinant viral vector is administered by triple suprachoroidal injections. In certain embodiments, the recombinant viral vector is administered by double suprachoroidal injections. In certain embodiments, the first injection in the right eye is administered in the superior temporal quadrant (i.e., between the 10 o'clock and 11 o'clock positions), and the second injection in the same eye is administered in the inferior nasal quadrant (i.e., between the 4 o'clock and 5 o'clock positions). In certain embodiments, the first injection in the right eye is administered in the inferior nasal quadrant (i.e., between the 4 o'clock and 5 o'clock positions), and the second injection in the same eye is administered in the superior temporal quadrant (i.e., between the 10 o'clock and 11 o'clock positions). In certain embodiments, the first injection in the left eye is administered in the superior temporal quadrant (i.e., between the 1 o'clock and 2 o'clock positions), and the second injection in the same eye is administered in the inferior nasal quadrant (i.e., between the 7 o'clock and 8 o'clock positions). In certain embodiments, the first injection in the left eye is administered in the inferior nasal quadrant (i.e., between the 7 o'clock and 8 o'clock positions), and the second injection in the same eye is administered in the superior temporal quadrant (i.e., between the 1 o'clock and 2 o'clock positions).

In certain embodiments, the recombinant viral vector is administered by a single suprachoroidal injection. In certain embodiments, the single injection in the right eye is administered in the superior temporal quadrant (i.e., between the 10 o'clock and 11 o'clock positions). In certain embodiments, the single injection in the right eye is administered in the inferior nasal quadrant (i.e., between the 4 o'clock and 5 o'clock positions). In certain embodiments, the single injection in the left eye is administered in the superior temporal quadrant (i.e., between the 1 o'clock and 2 o'clock positions). In certain embodiments, the single injection in the left eye is administered in the inferior nasal quadrant (i.e., between the 7 o'clock and 8 o'clock positions).

In one aspect, provided herein is a method of administration to the outer space of the sclera for treating neovascular age-related macular degeneration or diabetic retinopathy, comprising administering to the outer surface of the sclera in the eye of a human subject in need of treatment a recombinant viral vector provided herein such that the transgene product is expressed and results in treatment of neovascular age-related macular degeneration or diabetic retinopathy. In certain embodiments, the administering step is by the use of a juxtascleral drug delivery device that comprises a cannula whose tip can be inserted and kept in direct apposition to the scleral surface. In certain embodiments, the administering step comprises inserting and keeping the tip of the cannula in direct apposition to the scleral surface.

In another aspect, provided herein is a method for treating neovascular age-related macular degeneration or diabetic retinopathy, comprising administering to the outer surface of the sclera in the eye of a human subject in need of treatment a recombinant viral vector provided herein such that the transgene product is expressed and results in treatment of neovascular age-related macular degeneration or diabetic retinopathy. In certain embodiments, the administering step is by the use of a juxtascleral drug delivery device that comprises a cannula whose tip can be inserted and kept in direct apposition to the scleral surface. In certain embodiments, the administering step comprises inserting and keeping the tip of the cannula in direct apposition to the scleral surface

In one aspect, provided herein is a method of intravitreal administration for neovascular age-related macular degeneration or diabetic retinopathy, comprising administering to the vitreous cavity in the eye of a human subject in need of treatment a recombinant viral vector provided herein such that the transgene product is expressed and results in treatment of neovascular age-related macular degeneration or diabetic retinopathy. In certain embodiments, the administering step is by injecting the recombinant viral vector into the vitreous cavity using an intravitreal drug delivery device. In certain embodiments, the intravitreal drug delivery device is a microinjector.

In another aspect, provided herein is a method for treating neovascular age-related macular degeneration or diabetic retinopathy, comprising administering to the vitreous cavity in the eye of a human subject in need of treatment a recombinant viral vector provided herein such that the transgene product is expressed and results in treatment of neovascular age-related macular degeneration or diabetic retinopathy. In certain embodiments, the administering step is by injecting the recombinant viral vector into the vitreous cavity using an intravitreal drug delivery device. In certain embodiments, the intravitreal drug delivery device is a microinjector.

In one aspect, provided herein is a method of subretinal administration accompanied by vitrectomy for treating neovascular age-related macular degeneration or diabetic retinopathy, comprising administering to the subretinal space in the eye of a human subject in need of treatment a recombinant viral vector provided herein such that the transgene product is expressed and results in treatment of neovascular age-related macular degeneration or diabetic retinopathy, wherein the method comprises performing a vitrectomy on the eye of said human patient. In certain embodiments, the vitrectomy is a partial vitrectomy.

In another aspect, provided herein is a method for treating neovascular age-related macular degeneration or diabetic retinopathy, comprising administering to the subretinal space in the eye of a human subject in need of treatment a recombinant viral vector provided herein such that the transgene product is expressed and results in treatment of neovascular age-related macular degeneration or diabetic retinopathy, wherein the method comprises performing a vitrectomy on the eye of said human patient. In certain embodiments, the vitrectomy is a partial vitrectomy.

In one aspect, provided herein is a method of subretinal administration for treating neovascular age-related macular degeneration or diabetic retinopathy, comprising administering to the subretinal space peripheral to the optic disc, fovea and macula located in the back of the eye of a human subject in need of treatment a recombinant viral vector provided herein such that the transgene product is expressed and results in treatment of neovascular age-related macular degeneration or diabetic retinopathy, wherein the method does not comprise performing a vitrectomy on the eye of said human patient. In certain embodiments, the injecting step is by transvitreal injection. In certain embodiments, the method of transvitreal administration results in uniform expression of the transgene product throughout the eye (e.g. the expression level at the site of injection varies by less than 5%, 10%, 20%, 30%, 40%, or 50% as compared to the expression level at other areas of the eye).

In certain embodiments, the transvitreal injection comprises inserting a sharp needle into the sclera via the superior or inferior side of the eye and passing the sharp needle all the way through the vitreous to inject the recombinant viral vector to the subretinal space on the other side. In certain embodiments, a needle is inserted at the 2 or 10 o'clock position. In certain embodiments, the transvitreal injection comprises inserting a trochar into the sclera and inserting a cannula through the trochar and through the vitreous to inject the recombinant viral vector to the subretinal space on the other side.

In certain embodiments, the transgene product is an anti-hVEGF antibody. In certain embodiments, the anti-hVEGF antibody is an anti-hVEGF antigen-binding fragment.

In certain embodiments, the anti-hVEGF antigen-binding fragment is a Fab, F(ab′)₂, or single chain variable fragment (scFv). In certain embodiments, the anti-hVEGF antibody comprises a heavy chain comprising the amino acid sequence of SEQ ID NO:2 or SEQ ID NO:4, and a light chain comprising the amino acid sequence of SEQ ID NO:1, or SEQ ID NO:3. In certain embodiments, wherein the anti-hVEGF antibody comprises light chain CDRs 1-3 of SEQ ID NOs:14-16 and heavy chain CDRs 1-3 of SEQ ID NOs:17-19 or SEQ ID NOs:20, 18, and 21. In certain embodiments, the pathology of the eye is associated with nAMD, dry age-related macular degeneration (dry AMD), retinal vein occlusion (RVO), diabetic macular edema (DME), or diabetic retinopathy (DR). In certain embodiments, the pathology of the eye is associated with nAMD or DR.

In certain embodiments of the methods described herein, the administering step delivers a therapeutically effective amount of the recombinant viral vector to the retina of said human subject.

In certain embodiments of the methods described herein, the therapeutically effective amount of the transgene product is produced by human retinal cells of said human subject.

In certain embodiments of the methods described herein, the therapeutically effective amount of the transgene product is produced by human photoreceptor cells, horizontal cells, bipolar cells, amacrine cells, retina ganglion cells, and/or retinal pigment epithelial cells in the external limiting membrane of said human subject.

In certain embodiments of the methods described herein, the human photoreceptor cells are cone cells and/or rod cells.

In certain embodiments of the methods described herein, the retina ganglion cells are midget cells, parasol cells, bistratified cells, giant retina ganglion cells, photosensitive ganglion cells, and/or Müller glia.

In certain embodiments of the methods described herein, the recombinant viral vector is an rAAV vector (e.g., an rAAV8, rAAV2, rAAV2tYF, or rAAV5 vector).

In certain embodiments of the methods described herein, wherein the recombinant viral vector is an rAAV8 vector.

In certain embodiments of the methods described herein, delivering to the eye comprises delivering to the retina, choroid, and/or vitreous humor of the eye.

### 5.3.2 Target Patient Populations

The subjects treated in accordance with the methods described herein can be any mammals such as rodents, domestic animals such as dogs or cats, or primates, e.g. non-human primates. In a preferred embodiment, the subject is a human. In certain embodiments, the methods provided herein are for the administration to patients diagnosed with an ocular disease (for example, wet AMD, dry AMD, retinal vein occlusion (RVO), diabetic macular edema (DME), or diabetic retinopathy (DR) (in particular, wet AMD or DR)), in particular an ocular disease caused by increased neovascularization

In certain embodiments, the methods provided herein are for the administration to patients diagnosed with severe AMD. In certain embodiments, the methods provided herein are for the administration to patients diagnosed with attenuated AMD.

In certain embodiments, the methods provided herein are for the administration to patients diagnosed with severe wet AMD. In certain embodiments, the methods provided herein are for the administration to patients diagnosed with attenuated wet AMD.

In certain embodiments, the methods provided herein are for the administration to patients diagnosed with AMD who have been identified as responsive to treatment with an anti-VEGF antibody.

In certain embodiments, the methods provided herein are for the administration to patients diagnosed with AMD who have been identified as responsive to treatment with an anti-VEGF antigen-binding fragment.

In certain embodiments, the methods provided herein are for the administration to patients diagnosed with AMD who have been identified as responsive to treatment with an anti-VEGF antigen-binding fragment injected intravitreally prior to treatment with gene therapy.

In certain embodiments, the methods provided herein are for the administration to patients diagnosed with AMD who have been identified as responsive to treatment with LUCENTIS® (ranibizumab), EYLEA® (aflibercept), and/or AVASTIN® (bevacizumab).

In certain embodiments, a patient diagnosed with AMD is identified as responsive to treatment with an anti-VEGF antigen-binding fragment (e.g., ranibizumab) if the patient has improvement in fluid after intravitreal injection of the anti-VEGF antigen-binding fragment to the patient prior to treatment with gene therapy. In certain embodiments, a patient diagnosed with AMD is identified as responsive to treatment with an anti-VEGF antigen-binding fragment (e.g., ranibizumab) if the patient has improvement in fluid and has a central retinal thickness (CRT)<400 μm after intravitreal injection of the anti-VEGF antigen-binding fragment to the patient prior to treatment with gene therapy. In some embodiments, the anti-VEGF antigen-binding fragment is intravitreally injected to the patient at 0.5 mg per month for two months prior to treatment with gene therapy. In other embodiments, the anti-VEGF antigen-binding fragment is intravitreally injected to the patient at 0.5 mg per month for three months prior to treatment with gene therapy. In a preferred embodiment, a patient has improvement in fluid if he or she has an improvement in inner retinal (parafovea 3 mm) fluid of >50 μm or 30% relative to the level prior to the intravitreal injection of the anti-VEGF antigen-binding fragment, or has an improvement in center subfield thickness of >50 μm or 30% as determined by the CRC relative to the level prior to the intravitreal injection of the anti-VEGF antigen-binding fragment.

In certain embodiments, the methods provided herein are for the administration to patients diagnosed with AMD who have disease other than fluid contributing to an increase in CRT (i.e., pigment epithelial detachment (PED) or subretinal hyperreflective material (SHRM)) and who have <75 μm of fluid (intraretinal or subretinal), as determined by the CRC.

In certain embodiments of the methods described herein, the patient has a BCVA in the eye to be treated that is ≤20/20 and ≥20/400 before treatment. In certain embodiments, the patient has a BCVA in the eye to be treated that is ≤20/25 and ≥20/125 (≤83 and ≥44 ETDRS letters) before treatment. In a specific embodiment, the patient has a BCVA in the eye to be treated that is ≤20/63 and ≥20/400 before treatment.

In certain embodiments of the methods described herein, the patients has a negative or low serum titer result (≤300) for recombinant viral vector neutralizing antibodies (NAbs). In certain embodiments, the patients has a negative or low serum titer result (≤300) for AAV8 NAbs. In other embodiments, the patients has a serum titer result >300 for recombinant viral vector NAbs. In certain embodiments, the patients has a serum titer result >300 for AAV8 NAbs. In certain embodiments, the methods described herein are effective for treatment of AMD or DR in patients having a negative or low serum titer result (≤300) for AAV8 NAbs or in patients having a serum titer result >300 for recombinant viral vector NAbs. In certain embodiments, the methods described herein are as effective for treatment of AMD and DR in patients having a negative or low serum titer result (≤300) for AAV8 NAbs as in patients having a serum titer result >300 for recombinant viral vector NAbs.

In certain embodiments of the methods described herein, the patient is not concurrently having an anticoagulation therapy.

In certain embodiments, the methods provided herein are for the administration to patients diagnosed with severe diabetic retinopathy. In certain embodiments, the methods provided herein are for the administration to patients diagnosed with attenuated diabetic retinopathy.

In certain embodiments, the methods provided herein are for the administration to patients diagnosed with moderately-severe NPDR. In certain embodiments, the methods provided herein are for the administration to patients diagnosed with severe NPDR. In certain embodiments, the methods provided herein are for the administration to patients diagnosed with mild PDR. In certain embodiments, the methods provided herein are for the administration to patients diagnosed with moderate PDR.

In certain embodiments of the methods described herein, the patient has an Early Treatment Diabetic Retinopathy Study (ETDRS) BCVA letter score between ≤78 and >44 in the eye to be treated before treatment.

In certain embodiments, the methods provided herein are for the administration to patients whose ETDRS-DRSS Levels are 47, 53, 61 or 65. In certain embodiments, the methods provided herein are for the administration to patients whose ETDRS-DRSS Levels are Level 47. In certain embodiments, the methods provided herein are for the administration to patients whose ETDRS-DRSS Levels are Level 53. In certain embodiments, the methods provided herein are for the administration to patients whose ETDRS-DRSS Levels are Level 61. In certain embodiments, the methods provided herein are for the administration to patients whose ETDRS-DRSS Levels are Level 65.

In certain embodiments, the subject treated in accordance with the methods described herein is female. In certain embodiments, the subject treated in accordance with the methods described herein is male. In certain embodiments, the subject treated in accordance with the methods described herein can be of any age. In certain embodiments, the subject treated in accordance with the methods described herein is 18 years old or older. In certain embodiments, the subject treated in accordance with the methods described herein is between 18-89 years of age. In certain embodiments, the subject treated in accordance with the methods described herein is between 25-89 years of age. In certain embodiments, the subject treated in accordance with the methods described herein has DR secondary to diabetes mellitus Type 1. In certain embodiments, the subject treated in accordance with the methods described herein has DR secondary to diabetes mellitus Type 2. In certain embodiments, the subject treated in accordance with the methods described herein is 18 years old or older with DR secondary to diabetes mellitus Type 1 or Type 2. In certain embodiments, the subject treated in accordance with the methods described herein is between 18-89 years of age with DR secondary to diabetes mellitus Type 1 or Type 2.

In a specific embodiment, the subject treated in accordance with the methods described herein is a woman without childbearing potential.

In specific embodiments, the subject treated in accordance with the methods described herein is phakic. In other specific embodiments, the subject treated in accordance with the methods described herein is pseudophakic.

In certain embodiments, the subject treated in accordance with the methods described herein has a hemoglobin A1c≤12% (as confirmed by laboratory assessments).

In certain embodiments, the subject treated in accordance with the methods described herein has best-corrected visual acuity (BCVA) in the eye to be treated of ≥69 ETDRS letters (approximate Snellen equivalent 20/40 or better).

In certain embodiments, provided herein is a method for treating a subject with diabetic retinopathy (DR), wherein the subject has at least one eye with DR, the method comprising the steps of:
(1) determining the subject's ETDRS-DR Severity Scale (DRSS) Level, and
(2) if the subject's ETDRS-DRSS is Level 47, 53, 61 or 65 then administering to the subretinal space or the suprachoroidal space in the eye of the human subject an expression vector encoding an anti-human vascular endothelial growth factor (hVEGF) antibody.

In some embodiments, the method further comprises obtaining or having obtained a biological sample from the subject, and determining that the subject has a serum level of hemoglobin A1c of less than or equal to 10%.

In some embodiments, the method prevents progression to proliferative stages of retinopathy in the subject.

In certain embodiments, provided herein is a method for treating a subject with diabetic retinopathy, wherein the subject has at least one eye with moderately-severe non-proliferative diabetic retinopathy (NPDR), the method comprising the steps of:
(1) determining the subject's ETDRS-DR Severity Scale (DRSS) Level, and
(2) if the subject's ETDRS-DRSS is Level 47, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with severe NPDR, the method comprising the steps of:
(1) determining the subject's ETDRS-DR Severity Scale (DRSS) Level, and
(2) if the subject's ETDRS-DRSS is Level 53, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with mild proliferative diabetic retinopathy (PDR), the method comprising the steps of:
(1) determining the subject's ETDRS-DR Severity Scale (DRSS) Level, and
(2) if the subject's ETDRS-DRSS is Level 61, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with moderate PDR, the method comprising the steps of:
(1) determining the subject's ETDRS-DR Severity Scale (DRSS) Level, and
(2) if the subject's ETDRS-DRSS is Level 65, then administering to the subretinal space or the suprachoroidal space in the eye of the human subject an expression vector encoding an anti-human vascular endothelial growth factor (hVEGF) antibody.

In certain embodiments of the methods described herein, the patients has a negative or low serum titer result (≤300) for recombinant viral vector neutralizing antibodies (NAbs). In certain embodiments, the patients has a negative or low serum titer result (≤300) for AAV8 NAbs. In other embodiments, the patients has a serum titer result >300 for recombinant viral vector NAbs. In certain embodiments, the patients has a serum titer result >300 for AAV8 NAbs.

ETDRS-DR severity scale (DRSS) Levels are determined using standard 4-widefield digital stereoscopic fundus photographs or equivalent; they may also be measured by monoscopic or stereo photography in accordance with Li et al., 2010, Retina Invest Ophthalmol Vis Sci. 2010; 51:3184-3192, or an analogous method.

### 5.3.3 Dosage and Mode of Administration

Therapeutically effective doses of the recombinant vector should be administered subretinally and/or intraretinally (e.g., by subretinal injection via the transvitreal approach (a surgical procedure), or via the suprachoroidal space) in a volume ranging from ≥0.1 mL to ≤0.5 mL, preferably in 0.1 to 0.30 mL (100-300 μl), and most preferably, in a volume of 0.25 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera in a volume of 500 μl or less, for example, in a volume of 500 μl or less, for example, in a volume of 10-20 l, 20-50 μl, 50-100 μl, 100-200 μl, 200-300 μl, 300-400 μl, or 400-500 μl. Therapeutically effective doses of the recombinant vector may also be administered to the outer surface of the sclera in two or more injections of a volume of 500 μl or less, for example, a volume of 10-20 l, 20-50 μl, 50-100 μl, 100-200 μl, 200-300 μl, 300-400 μl, or 400-500 μl. The two or more injections may be administered during the same visit.

In certain embodiments, a therapeutically effective dose of the recombinant vector is administered to the subject as a single dosage form. In certain embodiments, the therapeutically effective dose of the recombinant vector is administered to the subject as a single injection. In certain embodiments, the therapeutically effective dose of the recombinant vector is administered to the subject as a single injection per eye.

In certain embodiments, the recombinant vector is administered suprachoroidally (e.g., by suprachoroidal injection). In a specific embodiment, suprachorodial administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13: 20-23; Goldstein, 2014, Retina Today 9(5): 82-87; Baldassarre et al., 2017; each of which is incorporated by reference herein in its entirety). The suprachoroidal drug delivery devices that can be used to deposit the expression vector in the subretinal space according to the embodiments described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters.

In a specific embodiment, the suprachoroidal drug delivery device is a syringe with a 1 millimeter 30 gauge needle. During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications. A max volume of 100 μl can be injected into the suprachoroidal space.

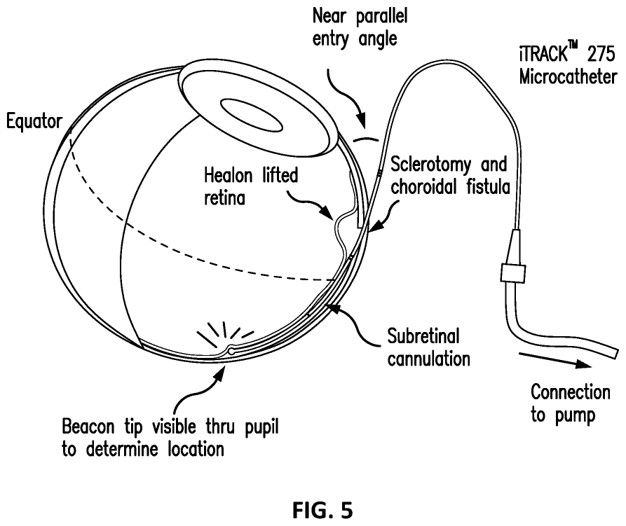

In certain embodiments, the recombinant vector is administered subretinally via the suprachoroidal space by use of a subretinal drug delivery device. In certain embodiments, the subretinal drug delivery device is a catheter which is inserted and tunneled through the suprachoroidal space around to the back of the eye during a surgical procedure to deliver drug to the subretinal space (see FIG. 5). This procedure allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery (such as suprachoroidal and intravitreal). Additionally, this method has shown to create blebs with less egress out the retinotomy site than the standard transvitreal approach. The subretinal drug delivery device originally manufactured by Janssen Pharmaceuticals, Inc. now by Orbit Biomedical Inc. (see, for example, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1) can be used for such purpose.

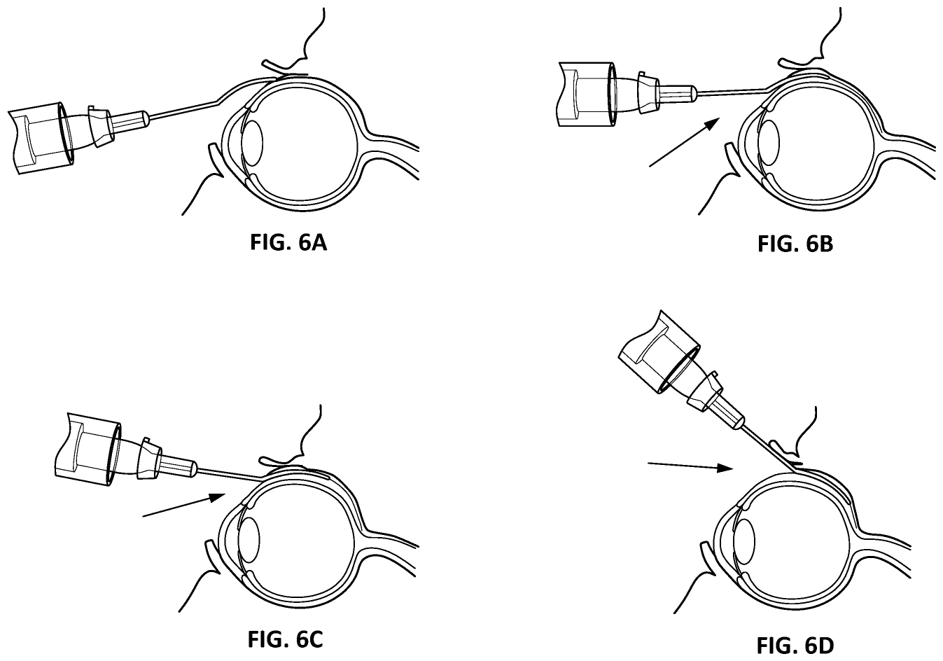

In certain embodiments, the recombinant vector is administered to the outer surface of the sclera (for example, by the use of a juxtascleral drug delivery device that comprises a cannula, whose tip can be inserted and kept in direct apposition to the scleral surface). In a specific embodiment, administration to the outer surface of the sclera is performed using a posterior juxtascleral depot procedure, which involves drug being drawn into a blunt-tipped curved cannula and then delivered in direct contact with the outer surface of the sclera without puncturing the eyeball. In particular, following the creation of a small incision to bare sclera, the cannula tip is inserted (see FIG. 6A). The curved portion of the cannula shaft is inserted, keeping the cannula tip in direct apposition to the scleral surface (see FIGS. 6B-6D). After complete insertion of the cannula (FIG. 6D), the drug is slowly injected while gentle pressure is maintained along the top and sides of the cannula shaft with sterile cotton swabs. This method of delivery avoids the risk of intraocular infection and retinal detachment, side effects commonly associated with injecting therapeutic agents directly into the eye.

Doses that maintain a concentration of the transgene product at a Cmin of at least 0.330 μg/mL in the Vitreous humour, or 0.110 μg/mL in the Aqueous humour (the anterior chamber of the eye) for three months are desired; thereafter, Vitreous Cmin concentrations of the transgene product ranging from 1.70 to 6.60 μg/mL, and/or Aqueous Cmin concentrations ranging from 0.567 to 2.20 μg/mL should be maintained. However, because the transgene product is continuously produced (under the control of a constitutive promoter or induced by hypoxic conditions when using an hypoxia-inducible promoter), maintenance of lower concentrations can be effective. Vitreous humour concentrations can be measured directly in patient samples of fluid collected from the vitreous humour or the anterior chamber, or estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

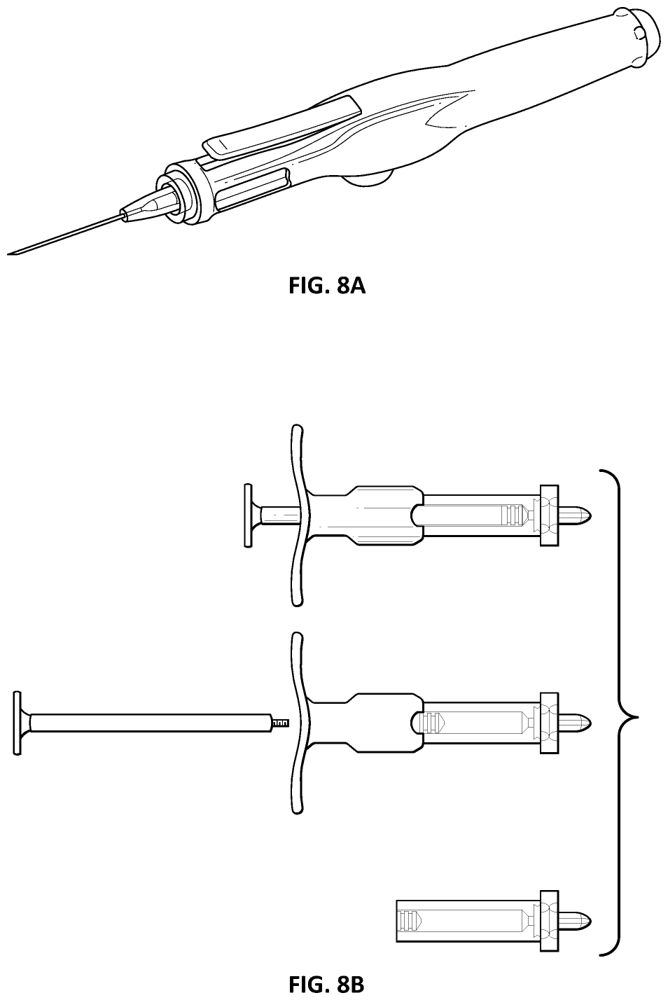

In certain embodiment, described herein is an micro volume injector delivery system, which is manufactured by Altaviz (see FIGS. 8A and 8B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. In certain embodiment, the micro volume injector delivery system can be used for micro volume injector is a micro volume injector with dose guidance and can be used with, for example, a suprachoroidal needle (for example, the Clearside® needle), a subretinal needle, an intravitreal needle, a juxtascleral needle, a subconjunctival needle, and/or intraretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for subretinal delivery).

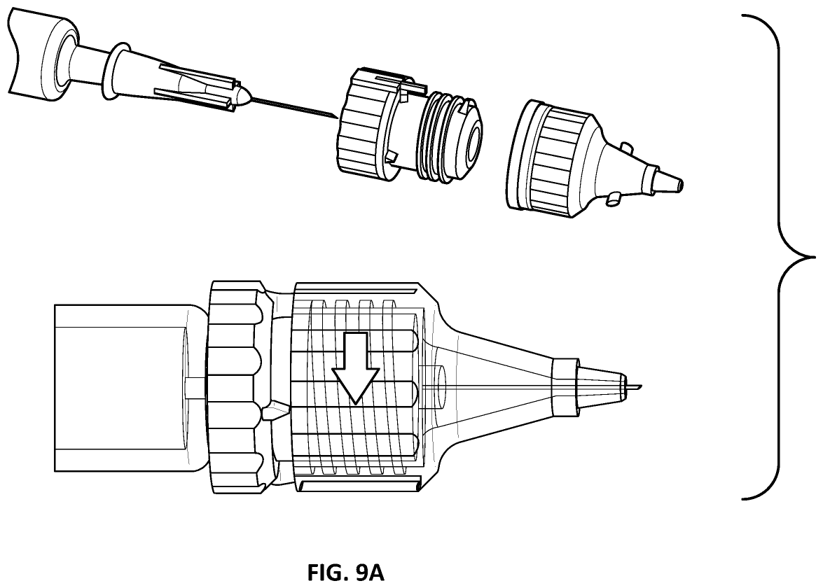

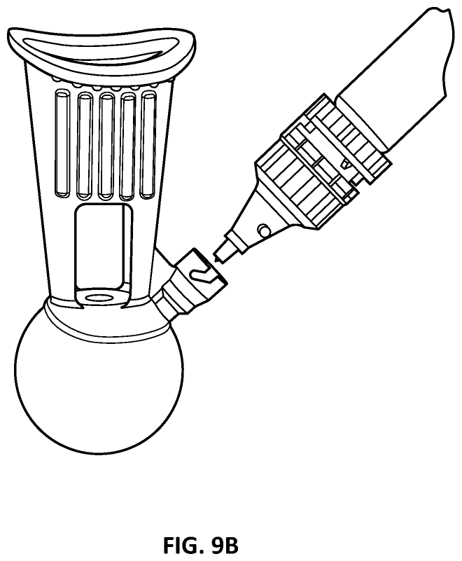

In certain embodiments of the methods described herein, the recombinant vector is administered suprachoroidally (e.g., by suprachoroidal injection). In a specific embodiment, suprachoroidal administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13: 20-23; Goldstein, 2014, Retina Today 9(5): 82-87; Baldassarre et al., 2017; each of which is incorporated by reference herein in its entirety). The suprachoroidal drug delivery devices that can be used to deposit the recombinant vector in the suprachoroidal space according to the embodiments described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters. In another embodiment, the suprachoroidal drug delivery device that can be used in accordance with the methods described herein comprises the micro volume injector delivery system, which is manufactured by Altaviz (see FIGS. 8A and 8B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a suprachoroidal needle (for example, the Clearside® needle) or a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for suprachoroidal delivery). In another embodiment, the suprachoroidal drug delivery device that can be used in accordance with the methods described herein is a tool that comprises a normal length hypodermic needle with an adaptor (and preferably also a needle guide) manufactured by Visionisti OY, which adaptor turns the normal length hypodermic needle into a suprachoroidal needle by controlling the length of the needle tip exposing from the adapter (see FIGS. 9A and 9B) (see, for example, U.S. Design Patent No. D878,575; and International Patent Application. Publication No. WO/2016/083669) In a specific embodiment, the suprachoroidal drug delivery device is a syringe with a 1 millimeter 30 gauge needle. During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of therapeutic product from all retinal cell layers and choroidal cells. Using this type of device and procedure allows for a quick and easy in-office procedure with low risk of complications. A max volume of 100 μl can be injected into the suprachoroidal space.

In a specific embodiment, the intravitreal administration is performed with a intravitreal drug delivery device that comprises the micro volume injector delivery system, which is manufactured by Altaviz (see FIGS. 8A and 8B) (see, e.g. International Patent Application Publication No. WO 2013/177215), United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a intravitreal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip. In a specific embodiment, the subretinal administration is performed with a subretinal drug delivery device that comprises the micro volume injector delivery system, which is manufactured by Altaviz (see FIGS. 8A and 8B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro volume injector is a micro volume injector with dose guidance and can be used with, for example, a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for suprachoroidal delivery).

In certain embodiments, the recombinant vector is administered to the outer surface of the sclera (for example, by the use of a juxtascleral drug delivery device that comprises a cannula, whose tip can be inserted and kept in direct apposition to the scleral surface). In a specific embodiment, administration to the outer surface of the sclera is performed using a posterior juxtascleral depot procedure, which involves drug being drawn into a blunt-tipped curved cannula and then delivered in direct contact with the outer surface of the sclera without puncturing the eyeball. In particular, following the creation of a small incision to bare sclera, the cannula tip is inserted (see FIG. 6A). The curved portion of the cannula shaft is inserted, keeping the cannula tip in direct apposition to the scleral surface (see FIGS. 6B-6D). After complete insertion of the cannula (FIG. 6D), the drug is slowly injected while gentle pressure is maintained along the top and sides of the cannula shaft with sterile cotton swabs. This method of delivery avoids the risk of intraocular infection and retinal detachment, side effects commonly associated with injecting therapeutic agents directly into the eye. In a specific embodiment, the juxtascleral administration is performed with a juxtascleral drug delivery device that comprises the micro volume injector delivery system, which is manufactured by Altaviz (see FIGS. 8A and 8B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro Volume Injector is a micro volume injector with dose guidance and can be used with, for example, a juxtascleral needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip.

In certain embodiments, dosages are measured by genome copies per ml or the number of genome copies administered to the eye of the patient (e.g., administered suprachoroidally, subretinally, intravitreally, juxtasclerally, subconjunctivally, and/or intraretinally (e.g., by suprachoroidal injection, subretinal injection via the transvitreal approach (a surgical procedure), subretinal administration via the suprachoroidal space, or a posterior juxtascleral depot procedure). In certain embodiments, 2.4×10¹¹ genome copies per ml to 1×10¹³ genome copies per ml are administered. In a specific embodiment, 2.4×10¹¹ genome copies per ml to 5×10¹¹ genome copies per ml are administered. In another specific embodiment, 5×10¹¹ genome copies per ml to 1×10¹² genome copies per ml are administered. In another specific embodiment, 1×10¹² genome copies per ml to 5×10¹² genome copies per ml are administered. In another specific embodiment, 5×10¹² genome copies per ml to 1×10¹³ genome copies per ml are administered. In another specific embodiment, about 2.4×10¹¹ genome copies per ml are administered. In another specific embodiment, about 5×10¹¹ genome copies per ml are administered. In another specific embodiment, about 1×10¹² genome copies per ml are administered. In another specific embodiment, about 5×10¹² genome copies per ml are administered. In another specific embodiment, about 1×10¹³ genome copies per ml are administered.

In certain embodiments, 1×10⁹ to 1×10¹² genome copies are administered. In specific embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered. In specific embodiments, 2×10¹¹ to 1×10¹² genome copies are administered. In a specific embodiment, about 3×10⁹ genome copies are administered (which corresponds to about 1.2×10¹⁰ genome copies per ml in a volume of 250 μl). In another specific embodiment, about 1×10¹⁰ genome copies are administered (which corresponds to about 4×10¹⁰ genome copies per ml in a volume of 250 μl). In another specific embodiment, about 6×10¹⁰ genome copies are administered (which corresponds to about 2.4×10¹¹ genome copies per ml in a volume of 250 μl). In another specific embodiment, about 1.6×10¹¹ genome copies are administered (which corresponds to about 6.2×10¹¹ genome copies per ml in a volume of 250 μl). In another specific embodiment, about 1.55×10¹¹ genome copies are administered (which corresponds to about 6.2×10¹¹ genome copies per ml in a volume of 250 μl). In another specific embodiment, about 1.6×10¹¹ genome copies are administered (which corresponds to about 6.4×10¹¹ genome copies per ml in a volume of 250 μl). In another specific embodiment, about 2.5×10¹¹ genome copies (which corresponds to about 1.0×10¹² in a volume of 250 μl) are administered.

In certain embodiments, about 3.0×10¹³ genome copies per eye are administered. In certain embodiments, up to 3.0×10¹³ genome copies per eye are administered.

In certain embodiments, about 6.0×10¹⁰ genome copies per eye are administered. In certain embodiments, about 1.6×10¹¹ genome copies per eye are administered. In certain embodiments, about 2.5×10¹¹ genome copies per eye are administered. In certain embodiments, about 5.0×10¹¹ genome copies per eye are administered. In certain embodiments, about 3×10¹² genome copies per eye are administered. In certain embodiments, about 1×10¹² genome copies per ml per eye are administered. In certain embodiments, about 2.5×10¹² genome copies per ml per eye are administered.

In certain embodiments, about 6.0×10¹⁰ genome copies per eye are administered by subretinal injection. In certain embodiments, about 1.6×10¹¹ genome copies per eye are administered by subretinal injection. In certain embodiments, about 2.5×10¹¹ genome copies per eye are administered by subretinal injection. In certain embodiments, about 3.0×10¹³ genome copies per eye are administered by subretinal injection. In certain embodiments, up to 3.0×10¹³ genome copies per eye are administered by subretinal injection.

In certain embodiments, about 2.5×10¹¹ genome copies per eye are administered by suprachoroidal injection. In certain embodiments, about 5.0×10¹¹ genome copies per eye are administered by suprachoroidal injection. In certain embodiments, about 3×10¹² genome copies per eye are administered by suprachoroidal injection. In certain embodiments, about 2.5×10¹¹ genome copies per eye are administered by a single suprachoroidal injection. In certain embodiments, about 5.0×10¹¹ genome copies per eye are administered by double suprachoroidal injections. In certain embodiments, about 3.0×10¹³ genome copies per eye are administered by suprachoroidal injection. In certain embodiments, up to 3.0×10¹³ genome copies per eye are administered by suprachoroidal injection. In certain embodiments, about 2.5×10¹² genome copies per ml per eye are administered by a single suprachoroidal injection in a volume of 100 μl. In certain embodiments, about 2.5×10¹² genome copies per ml per eye are administered by double suprachoroidal injections, wherein each injection is in a volume of 100 μl.

In certain embodiments, about 1.5×10¹¹ genome copies per administration, or per eye are administered by suprachoroidal injection. In certain embodiments, about 2.5×10¹¹ genome copies per administration, or per eye are administered by suprachoroidal injection. In certain embodiments, about 5.0×10¹¹ genome copies per administration, or per eye are administered by suprachoroidal injection. In certain embodiments, about 1.0×10¹² genome copies per administration, or per eye are administered by suprachoroidal injection. In certain embodiments, about 1.5×10¹² genome copies per administration, or per eye are administered by suprachoroidal injection. In certain embodiments, about 2.5×10¹¹ genome copies per eye are administered by a single suprachoroidal injection. In certain embodiments, about 2.5×10¹¹ genome copies per eye are administered by a single suprachoroidal injection in a volume of about 100 μl. In certain embodiments, about 5×10¹¹ genome copies per eye are administered by a single suprachoroidal injection. In certain embodiments, about 5×10¹¹ genome copies per eye are administered by a single suprachoroidal injection in a volume of about 100 μl. In certain embodiments, about 5×10¹¹ genome copies per administration, or per eye are administered by double suprachoroidal injections. In certain embodiments, about 5×10¹¹ genome copies per eye are administered by double suprachoroidal injections, wherein each injection is in a volume of 100 μl. In certain embodiments, about 1×10¹² genome copies per eye are administered by a single suprachoroidal injection. In certain embodiments, about 1×10¹² genome copies per eye are administered by a single suprachoroidal injection in a volume of about 100 μl. In certain embodiments, about 1.5×10¹² genome copies per eye are administered by a single suprachoroidal injection. In certain embodiments, about 1.5×10¹² genome copies per eye are administered by a single suprachoroidal injection in a volume of about 100 μl.

As used herein and unless otherwise specified, the term “about” means within plus or minus 10% of a given value or range.

In certain embodiments, the term “about” encompasses the exact number recited.

The term “between” as used in a phrase as such “between A and B” or “between A-B” refers to a range including both A and B.

In certain embodiments, an infrared thermal camera can be used to detect changes in the thermal profile of the ocular surface after the administering of a solution which is cooler than body temperature to detect changes in the thermal profile of the ocular surface that allows for visualization of the spread of the solution, e.g., within the SCS, and can potentially determine whether the administration was successfully completed. This is because in certain embodiments the formulation containing the recombinant vector to be administered is initially frozen, brought to room temperature (68-72° F.), and thawed for a short period of time (e.g., at least 30 minutes) before administration, and thus the formulation is colder than the human eye (about 92° F.) (and sometimes even colder than room temperature) at the time of injection. The drug product is typically used within 4 hours of thaw and the warmest the solution would be is room temperature. In a preferred embodiment, the procedure is videoed with infrared video.

Infrared thermal cameras can detect small changes in temperature. They capture infrared energy through a lens and convert the energy into an electronic signal. The infrared light is focused onto an infrared sensor array which converts the energy into a thermal image. The infrared thermal camera can be used for any method of administration to the eye, including any administration route described herein, for example, suprachoroidal administration, subretinal administration, subconjunctival administration, intravitreal administration, or administration with the use of a slow infusion catheter in to the suprachoroidal space. In a specific embodiment, the infrared thermal camera is an FUR T530 infrared thermal camera. The FUR T530 infrared thermal camera can capture slight temperature differences with an accuracy of 3.6° F. The camera has an infrared resolution of 76,800 pixels. The camera also utilizes a 240 lens capturing a smaller field of view. A smaller field of view in combination with a high infrared resolution contributes to more detailed thermal profiles of what the operator is imaging. However, other infrared camera can be used that have different abilities and accuracy for capturing slight temperature changes, with different infrared resolutions, and/or with different degrees of lens.

In a specific embodiment, the infrared thermal camera is an FLIR T420 infrared thermal camera. In a specific embodiment, the infrared thermal camera is an FLIR T440 infrared thermal camera. In a specific embodiment, the infrared thermal camera is an Fluke Ti400 infrared thermal camera. In a specific embodiment, the infrared thermal camera is an FLIRE60 infrared thermal camera. In a specific embodiment, the infrared resolution of the infrared thermal camera is equal to or greater than 75,000 pixels. In a specific embodiment, the thermal sensitivity of the infrared thermal camera is equal to or smaller than 0.05° C. at 30° C. In a specific embodiment, the field of view (FOV) of the infrared thermal camera is equal to or lower than 25°×25°.

In certain embodiments, an iron filer is used with the infrared thermal camera to detect changes in the thermal profile of the ocular surface. In a preferred embodiment, the use of an iron filter is able to a generate pseudo-color image, wherein the warmest or high temperature parts are colored white, intermediate temperatures are reds and yellows, and the coolest or low temperature parts are black. In certain embodiments, other types of filters can also be used to generate pseudo-color images of the thermal profile.

The thermal profile for each administration method can be different. For example, in one embodiment, a successful suprachoroidal injection can be characterized by: (a) a slow, wide radial spread of the dark color, (b) very dark color at the beginning, and (c) a gradual change of injectate to lighter color, i.e., a temperature gradient noted by a lighter color. In one embodiment, an unsuccessful suprachoroidal injection can be characterized by: (a) no spread of the dark color, and (b) a minor change in color localized to the injection site without any distribution. In certain embodiments, the small localized temperature drop is result from cannula (low temperature) touching the ocular tissues (high temperature). In one embodiment, a successful intravitreal injection can be characterized by: (a) no spread of the dark color, (b) an initial change to very dark color localized to the injection site, and (c) a gradual and uniform change of the entire eye to darker color. In one embodiment, an extraocular efflux can be characterized by: (a) quick flowing streams on outside on the exterior surface of the eye, (b) very dark color at the beginning, and (c) a quick change to lighter color.

### 5.3.4 Sampling and Monitoring of Efficacy

Effects of the methods of treatment provided herein on visual deficits may be measured by BCVA (Best-Corrected Visual Acuity), intraocular pressure, slit lamp biomicroscopy, and/or indirect ophthalmoscopy. Extraocular movement may also be assessed. The intraocular pressure measurements may be conducted using Tonopen or Goldmann applanation tonometry. The slit lamp examination may include an evaluation of the lids/lashes, conjunctiva/sclera, cornea, anterior chamber, iris, lens, and/or vitreous body.

In specific embodiments, effects of the methods provided herein on visual deficits may be measured by whether the human patient's eye that is treated by a method described herein achieves BCVA of greater than 43 letters post-treatment (e.g., 46-50 weeks or 98-102 weeks post-treatment). A BCVA of 43 letters corresponds to 20/160 approximate Snellen equivalent. In a specific embodiment, the human patient's eye that is treated by a method described herein achieves BCVA of greater than 43 letters post-treatment (e.g., 46-50 weeks or 98-102 weeks post-treatment).

In specific embodiments, effects of the methods provided herein on visual deficits may be measured by whether the human patient's eye that is treated by a method described herein achieves BCVA of greater than 84 letters post-treatment (e.g., 46-50 weeks or 98-102 weeks post-treatment). A BCVA of 84 letters corresponds to 20/20 approximate Snellen equivalent. In a specific embodiment, the human patient's eye that is treated by a method described herein achieves BCVA of greater than 84 letters post-treatment (e.g., 46-50 weeks or 98-102 weeks post-treatment). The BCVA testing may be conducted at a distance of 4 meters using ETDRS charts. For participants with reduced vision (inability to read ≥20 letters correctly at 4 meters), the BCVA testing may be conducted at a distance of 1 meter.

Effects of the methods of treatment provided herein on physical changes to eye/retina may be measured by SD-OCT (SD-Optical Coherence Tomography).

Efficacy may be monitored as measured by electroretinography (ERG).

Effects of the methods of treatment provided herein may be monitored by measuring signs of vision loss, infection, inflammation and other safety events, including retinal detachment.

Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458).

Effects of the methods provided herein may also be measured by a change from baseline in National Eye Institute Visual Functioning Questionnaire, the Rasch-scored version (NEI-VFQ-28-R) (composite score; activity limitation domain score; and socio-emotional functioning domain score). Effects of the methods provided herein may also be measured by a change from baseline in National Eye Institute Visual Functioning Questionnaire 25-item version (NEI-VFQ-25) (composite score and mental health subscale score). Effects of the methods provided herein may also be measured by a change from baseline in Macular Disease Treatment Satisfaction Questionnaire (MacTSQ) (composite score; safety, efficacy, and discomfort domain score; and information provision and convenience domain score).

In specific embodiments, the efficacy of a method described herein is reflected by an improvement in vision at about 4 weeks, 12 weeks, 6 months, 12 months, 24 months, 36 months, or at other desired timepoints. In a specific embodiment, the improvement in vision is characterized by an increase in BCVA, for example, an increase by 1 letter, 2 letters, 3 letters, 4 letters, 5 letters, 6 letters, 7 letters, 8 letters, 9 letters, 10 letters, 11 letters, or 12 letters, or more. In a specific embodiment, the improvement in vision is characterized by a 5%, 10%, 15%, 20%, 30%, 40%, 50% or more increase in visual acuity from baseline.

In specific embodiments, the efficacy of a method described herein is reflected by an reduction in central retinal thickness (CRT) at about 4 weeks, 12 weeks, 6 months, 12 months, 24 months, 36 months, or at other desired timepoint, for example, a 5%, 10%, 15%, 20%, 30%, 40%, 50% or more decrease in central retinal thickness from baseline.

In a specific embodiments, there is no inflammation in the eye after treatment or little inflammation in the eye after treatment (for example, an increase in the level of inflammation by 10%, 5%, 2%, 1% or less from baseline). Effects of the methods provided herein on visual deficits may be measured by OptoKinetic Nystagmus (OKN).

In specific embodiments, the proportion of subjects who experience ocular inflammation (for example, an increase in the level of inflammation in the eye by 10% or more, 5% or more, 2% or more, or 1% or more from baseline) following administration of an anti-VEGF treatment and a steroid treatment described herein is less than three-quarters, less than half, less than one-quarter, or less than one-tenth of all subjects in a population of subjects. In specific embodiments, the proportion of subjects who experience ocular inflammation (for example, an increase in the level of inflammation in the eye by 10% or more, 5% or more, 2% or more, or 1% or more from baseline) following administration of an anti-VEGF treatment and a steroid treatment described herein is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to the proportion in a reference population. In specific embodiments, the proportion of subjects who experience ocular inflammation (for example, an increase in the level of inflammation in the eye by 10% or more, 5% or more, 2% or more, or 1% or more from baseline) following administration of an anti-VEGF treatment and a steroid treatment described herein is reduced by between about 5% and about 10%, about 10% and about 15%, about 15% and about 20%, about 20% and about 25%, about 25% and about 30%, about 30% and about 35%, about 35% and about 40%, about 40% and about 45%, about 45% and about 50%, about 50% and about 55%, about 55% and about 60%, about 60% and about 65%, about 65% and about 70%, about 70% and about 75%, about 75% and about 80%, about 80% and about 85%, about 85% and about 90%, about 90% and about 95%, or about 95% and about 100% as compared to the proportion in a reference population. In certain embodiments, the reference population consists of individuals administered an ocular therapy for neovascular age-related macular generation or diabetic retinopathy, wherein the ocular therapy is not one of those described herein, for example in Sections 5.2, 5.3 and/or 5.4. In certain embodiments, the reference population consists of individuals administered an ocular therapy for neovascular age-related macular generation or diabetic retinopathy, such as an anti-VEGF treatment described herein in Sections 5.2 and/or Section 5.3, but who are not administered a steroid treatment described herein in Section 5.4.

Without being bound by theory, this visual acuity screening uses the principles of the OKN involuntary reflex to objectively assess whether a patient's eyes can follow a moving target. By using OKN, no verbal communication is needed between the tester and the patient. As such, OKN can be used to measure visual acuity in pre-verbal and/or non-verbal patients. In certain embodiments, OKN is used to measure visual acuity in patients that are 1 month old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In certain embodiments, an iPad is used to measure visual acuity through detection of the OKN reflex when a patient is looking at movement on the iPad.

Without being bound by theory, this visual acuity screening uses the principles of the OKN involuntary reflex to objectively assess whether a patient's eyes can follow a moving target. By using OKN, no verbal communication is needed between the tester and the patient. As such, OKN can be used to measure visual acuity in pre-verbal and/or non-verbal patients. In certain embodiments, OKN is used to measure visual acuity in patients that are less than 1.5 months old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In another specific embodiment, OKN is used to measure visual acuity in patients that are 1-2 months old, 2-3 months old, 3-4 months old, 4-5 months old, 5-6 months old, 6-7 months old, 7-8 months old, 8-9 months old, 9-10 months old, 10-11 months old, 11 months to 1 year old, 1-1.5 years old, 1.5-2 years old, 2-2.5 years old, 2.5-3 years old, 3-3.5 years old, 3.5-4 years old, 4-4.5 years old, or 4.5-5 years old. In another specific embodiment, OKN is used to measure visual acuity in patients that are 6 months to 5 years old. In certain embodiments, an iPad is used to measure visual acuity through detection of the OKN reflex when a patient is looking at movement on the iPad.

If the human patient is a child, visual function can be assessed using an optokinetic nystagmus (OKN)-based approach or a modified OKN-based approach.

Vector shedding may be determined for example by measuring vector DNA in biological fluids such as tears, serum or urine using quantitative polymerase chain reaction. In some embodiments, no vector gene copies are detectable in urine at any time point after administration of the vector. In some embodiments, less than 1000, less than 500, less than 100, less than 50 or less than 10 vector gene copies/5 μL are detectable by quantitative polymerase chain reaction in a biological fluid (e.g., tears, serum or urine) at any point after administration. In specific embodiments, 210 vector gene copies/5 μL or less are detectable in serum. In some embodiments, less than 1000, less than 500, less than 100, less than 50 or less than 10 vector gene copies/5 μL are detectable by quantitative polymerase chain reaction in a biological fluid (e.g., tears, serum or urine) by 1, 2, 3, 4, 5, 6, 7, 8, 9, 10, 11, 12, 13 or 14 weeks after administration. In specific embodiments, no vector gene copies are detectable in a biological fluid (e.g., tears, serum or urine) by Week 14 after administration of the vector. In some embodiments, no vector gene copies are detectable in a biological fluid (e.g., tears, serum or urine) at any time point after administration of the vector.

In some embodiments, patients treated in accordance with a method provided herein are monitored for the development of Center Involved-Diabetic Macular Edema (CI-DME), cataracts, neovascularization, retinal detachment, diabetes complications, vessel regression, area of leakage, and/or area of retinal nonperfusion. Development of CI-DME, cataracts, neovascularization, retinal detachment, diabetes complications, vessel regression, area of leakage, and area of retinal nonperfusion may be assessed by any method known in the art or provided herein. Diabetic complications developed in a subject may require panretinal photocoagulation (PRP), anti-VEGF therapy and/or surgical intervention). Diabetic complications may be sight-threatening. Cataracts developed in a subject may require surgery. In some embodiments, the vital signs (e.g., heart rate, blood pressure) of a patient treated in accordance with the methods provided herein may be monitored.

The safety of a method of treatment described herein may be assessed by assays known in the art. In certain embodiments, the safety of a method of treatment described herein is assessed by serum chemistry measurements of, e.g., levels of glucose, blood urea nitrogen, creatinine, sodium, potassium, chloride, carbon dioxide, calcium, total protein albumin total bilirubin, direct bilirubin, alkaline phosphatase, alanine aminotransferase, aspartate aminotransferase, and/or creatine kinase. In certain embodiments, the safety of a method of treatment described herein is assessed by hematological measurements of, e.g., platelets, hematocrit, hemoglobin, red blood cells, white blood cells, neutrophils, lymphocytes, monocytes, eosinophils, basophils, mean corpuscular volume, mean corpuscular hemoglobin and/or mean corpuscular hemoglobin concentration. In certain embodiments, the safety of a method of treatment described herein is assessed by urinalysis, e.g., a dipstick test for levels of glucose, ketones, protein, and/or blood (if warranted, a microscopic evaluation may be completed). In certain embodiments, the safety of a method of treatment described herein is assessed by measurements of coagulation (e.g., prothrombin time and/or partial thromboplastin time) or by measurements of hemoglobin Alc.

In certain embodiments, the effects of a method provided herein are determined by statistical analysis. Statistical inference may be done at a significance level of 2-sided α=0.2. Statistical endpoints may be summarized with a corresponding 80% confidence interval.

The effects of a method provided herein may be determined by Fisher's Exact test, wherein a treated population is tested against a historical rate of response (e.g., 5%) in an untreated population.

### 5.4 Steroid Regimes

In certain embodiments, provided herein is a method of treating neovascular age-related macular degeneration (nAMD) in a subject in need thereof, wherein the method comprises administering an anti-hVEGF treatment and a steroid treatment; wherein
the anti-hVEGF treatment comprises administering a therapeutically effective amount of a recombinant viral vector provided herein comprising a nucleotide sequence encoding an anti-hVEGF antigen-binding fragment to an eye of the subject; and
the steroid treatment comprises administering a therapeutically effective amount of a steroid to the eye of the subject.

In certain other embodiments, provided herein is a method of treating neovascular age-related macular degeneration (nAMD) or diabetic retinopathy (DR) in a subject in need thereof, wherein the method comprises administering an anti-hVEGF treatment and a steroid treatment; wherein
the anti-hVEGF treatment comprises administering a therapeutically effective amount of a recombinant viral vector provided herein comprising a nucleotide sequence encoding an anti-hVEGF antigen-binding fragment to the suprachoroidal space of an eye of the subject; and
the steroid treatment comprises administering a therapeutically effective amount of a steroid to the eye of the subject.

Also provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector provided herein comprising a nucleotide sequence encoding an anti-hVEGF antigen-binding fragment to an eye of the subject; and the steroid treatment comprises administering a therapeutically effective amount of triamcinolone acetonide to the eye of the subject.

Also provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector provided herein comprising a nucleotide sequence encoding an anti-hVEGF antigen-binding fragment to an eye of the subject; and the steroid treatment comprises administering a therapeutically effective amount of difluprednate to the eye of the subject.

In certain embodiments, the method is a method of treating neovascular age-related macular degeneration (nAMD). In certain embodiments, wherein the method is a method of treating diabetic retinopathy (DR).

In certain embodiments, the anti-hVEGF treatment comprises administering a recombinant viral vector described in Section 5.2.

In certain embodiments, the recombinant viral vector is administered as described in Section 5.3. In a specific embodiment, the recombinant viral vector is administered to the suprachoroidal space of the eye of the subject. In a specific embodiment, the recombinant viral vector is administered by injection into the suprachoroidal space of the eye using a suprachoroidal drug delivery device. In a specific embodiment, the suprachoroidal drug delivery device is a microinjector. In another embodiment, the recombinant viral vector is administered to the subretinal space of the eye of the subject.

In certain embodiments, the steroid treatment comprises topically administering a therapeutically effective amount of a steroid. In certain embodiments, topical administration of the steroid ameliorates or prevents intraocular inflammation. In certain embodiments, the steroid treatment comprises administering a therapeutically effective amount of a corticosteroid. In certain embodiments, administration of the corticosteroid ameliorates or prevents intraocular inflammation. In certain embodiments, the steroid treatment comprises topically administering a therapeutically effective amount of a corticosteroid. In certain embodiments, the corticosteroid is selected from the group consisting of cortisone, hydrocortisone, fludrocortisone acetate, prednisolone, prednisone, methylprednisolone, triamcinolone, dexamethasone, betamethasone, triamcinolone acetonide, difluprednate, and fluorometholone. In certain embodiments, the corticosteroid is triamcinolone acetonide. In certain embodiments, the corticosteroid is difluprednate. In certain embodiments, the steroid treatment comprises administering a therapeutically effective amount of triamcinolone acetonide. In certain embodiments, the steroid treatment comprises administering a therapeutically effective amount of difluprednate.

In certain embodiments provided herein, the anti-hVEGF treatment comprises administering a recombinant viral vector comprising a nucleotide sequence encoding an anti-hVEGF antigen-binding fragment to the suprachoroidal space of an eye of the subject; and the steroid treatment comprises administering triamcinolone acetonide to the eye of the subject. In certain embodiments, the triamcinolone acetonide is administered after administering the recombinant viral vector. In other embodiments, the triamcinolone acetonide is administered before administering the recombinant viral vector. In certain embodiments, the triamcinolone acetonide is administered to the eye of the subject within about 24 hours, about 20 hours, about 16 hours, about 12 hours, about 8 hours, about 4 hours, about 3 hours, about 2 hours, about 1 hour, about 50 minutes, about 40 minutes, about 30 minutes, about 20 minutes, about 10 minutes, about 9 minutes, about 8 minutes, about 7 minutes, about 6 minutes, about 5 minutes, about 4 minutes, about 3 minutes, about 2 minutes, or about 1 minute of administering the recombinant viral vector. In certain embodiments, the triamcinolone acetonide is administered to the eye of the subject about 24 hours, about 20 hours, about 16 hours, about 12 hours, about 8 hours, about 4 hours, about 3 hours, about 2 hours, about 1 hour, about 50 minutes, about 40 minutes, about 30 minutes, about 20 minutes, about 10 minutes, about 9 minutes, about 8 minutes, about 7 minutes, about 6 minutes, about 5 minutes, about 4 minutes, about 3 minutes, about 2 minutes, or about 1 minute before administering the recombinant viral vector. In certain embodiments, the triamcinolone acetonide is administered to the eye of the subject about 24 hours, about 20 hours, about 16 hours, about 12 hours, about 8 hours, about 4 hours, about 3 hours, about 2 hours, about 1 hour, about 50 minutes, about 40 minutes, about 30 minutes, about 20 minutes, about 10 minutes, about 9 minutes, about 8 minutes, about 7 minutes, about 6 minutes, about 5 minutes, about 4 minutes, about 3 minutes, about 2 minutes, or about 1 minute after administering the recombinant viral vector. In certain embodiments, the triamcinolone acetonide is administered to the eye of the subject about 20-24 hours, about 16-20 hours, about 12-16 hours, about 8-12 hours, about 4-8 hours, about 3-4 hours, about 2-3 hours, about 1-2 hours, about 50-60 minutes, about 40-50 minutes, about 30-40 minutes, about 20-30 minutes, about 10-20 minutes, about 9-10 minutes, about 8-9 minutes, about 7-8 minutes, about 6-7 minutes, about 5-6 minutes, about 4-5 minutes, about 3-4 minutes, about 2-3 minutes, about 1-2 minutes, or less than about 1 minute before administering the recombinant viral vector. In certain embodiments, the triamcinolone acetonide is administered to the eye of the subject about 20-24 hours, about 16-20 hours, about 12-16 hours, about 8-12 hours, about 4-8 hours, about 3-4 hours, about 2-3 hours, about 1-2 hours, about 50-60 minutes, about 40-50 minutes, about 30-40 minutes, about 20-30 minutes, about 10-20 minutes, about 9-10 minutes, about 8-9 minutes, about 7-8 minutes, about 6-7 minutes, about 5-6 minutes, about 4-5 minutes, about 3-4 minutes, about 2-3 minutes, about 1-2 minutes, or less than about 1 minute after administering the recombinant viral vector. In certain embodiments, the triamcinolone acetonide is administered by injection into the eye of the subject. In a specific embodiment, the triamcinolone acetonide is administered by a single injection into the eye of the subject. In a specific embodiment, the steroid treatment consists of a single injection of triamcinolone acetonide into the eye of the subject. In certain embodiments, the triamcinolone acetonide is administered in a different quadrant of the eye than is the recombinant viral vector. In certain embodiments, the triamcinolone acetonide is administered to the subtenon of the eye. In certain embodiments, the triamcinolone acetonide is administered at a dose of about 5 mg, about 10 mg, about 15 mg, about 20 mg, about 25 mg, about 30 mg, about 35 mg, about 40 mg, about 45 mg, about 50 mg, about 60 mg, about 70 mg, about 75 mg, about 80 mg, about 90 mg, about 100 mg, about 125 mg, about 150 mg, about 175 mg, or about 200 mg. In certain embodiments, the triamcinolone acetonide is administered at a dose of about 40 mg. In certain embodiments, the triamcinolone acetonide is administered at a dose of between about 5 mg and about 10 mg, about 10 mg and about 15 mg, about 15 mg and about 20 mg, about 20 mg and about 25 mg, about 25 mg and about 30 mg, about 30 mg and about 35 mg, about 35 mg and about 40 mg, about 40 mg and about 45 mg, about 45 mg and about 50 mg, about 50 mg and about 60 mg, about 60 mg and about 70 mg, about 70 mg and about 75 mg, about 75 mg and about 80 mg, about 80 mg and about 90 mg, about 90 mg and about 100 mg, about 100 mg and about 125 mg, about 125 mg and about 150 mg, about 150 mg and about 175 mg, or about 175 mg and about 200 mg. In certain embodiments, the triamcinolone acetonide is administered in a volume of about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, or about 2 mL. In certain embodiments, the triamcinolone acetonide is administered in a volume of about 1 mL. In certain embodiments, the triamcinolone acetonide is administered in a volume of between about 0.1 mL about 0.2 mL, about 0.2 mL and about 0.3 mL, about 0.3 mL and 0.4 mL, about 0.4 mL and 0.5 mL, about 0.5 mL and about 0.6 mL, about 0.6 mL and about 0.7 mL, about 0.7 mL and 0.8 mL, about 0.8 mL and about 0.9 mL, about 0.9 mL and about 1.0 mL, about 1 mL and about 1.1 mL, about 1.1 mL and about 1.2 mL, about 1.2 mL and 1.3 mL, about 1.3 mL and about 1.4 mL, about 1.4 mL and about 1.5 mL, about 1.5 mL and about 1.6 mL, about 1.6 mL and about 1.7 mL, about 1.7 mL and about 1.8 mL, about 1.8 mL and about 1.9 mL, or about 1.9 mL and about 2 mL.

In certain embodiments provided herein, the anti-hVEGF treatment comprises administering a recombinant viral vector comprising a nucleotide sequence encoding an anti-hVEGF antigen-binding fragment to the suprachoroidal space of an eye of the subject; and the steroid treatment comprises administering difluprednate to the eye of the subject. In certain embodiments, the difluprednate is administered daily to the eye of the subject. In certain embodiments, the steroid treatment comprises administering difluprednate four times daily. In certain embodiments, the difluprednate is administered four times daily for at least one week, at least two weeks, at least three weeks, or at least four weeks. In a specific embodiment, the difluprednate is administered four times daily for about four weeks. In certain embodiments, the steroid treatment comprises administering difluprednate three times daily. In certain embodiments, the difluprednate is administered three times daily for at least one day, at least two days, at least three days, at least four days, at least five days, at least six days, or at least one week. In a specific embodiment, the difluprednate is administered three times daily for about one week. In certain embodiments, the steroid treatment comprises administering difluprednate two times daily. In certain embodiments, the difluprednate is administered two times daily for at least one day, at least two days, at least three days, at least four days, at least five days, at least six days, or at least one week. In a specific embodiment, the difluprednate is administered two times daily for about one week. In certain embodiments, the steroid treatment comprises administering difluprednate one time daily. In certain embodiments, the difluprednate is administered one time daily for at least one day, at least two days, at least three days, at least four days, at least five days, at least six days, or at least one week. In a specific embodiment, the difluprednate is administered one time daily for about one week. In certain embodiments, the difluprednate is administered to the eye of the subject for a period of at least one week, at least two weeks, at least three weeks, at least four weeks, at least five weeks, at least six weeks, or at least seven weeks. In a specific embodiment, the difluprednate is administered to the eye of the subject for a period of about seven weeks. In another specific embodiment, the steroid treatment comprises administering difluprednate once on the first day of the steroid treatment, followed by four times daily for about four weeks, followed by three times daily for about one week, followed by two times daily for about one week, followed by one time daily for about one week. In yet another specific embodiment, the steroid treatment consists of administering difluprednate once on the first day of the steroid treatment, followed by four times daily for about four weeks, followed by three times daily for about one week, followed by two times daily for about one week, followed by one time daily for about one week. In certain embodiments, the difluprednate is administered in the form of a ophthalmic emulsion. In certain embodiments, the ophthalmic emulsion comprises 0.5 mg/mL (0.05%) difluprednate. In certain embodiments, each administration of difluprednate comprises instilling one drop of the ophthalmic emulsion in the eye of the subject. In certain embodiments, each administration of difluprednate consists of instilling one drop of the ophthalmic emulsion in the eye of the subject. In certain embodiments, difluprednate is first administered to the eye of the subject within about seven days, about six days, about five days, about four days, about three days, about two days, or about one day of administering the recombinant viral vector. In certain embodiments, difluprednate is first administered to the eye of the subject on the same day as the recombinant viral vector is administered. In certain embodiments, the first administration of difluprednate occurs after the first administration of the recombinant viral vector.

In certain embodiments, provided herein is a method of treating neovascular age-related macular degeneration (nAMD) or diabetic retinopathy (DR) in a subject in need thereof, wherein the method comprises administering an anti-hVEGF treatment and a steroid treatment; wherein
the anti-hVEGF treatment comprises administering a therapeutically effective amount of a recombinant viral vector provided herein comprising a nucleotide sequence encoding an anti-hVEGF antigen-binding fragment to the suprachoroidal space of an eye of the subject, wherein the recombinant viral vector is administered at a dose of at least about 5.0×10¹¹ genome copies per eye; and
the steroid treatment comprises administering a therapeutically effective amount of a topical steroid to the eye of the subject.

In certain embodiments, the recombinant viral vector is administered at a dose of between about 5.0×10¹¹ genome copies per eye and about 1.0×10¹² genome copies per eye. In certain embodiments, the recombinant viral vector is administered at a dose of at least about 1.0×10¹² genome copies per eye. In certain embodiments, the recombinant viral vector is administered at a dose of about 1.0×10¹² genome copies per eye. In certain embodiments, the recombinant viral vector is administered by multiple suprachoroidal injections. In certain embodiments, the recombinant viral vector is administered by triple suprachoroidal injections. In certain embodiments, the recombinant viral vector is administered by double suprachoroidal injections. In certain embodiments, the recombinant viral vector is administered by a single suprachoroidal injection. In certain embodiments, administration of the steroid ameliorates or prevents intraocular inflammation.

In certain embodiments, administration of the steroid ameliorates or prevents intraocular inflammation associated with the dose of the recombinant viral vector, the number of suprachoroidal injections, and/or the location of suprachoroidal injections. In certain embodiments, the steroid treatment comprises administering a therapeutically effective amount of a corticosteroid. In certain embodiments, the steroid treatment comprises topically administering a therapeutically effective amount of a steroid, for example, a corticosteroid. In certain embodiments, the steroid treatment comprises administering a therapeutically effective amount of a steroid, for example, a corticosteroid, to the subtenon of the eye. In certain embodiments, the corticosteroid is selected from the group consisting of cortisone, hydrocortisone, fludrocortisone acetate, prednisolone, prednisone, methylprednisolone, triamcinolone, dexamethasone, betamethasone, triamcinolone acetonide, difluprednate, and fluorometholone. In certain embodiments, the corticosteroid is difluprednate. In certain embodiments, the corticosteroid is triamcinolone acetonide.

In certain embodiments, provided herein is a method of treating neovascular age-related macular degeneration (nAMD) or diabetic retinopathy (DR) in a subject in need thereof, wherein the method comprises administering an anti-hVEGF treatment and a steroid treatment; wherein the anti-hVEGF treatment comprises administering a therapeutically effective amount of a recombinant viral vector provided herein comprising a nucleotide sequence encoding an anti-hVEGF antigen-binding fragment to the suprachoroidal space of an eye of the subject; and the steroid treatment comprises topically administering a therapeutically effective amount of a steroid to the eye of the subject. In certain embodiments, the recombinant viral vector is administered at a dose of at least about 5.0×10¹¹ genome copies per eye. In certain embodiments, the recombinant viral vector is administered at a dose of between about 5.0×10¹¹ genome copies per eye and about 1.0×10¹² genome copies per eye. In certain embodiments, the recombinant viral vector is administered at a dose of at least about 1.0×10¹² genome copies per eye. In certain embodiments, the recombinant viral vector is administered at a dose of about 1.0×10¹² genome copies per eye. In certain embodiments, the recombinant viral vector is administered by multiple suprachoroidal injections. In certain embodiments, the recombinant viral vector is administered by triple suprachoroidal injections. In certain embodiments, the recombinant viral vector is administered by double suprachoroidal injections. In certain embodiments, the recombinant viral vector is administered by a single suprachoroidal injection. In certain embodiments, topical administration of the steroid ameliorates or prevents intraocular inflammation. In certain embodiments, topical administration of the steroid ameliorates or prevents intraocular inflammation associated with the dose of the recombinant viral vector, the number of suprachoroidal injections, and/or the location of suprachoroidal injections. In certain embodiments, the steroid treatment comprises topically administering a therapeutically effective amount of a corticosteroid. In certain embodiments, the steroid treatment comprises administering a therapeutically effective amount of a steroid, for example, a corticosteroid, to the subtenon of the eye. In certain embodiments, the topical corticosteroid is selected from the group consisting of cortisone, hydrocortisone, fludrocortisone acetate, prednisolone, prednisone, methylprednisolone, triamcinolone, dexamethasone, betamethasone, triamcinolone acetonide, difluprednate, and fluorometholone. In certain embodiments, the topical corticosteroid is difluprednate. In certain embodiments, the corticosteroid administered to the subtenon of the eye is triamcinolone acetonide.

### 5.5 Combination Therapies

The methods of treatment provided herein may be combined with one or more additional therapies. In one aspect, the methods of treatment provided herein are administered with laser photocoagulation. In one aspect, the methods of treatment provided herein are administered with photodynamic therapy with verteporfin.

In one aspect, the methods of treatment provided herein are administered with intravitreal (IVT) injections with anti-VEGF agents, including but not limited to HuPTMFabVEGFi, e.g., HuGlyFabVEGFi produced in human cell lines (Dumont et al., 2015, supra), or other anti-VEGF agents such as pegaptanib, ranibizumab, aflibercept, or bevacizumab.

The additional therapies may be administered before, concurrently or subsequent to the gene therapy treatment.

The efficacy of the gene therapy treatment may be indicated by the elimination of or reduction in the number of rescue treatments using standard of care, for example, intravitreal injections with anti-VEGF agents, including but not limited to HuPTMFabVEGFi, e.g., HuGlyFabVEGFi produced in human cell lines, or other anti-VEGF agents such as pegaptanib, ranibizumab, aflibercept, or bevacizumab.

TABLE 3
TABLE OF SEQUENCES

| SEQ | | |
|---|---|---|
| ID | | |
| NO: | Description | Sequence |
| 1 | Ranibizumab Fab | DIQLTQSPSSLSASVGDRVTITCSASQDISNYLNWYQQKPGKAPKVLIYFT |
| | Amino Acid | SSLHSGVPSRFSGSGSGTDFTLTISSLQPEDFATYYCQQYSTVPWTFGQGT |
| | Sequence (Light | KVEIKRTVAAPSVFIFPPSDEQLKSGTASVVCLLNNFYPREAKVQWKVDNA |
| | chain) | LQSGNSQESVTEQDSKDSTYSLSSTLTLSKADYEKHKVYACEVTHQGLSSP |
| | | VTKSFNRGEC |
| 2 | Ranibizumab Fab | EVQLVESGGGLVQPGGSLRLSCAASGYDFTHYGMNWVRQAPGKGLEWVGWI |
| | Amino Acid | NTYTGEPTYAADFKRRFTFSLDTSKSTAYLQMNSLRAEDTAVYYCAKYPYY |
| | Sequence (Heavy | YGTSHWYFDVWGQGTLVTVSSASTKGPSVFPLAPSSKSTSGGTAALGCLVK |
| | chain) | DYFPEPVTVSWNSGALTSGVHTFPAVLQSSGLYSLSSVVTVPSSSLGTQTY |
| | | ICNVNHKPSNTKVDKKVEPKSCDKTHL |
| 3 | Bevacizumab Fab | DIQMTQSPSSLSASVGDRVTITCSASQDISNYLNWYQQKPGKAPKVLIYFT |
| | Amino Acid | SSLHSGVPSRFSGSGSGTDFTLTISSLQPEDFATYYCQQYSTVPWTFGQGT |
| | Sequence (Light | KVEIKRTVAAPSVFIFPPSDEQLKSGTASVVCLLNNFYPREAKVQWKVDNA |
| | chain) | LQSGNSQESVTEQDSKDSTYSLSSTLTLSKADYEKHKVYACEVTHQGLSSP |
| | | VTKSFNRGEC |
| 4 | Bevacizumab Fab | EVQLVESGGGLVQPGGSLRLSCAASGYTFTNYGMNWVRQAPGKGLEWVGWI |
| | Amino Acid | NTYTGEPTYAADFKRRFTFSLDTSKSTAYLQMNSLRAEDTAVYYCAKYPHY |
| | Sequence (Heavy | YGSSHWYFDVWGQGTLVTVSSASTKGPSVFPLAPSSKSTSGGTAALGCLVK |
| | chain) | DYFPEPVTVSWNSGALTSGVHTFPAVLQSSGLYSLSSVVTVPSSSLGTQTY |
| | | ICNVNHKPSNTKVDKKVEPKSCDKTHL |
| 5 | VEGF-A signal | MNFLLSWVHWSLALLLYLHHAKWSQA |
| | peptide | |
| 6 | Fibulin-1 signal | MERAAPSRRVPLPLLLLGGLALLAAGVDA |
| | peptide | |
| 7 | Vitronectin signal | MAPLRPLLILALLAWVALA |
| | peptide | |
| 8 | Complement | MRLLAKIICLMLWAICVA |
| | Factor H signal | |
| | peptide | |
| 9 | Opticin signal | MRLLAFLSLLALVLQETGT |
| | peptide | |
| 10 | Bevacizumab | gctagcgcca ccatgggctg gtcctgcatc atcctgttcc |
| | cDNA (Light | tggtggccac cgccaccggc gtgcactccg acatccagat |
| | chain) | gacccagtcc ccctcctccc tgtccgcctc cgtgggcgac |
| | | cgggtgacca tcacctgctc cgcctcccag gacatctcca |
| | | actacctgaa ctggtaccag cagaagcccg gcaaggcccc |
| | | caaggtgctg atctacttca cctcctccct gcactccggc |
| | | gtgccctccc ggttctccgg ctccggctcc ggcaccgact |
| | | tcaccctgac catctcctcc ctgcagcccg aggacttcgc |
| | | cacctactac tgccagcagt actccaccgt gccctggacc |
| | | ttcggccagg gcaccaaggt ggagatcaag cggaccgtgg |
| | | ccgccccctc cgtgttcatc ttccccccct ccgacgagca |
| | | gctgaagtcc ggcaccgcct ccgtggtgtg cctgctgaac |
| | | aacttctacc cccgggaggc caaggtgcag tggaaggtgg |
| | | acaacgccct gcagtccggc aactcccagg agtccgtgac |
| | | cgagcaggac tccaaggact ccacctactc cctgtcctcc |
| | | accctgaccc tgtccaaggc cgactacgag aagcacaagg |
| | | tgtacgcctg cgaggtgacc caccagggcc tgtcctcccc |
| | | cgtgaccaag tccttcaacc ggggcgagtg ctgagcggcc |
| | | gcctcgag |
| 11 | Bevacizumab | gctagcgcca ccatgggctg gtcctgcatc atcctgttcc |
| | CDNA (Heavy | tggtggccac cgccaccggc gtgcactccg aggtgcagct |
| | chain) | ggtggagtcc ggcggcggcc tggtgcagcc cggcggctcc |
| | | ctgcggctgt cctgcgccgc ctccggctac accttcacca |
| | | actacggcat gaactgggtg cggcaggccc ccggcaaggg |
| | | cctggagtgg gtgggctgga tcaacaccta caccggcgag |
| | | cccacctacg ccgccgactt caagcggcgg ttcaccttct |
| | | ccctggacac ctccaagtcc accgcctacc tgcagatgaa |
| | | ctccctgcgg gccgaggaca ccgccgtgta ctactgcgcc |
| | | aagtaccccc actactacgg ctcctcccac tggtacttcg |
| | | acgtgtgggg ccagggcacc ctggtgaccg tgtcctccgc |
| | | ctccaccaag ggcccctccg tgttccccct ggccccctcc |
| | | tccaagtcca cctccggcgg caccgccgcc ctgggctgcc |
| | | tggtgaagga ctacttcccc gagcccgtga ccgtgtcctg |
| | | gaactccggc gccctgacct ccggcgtgca caccttcccc |
| | | gccgtgctgc agtcctccgg cctgtactcc ctgtcctccg |
| | | tggtgaccgt gccctcctcc tccctgggca cccagaccta |
| | | catctgcaac gtgaaccaca agccctccaa caccaaggtg |
| | | gacaagaagg tggagcccaa gtcctgcgac aagacccaca |
| | | cctgcccccc ctgccccgcc cccgagctgc tgggcggccc |
| | | ctccgtgttc ctgttccccc ccaagcccaa ggacaccctg |
| | | atgatctccc ggacccccga ggtgacctgc gtggtggtgg |
| | | acgtgtccca cgaggacccc gaggtgaagt tcaactggta |
| | | cgtggacggc gtggaggtgc acaacgccaa gaccaagccc |
| | | cgggaggagc agtacaactc cacctaccgg gtggtgtccg |
| | | tgctgaccgt gctgcaccag gactggctga acggcaagga |
| | | gtacaagtgc aaggtgtcca acaaggccct gcccgccccc |
| | | atcgagaaga ccatctccaa ggccaagggc cagccccggg |
| | | agccccaggt gtacaccctg cccccctccc gggaggagat |
| | | gaccaagaac caggtgtccc tgacctgcct ggtgaagggc |
| | | ttctacccct ccgacatcgc cgtggagtgg gagtccaacg |
| | | gccagcccga gaacaactac aagaccaccc cccccgtgct |
| | | ggactccgac ggctccttct toctgtactc caagctgacc |
| | | gtggacaagt cccggtggca gcagggcaac gtgttctcct |
| | | gctccgtgat gcacgaggcc ctgcacaacc actacaccca |
| | | gaagtccctg tccctgtccc ccggcaagtg agcggccgcc |
| 12 | Ranibizumab | gagctccatg gagtttttca aaaagacggc acttgccgca |
| | cDNA (Light | ctggttatgg gttttagtgg tgcagcattg gccgatatcc |
| | chain comprising | agctgaccca gagcccgagc agcctgagcg caagcgttgg |
| | a signal | tgatcgtgtt accattacct gtagcgcaag ccaggatatt |
| | sequence) | agcaattatc tgaattggta tcagcagaaa ccgggtaaag |
| | | caccgaaagt tctgatttat tttaccagca gcctgcatag |
| | | cggtgttccg agccgtttta gcggtagcgg tagtggcacc |
| | | gattttaccc tgaccattag cagcctgcag ccggaagatt |
| | | ttgcaaccta ttattgtcag cagtatagca ccgttccgtg |
| | | gacctttggt cagggcacca aagttgaaat taaacgtacc |
| | | gttgcagcac cgagcgtttt tatttttccg cctagtgatg |
| | | aacagctgaa aagcggcacc gcaagcgttg tttgtctgct |
| | | gaataatttt tatccgcgtg aagcaaaagt gcagtggaaa |
| | | gttgataatg cactgcagag cggtaatagc caagaaagcg |
| | | ttaccgaaca ggatagcaaa gatagcacct atagcctgag |
| | | cagcaccctg accctgagca aagcagatta tgaaaaacac |
| | | aaagtgtatg cctgcgaagt tacccatcag ggtctgagca |
| | | gtccggttac caaaagtttt aatcgtggcg aatgctaata |
| | | gaagcttggt acc |
| 13 | Ranibizumab | gagctcatat gaaatacctg ctgccgaccg ctgctgctgg |
| | cDNA (Heavy | tctgctgctc ctcgctgccc agccggcgat ggccgaagtt |
| | chain comprising | cagctggttg aaagcggtgg tggtctggtt cagcctggtg |
| | a signal | gtagcctgcg tctgagctgt gcagcaagcg gttatgattt |
| | sequence) | tacccattat ggtatgaatt gggttcgtca ggcaccgggt |
| | | aaaggtctgg aatgggttgg ttggattaat acctataccg |
| | | gtgaaccgac ctatgcagca gattttaaac gtcgttttac |
| | | ctttagcctg gataccagca aaagcaccgc atatctgcag |
| | | atgaatagcc tgcgtgcaga agataccgca gtttattatt |
| | | gtgccaaata tccgtattac tatggcacca gccactggta |
| | | tttcgatgtt tggggtcagg gcaccctggt taccgttagc |
| | | agcgcaagca ccaaaggtcc gagcgttttt ccgctggcac |
| | | cgagcagcaa aagtaccagc ggtggcacag cagcactggg |
| | | ttgtctggtt aaagattatt ttccggaacc ggttaccgtg |
| | | agctggaata gcggtgcact gaccagcggt gttcatacct |
| | | ttccggcagt tctgcagagc agcggtctgt atagcctgag |
| | | cagcgttgtt accgttccga gcagcagcct gggcacccag |
| | | acctatattt gtaatgttaa tcataaaccg agcaatacca |
| | | aagtggataa aaaagttgag ccgaaaagct gcgataaaac |
| | | ccatctgtaa tagggtacc |
| 14 | Bevacizumab and | SASQDISNYLN |
| | Ranibizumab | |
| | Light Chain | |
| | CDR1 | |
| 15 | Bevacizumab and | FTSSLHS |
| | Ranibizumab | |
| | Light Chain | |
| | CDR2 | |
| 16 | Bevacizumab and | QQYSTVPWT |
| | Ranibizumab | |
| | Light Chain | |
| | CDR3 | |
| 17 | Bevacizumab | GYTFTNYGMN |
| | Heavy Chain | |
| | CDR1 | |
| 18 | Bevacizumab and | WINTYTGEPTYAADFKR |
| | Ranibizumab | |
| | Heavy Chain | |
| | CDR2 | |
| 19 | Bevacizumab | YPHYYGSSHWYFDV |
| | Heavy Chain | |
| | CDR3 | |
| 20 | Ranibizumab | GYDFTHYGMN |
| | Heavy Chain | |
| | CDR1 | |
| 21 | Ranibizumab | YPYYYGTSHWYFDV |
| | Heavy Chain | |
| | CDR3 | |
| 22 | Albumin signal | MKWVTFISLLFLFSSAYS |
| | peptide | |
| 23 | Chymotrypsinogen | MAFLWLLSCWALLGTTFG |
| | signal peptide | |
| 24 | Interleukin-2 | MYRMQLLSCIALILALVTNS |
| | signal peptide | |
| 25 | Trypsinogen-2 | MNLLLILTFVAAAVA |
| | signal peptide | |
| 26 | F2A site | LLNFDLLKLAGDVESNPGP |
| 27 | T2A site | (GSG) EGRGSLLTCGDVEENPGP |
| 28 | P2A site | (GSG) ATNFSLLKQAGDVEENPGP |
| 29 | E2A site | (GSG) QCTNYALLKLAGDVESNPGP |
| 30 | F2A site | (GSG) VKQTLNFDLLKLAGDVESNPGP |
| 31 | Furin linker | RKRR |
| 32 | Furin linker | RRRR |
| 33 | Furin linker | RRKR |
| 34 | Furin linker | RKKR |
| 35 | Furin linker | R-X-K/R-R |
| 36 | Furin linker | RXKR |
| 37 | Furin linker | RXRR |
| 38 | Ranibizumab Fab | MDIQLTQSPSSLSASVGDRVTITCSASQDISNYLNWYQQKPGKAPKVLIYF |
| | amino acid | TSSLHSGVPSRFSGSGSGTDFTLTISSLQPEDFATYYCQQYSTVPWTFGQG |
| | sequence (Light | TKVEIKRTVAAPSVFIFPPSDEQLKSGTASVVCLLNNFYPREAKVQWKVDN |
| | chain) | ALQSGNSQESVTEQDSKDSTYSLSSTLTLSKADYEKHKVYACEVTHQGLSS |
| | | PVTKSFNRGEC |
| 39 | Ranibizumab Fab | MEVQLVESGGGLVQPGGSLRLSCAASGYDFTHYGMNWVRQAPGKGLEWVGW |
| | amino acid | INTYTGEPTYAADFKRRFTFSLDTSKSTAYLQMNSLRAEDTAVYYCAKYPY |
| | sequence (Heavy | YYGTSHWYFDVWGQGTLVTVSSASTKGPSVFPLAPSSKSTSGGTAALGCLV |
| | chain) | KDYFPEPVTVSWNSGALTSGVHTFPAVLQSSGLYSLSSVVTVPSSSLGTQT |
| | | YICNVNHKPSNTKVDKKVEPKSCDKTHLRKRR |
| 40 | Ranibizumab Fab | MEVQLVESGGGLVQPGGSLRLSCAASGYDFTHYGMNWVRQAPGKGLEWVGW |
| | amino acid | INTYTGEPTYAADFKRRFTFSLDTSKSTAYLQMNSLRAEDTAVYYCAKYPY |
| | sequence (Heavy | YYGTSHWYFDVWGQGTLVTVSSASTKGPSVFPLAPSSKSTSGGTAALGCLV |
| | chain) | KDYFPEPVTVSWNSGALTSGVHTFPAVLQSSGLYSLSSVVTVPSSSLGTQT |
| | | YICNVNHKPSNTKVDKKVEPKSCDKTHL |
| 41 | AAV1 | MAADGYLPDWLEDNLSEGIREWWDLKPGAPKPKANQQKQDDGRGLVLPGYK |
| | | YLGPFNGLDKGEPVNAADAAALEHDKAYDQQLKAGDNPYLRYNHADAEFQE |
| | | RLQEDTSFGGNLGRAVFQAKKRVLEPLGLVEEGAKTAPGKKRPVEQSPQEP |
| | | DSSSGIGKTGQQPAKKRLNFGQTGDSESVPDPQPLGEPPATPAAVGPTTMA |
| | | SGGGAPMADNNEGADGVGNASGNWHCDSTWLGDRVITTSTRTWALPTYNNH |
| | | LYKQISSASTGASNDNHYFGYSTPWGYFDFNRFHCHFSPRDWQRLINNNWG |
| | | FRPKRLNFKLFNIQVKEVTTNDGVTTIANNLTSTVQVFSDSEYQLPYVLGS |
| | | AHQGCLPPFPADVFMIPQYGYLTLNNGSQAVGRSSFYCLEYFPSQMLRTGN |
| | | NFTFSYTFEEVPFHSSYAHSQSLDRLMNPLIDQYLYYLNRTQNQSGSAQNK |
| | | DLLFSRGSPAGMSVQPKNWLPGPCYRQQRVSKTKTDNNNSNFTWTGASKYN |
| | | LNGRESIINPGTAMASHKDDEDKFFPMSGVMIFGKESAGASNTALDNVMIT |
| | | DEEEIKATNPVATERFGTVAVNFQSSSTDPATGDVHAMGALPGMVWQDRDV |
| | | YLQGPIWAKIPHTDGHFHPSPLMGGFGLKNPPPQILIKNTPVPANPPAEFS |
| | | ATKFASFITQYSTGQVSVEIEWELQKENSKRWNPEVQYTSNYAKSANVDFT |
| | | VDNNGLYTEPRPIGTRYLTRPL |
| 42 | AAV2 | MAADGYLPDWLEDTLSEGIRQWWKLKPGPPPPKPAERHKDDSRGLVLPGYK |
| | | YLGPFNGLDKGEPVNEADAAALEHDKAYDRQLDSGDNPYLKYNHADAEFQE |
| | | RLKEDTSFGGNLGRAVFQAKKRVLEPLGLVEEPVKTAPGKKRPVEHSPVEP |
| | | DSSSGTGKAGQQPARKRLNFGQTGDADSVPDPQPLGQPPAAPSGLGTNTMA |
| | | TGSGAPMADNNEGADGVGNSSGNWHCDSTWMGDRVITTSTRTWALPTYNNH |
| | | LYKQISSQSGASNDNHYFGYSTPWGYFDFNRFHCHFSPRDWQRLINNNWGF |
| | | RPKRLNFKLFNIQVKEVTQNDGTTTIANNLTSTVQVFTDSEYQLPYVLGSA |
| | | HQGCLPPFPADVFMVPQYGYLTLNNGSQAVGRSSFYCLEYFPSQMLRTGNN |
| | | FTFSYTFEDVPFHSSYAHSQSLDRLMNPLIDQYLYYLSRTNTPSGTTTQSR |
| | | LQFSQAGASDIRDQSRNWLPGPCYRQQRVSKTSADNNNSEYSWTGATKYHL |
| | | NGRDSLVNPGPAMASHKDDEEKFFPQSGVLIFGKQGSEKTNVDIEKVMITD |
| | | EEEIRTTNPVATEQYGSVSTNLQRGNRQAATADVNTQGVLPGMVWQDRDVY |
| | | LQGPIWAKIPHTDGHFHPSPLMGGFGLKHPPPQILIKNTPVPANPSTTFSA |
| | | AKFASFITQYSTGQVSVEIEWELQKENSKRWNPEIQYTSNYNKSVNVDFTV |
| | | DTNGVYSEPRPIGTRYLTRNL |
| 43 | AAV3-3 | MAADGYLPDWLEDNLSEGIREWWALKPGVPQPKANQQHQDNRRGL |
| | | VLPGYKYLGPGNGLDKGEPVNEADAAALEHDKAYDQQLKAGDNPYLKYNHA |
| | | DAEFQERLQEDTSFGGNLGRAVFQAKKRILEPLGLVEEAAKTAPGKKGAVD |
| | | QSPQEPDSSSGVGKSGKQPARKRLNFGQTGDSESVPDPQPLGEPPAAPTSL |
| | | GSNTMASGGGAPMADNNEGADGVGNSSGNWHCDSQWLGDRVITTSTRTWAL |
| | | PTYNNHLYKQISSQSGASNDNHYFGYSTPWGYFDFNRFHCHFSPRDWQRLI |
| | | NNNWGFRPKKLSFKLFNIQVRGVTQNDGTTTIANNLTSTVQVFTDSEYQLP |
| | | YVLGSAHQGCLPPFPADVFMVPQYGYLTLNNGSQAVGRSSFYCLEYFPSQM |
| | | LRTGNNFQFSYTFEDVPFHSSYAHSQSLDRLMNPLIDQYLYYLNRTQGTTS |
| | | GTTNQSRLLFSQAGPQSMSLQARNWLPGPCYRQQRLSKTANDNNNSNFPWT |
| | | AASKYHLNGRDSLVNPGPAMASHKDDEEKFFPMHGNLIFGKEGTTASNAEL |
| | | DNVMITDEEEIRTTNPVATEQYGTVANNLQSSNTAPTTGTVNHQGALPGMV |
| | | WQDRDVYLQGPIWAKIPHTDGHFHPSPLMGGFGLKHPPPQIMIKNTPVPAN |
| | | PPTTFSPAKFASFITQYSTGQVSVEIEWELQKENSKRWNPEIQYTSNYNKS |
| | | VNVDFTVDTNGVYSEPRPIGTRYLTRNL |
| 44 | AAV4-4 | MTDGYLPDWLEDNLSEGVREWWALQPGAPKPKANQQHQDNARGLVLPGYKY |
| | | LGPGNGLDKGEPVNAADAAALEHDKAYDQQLKAGDNPYLKYNHADAEFQQR |
| | | LQGDTSFGGNLGRAVFQAKKRVLEPLGLVEQAGETAPGKKRPLIESPQQPD |
| | | SSTGIGKKGKQPAKKKLVFEDETGAGDGPPEGSTSGAMSDDSEMRAAAGGA |
| | | AVEGGQGADGVGNASGDWHCDSTWSEGHVTTTSTRTWVLPTYNNHLYKRLG |
| | | ESLQSNTYNGFSTPWGYFDFNRFHCHFSPRDWQRLINNNWGMRPKAMRVKI |
| | | FNIQVKEVTTSNGETTVANNLTSTVQIFADSSYELPYVMDAGQEGSLPPFP |
| | | NDVFMVPQYGYCGLVTGNTSQQQTDRNAFYCLEYFPSQMLRTGNNFEITYS |
| | | FEKVPFHSMYAHSQSLDRLMNPLIDQYLWGLQSTTTGTTLNAGTATTNFTK |
| | | LRPTNFSNFKKNWLPGPSIKQQGFSKTANQNYKIPATGSDSLIKYETHSTL |
| | | DGRWSALTPGPPMATAGPADSKFSNSQLIFAGPKQNGNTATVPGTLIFTSE |
| | | EELAATNATDTDMWGNLPGGDQSNSNLPTVDRLTALGAVPGMVWQNRDIYY |
| | | QGPIWAKIPHTDGHFHPSPLIGGFGLKHPPPQIFIKNTPVPANPATTFSST |
| | | PVNSFITQYSTGQVSVQIDWEIQKERSKRWNPEVQFTSNYGQQNSLLWAPD |
| | | AAGKYTEPRAIGTRYLTHHL |
| 45 | AAV5 | MSFVDHPPDWLEEVGEGLREFLGLEAGPPKPKPNQQHQDQARGLVLPGYNY |
| | | LGPGNGLDRGEPVNRADEVAREHDISYNEQLEAGDNPYLKYNHADAEFQEK |
| | | LADDTSFGGNLGKAVFQAKKRVLEPFGLVEEGAKTAPTGKRIDDHFPKRKK |
| | | ARTEEDSKPSTSSDAEAGPSGSQQLQIPAQPASSLGADTMSAGGGGPLGDN |
| | | NQGADGVGNASGDWHCDSTWMGDRVVTKSTRTWVLPSYNNHQYREIKSGSV |
| | | DGSNANAYFGYSTPWGYFDFNRFHSHWSPRDWQRLINNYWGFRPRSLRVKI |
| | | FNIQVKEVTVQDSTTTIANNLTSTVQVFTDDDYQLPYVVGNGTEGCLPAFP |
| | | PQVFTLPQYGYATLNRDNTENPTERSSFFCLEYFPSKMLRTGNNFEFTYNF |
| | | EEVPFHSSFAPSQNLFKLANPLVDQYLYRFVSTNNTGGVQFNKNLAGRYAN |
| | | TYKNWFPGPMGRTQGWNLGSGVNRASVSAFATTNRMELEGASYQVPPQPNG |
| | | MTNNLQGSNTYALENTMIFNSQPANPGTTATYLEGNMLITSESETQPVNRV |
| | | AYNVGGQMATNNQSSTTAPATGTYNLQEIVPGSVWMERDVYLQGPIWAKIP |
| | | ETGAHFHPSPAMGGFGLKHPPPMMLIKNTPVPGNITSFSDVPVSSFITQYS |
| | | TGQVTVEMEWELKKENSKRWNPEIQYTNNYNDPQFVDFAPDSTGEYRTTRP |
| | | IGTRYLTRPL |
| 46 | AAV6 | MAADGYLPDWLEDNLSEGIREWWDLKPGAPKPKANQQKQDDGRGLVLPGYK |
| | | YLGPFNGLDKGEPVNAADAAALEHDKAYDQQLKAGDNPYLRYNHADAEFQE |
| | | RLQEDTSFGGNLGRAVFQAKKRVLEPFGLVEEGAKTAPGKKRPVEQSPQEP |
| | | DSSSGIGKTGQQPAKKRLNFGQTGDSESVPDPQPLGEPPATPAAVGPTTMA |
| | | SGGGAPMADNNEGADGVGNASGNWHCDSTWLGDRVITTSTRTWALPTYNNH |
| | | LYKQISSASTGASNDNHYFGYSTPWGYFDFNRFHCHFSPRDWQRLINNNWG |
| | | FRPKRLNFKLFNIQVKEVTTNDGVTTIANNLTSTVQVFSDSEYQLPYVLGS |
| | | AHQGCLPPFPADVFMIPQYGYLTLNNGSQAVGRSSFYCLEYFPSQMLRTGN |
| | | NFTFSYTFEDVPFHSSYAHSQSLDRLMNPLIDQYLYYLNRTQNQSGSAQNK |
| | | DLLFSRGSPAGMSVQPKNWLPGPCYRQQRVSKTKTDNNNSNFTWTGASKYN |
| | | LNGRESIINPGTAMASHKDDKDKFFPMSGVMIFGKESAGASNTALDNVMIT |
| | | DEEEIKATNPVATERFGTVAVNLQSSSTDPATGDVHVMGALPGMVWQDRDV |
| | | YLQGPIWAKIPHTDGHFHPSPLMGGFGLKHPPPQILIKNTPVPANPPAEFS |
| | | ATKFASFITQYSTGQVSVEIEWELQKENSKRWNPEVQYTSNYAKSANVDFT |
| | | VDNNGLYTEPRPIGTRYLTRPL |
| 47 | AAV7 | MAADGYLPDWLEDNLSEGIREWWDLKPGAPKPKANQQKQDNGRGLVLPGYK |
| | | YLGPFNGLDKGEPVNAADAAALEHDKAYDQQLKAGDNPYLRYNHADAEFQE |
| | | RLQEDTSFGGNLGRAVFQAKKRVLEPLGLVEEGAKTAPAKKRPVEPSPQRS |
| | | PDSSTGIGKKGQQPARKRLNFGQTGDSESVPDPQPLGEPPAAPSSVGSGTV |
| | | AAGGGAPMADNNEGADGVGNASGNWHCDSTWLGDRVITTSTRTWALPTYNN |
| | | HLYKQISSETAGSTNDNTYFGYSTPWGYFDFNRFHCHFSPRDWQRLINNNW |
| | | GFRPKKLRFKLFNIQVKEVTTNDGVTTIANNLTSTIQVFSDSEYQLPYVLG |
| | | SAHQGCLPPFPADVFMIPQYGYLTLNNGSQSVGRSSFYCLEYFPSQMLRTG |
| | | NNFEFSYSFEDVPFHSSYAHSQSLDRLMNPLIDQYLYYLARTQSNPGGTAG |
| | | NRELQFYQGGPSTMAEQAKNWLPGPCFRQQRVSKTLDQNNNSNFAWTGATK |
| | | YHLNGRNSLVNPGVAMATHKDDEDRFFPSSGVLIFGKTGATNKTTLENVLM |
| | | TNEEEIRPTNPVATEEYGIVSSNLQAANTAAQTQVVNNQGALPGMVWQNRD |
| | | VYLQGPIWAKIPHTDGNFHPSPLMGGFGLKHPPPQILIKNTPVPANPPEVF |
| | | TPAKFASFITQYSTGQVSVEIEWELQKENSKRWNPEIQYTSNFEKQTGVDF |
| | | AVDSQGVYSEPRPIGTRYLTRNL |
| 48 | AAV8 | MAADGYLPDWLEDNLSEGIREWWALKPGAPKPKANQQKQDDGRGLVLPGYK |
| | | YLGPFNGLDKGEPVNAADAAALEHDKAYDQQLQAGDNPYLRYNHADAEFQE |
| | | RLQEDTSFGGNLGRAVFQAKKRVLEPLGLVEEGAKTAPGKKRPVEPSPQRS |
| | | PDSSTGIGKKGQQPARKRLNFGQTGDSESVPDPQPLGEPPAAPSGVGPNTM |
| | | AAGGGAPMADNNEGADGVGSSSGNWHCDSTWLGDRVITTSTRTWALPTYNN |
| | | HLYKQISNGTSGGATNDNTYFGYSTPWGYFDFNRFHCHFSPRDWQRLINNN |
| | | WGFRPKRLSFKLFNIQVKEVTQNEGTKTIANNLTSTIQVFTDSEYQLPYVL |
| | | GSAHQGCLPPFPADVFMIPQYGYLTLNNGSQAVGRSSFYCLEYFPSQMLRT |
| | | GNNFQFTYTFEDVPFHSSYAHSQSLDRLMNPLIDQYLYYLSRTQTTGGTAN |
| | | TQTLGFSQGGPNTMANQAKNWLPGPCYRQQRVSTTTGQNNNSNFAWTAGTK |
| | | YHLNGRNSLANPGIAMATHKDDEERFFPSNGILIFGKQNAARDNADYSDVM |
| | | LTSEEEIKTTNPVATEEYGIVADNLQQQNTAPQIGTVNSQGALPGMVWQNR |
| | | DVYLQGPIWAKIPHTDGNFHPSPLMGGFGLKHPPPQILIKNTPVPADPPTT |
| | | FNQSKLNSFITQYSTGQVSVEIEWELQKENSKRWNPEIQYTSNYYKSTSVD |
| | | FAVNTEGVYSEPRPIGTRYLTRNL |
| 49 | hu31 | MAADGYLPDWLEDTLSEGIRQWWKLKPGPPPPKPAERHKDDSRGLVLPGYK |
| | | YLGPGNGLDKGEPVNAADAAALEHDKAYDQQLKAGDNPYLKYNHADAEFQE |
| | | RLKEDTSFGGNLGRAVFQAKKRLLEPLGLVEEAAKTAPGKKRPVEQSPQEP |
| | | DSSAGIGKSGSQPAKKKLNFGQTGDTESVPDPQPIGEPPAAPSGVGSLTMA |
| | | SGGGAPVADNNEGADGVGSSSGNWHCDSQWLGDRVITTSTRTWALPTYNNH |
| | | LYKQISNSTSGGSSNDNAYFGYSTPWGYFDFNRFHCHFSPRDWQRLINNNW |
| | | GFRPKRLNFKLFNIQVKEVTDNNGVKTIANNLTSTVQVFTDSDYQLPYVLG |
| | | SAHEGCLPPFPADVFMIPQYGYLTLNDGGQAVGRSSFYCLEYFPSQMLRTG |
| | | NNFQFSYEFENVPFHSSYAHSQSLDRLMNPLIDQYLYYLSKTINGSGQNQQ |
| | | TLKFSVAGPSNMAVQGRNYIPGPSYRQQRVSTTVTQNNNSEFAWPGASSWA |
| | | LNGRNSLMNPGPAMASHKEGEDRFFPLSGSLIFGKQGTGRDNVDADKVMIT |
| | | NEEEIKTTNPVATESYGQVATNHQSAQAQAQTGWVQNQGILPGMVWQDRDV |
| | | YLQGPIWAKIPHTDGNFHPSPLMGGFGMKHPPPQILIKNTPVPADPPTAFN |
| | | KDKLNSFITQYSTGQVSVEIEWELQKENSKRWNPEIQYTSNYYKSNNVEFA |
| | | VSTEGVYSEPRPIGTRYLTRNL |
| 50 | hu32 | MAADGYLPDWLEDTLSEGIRQWWKLKPGPPPPKPAERHKDDSRGLVLPGYK |
| | | YLGPGNGLDKGEPVNAADAAALEHDKAYDQQLKAGDNPYLKYNHADAEFQE |
| | | RLKEDTSFGGNLGRAVFQAKKRLLEPLGLVEEAAKTAPGKKRPVEQSPQEP |
| | | DSSAGIGKSGSQPAKKKLNFGQTGDTESVPDPQPIGEPPAAPSGVGSLTMA |
| | | SGGGAPVADNNEGADGVGSSSGNWHCDSQWLGDRVITTSTRTWALPTYNNH |
| | | LYKQISNSTSGGSSNDNAYFGYSTPWGYFDFNRFHCHFSPRDWQRLINNNW |
| | | GFRPKRLNFKLFNIQVKEVTDNNGVKTIANNLTSTVQVFTDSDYQLPYVLG |
| | | SAHEGCLPPFPADVFMIPQYGYLTLNDGSQAVGRSSFYCLEYFPSQMLRTG |
| | | NNFQFSYEFENVPFHSSYAHSQSLDRLMNPLIDQYLYYLSKTINGSGQNQQ |
| | | TLKFSVAGPSNMAVQGRNYIPGPSYRQQRVSTTVTQNNNSEFAWPGASSWA |
| | | LNGRNSLMNPGPAMASHKEGEDRFFPLSGSLIFGKQGTGRDNVDADKVMIT |
| | | NEEEIKTTNPVATESYGQVATNHQSAQAQAQTGWVQNQGILPGMVWQDRDV |
| | | YLQGPIWAKIPHTDGNFHPSPLMGGFGMKHPPPQILIKNTPVPADPPTAFN |
| | | KDKLNSFITQYSTGQVSVEIEWELQKENSKRWNPEIQYTSNYYKSNNVEFA |
| | | VNTEGVYSEPRPIGTRYLTRNL |
| 51 | AAV9 | MAADGYLPDWLEDNLSEGIREWWALKPGAPQPKANQQHQDNARGLVLPGYK |
| | | YLGPGNGLDKGEPVNAADAAALEHDKAYDQQLKAGDNPYLKYNHADAEFQE |
| | | RLKEDTSFGGNLGRAVFQAKKRLLEPLGLVEEAAKTAPGKKRPVEQSPQEP |
| | | DSSAGIGKSGAQPAKKRLNFGQTGDTESVPDPQPIGEPPAAPSGVGSLTMA |
| | | SGGGAPVADNNEGADGVGSSSGNWHCDSQWLGDRVITTSTRTWALPTYNNH |
| | | LYKQISNSTSGGSSNDNAYFGYSTPWGYFDFNRFHCHFSPRDWQRLINNNW |
| | | GFRPKRLNFKLFNIQVKEVTDNNGVKTIANNLTSTVQVFTDSDYQLPYVLG |
| | | SAHEGCLPPFPADVFMIPQYGYLTLNDGSQAVGRSSFYCLEYFPSQMLRTG |
| | | NNFQFSYEFENVPFHSSYAHSQSLDRLMNPLIDQYLYYLSKTINGSGQNQQ |
| | | TLKFSVAGPSNMAVQGRNYIPGPSYRQQRVSTTVTQNNNSEFAWPGASSWA |
| | | LNGRNSLMNPGPAMASHKEGEDRFFPLSGSLIFGKQGTGRDNVDADKVMIT |
| | | NEEEIKTTNPVATESYGQVATNHQSAQAQAQTGWVQNQGILPGMVWQDRDV |
| | | YLQGPIWAKIPHTDGNFHPSPLMGGFGMKHPPPQILIKNTPVPADPPTAFN |
| | | KDKLNSFITQYSTGQVSVEIEWELQKENSKRWNPEIQYTSNYYKSNNVEFA |
| | | VNTEGVYSEPRPIGTRYLTRNL |
| 55 | Mutant | MYRMQLLLLIALSLALVINS |
| | interleukin-2 | |
| | signal peptide | |
| 56 | ITR.CB7.CI.aVE | ctgcgcgctc gctcgctcac tgaggccgcc cgggcaaagc |
| | GRv3.rBG.ITR | ccgggcgtcg ggcgaccttt ggtcgcccgg cctcagtgag |
| | | cgagcgagcg cgcagagagg gagtggccaa ctccatcact |
| | | aggggttcct tgtagttaat gattaacccg ccatgctact |
| | | tatctaccag ggtaatgggg atcctctaga actatagcta |
| | | gtcgacattg attattgact agttattaat agtaatcaat |
| | | tacggggtca ttagttcata gcccatatat ggagttccgc |
| | | gttacataac ttacggtaaa tggcccgcct ggctgaccgc |
| | | ccaacgaccc ccgcccattg acgtcaataa tgacgtatgt |
| | | tcccatagta acgccaatag ggactttcca ttgacgtcaa |
| | | tgggtggagt atttacggta aactgcccac ttggcagtac |
| | | atcaagtgta tcatatgcca agtacgcccc ctattgacgt |
| | | caatgacggt aaatggcccg cctggcatta tgcccagtac |
| | | atgaccttat gggactttcc tacttggcag tacatctacg |
| | | tattagtcat cgctattacc atggtcgagg tgagccccac |
| | | gttctgcttc actctcccca tctccccccc ctccccaccc |
| | | ccaattttgt atttatttat tttttaatta ttttgtgcag |
| | | cgatgggggc gggggggggg ggggggcgcg cgccaggcgg |
| | | ggcggggcgg ggcgaggggc ggggcggggc gaggcggaga |
| | | ggtgcggcgg cagccaatca gagcggcgcg ctccgaaagt |
| | | ttccttttat ggcgaggcgg cggcggcggc ggccctataa |
| | | aaagcgaagc gcgcggcggg cgggagtcgc tgcgcgctgc |
| | | cttcgccccg tgccccgctc cgccgccgcc tcgcgccgcc |
| | | cgccccggct ctgactgacc gcgttactcc cacaggtgag |
| | | cgggcgggac ggcccttctc ctccgggctg taattagcgc |
| | | ttggtttaat gacggcttgt ttcttttctg tggctgcgtg |
| | | aaagccttga ggggctccgg gagggccctt tgtgcggggg |
| | | gagcggctcg gggggtgcgt gcgtgtgtgt gtgcgtgggg |
| | | agcgccgcgt gcggctccgc gctgcccggc ggctgtgagc |
| | | gctgcgggcg cggcgcgggg ctttgtgcgc tccgcagtgt |
| | | gcgcgagggg agcgcggccg ggggcggtgc cccgcggtgc |
| | | ggggggggct gcgaggggaa caaaggctgc gtgcggggtg |
| | | tgtgcgtggg ggggtgagca gggggtgtgg gcgcgtcggt |
| | | cgggctgcaa ccccccctgc acccccctcc ccgagttgct |
| | | gagcacggcc cggcttcggg tgcggggctc cgtacggggc |
| | | gtggcgcggg gctcgccgtg ccgggcgggg ggtggcggca |
| | | ggtgggggtg ccgggcgggg cggggccgcc tcgggccggg |
| | | gagggctcgg gggaggggcg cggcggcccc cggagcgccg |
| | | gcggctgtcg aggcgcggcg agccgcagcc attgcctttt |
| | | atggtaatcg tgcgagaggg cgcagggact tcctttgtcc |
| | | caaatctgtg cggagccgaa atctgggagg cgccgccgca |
| | | ccccctctag cgggcgcggg gcgaagcggt gcggcgccgg |
| | | caggaaggaa atgggcgggg agggccttcg tgcgtcgccg |
| | | cgccgccgtc cccttctccc tctccagcct cggggctgtc |
| | | cgcgggggga cggctgcctt cgggggggac ggggcagggc |
| | | ggggttcggc ttctggcgtg tgaccggcgg ctctagagcc |
| | | tctgctaacc atgttcatgc cttcttcttt ttcctacagc |
| | | tcctgggcaa cgtgctggtt attgtgctgt ctcatcattt |
| | | tggcaaagaa ttcgctagcg ggcactttgc actggaactt |
| | | acaacacccg agcaaggacg cgactctcca ccatgtaccg |
| | | tatgcagctg ctgctgctga tcgcactgtc actggcactg |
| | | gttaccaact cagaagttca gctggttgaa tcaggcggcg |
| | | gcctggttca gcccggcggc tcactgcgtc tgtcatgtgc |
| | | agcatcaggc tacgatttca cccactacgg catgaactgg |
| | | gttcgtcagg cacccggcaa aggcctggaa tgggttggct |
| | | ggatcaacac ctacaccggc gaacccacct acgcagcaga |
| | | tttcaaacgt cgtttcacct tctcactgga tacctcaaaa |
| | | tcaaccgcat acctgcagat gaactcactg cgtgcagaag |
| | | ataccgcagt ttactactgt gcaaaatacc cctactacta |
| | | cggcacctca cactggtact tcgatgtttg gggccagggc |
| | | accctggtta ccgtttcatc agcatcaacc aaaggcccct |
| | | cagttttccc cctggcaccc tcatcaaaat caacctcagg |
| | | cggcaccgca gcactgggct gtctggttaa agattacttc |
| | | cccgaacccg ttaccgtttc atggaactca ggcgcactga |
| | | cctcaggcgt tcacaccttc cccgcagttc tgcagtcatc |
| | | aggcctgtac tcactgtcat cagttgttac cgttccctca |
| | | tcatcactgg gcacccagac ctacatctgt aacgttaacc |
| | | acaaaccctc aaacaccaaa gttgataaaa aagttgaacc |
| | | caaatcatgt gataaaaccc acctgcgtaa acgtcgtgca |
| | | cccgttaaac agaccctgaa cttcgatctg ctgaaactgg |
| | | caggcgatgt tgaatcaaac cccggcccca tgtaccgtat |
| | | gcagctgctg ctgctgatcg cactgtcact ggcactggtt |
| | | accaactcag atatccagct gacccagtca ccctcatcac |
| | | tgtcagcatc agttggcgat cgtgttacca tcacctgttc |
| | | agcatcacag gatatctcaa actacctgaa ctggtaccag |
| | | cagaaacccg gcaaagcacc caaagttctg atctacttca |
| | | cctcatcact gcactcaggc gttccctcac gtttctcagg |
| | | ctcaggctca ggcaccgatt tcaccctgac catctcatca |
| | | ctgcagcccg aagatttcgc aacctactac tgtcagcagt |
| | | actcaaccgt tccctggacc ttcggccagg gcaccaaagt |
| | | tgaaatcaaa cgtaccgttg cagcaccctc agttttcatc |
| | | ttccccccct cagatgaaca gctgaaatca ggcaccgcat |
| | | cagttgtttg tctgctgaac aacttctacc cccgtgaagc |
| | | aaaagttcag tggaaagttg ataacgcact gcagtcaggc |
| | | aactcacagg aatcagttac cgaacaggat tcaaaagatt |
| | | caacctactc actgtcatca accctgaccc tgtcaaaagc |
| | | agattacgaa aaacacaaag tttacgcatg tgaagttacc |
| | | caccagggcc tgtcatcacc cgttaccaaa tcattcaacc |
| | | gtggcgaatg ttgataaagc ggccgcggta cctctagagt |
| | | cgacccgggc ggcctcgagg acggggtgaa ctacgcctga |
| | | ggatccgatc tttttccctc tgccaaaaat tatggggaca |
| | | tcatgaagcc ccttgagcat ctgacttctg gctaataaag |
| | | gaaatttatt ttcattgcaa tagtgtgttg gaattttttg |
| | | tgtctctcac toggaagcaa ttcgttgatc tgaatttcga |
| | | ccacccataa tacccattac cctggtagat aagtagcatg |
| | | gcgggttaat cattaactac aaggaacccc tagtgatgga |
| | | gttggccact ccctctctgc gcgctcgctc gctcactgag |
| | | gccgggcgac caaaggtcgc ccgacgcccg ggctttgccc |
| | | gggcggcctc agtgagcgag cgagcgcgca g |
| 57 | Ranibizumab Fab | MYRMQLLLLIALSLALVTNSDIQLTQSPSSLSASVGDRVTITCSASQDISN |
| | Amino Acid | YLNWYQQKPGKAPKVLIYFTSSLHSGVPSRFSGSGSGTDFTLTISSLQPED |
| | Sequence (Signal | FATYYCQQYSTVPWTFGQGTKVEIKRTVAAPSVFIFPPSDEQLKSGTASVV |
| | Peptide and Light | CLLNNFYPREAKVQWKVDNALQSGNSQESVTEQDSKDSTYSLSSTLTLSKA |
| | chain) | DYEKHKVYACEVTHQGLSSPVTKSFNRGEC |
| 58 | Ranibizumab Fab | MYRMQLLLLIALSLALVTNSEVQLVESGGGLVQPGGSLRLSCAASGYDFTH |
| | Amino Acid | YGMNWVRQAPGKGLEWVGWINTYTGEPTYAADFKRRFTFSLDTSKSTAYLQ |
| | Sequence (Signal | MNSLRAEDTAVYYCAKYPYYYGTSHWYFDVWGQGTLVTVSSASTKGPSVFP |
| | Peptide and | LAPSSKSTSGGTAALGCLVKDYFPEPVTVSWNSGALTSGVHTFPAVLQSSG |
| | Heavy chain) | LYSLSSVVTVPSSSLGTQTYICNVNHKPSNTKVDKKVEPKSCDKTH |
| 59 | Optionally amino | LGETTRP |
| | acid insertion | |
| 60 | Optionally amino | LALGETTRP |
| | acid insertion | |
| 61 | Hyperglycosylate | DIQLTQSPSSLSASVGDRVTITCSASQDISNYLNWYQQKPGKAPKVLIYFT |
| | d Ranibizumab | SSLHSGVPSRFSGSGSGTDFTLTISSLQPEDFATYYCQQYSTVPWTFGQGT |
| | Fab Amino Acid | KVEIKRTVAAPSVFIFPPSDEQLKSGTASVVCLLNNFYPREAKVQWKVDNA |
| | Sequence (Light | LQSGNSNESVTEQDSKDSTYSLSSTLTLSKADYEKHKVYACNVTHQGLSSP |
| | chain) | VTKSFNRGEC |
| 62 | Hyperglycosylate | EVQLVESGGGLVQPGGSLRLSCAASGYDFTHYGMNWVRQAPGKGLEWVGWI |
| | d Ranibizumab | NTYTGEPTYAADFKRRFTFSLDTSKSTAYLQMNSLRAEDTAVYYCAKYPYY |
| | Fab Amino Acid | YGTSHWYFDVWGQGTNVTVSSASTKGPSVFPLAPSSKSTSGGTAALGCLVK |
| | Sequence (Heavy | DYFPEPVTVSWNSGALTSGVHTFPAVLQSSGLYSLSSVVTVPSSSLGTQTY |
| | chain) | ICNVNHKPSNTKVDKKVEPKSCDKTHL |
| 63 | Bevacizumab and | QQYSTVPWTF |
| | Ranibizumab | |
| | Light Chain | |
| | CDR3 | |

### 6. EXAMPLES

### 6.1 Example 1: Bevacizumab Fab cDNA-Based Vector

A bevacizumab Fab cDNA-based vector is constructed comprising a transgene comprising bevacizumab Fab portion of the light and heavy chain cDNA sequences (SEQ ID NOs. 10 and 11, respectively). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.2 Example 2: Ranibizumab cDNA-Based Vector

A ranibizumab Fab cDNA-based vector is constructed comprising a transgene comprising ranibizumab Fab light and heavy chain cDNAs (the portions of SEQ ID NOs. 12 and 13, respectively not encoding the signal peptide). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.3 Example 3: Hyperglycosylated Bevacizumab Fab cDNA-Based Vector

A hyperglycosylated bevacizumab Fab cDNA-based vector is constructed comprising a transgene comprising bevacizumab Fab portion of the light and heavy chain cDNA sequences (SEQ ID NOs. 10 and 11, respectively) with mutations to the sequence encoding one or more of the following mutations: L118N (heavy chain), E195N (light chain), or Q160N or Q160S (light chain). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.4 Example 4: Hyperglycosylated Ranibizumab cDNA-Based Vector

A hyperglycosylated ranibizumab Fab cDNA-based vector is constructed comprising a transgene comprising ranibizumab Fab light and heavy chain cDNAs (the portions of SEQ ID NOs. 12 and 13, respectively not encoding the signal peptide), with mutations to the sequence encoding one or more of the following mutations: L118N (heavy chain), E195N (light chain), or Q160N or Q160S (light chain). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.5 Example 5: Ranibizumab Based HuGlyFabVEGFi

A ranibizumab Fab cDNA-based vector (see Example 2) is expressed in the PER.C6® Cell Line (Lonza) in the AAV8 background. The resultant product, ranibizumab-based HuGlyFabVEGFi is determined to be stably produced. N-glycosylation of the HuGlyFabVEGFi is confirmed by hydrazinolysis and MS/MS analysis. See, e.g., Bondt et al., Mol. & Cell. Proteomics 13.11:3029-3039. Based on glycan analysis, HuGlyFabVEGFi is confirmed to be N-glycosylated, with 2,6 sialic acid a predominant modification. Advantageous properties of the N-glycosylated HuGlyFabVEGFi are determined using methods known in the art. The HuGlyFabVEGFi can be found to have increased stability and increased affinity for its antigen (VEGF). See Sola and Griebenow, 2009, J Pharm Sci., 98(4): 1223-1245 for methods of assessing stability and Wright et al., 1991, EMBO J. 10:2717-2723 and Leibiger et al., 1999, Biochem. J. 338:529-538 for methods of assessing affinity.

### 6.6 Example 6: Treatment of Wet AMD with Ranibizumab Based HuGlyFabVEGFi by Peripheral Injection

Based on determination of advantageous characteristics of ranibizumab-based HuGlyFabVEGFi (see Example 5), a ranibizumab Fab cDNA-based vector is deemed useful for treatment of wet AMD when expressed as a transgene. A subject presenting with wet AMD is administered AAV8 that encodes ranibizumab Fab at a dose sufficient to produce a concentration of the transgene product at a Cₘᵢₙ of at least 0.330 μg/mL in the Vitreous humour for three months. The administration is done by subretinal administration via peripheral injection into the retina (i.e., peripheral to the optic disc, fovea and macula located in the back of the eye), which is accomplished by transvitreal injection. Following treatment, the subject is evaluated for improvement in symptoms of wet AMD.

### 6.7 Example 7: a Randomized, Partially Masked, Controlled, Phase 2b Clinical Study to Evaluate the Safety and Efficacy of Construct II Gene Therapy in Participants with nAMD

### 6.7.1 Synopsis

### Primary Objectives.

To evaluate mean change in best-corrected visual acuity (BCVA) for Construct II compared with ranibizumab at Week 50.

### Secondary Objectives.

To evaluate the safety and tolerability of Construct II through Week 102. To evaluate the effect of Construct II on BCVA. To evaluate the effect of Construct II on central retinal thickness (CRT) as measured by spectral domain-optical coherence tomography (SD-OCT). To assess the need for supplemental anti-vascular endothelial growth factor (VEGF) therapy in the Construct II treatment arms. To assess aqueous protein concentrations of Construct II. To evaluate the immunogenicity of Construct II.

### Exploratory Objectives.

To evaluate changes over time in the area of geographic atrophy and to assess, in participants with no evidence at baseline, the incidence of new areas of geographic atrophy. To assess the proportion of participants with no fluid on SD-OCT. To assess aqueous VEGF-A concentrations. To evaluate visual function and treatment satisfaction using patient reported outcome (PRO) questionnaires

### Study Design.

This phase 2b partially masked, randomized, multicenter study will include 3 periods: an Active Run-in Period (i.e., screening), a Treatment Period, and an Extension Period. Participants who receive Construct II will be asked to participate in a long-term follow-up study after completion of or early discontinuation from the current study and will sign a separate informed consent for the follow-up study at that time.

The Active Run-in Period, which will last up to 10 weeks, will begin when the participant signs the informed consent form and will end once the participant has been evaluated for eligibility and has received 3 monthly intravitreal injections of ranibizumab 0.5 mg. The Treatment Period will last up to 12 months, beginning when the participant is randomized to study treatment and ending at Week 50. The Extension Period will last up to 12 months, beginning after Week 50 and ending at Week 102.

At Screening Visit 1 (Week −10), participants who meet the inclusion/exclusion criteria will enter the study and receive a 0.5-mg intravitreal injection of ranibizumab in the study eye. At Screening Visit 2 (Week −6), participants will receive a second 0.5-mg intravitreal injection of ranibizumab in the study eye. One week later, at Screening Visit 3 (Week −5), participants' anatomic response on SD-OCT will be evaluated against prespecified response criteria. Participants not meeting response criteria will be exited from the study. If participants meet all inclusion criteria, at Screening Visit 4 (Week −2), participants will be randomized. Any participants who withdraw or become ineligible for randomization during the Screening Period and have an adverse event (AE) associated with the intravitreal ranibizumab injections will be followed until the AE resolves (up to 30 days postinjection). Participants who are identified at Screening Visit 4 as being eligible will receive a third 0.5-mg intravitreal injection of ranibizumab in the study eye. Once the Central Reading Center (CRC) has verified the CRT, participants will be randomized (1:1:1) using an interactive response technology system to receive a single dose of Construct II (Dose 1), a single dose of Construct II (Dose 2), or monthly intravitreal ranibizumab 0.5 mg; Construct II will be administered by subretinal delivery. Participants will be stratified by baseline (Screening Visit 4) BCVA score (>58 letters vs ≤58 letters) in the randomization.

Participants randomized to the Construct II treatment arms will undergo the surgical procedure on Day 1 followed by visits on Day 2 and Day 8 to assess postoperative safety. At Week 2, participants will receive intravitreal ranibizumab to supplement any anti-VEGF that may have been removed during the vitrectomy surgery and to provide anti-VEGF therapy coverage while potential production of the gene therapy mediated protein escalates. The participants will then be seen at monthly intervals, beginning with Week 6, during which supplemental intravitreal ranibizumab 0.5-mg therapy may be administered if needed, as determined by the fully masked CRC evaluation of the SD-OCT data and the fully masked visual acuity assessor's evaluation of BCVA. Note that the SD-OCT and BCVA results from the masked assessors, together with predefined retreatment criteria, will inform the investigator's decision to provide supplemental anti-VEGF therapy.

Participants randomized to the ranibizumab control arm will have their first postrandomization visit at Week 2 and will receive intravitreal ranibizumab 0.5 mg. Following the Week 2 visit, the participants will have monthly (˜28 day) study visits during which they will receive an intravitreal injection of ranibizumab 0.5 mg.

At the Week 50 primary endpoint, participants in the ranibizumab control arm will be offered the opportunity to receive Construct II treatment if they still meet key inclusion/exclusion criteria. The treating physician will determine if the participant is eligible and a good candidate for the procedure. Qualified participants will then be administered the highest tolerated dose evaluated in this protocol. Participants in the ranibizumab control arm who switch to Construct II following Week 50 will follow the same visit schedule as the one started on Day 1 for participants originally randomized to receive Construct II. Those participants who either choose not to have treatment with Construct II or are ineligible for treatment with Construct II will be discontinued from the study.

Throughout the study, participants will be evaluated through the assessment of ocular and nonocular AEs including serious adverse events (SAEs) and adverse events of special interest (AESIs) (ocular inflammation deemed by the investigator to be unrelated to the surgical/study procedure and is graded as 2+ or greater on the ocular inflammation grading scales, ocular infections [including endophthalmitis], retinal tears or detachment, retinal thinning, and new arterial thromboembolic events [nonfatal stroke, nonfatal myocardial infarction, or vascular death (including deaths of unknown cause)]), as well as assessments of clinical laboratory testing (chemistry, hematology, coagulation, urinalysis), ocular examinations and imaging (BCVA, intraocular pressure, slit-lamp biomicroscopy, indirect ophthalmoscopy, fluorescein angiography [FA], fundus autofluorescence [FAF], and SD-OCT), and vital signs. Note that AEs will be collected at all study visits. Immunogenicity to the vector and transgene product (TP) of Construct II will also be assessed. Patient reported outcomes will be collected using the supplemented National Eye Institute Visual Functioning Questionnaire 25-item version (NEI-VFQ-25) (also comprises the Rasch-scored version, NEI-VFQ-28-R) and Macular Disease Treatment Satisfaction Questionnaire (MacTSQ).

Planned safety monitoring of the study participants will be conducted on an ongoing basis. These include reviews conducted by the partially masked Medical Monitor and routine reviews conducted by the partially masked Sponsor's Internal Safety Committee. Separately, an Independent Data Monitoring Committee (IDMC) will also be established and will meet on a periodic basis to independently review the clinical data. If unmasked reviews are needed to understand a potential safety signal, these reviews will be conducted by the IDMC.

### Diagnosis and Main Criteria for Inclusion.

To be eligible for enrollment in this study, participants, aged ≥50 and ≤89 years, must have a diagnosis of subfoveal choroidal neovascularization secondary to age-related macular degeneration in the study eye. Optical coherence tomography documentation from a current image of center subfield fluid must be confirmed by the CRC. Participants must have a BCVA letter score in the study eye between ≤78 and >44 and be pseudophakic (status postcataract surgery) in the study eye. Participants also must be willing and able to provide written, signed informed consent for this study after the nature of the study has been explained, and prior to any research-related procedures being conducted.

### Investigational Product, Dosage, and Mode of Administration.

Construct II Dose 1: 1.6×10¹¹ GC/eye (6.2×10¹¹ GC/mL). Construct II Dose 2: 2.5×10¹¹ GC/eye (1.0×10¹² GC/mL). Construct II is administered via subretinal delivery (250 μL in a single dose).

### Duration of Treatment.

In the Construct II treatment arms: 1 day. In the ranibizumab control arm: 50 weeks

Reference Therapy, Dosage and Mode of Administration.

Ranibizumab (LUCENTIS®, Genentech) 0.5 mg (0.05 mL of 10 mg/mL solution) will be administered by intravitreal injection approximately every 28 days.

Intravitreal ranibizumab 0.5 mg will also be administered as supplemental anti-VEGF therapy in all treatment arms during the Run-in Period (Screening Visits 1, 2, and 4) and at Week 2. Participants in the Construct II arm will be evaluated for intravitreal ranibizumab 0.5 mg as supplemental anti-VEGF therapy starting at Week 6 according to retreatment criteria; participants in the ranibizumab control arm who switch to Construct II after Week 50 will receive intravitreal ranibizumab 0.5 mg at Week 54 and will be evaluated for intravitreal ranibizumab 0.5 mg as supplemental anti-VEGF therapy starting at Week 58 according to retreatment criteria.

### Criteria for Evaluation.

### Primary Endpoint:

Mean change from baseline in BCVA to Week 50 (as the average of Week 46 and Week 50) based on the Early Treatment Diabetic Retinopathy Study (ETDRS) score

### Secondary Endpoints:

Incidences of ocular and nonocular AEs over 50 weeks.

Incidences of ocular and nonocular AEs over 102 weeks.

Mean change from baseline in BCVA to Week 102 (as the average of Week 98 and Week 102).

Proportion of participants with BCVA of 43 letters (20/160 approximate Snellen equivalent) or worse at Week 50 (as the average of Week 46 and Week 50) and Week 102 (as the average of Week 98 and Week 102).

Proportion of participants with BCVA of 84 letters (20/20 approximate Snellen equivalent) or better at Week 50 (as the average of Week 46 and Week 50) and Week 102 (as the average of Week 98 and Week 102).

Proportion of participants (1) gaining or losing ≥15, ≥10, ≥5, or ≥0 letters; (2) maintaining vision (not losing ≥15 letters) compared with baseline as per BCVA at Week 50 (as the average of Week 46 and Week 50) and Week 102 (as the average of Week 98 and Week 102).

Mean change from baseline in BCVA to Week 50 (as the average of Week 46 and Week 50) for participants who received ≤2 supplemental anti-VEGF injections, 2 supplemental anti-VEGF injections, 1 supplemental anti-VEGF injection, or 0 supplemental anti-VEGF injections (Construct II randomized participants).

Mean change from Week 50 to Week 102 (as the average of Week 98 and Week 102) in BCVA (control arm participants who switch to Construct II).

Mean change from baseline in CRT as measured by SD-OCT to Week 50 (as the average of Week 46 and Week 50) and Week 102 (as the average of Week 98 and Week 102).

Mean change from Week 50 to Week 102 (as the average of Week 98 and Week 102) in CRT as measured by SD-OCT (control arm participants who switch to Construct II).

Proportion of participants who have a reduction of ≥50% in supplemental anti-VEGF injection rate through Week 50 and Week 102 compared with the prior 50 weeks preceding the first intravitreal ranibizumab injection received as part of the Active Run-in Period (Construct II randomized participants).

Mean reduction in supplemental anti VEGF injection rate through Week 50 and Week 102 compared with the prior 50 weeks preceding the first ranibizumab injection received as part of the Active Run-in Period (Construct II randomized participants).

Mean number of supplemental anti-VEGF injections in the Construct II arms through Week 50 and Week 102; Mean number of supplemental anti-VEGF injections after Week 50 through Week 102 relative to the prior 50 weeks in the study (control arm participants who switch to Construct II).

Time to first supplemental anti-VEGF injection after the Week 2 injection in the Construct II arms.

Proportion of participants in the Construct II arms who receive supplemental anti-VEGF injection after Week 2 through Week 26, after Week 26 through Week 50, after Week 50 through Week 74, after Week 74 through Week 102, after Week 2 through Week 50, and after Week 2 through Week 10

Aqueous Construct II TP concentrations at assessed time points; Immunogenicity measurements (serum neutralizing antibodies to AAV8 and serum antibodies to Construct II TP) at assessed time points.

### Exploratory Endpoints:

Mean change from baseline in area of geographic atrophy based on FAF at assessed time points.

Incidence of new area of geographic atrophy by FAF (in participants with no geographic atrophy at baseline).

Incidence of retinal thinning in the area of the bleb.

Proportion of participants with no fluid on SD-OCT.

VEGF-A concentrations (aqueous) at assessed time points.

Mean change from baseline in NEI-VFQ-28-R (composite score; activity limitation domain score; and socio-emotional functioning domain score) at assessed time points.

Mean change from baseline in NEI-VFQ-25 (composite score and mental health subscale score) at assessed time points.

Mean change from baseline in MacTSQ (composite score; safety, efficacy, and discomfort domain score; and information provision and convenience domain score) at assessed time points.

TABLE 4
Objectives and Endpoints

| | |
|---|---|
| Objectives | Endpoints |

Primary

| | | |
|---|---|---|
| Efficacy | To evaluate mean change | Mean change from baseline in BCVA to |
| | in BCVA for Construct II | Week 50 (as the average of Week 46 and |
| | compared with | Week 50) based on the ETDRS score |
| | ranibizumab at Week 50 | |

Secondary

| | | |
|---|---|---|
| Safety | To evaluate the safety and | Incidences of ocular and nonocular AEs |
| | tolerability of Construct II | over 50 weeks |
| | through Week 102 | Incidences of ocular and nonocular AEs |
| | | over 102 weeks |
| Efficacy | To evaluate the | Mean change from baseline in BCVA to |
| | effect of Construct | Week 102 (as the average of Week 98 and |
| | II on BCVA | Week 102) |
| | | Proportion of participants with BCVA of 43 |
| | | letters (20/160 approximate Snellen |
| | | equivalent) or worse at Week 50 (as the |
| | | average of Week 46 and Week 50) and Week |
| | | 102 (as the average of Week 98 and Week |
| | | 102) |
| | | Proportion of participants with BCVA of 84 |
| | | letters (20/20 approximate Snellen |
| | | equivalent) or better at Week 50 (as the |
| | | average of Week 46 and Week 50) and Week |
| | | 102 (as the average of Week 98 and Week |
| | | 102) |
| | | Proportion of participants (1) gaining or |
| | | losing ≥15, ≥10, ≥5, or ≥0 letters; (2) |
| | | maintaining vision (not losing ≥15 letters) |
| | | compared with baseline as per BCVA at |
| | | Week 50 (as the average of Week 46 and |
| | | Week 50) and Week 102 (as the average of |
| | | Week 98 and Week 102) |
| | | Mean change from baseline in BCVA to |
| | | Week 50 (as the average of Week 46 and |
| | | Week 50) for participants who received ≤2 |
| | | supplemental anti-VEGF injections, 2 |
| | | supplemental anti-VEGF injections, 1 |
| | | supplemental anti-VEGF injection, or 0 |
| | | supplemental anti- VEGF injections |
| | | (Construct II randomized participants) |
| | | Mean change from Week 50 to Week 102 (as |
| | | the average of Week 98 and Week 102) in |
| | | BCVA (control arm participants who switch to |
| | | Construct II) |
| Efficacy | To evaluate the effect of | Mean change from baseline in CRT as |
| | Construct II on CRT as | measured by SD-OCT to Week 50 (as the |
| | measured by SD-OCT | average of Week 46 and Week 50) and Week |
| | | 102 (as the average of Week 98 and Week |
| | | 102) |
| | | Mean change from Week 50 to Week 102 (as |
| | | the average of Week 98 and Week 102) in |
| | | CRT as measured by SD-OCT (control arm |
| | | participants who switch to Construct II) |
| Efficacy | To assess the need | Proportion of participants who have a |
| | for supplemental | reduction of ≥50% in supplemental anti- |
| | anti-VEGF therapy | VEGF injection rate through Week 50 and |
| | in the Construct II | Week 102 compared with the prior 50 weeks |
| | treatment arms | preceding the first ranibizumab injection |
| | | received as part of the Active Run-in Period |
| | | (Construct II randomized participants) |
| | | Mean reduction in supplemental anti- |
| | | VEGF injection rate through Week 50 |
| | | and Week 102 compared with the prior |
| | | 50 weeks preceding the first ranibizumab |
| | | injection received as part of the Active |
| | | Run-in Period (Construct II randomized |
| | | participants) |
| | | Mean number of supplemental anti-VEGF |
| | | injections in the Construct II arms through |
| | | Week 50 and Week 102 |
| | | Mean number of supplemental anti-VEGF |
| | | injections after Week 50 through Week 102 |
| | | relative to the prior 50 weeks in the study |
| | | (control arm participants who switch to |
| | | Construct II) |
| | | Time to first supplemental anti-VEGF |
| | | injection after the Week 2 injection in the |
| | | Construct II arms |
| | | Proportion of participants in the Construct |
| | | II arm who receive supplemental anti- |
| | | VEGF injection after Week 2 through |
| | | Week 26, after Week 26 through Week 50, |
| | | after Week 50 through Week 74, after |
| | | Week 74 through Week 102, after Week 2 |
| | | through Week 50, and after Week 2 |
| | | through Week 102 |
| Pharmacodynamics | To assess aqueous | Aqueous Construct II TP concentrations at |
| | protein concentrations | assessed time points |
| | of Construct II | |
| Immunogenicity | To evaluate the | Immunogenicity measurements (serum |
| | immunogenicity of | neutralizing antibodies to AAV8 and |
| | Construct II | serum antibodies to Construct II TP) at |
| | | assessed time points |
| Efficacy | To evaluate changes over | Mean change from baseline in area of |
| | time in the area of | geographic atrophy based on FAF at |
| | geographic atrophy and to | assessed time points |
| | assess, in participants with | Incidence of new area of geographic atrophy |
| | no evidence at baseline, the | by FAF (in participants with no geographic |
| | incidence of new areas of | atrophy at baseline) |
| | geographic atrophy | Incidence of retinal thinning in the area |
| | | of the bleb |
| Efficacy | To assess the | Proportion of participants with no fluid |
| | proportion of | on SD-OCT |
| | participants with no | |
| | fluid on SD-OCT | |
| Biomarkers | To assess aqueous | VEGF-A concentrations (aqueous) at |
| | VEGF-A | assessed time points |
| | concentrations | |
| PRO | To evaluate visual function | Mean change from baseline in NEI- |
| Questionnaires | and treatment satisfaction | VFQ-28-R (composite score; activity |
| | using PRO questionnaires | limitation domain score; and socio- |
| | | emotional functioning domain score) at |
| | | assessed time points |
| | | Mean change from baseline in NEI-VFQ-25 |
| | | (composite score and mental health subscale |
| | | score) at assessed time points |
| | | Mean change from baseline in MacTSQ |
| | | (composite score; safety, efficacy, and |
| | | discomfort domain score; and information |
| | | provision and convenience domain score) at |
| | | assessed time points |

AAV8 = adeno-associated virus serotype 8;
AE = adverse event;
BCVA = best-corrected visual acuity;
CRT = central retinal thickness;
ETDRS = Early Treatment Diabetic Retinopathy Study;
FAF = fundus autofluorescence;
MacTSQ = Macular Disease Treatment Satisfaction Questionnaire;
NEI-VFQ-25 = National Eye Institute Visual Functioning Questionnaire 25-item Version;
NEI-VFQ-28-R = National Eye Institute Visual Functioning Questionnaire 28-item Rasch-scored Version;
PRO = patient reported outcome;
SD-OCT = spectral domain-optical coherence tomography;
TP = transgene product;
VEGF = vascular endothelial growth factor

### 6.7.2 Study Design

### Overall Study Design

This phase 2b partially masked, randomized, multicenter study will include 3 periods: an Active Run-in Period (i. e., screening), a Treatment Period, and an Extension Period. Participants who receive Construct II will be asked to participate in a long-term follow-up study after completion of or early discontinuation from the current study and will sign a separate informed consent for the follow-up study at that time.

The Active Run-in Period, which will last up to 10 weeks, will begin when the participant signs the Informed consent form (ICF) and will end once the participant has been evaluated for eligibility and has received 3 monthly injections of intravitreal ranibizumab. The Treatment Period will last up to 12 months, beginning when the participant is randomized to study treatment and ending at Week 50. The Extension Period will last up to 12 months, beginning after Week 50 and ending at Week 102.

At Screening Visit 1 (Week −10), participants who meet the inclusion/exclusion criteria will enter the study and receive a 0.5-mg intravitreal injection of ranibizumab in the study eye. At Screening Visit 2 (Week −6), participants will receive a second 0.5-mg intravitreal injection of ranibizumab in the study eye. One week later, at Screening Visit 3 (Week −5), participants' anatomic response on SD-OCT will be evaluated against prespecified response criteria. Participants not meeting response criteria will be exited from the study. If participants meet all inclusion criteria, at Screening Visit 4 (Week −2), participants will be randomized. Any participants who withdraw or become ineligible for randomization during the Screening Period and have an AE associated with the intravitreal ranibizumab injections will be followed until the AE resolves (up to 30 days postinjection). Participants who are identified at Screening Visit 4 as being eligible will receive a third 0.5-mg intravitreal injection of ranibizumab in the study eye. Once the Central Reading Center (CRC) has verified the central retinal thickness (CRT), participants will be randomized (1:1:1) using an interactive response technology (TRT) system to receive a single dose of Construct II (Dose 1), a single dose of Construct II (Dose 2), or monthly intravitreal ranibizumab 0.5 mg; Construct II will be administered by subretinal delivery. Participants will be stratified by baseline (Screening Visit 4) best-corrected visual acuity (BCVA) score (>58 letters vs ≤58 letters) in the randomization.

Participants randomized to the Construct II treatment arms will undergo the surgical procedure on Day 1 followed by visits on Day 2 and Day 8 to assess postoperative safety. At Week 2, participants will receive intravitreal ranibizumab to supplement any anti-VEGF that may have been removed during the vitrectomy surgery to provide anti-VEGF therapy coverage while potential production of the gene therapy mediated protein escalates. The participants will then be seen at monthly intervals, beginning with Week 6, during which supplemental intravitreal ranibizumab 0.5-mg therapy may be administered if needed, as determined by the fully masked CRC evaluation of the SD-OCT data and the fully masked VA assessor's evaluation of BCVA. Note that the SD-OCT and BCVA results, together with predefined retreatment criteria, will inform the investigator's decision to provide supplemental anti-VEGF therapy.

Participants randomized to the ranibizumab control arm will have their first postrandomization visit at Week 2 and will receive intravitreal ranibizumab 0.5 mg. Following the Week 2 visit, the participants will have monthly (˜28 day) study visits during which they will receive an injection of ranibizumab 0.5 mg.

At the Week 50 primary endpoint, participants in the ranibizumab control arm will be offered the opportunity to receive Construct II treatment if they still meet key inclusion/exclusion criteria. The treating physician will determine if the participant is eligible and a good candidate for the procedure. Qualified participants will then be administered the highest tolerated dose evaluated in this protocol. Participants in the ranibizumab control arm who switch to Construct II following Week 50 will follow the same visit schedule as the one started on Day 1 for participants originally randomized to receive Construct II. Those participants who either choose not to have treatment with Construct II or are ineligible for treatment with Construct II will be discontinued from the study.

Throughout the study, participants will be evaluated through the assessment of ocular and nonocular AEs including serious adverse events (SAEs) and adverse events of special interest (AESIs) (ocular inflammation deemed by the investigator to be unrelated to the surgical/study procedure and is graded as 2+ or greater on the ocular inflammation grading scales, ocular infections [including endophthalmitis], retinal tears or detachment, retinal thinning, and new arterial thromboembolic events [nonfatal stroke, nonfatal myocardial infarction, or vascular death (including deaths of unknown cause)]), as well as assessments of clinical laboratory testing (chemistry, hematology, coagulation, urinalysis), ocular examinations and imaging (BCVA, IOP, slit-lamp biomicroscopy, indirect ophthalmoscopy, fluorescein angiography [FA], fundus autofluorescence [FAF], and SD-OCT), and vital signs. Note that AEs will be collected at all study visits. Immunogenicity to the vector and TP of Construct II will also be assessed. Patient reported outcomes (PROs) will be collected using the supplemented National Eye Institute Visual Functioning Questionnaire 25-item version (NEI-VFQ-25) (also comprises the Rasch-scored version, NEI-VFQ-28-R) and Macular Disease Treatment Satisfaction Questionnaire (MacTSQ).

Planned safety monitoring of the study participants will be conducted on an ongoing basis. These include reviews conducted by the partially masked Medical Monitor and routine reviews conducted by the partially masked Sponsor's Internal Safety Committee (ISC). Separately, an Independent Data Monitoring Committee (IDMC) will also be established and will meet on a periodic basis to independently review the clinical data. If unmasked reviews are needed to understand a potential safety signal, these reviews will be conducted by the IDMC.

### 6.7.3 Study Population

### (a) General Considerations

Approximately 300 participants with nAMD who meet the inclusion/exclusion criteria will be randomized. It is expected that up to 50 study centers in the United States will participate in this study. Prospective approval of protocol deviations to recruitment and enrollment criteria, also known as protocol waivers or exemptions, is not permitted.

### (b) Inclusion Criteria

Participants must meet all the following criteria in order to be eligible for this study:

1. Males or females aged ≥50 years and ≤89 years.

2. An Early Treatment Diabetic Retinopathy Study (ETDRS) BCVA letter score between ≤78 and ≥44 in the study eye at Screening Visit 1.

3. If both eyes are eligible, the study eye must be the participant's worse-seeing eye, as determined by the investigator prior to randomization.

4. Must have a diagnosis of subfoveal CNV secondary to AMD in the study eye, along with fluid within the parafovea (3-mm center of the macula, based on the early treatment diabetic retinopathy grid) at Screening Visit 1. CNV lesion characteristics as assessed by the CRC: lesion size needs to be less than 10-disc areas (typical disc area=2.54 mm²).

5. Must be pseudophakic (at least 12 weeks postcataract surgery) in the study eye

6. Must be willing and able to comply with all study procedures and be available for the duration of the study.

7. Women must be postmenopausal (defined as being at least 12 consecutive months without menses) or surgically sterilized (ie, having a bilateral tubal ligation/bilateral salpingectomy, bilateral tubal occlusive procedure, hysterectomy, or bilateral oophorectomy). If not, women must have a negative serum pregnancy test at Screening Visit 1, have negative urine pregnancy test results at Screening Visit 4, and be willing to have additional pregnancy tests during the study.

8. Women of childbearing potential (and their male partners) must be willing to use a highly effective method of contraception and male participants engaged in a sexual relationship with a woman of childbearing potential must be willing to use condoms from Screening Visit 1 until 24 weeks after Construct II administration. For the purpose of this study, highly effective methods of contraception for women of childbearing potential include the following: combined hormonal contraception associated with inhibition of ovulation (oral, intravaginal, transdermal); progestogen-only hormonal contraception associated with inhibition of ovulation (oral, injectable, implantable); intrauterine device; intrauterine hormone-releasing system; bilateral tubal occlusion; vasectomized partner; or sexual abstinence, when it is preferred and usual lifestyle of the participant.

9. Must be willing and able to provide written, signed informed consent.

10. Based on the Screening Visit 3 SD-OCT, participants must have improvement in fluid (see Response Criterion below) and have a CRT <400 μm. Note that, if the participant has disease other than fluid contributing to an increase (ie, PED or SHRM) in CRT, they will be enrolled if they have <75 μm of fluid (intraretinal or subretinal), as determined by the CRC. Response Criterion: Subjects must have an improvement in inner retinal (parafovea 3 mm) fluid relative to Screening Visit 1 of >50 μm or 30%; or an improvement in center subfield thickness of >50 μm or 30% as determined by the CRC.

### (c) Exclusion Criteria

Participants are excluded from the study if any of the following criteria apply:

1. CNV or macular edema in the study eye secondary to any causes other than AIMD.

2. Subfoveal fibrosis or atrophy as determined by the CRC.

3. Participants who required >10 anti-VEGF injections in the 12 months prior to the Screening Visit 1.

4. Any condition in the investigator's opinion that could limit VA improvement in the study eye.

5. Active or history of retinal detachment in the study eye.

6. Advanced glaucoma in the study eye defined as IOP of >23 mmHg not controlled by 2 IOP-lowering medications or any invasive procedure to treat glaucoma (e.g., shunt, tube, or MIGS devices; selective laser trabeculectomy and argon laser trabeculoplasty are permitted).

7. Any condition in the study eye that, in the opinion of the investigator, may increase the risk to the participant, require either medical or surgical intervention during the course of the study to prevent or treat vision loss, or interfere with study procedures or assessments.

8. History of intraocular surgery in the study eye within 12 weeks prior to Screening Visit 1. Yttrium aluminum garnet capsulotomy is permitted if performed >10 weeks prior to the Screening Visit 1.

9. History of intravitreal therapy in the study eye, such as intravitreal steroid injection or investigational product, other than anti-VEGF therapy, in the 6 months prior to Screening Visit 1.

10. Presence of any implant in the study eye at Screening Visit 1 (excluding intraocular lens).

11. History of malignancy or hematologic malignancy that may compromise the immune system requiring chemotherapy and/or radiation in the 5 years prior to Screening Visit 1. Localized basal cell carcinoma will be permitted.

12. Receipt of any investigational product within the 30 days of enrollment or 5 half-lives of the investigational product, whichever is longer.

13. Received gene therapy.

14. History of retinal toxicity caused by a therapy, or concomitant therapy with any drug that may affect VA or with known retinal toxicity, e.g., chloroquine or hydroxychloroquine.

15. Ocular or periocular infection in the study eye that may interfere with the surgical procedure.

16. Myocardial infarction, cerebrovascular accident, or transient ischemic attack within the past 6 months.

17. Uncontrolled hypertension (systolic blood pressure [BP]>180 mmHg, diastolic BP >100 mmHg) despite maximal medical treatment.

18. Any participant with the following laboratory values at Screening Visit 1 will be withdrawn from study:
Aspartate aminotransferase (AST)/alanine aminotransferase (ALT) >2.5× upper limit of normal (ULN).
Total bilirubin >1.5×ULN, unless the participant has a previously known history of Gilbert's syndrome and a fractionated bilirubin that shows conjugated bilirubin <35% of total bilirubin.
Prothrombin time >1.5×ULN, unless the participant is anticoagulated. Participants who are anticoagulated will be monitored by local labs and managed per local practice to hold or bridge anticoagulant therapy for the study procedure; consultation with the Medical Monitor is also required.
Hemoglobin <10 g/dL for male participants and <9 g/dL for female participants.
Platelets <100×103/μL.
Estimated glomerular filtration rate <30 mL/min/1.73 m2.

19. Any concomitant treatment that, in the opinion of the investigator, may interfere with ocular surgical procedure or healing process.

20. Known hypersensitivity to ranibizumab or any of its components.

21. Has a serious, chronic, or unstable medical or psychological condition that, in the opinion of the investigator or Sponsor, may compromise the participant's safety or ability to complete all assessments and follow-up in the study.

22. Currently taking anticoagulation therapy for which holding anticoagulation therapy for Construct II administration is not indicated or considered to be unsafe in the opinion of the treating investigator (ie, retinal surgeon), as well as the physician prescribing anticoagulation for the participant.

(d) Inclusion Criteria for Participants in the Control Arm to Obtain Construct II after Week 50.

1. Study eye will be the eye that qualified at randomization.

2. Participant has a CRT <400 μm of subretinal/intraretinal fluid or (in cases where a participant may have nonfluid elevation in the CRT, eg, pigment epithelial defect)<75 μm of excess fluid, as confirmed by the masked CRC.

3. Women of childbearing potential (and their male partners) must be willing to use a highly effective method of contraception and male participants engaged in a sexual relationship with a woman of childbearing potential must be willing to use condoms from the surgical visit until 24 weeks after Construct II administration. For the purpose of this study, highly effective methods of contraception for women of childbearing potential include the following: combined hormonal contraception associated with inhibition of ovulation (oral, intravaginal, transdermal); progestogen-only hormonal contraception associated with inhibition of ovulation (oral, injectable, implantable); intrauterine device; intrauterine hormone-releasing system; bilateral tubal occlusion; vasectomized partner; or sexual abstinence, when it is preferred and usual lifestyle of the participant.

4. Women of childbearing potential must have a negative urine pregnancy test at Week 52 and be willing to have additional pregnancy tests during the study.

(e) Exclusion Criteria for Participants in the Control Arm to Obtain Construct II after Week 50.

1. CNV or macular edema in the study eye secondary to any causes other than AMD.

2. Subfoveal fibrosis or atrophy as determined by the CRC, or any condition preventing VA improvement in the study eye.

3. Ocular or periocular infection in the study eye that may interfere with the surgical procedure.

4. Myocardial infarction, cerebrovascular accident, or transient ischemic attacks since randomization.

5. Uncontrolled hypertension (systolic BP >180 mmHg, diastolic BP >100 mmHg) despite maximal medical treatment.

6. Any concomitant treatment that, in the opinion of the investigator, may interfere with ocular surgical procedure or healing process.

7. History of malignancy or hematologic malignancy that may compromise the immune system requiring chemotherapy and/or radiation in the past year. Localized basal cell carcinoma will be permitted.

8. Currently taking anticoagulation therapy for which holding anticoagulation therapy for Construct II administration is not indicated or considered to be unsafe in the opinion of the treating investigator as well as the physician prescribing anticoagulation for the participant.

### 6.7.4 Study Intervention

Study intervention is defined as any investigational intervention(s), marketed product(s), placebo, or medical device(s) intended to be administered to a study participant according to the study protocol.

### (a) Study Intervention(s) Administered

Eligible participants will be randomized 1:1:1 to receive a single dose of Construct II (Dose 1), a single dose of Construct II (Dose 2), or monthly intravitreal injections of ranibizumab.

Participants in either of the Construct II arms will receive Construct II on Day 1 via subretinal delivery in an operating room. During the study, participants in the Construct II arms will receive ranibizumab 0.5 mg, administered by intravitreal injection, on Screening Visits 1, 2, and 4, at Week 2, and then as needed every 4 weeks starting at Week 6.

Participants in the ranibizumab control arm will receive ranibizumab 0.5 mg, administered by intravitreal injection, on Screening Visits 1, 2, and 4, at Week 2, and then monthly (˜28 days) thereafter.

TABLE 5
Study Intervention(s) Administered

| | | | Ranibizumab |
|---|---|---|---|
| Arm Name | Construct II Dose 1 | Construct II Dose 2 | (LUCENTIS) |
| Type | Gene therapy | | Drug |
| Dose | Solution | | |
| Formulation | | | |
| Unit Dose | 6.2 × 10¹¹ GC/mL | 1.0 × 10¹² GC/mL | 10 mg/mL |
| Strength | | | |
| Dose Level(s) | 250 μL | 250 μL | 0.5 mg (0.05 mL of 10 |
| | (1.6 × 10¹¹ GC/eye) | (2.5 × 10¹¹ GC/eye) | mg/mL solution) once a |
| | one-time dose | one-time dose | month (approximately |
| | | | every 28 days) |
| Route of | Subretinal delivery | | Intravitreal injection |
| Administration | | | |

| Physical | Construct II investigational product is supplied | LUCENTIS is |
|---|---|---|
| Description | as a frozen, sterile, single-use solution of the | supplied as a |
| | AAV vector active ingredient in a formulation | preservative-free, |
| | buffer. The solution appears clear to opalescent, | sterile solution in a |
| | colorless, and free of visible particulates at | single-use container |
| | room temperature. | designed to deliver |
| | | 0.05 mL of 10 mg/mL |
| | | LUCENTIS (0.5 mg |
| | | dose prefilled syringe |
| | | or vial) aqueous |
| | | solution. The solution |
| | | appears colorless to |
| | | pale yellow. |
| Manufacturer | Advanced Bioscience Laboratories, Inc | Genentech, Inc |
| Packaging and | Construct II will be supplied as a sterile, single- | Study intervention will be |
| Labeling | use solution in 2-mL Crystal Zenith ® vials | obtained in commercial |
| | sealed with latex free rubber stoppers and | packaging, either the |
| | aluminum flip-off seals. Each vial will be | prefilled syringe (NDC |
| | labeled as required per country regulatory | 50242-080-03) or single- |
| | requirements. | use 2-mL glass vial (NDC |

50242-080-02) designed
to deliver 0.05 mL of
10 mg/mL ranibizumab
solution.

### 6.7.5 Ocular Inflammation Grading Scale

Ocular inflammation will be assessed during slit-lamp biomicroscopy and independent ophthalmoscopy and graded using the following scales. The standard practice for slit-lamp biomicroscopy and indirect ophthalmoscopy assessment should be used.

TABLE 6
Grading Scale for Ocular Inflammation: Anterior
Chamber Cells and Anterior Chamber Flare
Anterior Chamber Cells

| Grade | Cells in Field (1 mm × 1 mm slit beam) |
|---|---|
| 0 | None |
| +0.5 | 1-5 |
| +1 | 6-15 |
| +2 | 16-25 |
| +3 | 26-50 |
| +4 | >50 |

Anterior Chamber Flare

| Grade | Description |
|---|---|
| 0 | None |
| +1 | Trace |
| +2 | Moderate (iris and lens detail clear) |
| +3 | Marked (iris and lens detail hazy) |
| +4 | Intense (fibrin or plastic aqueous) |

Source: Jabs et al., 2005, Am J Ophthalmol 140(3): 509-516.

TABLE 7
Grading Scale for Vitreous Haze

| Grade | Amount of Vitreal Haze |
|---|---|
| 0 | None |
| +0.5 | Trace |
| +1 | Clear optic disc and vessels; hazy nerve fiber layer |
| +2 | Hazy optic disc and vessels |
| +3 | Optic disk visible |
| +4 | Optic disc not visible |

### 6.8 Example 8: a Phase 2, Randomized, Dose-Escalation, Ranibizumab-Controlled Study to Evaluate the Efficacy, Safety, and Tolerability of Construct II Gene Therapy Delivered Via One or Two Suprachoroidal Space (SCS) Injections in Participants with Neovascular Age-Related Macular Degeneration (nAMD)

### 6.8.1 Synopsis

### (a) Objectives and Endpoints

TABLE 8
Objectives and Endpoints

| | | |
|---|---|---|
| Measure | Objectives | Endpoints |

Primary

| | | |
|---|---|---|
| Efficacy | To evaluate the mean change in BCVA | Mean change from baseline in |
| | for Construct II compared with | BCVA to Week 40 based on |
| | ranibizumab monthly at Week 40 | the ETDRS score |

Secondary

| | | |
|---|---|---|
| Safety | To evaluate the safety and tolerability | Incidences of overall and ocular |
| | of Construct II | AEs and SAEs through |
| | To evaluate the effect of Construct II | Week 52 |
| | on CNV lesion growth and leakage | Vector shedding analysis in |
| | as measured by FA | serum, urine, and tears |
| | | Mean change from baseline in |
| | | CNV lesion size and leakage |
| | | area based on FA at Week 40 |
| | | and Week 52 |
| Efficacy | To evaluate the effect of Construct II | Mean change from baseline in |
| | on BCVA | BCVA to Week 52 |
| | | Proportion of participants |
| | | (1) gaining or losing ≥15, ≥10, ≥5, |
| | | or ≥0 letters; |
| | | (2) maintaining vision (not |
| | | losing ≥15 letters) compared |
| | | with baseline as per BCVA at |
| | | Week 40 and Week 52 |
| | | Mean change from baseline in |
| | | BCVA to Week 40 and |
| | | Week 52 for participants who |
| | | received ≤2 supplemental |
| | | anti-VEGF injections, |
| | | 2 supplemental anti-VEGF |
| | | injections, 1 supplemental |
| | | anti-VEGF injection, or |
| | | 0 supplemental anti-VEGF |
| | | injections (Construct II |
| | | randomized participants) |
| | To evaluate the effect of Construct II | Mean change from baseline in |
| | on CRT, as measured by SD-OCT | CRT as measured by SD-OCT |
| | | to Week 40 and Week 52 |
| | To assess the need for supplemental | Annualized supplemental |
| | anti-VEGF therapy in participants | anti-VEGF injection rate |
| | who receive Construct II treatment | through Week 40 and Week |
| | | 52 |
| | | Proportion of participants who |
| | | have a reduction of ≥50% in |
| | | the annualized supplemental |
| | | anti-VEGF injection rate |
| | | through Week 40 and Week |
| | | 52 compared with the prior |
| | | 52 weeks preceding the first |
| | | intravitreal ranibizumab |
| | | injection received as part of |
| | | the Screening Period |
| | | (Construct II randomized |
| | | participants) |
| | | Mean reduction in the annualized |
| | | supplemental anti-VEGF |
| | | injection rate through Week |
| | | 40 and Week 52 compared |
| | | with the prior 52 weeks |
| | | preceding the first |
| | | ranibizumab injection |
| | | received as part of the |
| | | Screening Period (Construct |
| | | II randomized participants) |
| | | Time to first supplemental |
| | | anti-VEGF injection |
| Pharmacodynamics | To evaluate the concentration of | Mean change from baseline in |
| | Construct II TP in aqueous humor | aqueous Construct II TP |
| | | concentrations over time |
| Immunogenicity | To evaluate the immunogenicity of | Immunogenicity measurements |
| | Construct II | (AAV8: NAbs, TAbs, and |
| | | ELISpot; Construct II protein: |
| | | TAbs and ELISpot) |

Exploratory

| | | |
|---|---|---|
| Efficacy | To evaluate the effect of Construct II | Proportion of participants with no |
| | on fluid accumulation as assessed | fluid on SD-OCT |
| | by SD-OCT | Proportion of participants with |
| | | stable fluid on SD-OCT |
| | | within 30 μm of baseline |
| Safety | To assess changes in visual function by | Changes in visual field testing |
| | visual fields | over time |
| | To evaluate the incidences of new | Incidence of new area of |
| | areas of geographic atrophy, as | geographic atrophy by FAF |
| | assessed by FAF | (in participants with no |
| | | geographic atrophy at |
| | | baseline) |
| Biomarker | To assess aqueous humor VEGF | VEGF-A concentrations |
| | concentrations | (aqueous) at assessed time |
| | | points |

AAV8 = adeno-associated virus serotype 8;
AE = adverse event;
BCVA = best-corrected visual acuity;
CNV = choroidal neovascularization;
CRT = central retinal thickness;
ELISpot = enzyme-linked ImmunoSpot;
ETDRS = Early Treatment Diabetic Retinopathy Study;
FA = fluorescein angiography;
FAF = fundus autofluorescence;
NAbs = neutralizing antibodies;
SAE = serious adverse event;
SCS = suprachoroidal space;
SD-OCT = spectral domain-optical coherence tomography;
TAbs = total binding antibodies;
TP = transgene product;
VEGF = vascular endothelial growth factor

### (b) Study Design

In this phase 2, randomized (3:1), dose-escalation, ranibizumab-controlled, study, approximately 40 participants with nAMD will be enrolled into 2 dose cohorts. Within each dose cohort, participants will receive a one-time administration of Construct II in the SCS (n=15 participants) or an intravitreal injection of ranibizumab 0.5 mg every 4 weeks up to Week 52 (n=5 participants).

Participants who receive Construct II will strongly be encouraged to enroll in a long-term follow-up study after completion of the current study at Week 52 (or early discontinuation) and will sign a separate informed consent for the follow-up study at that time. Participants in the ranibizumab control arm will be offered an opportunity following the Week 52 visit to be included in a future Construct II dose cohort.

Screening will comprise 3 visits to select for eligible participants with qualifying AAV8 neutralizing antibodies (NAbs) titers (Visit 1) who demonstrate anatomic responsiveness to ranibizumab during a ranibizumab run-in phase (Visits 2 and 3). During Visit 1, participants who sign the informed consent form (ICF) will be evaluated for eligibility and will have serum samples collected to screen for pre-existing NAbs or will confirm NAb status from a NAb screening protocol. Participants who have negative or low (≤300) titer results for serum AAV8 NAbs will return to the study center to confirm the remaining inclusion/exclusion criteria. Participants continuing to meet eligibility criteria will receive a 0.5-mg intravitreal injection of ranibizumab in the study eye at Visit 2 (Day 1). At Visit 3 (Week 1), participants will be evaluated by spectral domain-optical coherence tomography (SD-OCT) to confirm their anatomic response to the screening anti-VEGF injection via comparison against their Day 1 SD-OCT assessment taken prior to the screening ranibizumab injection. Anatomic response will be determined by a central reading center (CRC) according to pre-specified criteria. Once the CRC has verified anatomic eligibility, 2 sentinel participants in each cohort will be randomized one to Construct II or ranibizumab control. Participants who do not have an anatomic response will be considered screen failures. For screen-failed participants, anyone who has an AE associated with the ranibizumab injections on Day 1 will be followed until the AE resolves (up to 30 days post injection).

At the Week 2 visit, Construct II randomized participants will receive either 1 or 2 injections of Construct II, depending on dose level, administered at the study center by SCS delivery using the Clearside SCS Microinjector™ investigational device; note that the Treatment Period of the study begins at the time of Construct II administration. All investigators will be trained on the SCS procedure. A detailed description of the procedure can be found in the SCS Administration Manual. Following Construct II administration to the sentinel participant who is randomized to Construct II, a 2-week observation period will be conducted for safety. The Sponsor's Internal Safety Committee (ISC) will review the safety data for this participant and, if there are no safety concerns, up to 18 additional participants (14 Construct II and 4 ranibizumab controls) may be randomized. If no safety review triggers (SRTs) are observed, then, following a 2-week observation period for the last dosed participant within the cohort, all available safety data will be evaluated by the Independent Data Monitoring Committee (IDMC). Additionally, if any event meets the criteria of a Stopping Rule, dosing of any new participants will be suspended until a complete review of all safety data has been performed. At any given IDMC meeting, whether planned or called for due to an SRT, the IDMC may recommend stopping the study, proceeding to the next dosing cohort, or proceeding to a lower dose (up to a half-log).

Participants randomized to Construct II will have 2 visits for post injection safety (1-day post procedure and 1-week post procedure). Starting 2 weeks after Construct II administration, participants will have monthly study visits and may receive intravitreal ranibizumab supplemental therapy if they meet predefined supplemental injection criteria. For participants in the Construct II treatment arms, immunogenicity to the vector (as assessed by AAV8 NAbs, AAV8 TAbs, antibodies to Construct II protein, and enzyme-linked ImmunoSpot [ELISpot]), VEGF-A concentrations, and anti-Construct II antibodies will be assessed throughout the study.

Participants randomized to the ranibizumab control arm will have their first post randomization visit at Week 4 and will receive intravitreal ranibizumab 0.5 mg. Following the Week 4 visit, the participants will have monthly (˜every 28 days) study visits during which they will receive an intravitreal injection of ranibizumab 0.5 mg.

Efficacy will be the primary focus of the initial 40 weeks (primary study period). Following completion of the primary study period, participants will continue to be assessed until Week 52. At the end of the Week 52 study visit, participants who received Construct II will be invited to enroll into a long-term follow-up study, while participants who were in the ranibizumab control arm, if eligible, will be offered an opportunity to be included in a future Construct II dose cohort. Participants will be evaluated for safety through the assessment of AEs, including SAEs and adverse events of special interest (AESIs) (ocular inflammation deemed by the investigator to be unrelated to the surgical/study procedure and graded as 2+ or greater on the ocular inflammation grading scales, ocular infections [including endophthalmitis], retinal tears or detachment, retinal thinning, and new arterial thromboembolic events [nonfatal stroke, nonfatal myocardial infarction, or vascular death (including deaths of unknown cause)]), as well as assessments of clinical laboratory tests (chemistry, hematology, coagulation, urinalysis), and ocular examinations and imaging (BCVA, IOP, slit-lamp biomicroscopy, indirect ophthalmoscopy, fluorescein angiography [FA], ultra-wide field Optos fundus auto fluorescence [FAF], ultra-wide field Optos color fundus photography [CFP], Humphrey visual field 120, ormicroperimetry, and SD-OCT).

Note that AEs will be collected at all study visits. Participants who show evidence of new retinal hypo/hyper pigmentation changes as compared with baseline will be monitored using SD-OCT scans. Radial SD-OCT scans that transverse the margin of the hypo/hyper pigmentary area will be captured when possible.

Planned safety monitoring of the study participants will be conducted on an ongoing basis. The monitoring will include reviews conducted by the Medical Monitor and routine reviews conducted by the Sponsor's ISC. Separately, an IDMC will also be established and will meet on a periodic basis to independently review the clinical data.

### 6.8.2 Inclusion Criteria

All Participants Entering the Study. Participants are eligible to be included in the study only if all of the following criteria apply:

1. Males or females, aged ≥50 years and ≤89 years.

2. Must have a diagnosis of subfoveal CNV secondary to AMD in the study eye, along with retinal fluid (either subretinal or intraretinal) within the parafovea (3-mm center of the macula, based on the early treatment diabetic retinopathy grid), as assessed by the CRC. CNV lesion characteristics: lesion size needs to be less than 10-disc areas (typical disc area=2.54 mm2).

3. May be phakic or pseudophakic.

4. Must have a negative or low serum titer result (≤300) for AAV8 NAbs.

5. BCVA between ≤20/25 and >20/125 (≤83 and ≥44 Early Treatment Diabetic Retinopathy Study [ETDRS] letters) in the study eye.

6. Based on the SD-OCT image obtained at Week 1, participants must have improvement in fluid (see Response Criterion below) and have a central retinal thickness (CRT)<400 μm. Note that, if the participant has disease other than fluid contributing to an increase (ie, PED or SHIRM) in CRT, they will be enrolled if they have <75 μm of total fluid (intraretinal or subretinal), as determined by the CRC. Response Criterion: Participants must have an improvement in inner retinal (parafovea 3 mm) fluid relative to Visit 2 of >50 μm or 50%; or an improvement in center subfield thickness of >50 μm or 50%, as determined by the CRC.

7. If both eyes are eligible, the study eye must be the participant's worse-seeing eye, as determined by the investigator.

8. Women must be postmenopausal (defined as being at least 12 consecutive months without menses) or surgically sterilized (i.e., having a bilateral tubal ligation/bilateral salpingectomy, bilateral tubal occlusive procedure, hysterectomy, or bilateral oophorectomy). If not, women must have negative serum and urine pregnancy tests at Day 1 and be willing to undergo additional pregnancy testing during the study.

9. Women of childbearing potential (WOCBP) (and their male partners) must be willing to use a highly effective method of contraception and male participants engaged in a sexual relationship with a WOCBP must be willing to use condoms from Week 2 until 24 weeks after Construct II administration.

10. Must be willing and able to provide signed informed consent, comply with all study procedures, and be available for the duration of the study.

### 6.8.3 Exclusion Criteria

Participants are excluded from the study if any of the following criteria apply:
1. CNV or macular edema in the study eye secondary to any causes other than AMD.
2. Subfoveal fibrosis or atrophy, as determined by the CRC.
3. Participants who required >10 anti-VEGF injections in the 12 months prior to Visit 2.
4. Participants who had a prior vitrectomy.
5. Any condition in the investigator's opinion that could limit VA improvement in the study eye.
6. Active or history of retinal detachment in the study eye.
7. Advanced glaucoma in the study eye, defined as IOP of >23 mmHg not controlled by 2 IOP-lowering medications or any invasive procedure to treat glaucoma (eg, shunt, tube, or MIGS devices; however, selective laser trabeculectomy and argon laser trabeculoplasty are permitted).
8. Any condition in the study eye that, in the opinion of the investigator, may increase the risk to the participant, require either medical or surgical intervention during the course of the study to prevent or treat vision loss, or interfere with study procedures or assessments.
9. History of intravitreal therapy in the study eye, such as intravitreal steroid injection or investigational product, other than anti-VEGF therapy, in the 6 months prior to Visit 2.
10. Presence of an implant in the study eye at screening (excluding an intraocular lens).
11. History of malignancy requiring chemotherapy and/or radiation in the 5 years prior to screening. Localized basal cell carcinoma will be permitted.
12. Received any gene therapy.
13. History of therapy known to have caused retinal toxicity, or concomitant therapy with any drug that may affect VA or with known retinal toxicity, e.g., chloroquine or hydroxychloroquine.
14. Any concomitant treatment that, in the opinion of the investigator, may interfere with the ocular procedure or healing process.
15. Known hypersensitivity to ranibizumab or any of its components or past hypersensitivity (in the investigator's opinion) to agents like Construct II.
16. Has a serious, chronic, or unstable medical or psychological condition that, in the opinion of the investigator, may compromise the participant's safety or ability to complete all assessments and follow-up in the study.
17. Any condition preventing visualization of the fundus or VA improvement in the study eye, e.g., cataract, vitreous opacity, fibrosis, atrophy, or retinal epithelial tear in the center of the fovea.
18. History of intraocular surgery in the study eye within 12 weeks prior to Visit 2. Yttrium aluminum garnet capsulotomy is permitted if performed >10 weeks prior to Visit 2.
19. Receipt of any investigational product within 30 days of Visit 2 or 5 half-lives of the investigational product, whichever is longer.
20. Ocular or periocular infection in the study eye that may interfere with the administration of Construct II.
21. Myocardial infarction, cerebrovascular accident, or transient ischemic attacks within the 6 months prior to Visit 2.
22. Uncontrolled hypertension (systolic blood pressure [BP]>180 mmHg, diastolic BP >100 mmHg) despite maximal medical treatment.
23. Any participant with the following laboratory values collected at Visit 2 and confirmed at Visit 3:
   Aspartate aminotransferase (AST)/alanine aminotransferase (ALT) >2.5× upper limit of normal (ULN).
   Total bilirubin >1.5×ULN, unless the participant has a previously known history of Gilbert's syndrome and a fractionated bilirubin that shows conjugated bilirubin <35% of total bilirubin.
   Prothrombin time >1.5×ULN, unless the participant is anticoagulated.
   Hemoglobin <10 g/dL for male participants and <9 g/dL for female participants.
   Platelets <100×10³ μL.
   Estimated glomerular filtration rate <30 mL/min/1.73 m2.

### 6.8.4 Study Intervention(s) Administered

Eligible participants will be assigned either to receive Construct II (Dose 1 or Dose 2) or ranibizumab in the study eye. Information regarding Construct II and ranibizumab follows.

TABLE 9
Information regarding Construct II and ranibizumab

| | | | Ranibizumab |
|---|---|---|---|
| Arm Name | Construct II Dose 1 | Construct II Dose 2 | (LUCENTIS) |
| Type | Gene therapy (AAV8.CB7.CI.amd42.RBG) | | Drug (control |
| | | | treatment arm and |
| | | | run-in/rescue) |
| Dose | Solution | | |
| Formulation | | | |
| Unit Dose | 1.0 × 10¹² GC/mL | 2.5 × 10¹² GC/mL | 10 mg/mL |
| Strength | | | |
| Dose Level(s) | 100 μL | 100 μL | 0.5 mg (0.05 mL of |
| | (2.5 × 10¹¹ GC/eye) | (5.0 × 10¹¹ GC/eye) | 10 mg/mL solution) |
| | delivered via a single | delivered via | once at Visit 2 or as |
| | SCS injection | 2 SCS injections at the | rescue starting 2 |
| | | same visit | weeks post Construct |
| | | | II administration, |
| | | | provided according to |
| | | | rescue criteria |

| Route of | Suprachoroidal space injection(s) in the study eye | Intravitreal injection |
|---|---|---|
| Administration | using the Clearside SCS Microinjector ™ | in the study eye |
| | investigational device | |
| Physical | Construct II investigational product is supplied as a | LUCENTIS is |
| Description | frozen, sterile, single-use solution of the AAV vector | supplied as a |
| | active ingredient (AAV8.CB7.CI.amd42.RBG) in a | preservative-free, |
| | formulation buffer. The solution appears clear to | sterile solution in a |
| | opalescent, colorless, and free of visible particulates | single-use container |
| | at room temperature. | designed to deliver |
| | | 0.05 mL of 10 mg/mL |
| | | LUCENTIS (0.5-mg |
| | | dose prefilled syringe |
| | | or vial) aqueous |
| | | solution. The solution |
| | | appears colorless to |
| | | pale yellow. |
| Manufacturer | Advanced BioScience Laboratories, Inc | Genentech, Inc |
| Packaging and | Construct II will be supplied as a sterile, single-use | Study intervention |
| Labeling | solution in 2-mL Crystal Zenith ® vials sealed with | will be obtained in |
| | latex-free rubber stoppers and aluminum flip-off | commercial |
| | seals. Each vial will be labeled as required per | packaging, either the |
| | country regulatory requirements. | prefilled syringe |

(NDC 50242-080-03)
or single-use 2-mL
glass vial (NDC
50242-080-02)
designed to deliver
0.05 mL of 10 mg/mL
ranibizumab solution.

### 6.9 Example 9: a Phase 2, Randomized, Dose-Escalation, Ranibizumab-Controlled Study to Evaluate the Efficacy, Safety, and Tolerability of Construct II Gene Therapy Delivered Via One or Two Suprachoroidal Space (SCS) Injections in Participants with Neovascular Age-Related Macular Degeneration (nAMD)

### 6.9.1 Synopsis

This example provides an overview of a phase 2a, dose assessment of Construct II gene therapy in participants with age-related macular degeneration.

### (a) Objectives and Endpoints

TABLE 10
Objectives and Endpoints

| | | |
|---|---|---|
| Measure | Objectives | Endpoints |

Primary

| | | |
|---|---|---|
| Efficacy | To evaluate the mean change | Mean change from baseline in BCVA to |
| | in BCVA for Construct II | Week 40 based on the ETDRS score |
| | compared with | |
| | ranibizumab monthly at | |
| | Week 40 | |

Secondary

| | | |
|---|---|---|
| Safety | To evaluate the safety and | Incidences of overall and ocular AEs and |
| | tolerability of Construct II | SAEs through Week 52 |
| | | Vector shedding analysis in serum, urine, |
| | | and tears |
| | To evaluate the incidences of | Proportion of participants who experience |
| | ocular inflammation | ocular inflammation following SCS |
| | following SCS | administration |
| | administration of | Mean change from baseline in CNV lesion |
| | Construct II | size and leakage area to Week 52 |
| | To evaluate the effect of | based on FA |
| | Construct II on CNV | |
| | lesion growth and leakage | |
| | as measured by FA | |
| Efficacy | To evaluate the effect of | Mean change from baseline in BCVA to |
| | Construct II on BCVA | Week 52 |
| | | Proportion of participants (1) gaining or |
| | | losing ≥15, ≥10, ≥5, or ≥0 letters; |
| | | (2) maintaining vision (not losing ≥15 |
| | | letters) compared with baseline as per |
| | | BCVA at Week 40 and Week 52 |
| | | Mean change from baseline in BCVA to |
| | | Week 40 and Week 52 for participants |
| | | who received ≤2 supplemental |
| | | anti-VEGF injections, 2 supplemental |
| | | anti-VEGF injections, 1 supplemental |
| | | anti-VEGF injection, or |
| | | 0 supplemental anti-VEGF injections |
| | | (Construct II randomized participants) |
| | To evaluate the effect of | Mean change from baseline in CRT as |
| | Construct II on CRT, as | measured by SD-OCT to Week 40 and |
| | measured by SD-OCT | Week 52 |
| | To assess the need for | Annualized supplemental anti-VEGF |
| | supplemental anti-VEGF | injection rate through Week 40 and |
| | therapy in participants | Week 52 |
| | who receive Construct II | Proportion of participants who have a |
| | treatment | reduction of ≥50% and ≥75% in the |
| | | annualized supplemental anti-VEGF |
| | | injection rate through Week 40 and |
| | | Week 52 compared with the prior |
| | | 52 weeks preceding the first |
| | | intravitreal ranibizumab injection |
| | | received as part of the Screening |
| | | Period (Construct II randomized |
| | | participants) |
| | | Proportion of subjects with 0, ≤1, and ≤2 |
| | | supplemental injections through |
| | | Week 52 |
| | | Mean percent reduction in the annualized |
| | | supplemental anti-VEGF injection rate |
| | | through Week 40 and Week 52 |
| | | compared with the prior 52 weeks |
| | | preceding the first ranibizumab |
| | | injection received as part of the |
| | | Screening Period (Construct II |
| | | randomized participants) |
| | | Time to first supplemental anti-VEGF |
| | | injection |
| Pharmacodynamics | To evaluate the concentration | Mean change from baseline in aqueous |
| | of Construct II TP in | Construct II TP concentrations over |
| | aqueous humor and serum | time |
| | | Mean change from baseline in serum |
| | | Construct II TP concentrations over |
| | | time |

Exploratory

| | | |
|---|---|---|
| Efficacy | To evaluate the effect of | Proportion of participants with no fluid on |
| | Construct II on fluid | SD-OCT |
| | accumulation as assessed | Proportion of participants with stable fluid |
| | by SD-OCT | on SD-OCT within 30 μm of baseline |
| Safety | To assess changes in visual | Changes in visual field testing over time |
| | function by visual fields | |
| | To evaluate the incidences of | Incidence of new area of geographic |
| | new areas of geographic | atrophy by FAF (in participants with |
| | atrophy, as assessed by | no geographic atrophy at baseline) |
| | FAF | |
| Biomarker | To evaluate the effect of | Association of positive serum AAV8 NAb |
| | positive serum adeno- | results with aqueous humor and serum |
| | associated virus serotype 8 | Construct II TP concentrations |
| | (AAV8) neutralizing | |
| | antibody (NAb) results on | |
| | the concentration of | |
| | Construct II TP in aqueous | |
| | humor and serum | |
| Immunogenicity | To evaluate the | Immunogenicity measurements (AAV8: |
| | immunogenicity of | NAbs, TAbs, and ELISpot; Construct |
| | Construct II | II protein: TAbs and ELISpot) |

AAV8 = adeno-associated virus serotype 8;
AE = adverse event;
BCVA = best-corrected visual acuity;
CNV = choroidal neovascularization;
CRT = central retinal thickness;
ELISpot = enzyme-linked ImmunoSpot;
ETDRS = Early Treatment Diabetic Retinopathy Study;
FA = fluorescein angiography;
FAF = fundus autofluorescence;
NAbs = neutralizing antibodies;
SAE = serious adverse event;
SCS = suprachoroidal space;
SD-OCT = spectral domain-optical coherence tomography;
TAbs = total binding antibodies;
TP = transgene product;
VEGF = vascular endothelial growth factor

### (b) Study Design

In this phase 2, randomized (3:1), dose-escalation, ranibizumab-controlled (Cohorts 1 and 2), study, approximately 115 participants with nAMD will be enrolled into 3 dose cohorts. Within each dose cohort, participants will receive a one-time administration of Construct II in the SCS (n=15 each in Cohorts 1, 2, and 4; n=20 each in Cohort 3 and 5; and n ˜20 in Cohort 6) or an intravitreal injection of ranibizumab 0.5 mg every 4 weeks up to Week 52 (n=5 participants).

Cohorts 1-5: Participants in Cohorts 1 and 2 will have negative or low serum titer results (≤300) for AAV8 NAbs and will be randomized to receive either Construct II (at a dose of 2.5×10¹¹ GC/eye [Cohort 1] or 5.0×10¹¹ GC/eye [Cohort 2]) or ranibizumab 0.5 mg. All participants in Cohort 3 will have a serum titer result >300 for AAV8 NAbs and will receive Construct II at a dose of 5.0×10¹¹ GC/eye (i.e., the same dose level given to participants in Cohort 2). All participants in Cohort 4 will have a negative or low serum titer result (≤300) for AAV8 NAbs and all participants in Cohort 5 will have a serum titer result >300 for AAV8 NAbs. The participants in both Cohorts 4 and 5 will receive a dose level of Construct II (1.0×10¹² GC/eye) that is higher than either of the doses tested in Cohorts 1 through 3. Enrollment of participants in Cohorts 3 and 5 will be stratified by screening serum AAV8 NAb titers.

Participants who receive Construct II will strongly be encouraged to enroll in a long-term follow-up study after completion of the current study at Week 52 (or early discontinuation) and will sign a separate informed consent for the follow-up study at that time. Participants in the ranibizumab control arm will be offered an opportunity following the Week 52 visit to be included in a future Construct II dose cohort.

Screening will comprise 3 visits to select for eligible participants with qualifying AAV8 neutralizing antibodies (NAbs) titers (Screening Visit 1) who demonstrate anatomic responsiveness to ranibizumab during a ranibizumab run-in phase (Screening Visits 2 and 3). During Screening Visit 1, participants who sign the informed consent form (ICF) will be evaluated for eligibility and will confirm NAb status from a NAb screening protocol. Participants who have negative or low (≤300) titer results for serum AAV8 NAbs will return to the study center to confirm the remaining inclusion/exclusion criteria. Participants continuing to meet eligibility criteria will receive a 0.5-mg intravitreal injection of ranibizumab in the study eye at Screening Visit 2 (Day 1). At Screening Visit 3 (Week 1), participants will be evaluated by spectral domain-optical coherence tomography (SD-OCT) to confirm their anatomic response to the screening anti-VEGF injection via comparison against their Day 1 SD-OCT assessment taken prior to the screening ranibizumab injection. Anatomic response will be determined by a central reading center (CRC) according to pre-specified criteria. To be randomized (Cohorts 1 and 2) or enrolled (Cohorts 3-5) the CRC must verify anatomic eligibility. Participants who do not have an anatomic response will be considered screen failures. For screen-failed participants, anyone who has an AE associated with the ranibizumab injections on Day 1 will be followed until the AE resolves (up to 30 days post injection).

At the Week 2 visit, Construct II randomized participants will receive either 1 or 2 injections of Construct II, depending on dose level, administered at the study center by SCS delivery using the Clearside SCS Microinjector™ investigational device; note that the Treatment Period of the study begins at the time of Construct II administration. All investigators will be trained on the SCS procedure. A detailed description of the procedure can be found in the SCS Administration Manual.

Participants receiving Construct II (Cohorts 1-5) will have 2 visits for postinjection safety (1 day postprocedure and 1 week postprocedure), followed by additional follow-up visits 2 weeks and 4 weeks after Construct II administration. Starting 6 weeks after Construct II administration (Week 8), Construct II participants will have monthly study visits. Starting 2 weeks after Construct II administration (Week 4), study participants may receive intravitreal ranibizumab supplemental therapy if they meet predefined supplemental injection criteria.

Participants randomized to the ranibizumab control arm (Cohorts 1 and 2) will have their first postrandomization visit at Week 4 and will receive intravitreal ranibizumab 0.5 mg. Participants will have monthly (˜ every 28 days) study visits during which they will receive an intravitreal injection of ranibizumab 0.5 mg.

Enrollment will begin with the randomization of 2 sentinel participants in Cohort 1: one sentinel participant will be randomized to Construct II at dose level 1 and the other to ranibizumab control. The sentinel participant who is randomized to and receives Construct II will be observed for safety during the 2-week postadministration period. Following this, the Sponsor's Internal Safety Committee (ISC) will review the safety data for the sentinel participant receiving Construct II and, if no safety review triggers (SRTs) are observed, the remaining participants within Cohort 1 will be randomized to either Construct II dose level 1 (n=14) or ranibizumab control (n=4).

Once Cohort 1 is fully enrolled and all participants have completed the 2-week postadministration safety visit, the Independent Data Monitoring Committee (IDMC) will review the available cumulative safety data from Cohort 1 (dose level 1) to determine if enrollment of dose level 2 may be initiated. During any safety review, the IDMC may recommend halting dosing, proceeding with the same or lower dose level, or proceeding to the next planned dose level. If the decision is made to dose escalate, randomization of 2 sentinel participants in Cohort 2 will begin, again with one randomized to Construct II (this time at dose level 2) and the other to ranibizumab control. Following the 2-week postadministration safety visit for the sentinel participant who is randomized to and receives Construct II, the ISC will review that subject's safety data and, if there are no observed SRTs, the remaining participants within Cohort 2 will be randomized to either Construct II dose level 2 (n=14) or ranibizumab control (n=4). In addition, simultaneous enrollment of Cohort 3 (n=20) may also begin, with all participants in the cohort receiving Construct II at dose level 2 (ie, the same dose level of Construct II evaluated in Cohort 2).

Once Cohort 2 is fully enrolled and all participants have completed the 2-week postadministration safety visit, the IDMC will review the available cumulative safety data to determine if enrollment of dose level 3 may be initiated. If the decision is made to dose escalate, enrollment of a single sentinel participant in Cohort 4 will begin; this participant will receive Construct II at dose level 3. Note that initiation of enrollment in Cohort 4 is predicated only on completion of dosing of all participants in Cohort 2 (ie, enrollment in Cohort 3 may still be ongoing at the time enrollment in Cohort 4 begins). Following the 2-week postadministration safety visit, the ISC will review the safety data for the sentinel participant in Cohort 4 and, if there are no observed SRTs, the remaining participants within Cohort 4 will be enrolled (n=14). Additionally, provided Cohort 3 enrollment has completed, simultaneous enrollment of Cohort 5 (n=20) may also begin, with all participants in the cohort receiving Construct II at dose level 3 (ie, the same dose level of Construct II evaluated in Cohort 4).

The dose escalation plan is designed to ensure that eligible participants having a negative or low serum titer result (≤300) for screening serum AAV8 NAbs will complete dosing at a given dose level of Construct II, followed by IDMC review, before escalation may occur to the next dose level of Construct II. Prior to any dose escalation, the IDMC will review available cumulative safety data, inclusive of the 2-week postadministration safety visit from the last dosed participant within the dose level having a negative or low serum titer result for screening serum AAV8 NAbs.

For participants in the Construct II treatment arms, immunogenicity to the vector (as assessed by anti-AAV8 antibodies [serum], anti-Construct II TP antibodies [serum], and enzyme-linked ImmunoSpot [ELISpot][whole blood]), and Construct II TP concentrations (aqueous humor and serum) will be assessed throughout the study.

Cohort 6: Cohort 6 will evaluate the efficacy, safety, and tolerability of SCS administration of Construct II at the highest dose level tested in the study, 1.0×10¹² GC/eye. There will not be a separate control arm in Cohort 6 (the ranibizumab control arm combined from Cohorts 1 and 2 will be used for analysis purposes). AAV8 NAbs will be measured as part of the baseline laboratory testing in Cohort 6, but will no longer be used for screening purposes to determine eligibility and enrollment.

Approximately 20 eligible participants with nAMD will be enrolled upon signing an informed consent. Eligibility will be determined during the screening period based primarily on demonstrated anatomic responsiveness to ranibizumab during 2 ranibizumab run-in injections and stricter requirements related to intraretinal fluid levels.

Eligible participants will receive a 0.5-mg intravitreal injection of ranibizumab in the study eye at Visit 1 (Week −4). Based on the Visit 2 (Week −3) SD-OCT, there must be improvement in fluid relative to Visit 1, as determined by the CRC, to confirm anatomic response to ranibizumab. Participants who do not have an anatomic response will be considered screen failures. For screen-failed participants, anyone who has an AE associated with the ranibizumab run-in injections will be followed until the AE resolves (up to 30 days postinjection).

At Visit 3 (Day 1), participants who continue to meet all entry criteria will receive a second 0.5-mg intravitreal injection of ranibizumab, and will be scheduled to receive a single 1.0×10¹² GC/eye dose of Construct II administered via SCS injection. Participants will be randomized to one of 2 different steroid regimens (Group 1 and Group 2), to be administered following SCS Construct II administration, 10 participants in each group.

Participants will have their study intervention administered at the study center at the Week 2 visit. Construct II will be given as single 100-μL SCS injection using the Clearside SCS Microinjector investigational device. All investigators will be trained on the SCS procedure. A detailed description of the procedure can be found in the SCS Administration Manual to be given to the investigators.

Group 1 participants will undergo subtenon injection of triamcinolone acetonide injectable suspension, USP (i.e. KENALOG®-40) immediately after SCS administration of Construct II. Group 2 participants will start a 7-week regimen of difluprednate ophthalmic emulsion, 0.05% (i.e. DUREZOL®) treatment beginning the day of SCS administration of Construct II (Week 2 visit). Participants will have 2 visits for postinjection safety (1 day postprocedure and 1 week postprocedure), followed by additional follow-up visits 2 weeks and 4 weeks after Construct II administration. Cohort 6 participants will have a scheduled intravitreal ranibizumab injection at Week 4 and beginning at Week 8, may receive supplemental ranibizumab as needed based on prespecified retreatment criteria determined by SD-OCT and BCVA evaluation.

All Cohorts: Efficacy will be the primary focus of the initial 40 weeks (primary study period). Following completion of the primary study period, participants will continue to be assessed until Week 52. At the end of the Week 52 study visit, participants who received Construct II will be invited to enroll into a long-term follow-up study, while participants who were in the ranibizumab control arm, if eligible, will be offered an opportunity to be included in a future Construct II dose cohort. Participants will be evaluated for safety through the assessment of AEs, including SAEs and adverse events of special interest (AESIs) (ocular inflammation deemed by the investigator to be unrelated to the surgical/study procedure and graded as 2+ or greater on the ocular inflammation grading scales, ocular infections [including endophthalmitis], retinal tears or detachment, retinal thinning, and new arterial thromboembolic events [nonfatal stroke, nonfatal myocardial infarction, or vascular death (including deaths of unknown cause)]), as well as assessments of clinical laboratory tests (chemistry, hematology, coagulation, urinalysis), and ocular examinations and imaging (BCVA, IOP, slit-lamp biomicroscopy, indirect ophthalmoscopy, fluorescein angiography [FA], ultra-wide field Optos fundus auto fluorescence [FAF], ultra-wide field Optos color fundus photography [CFP], Humphrey visual field 120, ormicroperimetry, and SD-OCT). Note that AEs will be collected at all study visits. Participants who show evidence of new retinal hypo/hyper pigmentation changes as compared with baseline will be monitored using SD-OCT scans. Radial SD-OCT scans that transverse the margin of the hypo/hyper pigmentary area will be captured when possible.

Planned safety monitoring of the study participants will be conducted on an ongoing basis. The monitoring will include reviews conducted by the Medical Monitor and routine reviews conducted by the Sponsor's ISC. Separately, an IDMC will also be established and will meet on a periodic basis to independently review the clinical data.

### 6.9.2 Inclusion Criteria

### (a) Cohorts 1-5

Participants are eligible to be included in the study only if all of the following criteria apply:
1. Males or females, aged ≥50 years and ≤89 years.
2. Must have a diagnosis of CNV secondary to AMD in the study eye, have received anti-VEGF therapy in the past and been responsive, and have retinal fluid (either subretinal or intraretinal) within the parafovea (3-mm center of the macula, based on the early treatment diabetic retinopathy grid) at study entry (Day 1), as assessed by the CRC.
   CNV lesion characteristics: lesion size needs to be less than 10-disc areas (typical disc area=2.54 mm2).
3. May be phakic or pseudophakic.
4. Participants in Cohorts 1, 2, and 4 must have a negative or low serum titer result (≤300) for AAV8 NAbs. Participants in Cohorts 3 and 5 must have a serum titer result >300 for AAV8 NAbs.
5. BCVA between ≤20/25 and >20/125 (≤83 and >44 Early Treatment Diabetic Retinopathy Study [ETDRS] letters) in the study eye.
6. Based on the SD-OCT image obtained at Week 1, participants must have improvement in fluid (see Response Criterion below) and have a central retinal thickness (CRT)<400 μm. Note that, if the participant has disease other than fluid contributing to an increase (ie, PED or SHRM) in CRT, they will be enrolled if they have <75 μm of total fluid (intraretinal or subretinal), as determined by the CRC.
   Response Criterion: Participants must have an improvement in inner retinal (parafovea 3 mm) fluid relative to Screening Visit 2 of >50 μm or, if the participant has <50 μm of fluid, then any improvement in fluid, as determined by the CRC.
7. If both eyes are eligible, the study eye must be the participant's worse-seeing eye, as determined by the investigator.
8. Women must be postmenopausal (defined as being at least 12 consecutive months without menses) or surgically sterilized (i.e., having a bilateral tubal ligation/bilateral salpingectomy, bilateral tubal occlusive procedure, hysterectomy, or bilateral oophorectomy).
9. Male participants engaged in a sexual relationship with a WOCBP must be willing to use condoms (and their partners to use a medically accepted method of contraception) from day of Construct II administration until 4 weeks after Construct II administration.
10. Must be willing and able to provide signed informed consent, comply with all study procedures, and be available for the duration of the study.

### (b) Cohort 6

Participants are eligible to be included in the study only if all of the following criteria apply:
1. Males or females, aged ≥50 years and ≤89 years.
2. Must have a diagnosis of CNV secondary to AMD in the study eye, have received anti-VEGF therapy in the past and been responsive, and have retinal fluid (either subretinal or intraretinal) within the parafovea (3-mm center of the macula, based on the early treatment diabetic retinopathy grid) at study entry (Day 1), as assessed by the CRC. CNV lesion characteristics: lesion size needs to be less than 10-disc areas (typical disc area=2.54 mm2).
3. May be phakic or pseudophakic.
4. BCVA between <20/25 and >20/125 (≤83 and >44 Early Treatment Diabetic Retinopathy Study [ETDRS] letters) in the study eye.
5. Must have a CRT <400 μm at Screening Visit 1, as determined by the CRC. Note that, if the study eye has disease other than fluid contributing to an increase (ie, PED or SHRM) in CRT, the participant will be enrolled only if the study eye has <50 μm of fluid in the inner retina (parafovea 3 mm), as determined by the CRC.
   Response Criterion: Based on the Screening Visit 2 SD-OCT, there must be improvement in fluid relative to Screening Visit 1, in which there is ≤50 μm* of remaining fluid height in the inner retina (parafovea 3 mm) of the study eye or, if the study eye has <50 μm of fluid at Screening Visit 1, then the study eye must have any improvement in fluid.
   * maximum fluid height of 50 μm can be anywhere in the 3 mm region relative to Screening Visit 1
6. If both eyes are eligible, the study eye must be the participant's worse-seeing eye, as determined by the investigator.
7. Women must be postmenopausal (defined as being at least 12 consecutive months without menses) or surgically sterilized (i.e., having a bilateral tubal ligation/bilateral salpingectomy, bilateral tubal occlusive procedure, hysterectomy, or bilateral oophorectomy).
8. Male participants engaged in a sexual relationship with a WOCBP must be willing to use condoms (and their partners to use a medically accepted method of contraception) from day of Construct II administration until 4 weeks after Construct II administration.
9. Must be willing and able to provide signed informed consent, comply with all study procedures, and be available for the duration of the study.

### 6.9.3 Exclusion Criteria

### (a) Cohorts 1-5

Participants are excluded from the study if any of the following criteria apply:
1. CNV or macular edema in the study eye secondary to any causes other than AMD.
2. Subfoveal fibrosis or atrophy, as determined by the CRC.
3. Participants who required >10 anti-VEGF injections in the 12 months prior to Screening Visit 2.
4. Study eye with an nAMD diagnosis >4 years from Day 1.
5. Participants who had a prior vitrectomy.
6. Any condition in the investigator's opinion that could limit VA improvement in the study eye.
7. Active or history of retinal detachment in the study eye.
8. Advanced glaucoma in the study eye, defined as IOP of >23 mmHg not controlled by 2 IOP-lowering medications or any invasive procedure to treat glaucoma (eg, shunt, tube, or MIGS devices; however, selective laser trabeculectomy and argon laser trabeculoplasty are permitted).
9. Any condition in the study eye that, in the opinion of the investigator, may increase the risk to the participant, require either medical or surgical intervention during the course of the study to prevent or treat vision loss, or interfere with study procedures or assessments.
10. History of intravitreal therapy in the study eye, such as intravitreal steroid injection or investigational product, other than anti-VEGF therapy, in the 6 months prior to Screening Visit 2.
11. Presence of an implant in the study eye at screening (excluding an intraocular lens).
12. History of malignancy requiring chemotherapy and/or radiation in the 5 years prior to screening. Localized basal cell carcinoma will be permitted.
13. Received any gene therapy.
14. History of therapy known to have caused retinal toxicity, or concomitant therapy with any drug that may affect VA or with known retinal toxicity, e.g., chloroquine or hydroxychloroquine.
15. Any concomitant treatment that, in the opinion of the investigator, may interfere with the ocular procedure or healing process.
16. Known hypersensitivity to ranibizumab or any of its components or past hypersensitivity (in the investigator's opinion) to agents like Construct II.
17. Has a serious, chronic, or unstable medical or psychological condition that, in the opinion of the investigator, may compromise the participant's safety or ability to complete all assessments and follow-up in the study.
18. Any condition preventing visualization of the fundus or VA improvement in the study eye, e.g., cataract, vitreous opacity, fibrosis, atrophy, or retinal epithelial tear in the center of the fovea.
19. History of intraocular surgery in the study eye within 12 weeks prior to Screening Visit 2. Yttrium aluminum garnet capsulotomy is permitted if performed >10 weeks prior to Screening Visit 2.
20. Receipt of any investigational product within 30 days of Screening Visit 2 or 5 half-lives of the investigational product, whichever is longer.
21. Ocular or periocular infection in the study eye that may interfere with the administration of Construct II.
22. Myocardial infarction, cerebrovascular accident, or transient ischemic attacks within the 6 months prior to Screening Visit 2.
23. Uncontrolled hypertension (systolic blood pressure [BP]>180 mmHg, diastolic BP >100 mmHg) despite maximal medical treatment.
24. Any participant with the following laboratory values collected at Screening Visit 2 and evaluated prior to randomization or enrollment that, in the opinion of the investigator and Medical Monitor, may compromise the participant's safety or ability to complete all assessments and follow-up in the study. In the event Screening Visit 2 test results are unavailable prior to randomization or enrollment, the investigator, with the approval of the Medical Monitor, may consider historical laboratory test results collected any time within the 6 months prior to Screening Visit 2.

### (b) Cohort 6

Participants are excluded from the study if any of the following criteria apply:
1. CNV or macular edema in the study eye secondary to any causes other than AMD.
2. Subfoveal fibrosis or atrophy, as defined by:
   a. Central subfield fibrosis (central 1 mm) in the study eye, as determined by the CRC.
   b. Any central subfield atrophy (central 1 mm) in the study eye (eg, incomplete RPE and outer retinal atrophy [iRORA], or complete RPE and outer retinal atrophy), as determined by the CRC.
3. Participants who required >10 anti-VEGF injections in the 12 months prior to Screening Visit 2.
4. Study eye with an nAMD diagnosis >4 years from Day 1.
5. Participants who had a prior vitrectomy.
6. Any condition in the investigator's opinion that could limit VA improvement in the study eye.
7. Active or history of retinal detachment in the study eye.
8. Any serous PED >400 μm in the study eye at Screening Visit 1, as assessed by the CRC.
9. Any nonserous fibrovascular/vascular PED >200 μm (peak within the central 5 mm) in the study eye at Screening Visit 1, as assessed by the CRC.
10. Clinically significant ERM of Grade 2 or higher severity in the study eye at Screening Visit 1.
11. Active or history of glaucoma or ocular hypertension in the study eye, defined as IOP >21 mmHg.
12. High myopia with spherical equivalent of the refractive error in the study eye demonstrating ≥−8.00 diopters or an axial length >26 mm.
13. Any condition in the study eye that, in the opinion of the investigator, may increase the risk to the participant, require either medical or surgical intervention during the course of the study to prevent or treat vision loss, or interfere with study procedures or assessments.
14. History of intravitreal therapy in the study eye, such as intravitreal steroid injection or IP, other than an intravitreal therapy for nAMD, in the 6 months prior to Screening Visit 1.
15. Presence of an implant in the study eye at screening (excluding an intraocular lens).
16. History of malignancy requiring chemotherapy and/or radiation in the 5 years prior to screening. Localized basal cell carcinoma will be permitted.
17. Received any gene therapy.
18. History of therapy known to have caused retinal toxicity, or concomitant therapy with any drug that may affect VA or with known retinal toxicity, e.g., chloroquine or hydroxychloroquine.
19. Any concomitant treatment that, in the opinion of the investigator, may interfere with the ocular procedure or healing process.
20. Known hypersensitivity to ranibizumab or any of its components or past hypersensitivity (in the investigator's opinion) to agents like Construct II.
21. Has a serious, chronic, or unstable medical or psychological condition that, in the opinion of the investigator, may compromise the participant's safety or ability to complete all assessments and follow-up in the study.
22. Any condition preventing visualization of the fundus or VA improvement in the study eye, eg, cataract, vitreous opacity, fibrosis, atrophy, or retinal epithelial tear in the center of the fovea.
23. History of intraocular surgery in the study eye within 12 weeks prior to Screening Visit 1. Yttrium aluminum garnet capsulotomy is permitted if performed >10 weeks prior to Screening Visit 1.
24. Receipt of any investigational product within 30 days of Screening Visit 1 or 5 half-lives of the investigational product, whichever is longer.
25. Ocular or periocular infection in the study eye that may interfere with the administration of Construct II.
26. Myocardial infarction, cerebrovascular accident, or transient ischemic attacks within the 6 months prior to Screening Visit 1.
27. Uncontrolled hypertension (systolic BP >180 mmHg, diastolic BP >100 mmHg) despite maximal medical treatment.
28. Any participant with any abnormal laboratory values collected at Screening Visit 1 and evaluated prior to randomization or enrollment that, in the opinion of the investigator and Medical Monitor, may compromise the participant's safety or ability to complete all assessments and follow-up in the study. In the event Screening Visit 1 test results are unavailable prior to randomization or enrollment, the investigator, with the approval of the Medical Monitor, may consider historical laboratory test results collected any time within the 6 months prior to Screening Visit 1.

### 6.9.4 Study Intervention(s) Administered

Eligible participants will be assigned either to receive Construct II (Dose 1, Dose 2, or Dose 3) or ranibizumab in the study eye. Cohort 1 comprises patients having negative or low (<300) NAb serum titers for AAV8, and receives Dose 1 (2.5×10¹¹ GC/eye) in a single 100 μL SCS injection. Cohort 2 comprises patients having negative or low (<300) NAb serum titers for AAV8, and receives Dose 2 (5.0×10¹¹ GC/eye) in a two 100 μL SCS injections, for a total volume injected of 200 μL at the same visit. Cohort 3 comprises patients having positive or higher (>300) NAb serum titers for AAV8, and receives Dose 2 (5.0×10¹¹ GC/eye) in a single 100 μL SCS injection. Cohort 4 comprises patients having negative or low (<300) NAb serum titers for AAV8, and receives Dose 3 (1.0×10¹² GC/eye) in a single 100 μL SCS injection. Cohort 5 comprises patients having positive or higher (>300) NAb serum titers for AAV8, and receives Dose 3 (1.0×10¹² GC/eye) in a single 100 μL SCS injection. Cohort 6 receives Dose 3 (1.0×10¹² GC/eye) in a single 100 μL SCS injection and a steroid treatment regime described herein below. Information regarding Construct II and ranibizumab follows.

### (a) Cohorts 1-5

TABLE 11
Information regarding Construct II and ranibizumab

| Arm | Construct | | | Construct | Ranibizumab |
|---|---|---|---|---|---|
| Name | II Dose 1 | Construct II Dose 2 | | Dose 3 | (LUCENTIS) |
| Type | Gene therapy (AAV8.CB7.CI.amd42.RBG) | | | | Drug (control |
| | | | | | treatment arm and |
| | | | | | run-in/rescue) |
| Dose | Solution | | | | Solution |
| Formulation | | | | | |
| Unit Dose | Cohort 1: | Cohort 2: | Cohort 3: | Cohorts 4&5: | 10 mg/mL |
| Strength | 2.5 × 10¹² | 2.5 × 10¹² | 3.0 × 10¹³ | 3.0 × 10¹³ | |
| | GC/mL | GC/mL | GC/mL | GC/mL | |
| | | | (diluted to | (diluted to | |
| | | | 5.0 × 10¹² | 1.0 × 10¹³ | |
| | | | GC/mL) | GC/mL) | |
| Dose | 100 μL | 100 μL | 100 μL | 100 μL | 0.5 mg (0.05 mL of |
| Level(s) | (2.5 × 10¹¹ | (5.0 × 10¹¹ | (5.0 × 10¹¹ | (1.0 × 10¹² | 10 mg/mL solution) |
| | GC/eye) | GC/eye) | GC/eye) | GC/eye) | once at Visit 2 or as |
| | delivered via | delivered via | delivered via | delivered via | rescue starting 2 |
| | a single SCS | 2 SCS | a single SCS | a single SCS | weeks post |
| | injection | injections at | injection | injection | Construct II |
| | (100-μL total | the same visit | (100-μL total | (100-μL total | administration, |
| | volume) | (200-μL total | volume) | volume) | provided according |
| | | volume) | | | to rescue criteria |

| Route of | Suprachoroidal space injection(s) in the study eye using the | Intravitreal |
|---|---|---|
| Administration | Clearside SCS Microinjector ™ investigational device | injection in the |
| | | study eye |
| Physical | Construct II investigational product is supplied as a frozen, | LUCENTIS is |
| Description | sterile, single-use solution of the AAV vector active ingredient | supplied as a |
| | (AAV8.CB7.CI.amd42.RBG) in a formulation buffer. The | preservative-free, |
| | solution appears clear to opalescent, colorless, and free of | sterile solution in a |
| | visible particulates at room temperature. | single-use |
| | | container designed |
| | | to deliver 0.05 mL |
| | | of 10 mg/mL |
| | | LUCENTIS (0.5- |
| | | mg dose prefilled |
| | | syringe or vial) |
| | | aqueous solution. |
| | | The solution |
| | | appears colorless to |
| | | pale yellow. |
| Manufacturer | Advanced BioScience Laboratories, Inc | Genentech, Inc |
| Packaging | Construct II will be supplied as a sterile, single-use solution in | Study intervention |
| and | 2-mL Crystal Zenith ® vials sealed with latex-free rubber | will be obtained in |
| Labeling | stoppers and aluminum flip-off seals. Each vial will be labeled | commercial |
| | as required per country regulatory requirements. | packaging, either |
| | | the prefilled |
| | | syringe (NDC |
| | | 50242-080-03) or |
| | | single-use 2-mL |
| | | glass vial (NDC |
| | | 50242-080-02) |
| | | designed to deliver |
| | | 0.05 mL of |
| | | 10 mg/mL |
| | | ranibizumab |
| | | solution. |

### (b) Cohort 6

Approximately 20 eligible participants will be selected for Cohort 6.

Cohort 6 participants will be randomized to receive either subtenon injection of KENALOG-40 (Group 1) or DUREZOL (Group 2) steroid treatment in the study eye following SCS administration of Construct II.

TABLE 12
Information regarding Construct II, ranibizumab,
KENALOG-40, and DUREZOL for Cohort 6 follows.

| | | Ranibizumab | | |
|---|---|---|---|---|
| | Construct II Dose 3 | (LUCENTIS) | | |
| Intervention | (Group 1 and | (Group 1 and | KENALOG-40 | DUREZOL |
| Name | Group 2) | Group 2) | (Group 1) | (Group 2) |
| Type | Gene therapy | Drug (run-in and | Drug (synthetic | Drug (topical |
| | (AAV8.CB7.CI.amd42.RBG) | post Construct II | glucocorticoid | corticosteroid) |
| | | supplemental | corticosteroid) | |
| | | treatment) | | |
| IP or | IP | NIMP/AxMP | NIMP/AxMP | NIMP/AxMP |
| NIMP/AxMP | | | | |
| Dose | Solution | Solution | Injectable | Ophthalmic |
| Formulation | | | suspension | emulsion |
| Unit Dose | 3.0 × 10¹³ GC/mL | 10 mg/mL | 40 mg/mL | 0.5 mg/mL |
| Strength | (diluted to | | | (0.05%) |
| | 1.0 × 10¹³ GC/mL) | | | |
| Dose Level(s) | 1.0 × 10¹² GC/eye | 0.5 mg (0.05 mL | 40 mg (1 mL of | 1 drop |
| | delivered via a | of 10 mg/mL | 40 mg/mL | (see below |
| | single 100 μL SCS | solution) once at | suspension) | for dosing |
| | injection (100-μL | Week −4 and Day | immediately | regimen) |
| | total volume) | 1 (run-in), Week 4 | following SCS | |
| | | (2 weeks post | Construct II | |
| | | Construct II | injection (Week 2 | |
| | | treatment), and | visit) | |
| | | PRN starting at | | |
| | | Week 8 | | |
| Route of | Suprachoroidal | Intravitreal | Approximately | Topical |
| Administration | space injection in | injection in the | 1 mL delivered | administration |
| | the study eye using | study eye | once via a single | of 1 drop |
| | the Clearside SCS | | subtenon injection | into the |
| | Microinjector ® | | immediately after | conjunctival |
| | investigational | | SCS Construct II | sac of the |
| | device | | administration, in a | study eye |
| | | | study eye quadrant | |
| | | | separate to that | |
| | | | used for Construct | |
| | | | II | |
| Physical | Construct II is an | LUCENTIS is | KENALOG-40 is | DUREZOL |
| Description | investigational | supplied as a | supplied as a | (difluprednate |
| | product is supplied | preservative-free, | sterile aqueous | ophthalmic |
| | as a frozen, sterile, | sterile solution in | suspension. | emulsion) |
| | single-use solution | a single-use | | 0.05% is |
| | of the AAV vector | container designed | | supplied as a |
| | active ingredient | to deliver 0.05 mL | | sterile, |
| | (AAV8.CB7.CI.amd42.RBG) | of 10 mg/mL | | preserved, |
| | in a formulation | LUCENTIS | | aqueous |
| | buffer. The solution | (0.5 mg dose | | topical |
| | appears clear to | prefilled syringe | | ophthalmic |
| | opalescent, | or vial) aqueous | | emulsion |
| | colorless, and free | solution. The | | |
| | of visible | solution appears | | |
| | particulates at room | colorless to pale | | |
| | temperature. | yellow. | | |
| Manufacturer | Advanced | Genentech, Inc | Bristol-Myers | Alcon ®, a |
| | BioScience | | Squibb Company | Novartis |
| | Laboratories, Inc | | | Company |
| Packaging and | Construct II will be | Ranibizumab will | KENALOG-40 | DUREZOL |
| Labeling | supplied as a | be obtained in | Injection | (difluprednate |
| | sterile, single-use | commercial | (triamcinolone | ophthalmic |
| | solution in 2-mL | packaging, either | acetonide injectable | emulsion) |
| | Crystal Zenith ® | the prefilled | suspension, USP) is | 0.05% is a |
| | vials sealed with | syringe or single- | supplied in vials | sterile, |
| | latex-free rubber | use 2-mL glass | providing 40 mg | aqueous |
| | stoppers and | vial designed to | triamcinolone | topical |
| | aluminum flip-off | deliver 0.05 mL of | acetonide per mL. | ophthalmic |
| | seals. Each vial will | 10 mg/mL | 40 mg/mL, 1 mL | emulsion |
| | be labeled as | ranibizumab | vial | supplied in |
| | required per | solution. | | an opaque |
| | country regulatory | | | plastic bottle |
| | requirements. | | | with a |
| | | | | controlled |
| | | | | drop tip and a |
| | | | | pink cap; 5 |
| | | | | mL in an |
| | | | | 8-mL bottle |

AAV = adeno-associated virus; AAV8 = adeno-associated virus serotype 8; AxMP = auxiliary medicinal product; IP = investigational product; NIMP = noninvestigational medicinal product; PRN = as needed; SCS = suprachoroidal space; USP = United States Pharmacopeia.

Steroid Regimens: Cohort 6 participants will be randomized to receive one of 2 different steroid regimens to manage the risk of ocular inflammation after SCS administration of Construct II as follows:

Group 1 (subtenon injection of KENALOG-40): Immediately after SCS administration of Construct II at the Week 2 visit, the treating investigator will administer a one-time single injection of KENALOG-40 (1 mL of 40 mg/mL suspension, 40 mg) into the subtenon of the study eye, in a quadrant separate to that used for Construct II.

Group 2 (DUREZOL): Beginning the day of SCS administration of Construct II (Week 2 visit), participants will instill 1 drop of difluprednate ophthalmic emulsion 0.05% (DUREZOL) in the study eye, then instill drops daily in the study eye starting the next day, gradually decreasing in a tapered regimen over a total of 7 weeks as follows:
4 times daily for 4 weeks, followed by
3 times daily for 1 week, followed by
2 times daily for 1 week, followed by
1 time daily for 1 week.

For any ocular inflammatory events occurring during or after the prophylaxis treatment for Cohort 6 participants (either Group 1 or Group 2), the Sponsor Clinical Development Lead should be contacted for management/treatment recommendations, to potentially include DUREZOL drops.

### 6.9.5 Results

As of Aug. 1, 2022, suprachoroidal delivery of Construct II has been well tolerated across 85 patients dosed in Cohorts 1-5. Fifteen SAEs were reported, none of which were considered related to Construct II. For the total group of Cohorts 1-4 (n=65), all common treatment emergent adverse events (TEAEs) through 6 months in the study eye included conjunctival hemorrhage, dry eye, episcleritis, and conjunctival hyperemia. Mild intraocular inflammation was reported at similar incidence in the first and second dose levels, with a slight increase in incidence in mild to moderate inflammation seen at the third dose level (Cohort 4). All intraocular inflammation resolved with topical corticosteroids.

Patients treated with Construct II continue to demonstrate stable Best Corrected Visual Acuity (BCVA) and central retinal thickness (CRT) at 6 months. In addition, a meaningful reduction in anti-vascular endothelial growth factor (anti-VEGF) treatment burden following administration of Construct II compared to mean annualized injection rate during the 12 months prior to administration was observed and ranged from −63.8% to −84.7% across all cohorts. The highest reduction in treatment burden was observed in the third dose level, with patients receiving a mean of 1.3 injections over six months following administration of Construct II, which represents an 84.7% reduction in anti-VEGF treatment burden. Ten out of 15 patients (67%) in the third dose level received no anti-VEGF injections over six months following Construct II administration. In these patients, visual acuity and CRT was observed to be stable over six months.

Surprisingly, the data from the second dose level (Cohorts 2 and 3) suggests there is no meaningful difference in safety and vision outcomes for patients who are neutralizing antibody (NAb) positive.

### 6.9.6 Interim Data

Construct II was well tolerated in patients receiving Dose 3 (1.0×10¹² GC/eye), with no drug-related serious adverse events. Time of post-administration follow up ranged from six weeks to six months.

As shown in Table 13 below, mild and similar intraocular inflammatory (101) was observed across doses in patients from Cohorts 1-3 (Doses 1 (2.5×10¹¹ GC/eye) and 2 (5.0×10¹¹ GC/eye)). Patients from Cohort 4 (Dose 3 (1.0×10¹² GC/eye)) developed mild to moderate IOI with increased incidence as compared to patients receiving Doses 1 and 2.

TABLE 13
Cohorts 1-4 Interim Safety Summary

| Cohort 1 to 4: Common | Cohort 1 | | Cohort 2 | | Cohort 3 | | Cohort 4 | | | |
|---|---|---|---|---|---|---|---|---|---|---|
| Ocular TEAEs¹ in the | Dose 1 | | Dose 2 | | Dose 2 | | Dose 3 | | | |
| Study Eye through 6 | NAb− | | NAb− | | NAb+ | | NAb− | | Total | |
| Months | (N = 15) | | (N = 15) | | (N = 20) | | (N = 15) | | (N = 65) | |
| Intraocular | 4 | (26.7%) | 3 | (20.0%) | 2 | (10.0%) | 6 | (40.0%) | 15 | (23.1. %) |
| Inflammation² | | | | | | | | | | |
| Conjunctival | 5 | (33.3%) | 2 | (13.3%) | 3 | (15.0%) | 1 | (6.7%) | 11 | (16.9%) |
| Hemorrhage | | | | | | | | | | |
| Intraocular Pressure | 1 | (6.7%) | 2 | (13.3%) | 3 | (15.0%) | 3 | (15.0%) | 9 | (13.8%) |
| Increased³ | | | | | | | | | | |
| Conjunctival | 2 | (13.3%) | 1 | (6.7%) | 1 | (5.0%) | 3 | (20.0%) | 7 | (10.8%) |
| Hyperemia | | | | | | | | | | |

| | | | | | | | | | |
|---|---|---|---|---|---|---|---|---|---|
| Episcleritis⁴ | 0 | 3 | (20.0%) | 2 | (10.0%) | 2 | (13.3%) | 7 | (10.8%) |

¹Includes AEs for total group ≥10% with onset up to 6 m visit.
²All cases were mild to moderate (range +0.5 to 2+), most presented 2-6 weeks post injection, predominantly as anterior cells on slit lamp examination. Resolved on topical corticosteroids.
³Intraocular pressure increased and ocular hypertension have been combined into one group. All mild to moderate and all controlled.
⁴All mild (grade 1), presented 2-6 weeks post injection and resolved on topical corticosteroid or NSAID treatment.

Short-course of ocular steroid prophylaxis meaningfully reduced the occurrence of mild to moderate intraocular inflammation seen in previous cohorts. Table 14 below included all Dose 3 (1.0×10¹² GC/eye) cohorts and compared without prophylaxis (PPX) steroid in the left column to with short course prophylaxis steroid in Cohort 6 overall. Cohort 6 was split by route of prophylaxis steroid, of either one-time periocular steroid or short course topical steroid taper. In all patients who received the short-course (seven-week) prophylactic topical steroid eye drops, there were zero cases of intraocular inflammation.

TABLE 14
Cohorts 4-6 Interim Safety Summary

| | No PPX Steroid | | w/PPX Steroid | | | | |
|---|---|---|---|---|---|---|---|
| | 26 Weeks | | 6-26 Weeks | | | | |
| | Follow-up | | Follow-up | | One-time | | |
| Common Ocular | Cohorts 4 & 5 | | Cohort 6 | | Subtenon | | Topical |
| TEAEs¹ in the | Dose Level 3 | | Dose Level 3 | | Steroid | | Steroid |
| Study Eye | (N = 35) | | (N = 20) | | (N = 10) | | (N = 10) |
| Conjunctival | 14 | (40%) | 1 | (5.0%) | 1 | (10.0%) | 0 |
| Hyperemia | | | | | | | |
| Episcleritis² | 13 | (37.1%) | 5 | (25.0%) | 2 | (20.0%) | 3 (30.0%) |
| Intraocular | 7 | (20.0%) | 2 | (10.0%)³ | 2 | (20.0%)³ | 0 |
| Inflammation | | | | | | | |
| Intraocular | 5 | (14.3%) | 1 | (5.0%) | 1 | (10.0%) | 0 |
| Pressure | | | | | | | |
| Increased⁴ | | | | | | | |
| Conjunctival | 3 | (8.6%) | 1 | (5.0%) | 1 | (10.0%) | 0 |
| Hemorrhage | | | | | | | |

¹Includes AEs ≥10% of the total groups. TEAE: Treatment-Emergent Adverse Event.
²All mild to moderate (grade 1 and 2), presented within 1 week to 26 weeks post injection and have resolved or are tapering off topical corticosteroids.
³Two cases were mild (0.5+ and 1+), presented 2-14 weeks post injection as anterior cells on slit lamp examination with no vitreous involvement, and have resolved on topical corticosteroids by the next visit.
⁴Intraocular pressure increased and ocular hypertension have been combined into one group. All mild to moderate and all controlled.

Therefore, short course prophylactic ocular steroids successfully mitigated the risk of inflammation.

### 6.10 Example 10: An Open-Label Phase 2A Dose Assessment of Construct 11 Gene Therapy in Participants with Diabetic Retinopathy

This example provides an overview of a phase 2a, dose assessment of Construct II gene therapy in participants with diabetic retinopathy (DR). The sustained, stable expression of the Construct II transgene product following a one-time gene therapy treatment for DR could potentially reduce the treatment burden of currently available therapies while maintaining vision with a favorable benefit:risk profile. The current proof of concept study is intended to evaluate the safety and efficacy of Construct II gene therapy at 2 different dose levels in participants with DR.

### 6.10.1 Objectives and Endpoints

TABLE 15
Primary and Secondary Objectives and Endpoints

| | |
|---|---|
| Objectives | Endpoints |

Primary

| | | |
|---|---|---|
| Efficacy | To evaluate the effect of | Proportion of participants achieving a 2-step |
| | Construct II on the | or greater improvement in ETDRS-DRSS on |
| | ETDRS-DRSS at Week 24 | 4-widefield digital stereoscopic fundus |
| | | photography at Week 24 |

Secondary

| | | |
|---|---|---|
| Efficacy | To evaluate the effect of | Proportion of participants achieving a 2-step |
| | Construct II on the | or greater improvement in ETDRS-DRSS on |
| | ETDRS-DRSS at | 4-widefield digital stereoscopic fundus |
| | additional time points | photography at Week 12 |
| | | Proportion of participants achieving a 0-step |
| | | (no change), 1-step, 2-step, or 3-step |
| | | improvement in ETDRS-DRSS on |
| | | 4-widefield digital stereoscopic fundus |
| | | photography at Week 12 and Week 24 |
| | | Proportion of participants achieving a 1-step or |
| | | greater, or a 3-step or greater improvement in |
| | | ETDRS-DRSS on 4-widefield digital |
| | | stereoscopic fundus photography at Week 12 |
| | | and Week 24 |
| | | Proportion of participants with a 1-step or |
| | | greater, a 2-step or greater, or a 3-step or |
| | | greater worsening in ETDRS-DRSS on |
| | | 4-widefield digital stereoscopic fundus |
| | | photography at Week 12 and Week 24 |
| | | Proportion of participants graded as Level 61 or |
| | | 65 (PDR) at baseline achieving regression to |
| | | Level 47 or 53 (NPDR) |
| Safety/ | To assess the safety, | Proportion of phakic participants with |
| Immunogenicity | tolerability, and | cataracts meeting the protocol-specified |
| | immunogenicity of | criteria for removal at either Week 18 or |
| | Construct II | Week 24, or at an unscheduled visit prior to |
| | | Week 18 |
| | | Incidences of ocular and systemic AEs |
| | | Immunogenicity measurements (serum |
| | | neutralizing antibodies to AAV8 and serum |
| | | antibodies to Construct II TP) over 24 weeks |
| Safety/Efficacy | To evaluate the need | Proportion of participants requiring any |
| | for additional SOC | additional intervention for diabetic |
| | intervention due to | complications to Week 24 |
| | diabetic complications | Proportion of participants with any sight- |
| | | threatening diabetes complications to Week 24 |
| | | Proportion of participants developing diabetic |
| | | complications (eg, CI-DME or |
| | | neovascularization) requiring anti-VEGF |
| | | treatment per SOC through Week 24; for this |
| | | population, the following endpoints will be |
| | | evaluated: |
| | | Number of anti-VEGF injections received |
| | | up to Week 24 |
| | | Duration of time from study intervention |
| | | (Day 1) to first anti-VEGF administration |
| | | per SOC |
| | | Proportion of participants developing diabetic |
| | | complications (eg, neovascularization due to |
| | | DR) requiring PRP per SOC through |
| | | Week 24; for this population, the following |
| | | endpoints will be evaluated: |
| | | Duration of time from study intervention |
| | | (Day 1) to first PRP |
| | | Proportion of participants requiring more |
| | | than 1 PRP |
| | | Proportion of participants developing diabetic |
| | | complications (eg, retinal detachment) |
| | | requiring surgical intervention (pneumatic |
| | | retinopexy, cryopexy, or scleral buckle) per |
| | | SOC; for this population, the following |
| | | endpoint will be evaluated: |
| | | Duration of time from study intervention |
| | | (Day 1) to surgical intervention |
| Pharmacodynamics | To measure aqueous | Aqueous Construct II TP concentrations at |
| | and serum Construct | assessed time points |
| | II TP concentrations | Serum Construct II TP concentrations at |
| | | assessed time points |

Exploratory

| | | |
|---|---|---|
| Efficacy/Safety | To evaluate the | Proportion of participants with visual stability |
| | effect of Construct II | (within 5 ETDRS letters or ±5 ETDRS letters) |
| | on vision outcomes | from baseline to Week 24 |
| | in all evaluable | Proportion of participants with vision gain or |
| | participants | vision loss >5 ETDRS letters from baseline to |
| | | Week 24 |
| | To evaluate the | Mean change in CST on SD-OCT at Week 12 |
| | effect of Construct | and Week 24 |
| | II on anatomic | Proportion of participants achieving ≤290 μm |
| | outcomes evaluated | CST on SD-OCT at Week 12 and Week 24 |
| | using SD-OCT in all | Proportion of participants with clinically |
| | evaluable participants | significant macular thickening in CST ≥30 |
| | | μm from baseline at Week 12 and Week 24 |
| | | Mean change in macular volume and percent |
| | | reduction in macular volume from baseline |
| | | based on SD-OCT, as determined by the CRC |
| | To assess evidence | Proportion of participants graded as Level 61 |
| | of vessel regression | or 65 at baseline with evidence of vessel |
| | for participants with | regression at Week 24 based on FA |
| | baseline PDR (Level | |
| | 61 or 65) | |
| | To assess changes in | Proportion of participants graded as Level 61 |
| | the area of leakage | or 65 at baseline with change in area of |
| | for participants with | leakage from baseline to Week 24 based on |
| | baseline PDR (Level | FA |
| | 61 or 65) | |
| | To assess changes | Mean change from baseline in the area of |
| | from baseline in the | retinal nonperfusion at Week 24 based on FA |
| | area of retinal | in all evaluable participants |
| | nonperfusion in all | |
| | evaluable participants | |
| Biomarkers | To measure aqueous | VEGF-A concentration in aqueous fluid at |
| | VEGF-A concentration | assessed time points |

AAV8 = adeno-associated virus serotype 8;
AE = adverse event;
CI-DME = center involved-diabetic macular edema;
CRC = central reading center;
CST = central subfield thickness;
DR = diabetic retinopathy;
DRSS = Diabetic Retinopathy Severity Scale;
ETDRS = Early Treatment Diabetic Retinopathy Study;
FA = fluorescein angiography;
PDR = proliferative diabetic retinopathy;
PRP = panretinal photocoagulation;
SD-OCT = spectral domain-optical coherence tomography;
SOC = standard of care;
TP = transgene product;
VEGF = vascular endothelial growth factor

### 6.10.2 Inclusion Criteria

Participants must meet all the following criteria in order to be eligible for this study. All ocular criteria refer to the study eye: (1) men or women between 18-89 years of age with DR secondary to diabetes mellitus Type 1 or 2. Participants must have a hemoglobin A1c≤10% (as confirmed by laboratory assessments obtained at Screening or by a documented laboratory report dated within 60 days prior to Screening); (2) participant deemed to be an appropriate surgical candidate, per the investigator; (3) study eye with moderately-severe NPDR, severe NPDR, mild PDR, or moderate PDR (ETDRS-DRSS Levels 47, 53, 61, or 65 using standard 4-widefield digital stereoscopic fundus photographs, as determined by the CRC) for which PRP or anti-VEGF injections can be safely deferred, in the opinion of the investigator, for at least 6 months after Screening; (4) no evidence in the study eye of high-risk characteristics typically associated with vision loss, per the investigator, including the following: (i) new vessels within 1-disc area of the optic nerve, or vitreous or preretinal hemorrhage associated with less extensive new vessels at the optic disc, or with new vessels elsewhere that are half a disc area or more in size, and (ii) no evidence in the study eye of anterior segment (eg, iris or angle) neovascularization on clinical examination; (5) best-corrected visual acuity (BCVA) in the study eye of >69 ETDRS letters (approximate Snellen equivalent 20/40 or better); note: if both eyes are eligible, the study eye must be the participant's worse-seeing eye, as determined by the investigator prior to enrollment; (6) prior history of CI-DME in the study eye is acceptable if no intravitreal anti-VEGF or short-acting steroid injections have been given within the last 6 months, AND no more than 10 documented injections have been given in the 3 years prior to Screening; (7) sexually active male participants with female partners of childbearing potential must be willing to use condoms plus a medically accepted form of partner contraception from Screening until 24 weeks after vector administration; (9) must be willing and able to comply with all study procedures and be available for the duration of the study; (10) must be willing and able to provide written, signed informed consent.

### 6.10.3 Exclusion Criteria

Participants are excluded from the study if any of the following criteria apply: (1) women of childbearing potential, defined as neither postmenopausal nor surgically sterile. Postmenopausal is defined to be documented 12 consecutive months without menses. Surgically sterile is defined as having bilateral tubal ligation/bilateral salpingectomy, bilateral tubal occlusive procedure, hysterectomy, or bilateral oophorectomy; (2) presence of any active CI-DME, as determined by the investigator, on clinical examination or within the center subfield of the study eye using the following threshold: Heidelberg Spectralis: 320 m; (3) neovascularization in the study eye from a cause other than DR, per investigator; (4) evidence in the study eye, as determined by the investigator, of ischemia in the study eye involving >50% of the peripheral retina, or the fovea or papillomacular area on baseline FA; (5) evidence in the study eye of optic nerve pallor on clinical exam, as determined by investigator; (6) any evidence of or documented history of PRP or retinal laser in the study eye; (7) ocular or periocular infection in the study eye that may interfere with the surgical procedure; (8) any ocular condition in the study eye that could require surgical intervention within the 6 months after Screening (vitreous hemorrhage, cataract that does not meet the inclusion criteria, retinal traction, epiretinal membrane, etc) or any condition in the study eye that may, in the opinion of the investigator, increase the risk to the participant, require either medical or surgical intervention during the study to prevent or treat vision loss, or interfere with the study procedures or assessments; (9) active or history of retinal detachment in the study eye; (10) presence of an implant in the study eye at Screening (excluding intraocular lens [IOL]); (11) for phakic participants, Pentacam Nuclear Staging score ≥1 as scanned by the Pentacam device and verified by the CRC, or not meeting other baseline cataract criteria; (12) advanced glaucoma in the study eye (ie, uncontrolled, despite 2 or more drop treatments or an intervention such as a tube or shunt), as assessed through consultation with the participant's glaucoma specialist or documented history of glaucoma surgery; (13) history of intraocular surgery in the study eye within 12 weeks prior to Screening; yttrium aluminum garnet (YAG) capsulotomy is permitted if performed >10 weeks prior to Screening; (14) history of intravitreal therapy in the study eye, including anti-VEGF therapy, within 6 months prior to Screening, and documentation of more than 10 prior anti-VEGF or short-acting steroid intravitreal injections in the study eye for DME within 3 years of Screening; (15) any prior intravitreal steroid injection in the study eye within 6 months prior to Screening, administration in the study eye of Ozurdex® within 12 months prior to Screening, or administration in the study eye of Iluvien® within 36 months prior to Screening; (16) any prior systemic anti-VEGF treatment within the 6 months prior to or plans to use systemic anti-VEGF therapy during the next 6 months after Screening; (17) history of therapy known to have caused retinal toxicity, or concomitant therapy with any drug that may affect VA or with known retinal toxicity, e.g., chloroquine or hydroxychloroquine; (18) myocardial infarction, cerebrovascular accident, or transient ischemic attacks within the 6 months prior to Screening; (19) uncontrolled hypertension (systolic blood pressure [BP]>180 mmHg, diastolic BP>100 mmHg) despite maximal medical treatment; note that if BP is brought below 180/100 mmHg and stabilized by antihypertensive treatment as determined by the investigator and/or primary care physician, the participant can be rescreened for eligibility; (20) a systemic condition that, in the opinion of the investigator, would preclude participation in the study (poor glycemic control, uncontrolled hypertension, etc); (21) any concomitant treatment that, in the opinion of the investigator, may interfere with the ocular surgical procedure or the healing process; (22) history of malignancy or hematologic malignancy that may compromise the immune system requiring chemotherapy and/or radiation in the 5 years prior to Screening. Localized basal cell carcinoma will be permitted; (23) has a serious, chronic, or unstable medical or psychological condition that, in the opinion of the investigator, may compromise the participant's safety or ability to complete all assessments and follow-up in the study; (24) any participant with the following laboratory values at Screening will be withdrawn from the study: (i) aspartate aminotransferase (AST)/alanine aminotransferase (ALT) >2.5×upper limit of normal (ULN), (ii) total bilirubin >1.5×ULN, unless the participant has a previously known history of Gilbert's syndrome and a fractionated bilirubin that shows conjugated bilirubin <35% of total bilirubin, (iii) prothrombin time >1.5×ULN, unless the participant is anticoagulated. Participants who are anticoagulated will be monitored by local labs and managed per local practice to hold or bridge anticoagulant therapy for the study procedure; consultation with the Medical Monitor is required if the participant is anticoagulated, (iv) hemoglobin <10 g/dL for male participants and <9 g/dL for female participants, (v) Platelets <100×103/μL, (vi) estimated glomerular filtration rate <30 mL/min/1.73 m²; (25) history of chronic renal failure requiring dialysis or kidney transplant; (26) initiation of intensive insulin treatment (pump or multiple daily injections) within the 6 months prior to Screening or plans to do so within 6 months of Screening; (27) currently taking anticoagulation therapy for which holding anticoagulation therapy for Construct II administration is not indicated or considered to be unsafe in the opinion of the treating investigator (ie, retinal surgeon), as well as the physician prescribing anticoagulation for the participant, as verified by the Medical Monitor; (28) participation in any other gene therapy study, including Construct II, or receipt of any investigational product within 30 days prior to enrollment or 5 half-lives of the investigational product, whichever is longer, or any plans to use an investigational product within 6 months following enrollment; (29) known hypersensitivity to ranibizumab or any of its components.

### 6.10.4 Study Intervention

Study intervention is defined as any investigational intervention(s), marketed product(s), placebo, or medical device(s) intended to be administered to a study participant according to the study protocol.

Eligible participants will be assigned to receive a single dose of either Construct II (Dose 1) or a single dose of Construct II (Dose 2). All participants will receive study intervention on Day 1 via subretinal delivery in an operating room.

TABLE 16
Summary of Study Intervention(s)

| Arm Name | Construct II Dose 1 | Construct II Dose 2 |
|---|---|---|
| Type | Gene therapy (AAV8.CB7.CI.amd42.rBG) | |
| Dose Formulation | Solution | |
| Unit Dose Strength | 6.2 × 10¹¹ GC/mL | 1.0 × 10¹² GC/mL |
| Dosage Level(s) | 250 μL | 250 μL |
| | (1.6 × 10¹¹ GC/eye) | (2.5 × 10¹¹ GC/eye) |
| | one-time dose | one-time dose |

| | |
|---|---|
| Route of | Subretinal delivery |
| Administration | |
| Physical | Construct II investigational product is supplied as a frozen, |
| Description | sterile, single- use solution of the AAV vector active ingredient |
| | (AAV8.CB7.CI.amd42.rBG) in a formulation buffer. The solution |
| | appears clear to opalescent, colorless, and free of visible |
| | particulates at room temperature. |
| Packaging | Study intervention will be supplied as a sterile, single-use solution |
| and Labeling | in 2-mL Crystal Zenith ® vials sealed with latex free rubber stoppers |
| | and aluminum flip-off seals. Each vial will be labeled as required |
| | per applicable regulatory requirements. |

### 6.10.5 Prior and Concomitant Therapy

### (a) Medications and Therapies

The following medications are prohibited prior to entry into the study:
Any prior systemic or ocular anti-VEGF treatment in the study eye within the 6 months prior to Screening.
More than 10 prior, documented, anti-VEGF or short-acting steroid intravitreal injections in the study eye for DME within 3 years of Screening.
Any prior intravitreal short-acting steroid injection in the study eye within 6 months prior to Screening, administration in the study eye of Ozurdex within 12 months prior to Screening, or administration in the study eye of Iluvien within 36 months prior to Screening.
Initiation of intensive insulin treatment (pump or multiple daily injections) within the 6 months prior to Screening; for participants meeting this criterion, modification of the regimen is permitted during the study, as recommended and documented by their primary care provider or other treatment provider.
Participants must not have used any concomitant treatment that, in the opinion of the investigator, could interfere with Construct II administration or the healing process.
Participants are prohibited from taking anticoagulation therapy for which holding anticoagulation therapy for Construct II administration is not indicated or considered to be unsafe in the opinion of the treating investigator (ie, retinal surgeon), as well as the physician prescribing anticoagulation for the participant.
Participants must not have used any investigational product within 30 days prior to enrollment or within 5 half-lives of the investigational product, whichever is longer.

The following concomitant medications are prohibited during the study:
Anti-VEGF therapy in the study eye during the 6 months after Screening, except for treatment of ocular diabetes complications.
Initiation of intensive insulin treatment (pump or multiple daily injections) is not allowed during the study; as indicated previously, modification of the treatment regimen is allowed during the study if initiation of treatment occurred at least 6 months prior to Screening.

Postoperative care for participants receiving Construct II is described in the Procedures Manual. There are no other restrictions on prior or concomitant therapy in this study.

### (b) Treatment of Ocular Diabetes Complications

All complications of ocular diabetes will be managed in accordance with each study centers SOC and must be documented as an AE.

During the study, participants who develop diabetic complications requiring anti-VEGF treatment per SOC may be administered therapy as required. If needed, the study centers will provide their own supply of FDA-approved anti-VEGF therapy. The number of anti-VEGF injections received, and the timing of all administrations, must also be recorded in the source documents and eCRF.

Participants who develop diabetic complications requiring PRP SOC must have the time of PRP recorded in the source documents and eCRF.

Participants who develop diabetic complications requiring surgical intervention SOC (either pneumatic retinopexy, cryopexy, or scleral buckle) must have the type of intervention and the time of intervention recorded in the source documents and eCRF.

### (c) Intervention for Cataract Formation

### Baseline Screening for Phakic Participants

During the Screening visit, a series of assessments will be completed to determine eligibility and establish the participant's baseline cataract status for phakic participants only. These assessments include the following: (1) assessing the participant's symptoms per SOC; (2) performing a clinical examination to determine whether any clinically significant cataract, per cataract investigator, is present; (3) imaging the lens nucleus with the Oculus Pentacam Nuclear Staging (PNS) system. Pentacam grade ≤1 is acceptable for inclusion into the study. Pentacam eligibility should be determined at the site, and Pentacam scan should be submitted to the CRC for verification; and (4) imaging the participant's cortex and posterior capsule of the lens with standardized red reflex anterior segment photographs, which will be submitted to the CRC for grading and confirmation of study eligibility. Any subject with either cortical or posterior subcapsular lens image grade ≥Level 2 AREDS (mild opacities) will not be eligible.

### On-Study Cataract Evaluation and Intervention for Phakic Participants

During the study, the retina investigator and cataract investigator will continue to assess participants for the presence of cataracts meeting the criteria for removal specified below.

The criterion for medically indicated cataract extraction, which is to be reported as an AE, is as follows: the retina investigator is unable to adequately view and/or image the retina in order to safely monitor and manage diabetic eye disease and/or general retinal status.

If the criterion for medically indicated cataract extraction is met at any postbaseline visit, an unscheduled visit for cataract extraction surgery will be scheduled as soon as possible by the study coordinator with the cataract investigator.

If the criterion for medically indicated cataract extraction is not met, but the participant meets either of the following two secondary criteria at any postbaseline visit, (BCVA decrease or participant-reported, described below), the study coordinator should schedule an unscheduled visit as soon as possible to obtain confirmatory Pentacam and CRC-graded lens photos (if not already available at that visit):

1. BCVA decrease: a decrease in BCVA of >5 ETDRS letters, relative to the best value recorded during the study (baseline or postbaseline) believed to be the result of worsening of cataract.

2. Participant-reported: visual symptoms resulting in lifestyle impairment as reported by the participant believed to be the result of worsening of cataract.

If during the unscheduled visit, a change in nuclear sclerosis from baseline on Pentacam Nuclear Staging of ≥1 grade or CRC-graded cortical or posterior subcapsular red reflex lens imaging of moderate cataract (ie, 5% involvement of central 5 mm) is confirmed, the secondary criteria for cataract extraction gas been met. This should be reported as an AE, and the study coordinator should schedule the unscheduled visit for cataract extraction as soon as possible by the cataract investigator.

A monofocal, 1-piece acrylic IOL is the lens of choice for use in this study. In some instances, a toric (astigmatism-correcting) IOL could be considered, but any difference in cost between a monofocal IOL and a toric lens is the responsibility of the participant unless otherwise approved by the Sponsor and the Medical Monitor. Multifocal or other premium IOLs are excluded during the study, as they may diminish the ability to accurately track any changes in retinal pathology. Silicone optic IOLs will not be used because of their potential to complicate any subsequent retinal procedures. The cataract surgeon may provide the participant with a recommendation that is most likely to provide optimal postoperative VA and visual function.

A postoperative, SOC protocol intended to limit complications will be followed. The preferred SOC protocol includes: fluroquinolone drops 4-times daily for 1 week, Ilevro (nepafenac) 2-times daily for 1 month, and a steroid taper with prednisolone acetate starting with 4-times daily for 1 week, tapering down 1 week at a time to 3-times daily, 2-times daily, and, finally, 1-time daily. For participant safety, alternative postoperative protocols may be used where appropriate, and with approval by the Medical Monitor.

### 6.11 Example 11: a Phase 2, Randomized, Dose-Escalation, Observation-Controlled Study to Evaluate the Efficacy, Safety, and Tolerability of Construct II Gene Therapy Delivered Via One or Two Suprachoroidal Space (SCS) Injections in Participants with Diabetic Retinopathy (DR) without Center Involved-Diabetic Macular Edema (CI-DME)

This example provides an overview of a phase 2a, dose assessment of Construct II gene therapy in participants with diabetic retinopathy (DR).

### 6.11.1 Objectives and Endpoints

TABLE 17
Objectives and Endpoints

| | |
|---|---|
| Objectives | Endpoints |

Primary

| | | |
|---|---|---|
| Efficacy | To evaluate the effect of | Proportion of participants achieving a 2-step |
| | Construct II on DR by the | or greater improvement in DR by |
| | ETDRS-DRSS at Week 48 | ETDRS-DRSS on 4-widefield digital |
| | | stereoscopic fundus photography at Week 48 |

Secondary

| | | |
|---|---|---|
| Efficacy | To evaluate the effect of | Proportion of participants achieving a 2-step |
| | Construct II on DR | or greater improvement in DR per |
| | (ETDRS-DRSS) over time | ETDRS-DRSS on 4-widefield digital |
| | | stereoscopic fundus photography at Week 4, |
| | | Week 12, and Week 24 |
| | | Proportion of participants achieving a 0-step |
| | | (no change), a 1-step or greater, or a 3-step |
| | | or greater improvement in DR per |
| | | ETDRS-DRSS on 4-widefield digital |
| | | stereoscopic fundus photography at Week 4, |
| | | Week 12, Week 24, and Week 48 |
| | | Proportion of participants with a 1-step or |
| | | greater, a 2-step or greater, or a 3-step or |
| | | greater worsening in DR per ETDRS-DRSS |
| | | on 4-widefield digital stereoscopic fundus |
| | | photography at Week 4, Week 12, Week 24, |
| | | and Week 48 |
| | | Proportion of participants graded as Level |
| | | 61 (PDR) at baseline achieving regression |
| | | to Level 47 or 53 (NPDR) at Week 24 and |
| | | Week 48 |
| Safety/ | To assess the safety, | Incidences of overall and ocular AEs |
| Immunogenicity | tolerability, and | Immunogenicity measurements (AAV8: |
| | immunogenicity of | NAbs, TAbs, and ELISpot; Construct II TP: |
| | Construct II | anti-Construct II TP antibodies and |
| | | ELISpot) over 48 weeks |
| Safety/Efficacy | To evaluate the need | Proportion of participants requiring any |
| | for additional SOC | additional intervention for ocular diabetic |
| | intervention due to | complications to Week 48 |
| | ocular diabetic | Proportion of participants with any sight- |
| | complications | threatening ocular diabetic complications to |
| | | Week 48 |
| | | Proportion of participants developing ocular |
| | | diabetic complications (eg, CI-DME or |
| | | neovascularization) requiring anti-VEGF |
| | | treatment per SOC through Week 48; for |
| | | this population, the following endpoints will |
| | | be evaluated: |
| | | Number of anti-VEGF injections received |
| | | Duration of time from study intervention |
| | | (Day 1) to first anti-VEGF |
| | | administration per SOC |
| | | Proportion of participants developing ocular |
| | | diabetic complications (eg, |
| | | neovascularization due to DR) requiring |
| | | PRP per SOC through Week 48; for this |
| | | population, the following endpoints will be |
| | | evaluated: |
| | | Duration of time from study intervention |
| | | (Day 1) to first PRP |
| | | Proportion of participants requiring more than 1 PRP |
| | | Proportion of participants developing ocular |
| | | diabetic complications (eg, retinal |
| | | detachment) requiring surgical intervention |
| | | (pneumatic retinopexy, cryopexy, or scleral |
| | | buckle) per SOC; for this population, the |
| | | following endpoint will be evaluated: |
| | | Duration of time from study intervention |
| | | (Day 1) to surgical intervention |
| Pharmacodynamics | To measure aqueous | Aqueous Construct II TP concentration at |
| | and serum Construct II | assessed time points |
| | TP concentrations | Serum Construct II TP concentration at |
| | | assessed time points |

Exploratory

| | | |
|---|---|---|
| Efficacy/Safety | To evaluate the effect of | Proportion of participants with visual |
| | Construct II on vision | stability (within 5 ETDRS letters or ±5 |
| | outcomes (BCVA in all | ETDRS letters) from baseline to Week 48 |
| | Construct II treated | Proportion of participants with vision gain |
| | participants) | or vision loss >5 ETDRS letters from |
| | | baseline to Week 48 |
| | To evaluate the effect of | Proportion of participants with clinically |
| | Construct II on visual | significant changes in visual field from |
| | field in all Construct II | baseline to Week 48, as determined by the |
| | treated participants | investigator |
| | To evaluate the effect of | Mean change from baseline in CST on |
| | Construct II on | SD-OCT at Week 24 and Week 48 |
| | anatomic outcomes | Proportion of participants achieving ≤290 |
| | assessed using SD-OCT | μm in CST on SD-OCT at Week 24 and Week 48 |
| | in all Construct II | Proportion of participants with clinically |
| | treated participants | significant macular thickening in CST ≥30 |
| | | μm from baseline at Week 24 and |
| | | Week 48, as determined by the CRC |
| | | Mean change in macular volume and |
| | | percent reduction in macular volume at |
| | | Week 48 relative to baseline on SD-OCT, as |
| | | determined by the CRC |
| | To assess evidence of | Proportion of participants graded as Level |
| | vessel regression on FA | 61 at baseline with evidence of vessel |
| | for participants with | regression at Week 24 and Week 48 based |
| | baseline PDR (Level 61) | on FA, as determined by the CRC |
| | To assess changes in the | Proportion of participants graded as |
| | area of leakage on FA | Level 61 at baseline with change in the area |
| | for participants with | of leakage from baseline to Week 24 and |
| | baseline PDR (Level 61) | Week 48 based on FA, as determined by the CRC |
| | To assess changes from | Mean change from baseline in the area of |
| | baseline in the area of | retinal nonperfusion at Week 24 and Week |
| | retinal nonperfusion on | 48 based on FA in all evaluable participants, |
| | Optos widefield FA in | as determined by the CRC |
| | all evaluable participants | |

AAV8 = adeno-associated virus serotype 8;
AE = adverse event;
BCVA = best-corrected visual acuity;
CI-DME = center involved-diabetic macular edema;
CRC = central reading center;
CST = central subfield thickness;
DR = diabetic retinopathy;
DRSS = Diabetic Retinopathy Severity Scale;
ELISpot = enzyme-linked ImmunoSpot;
ETDRS = Early Treatment Diabetic Retinopathy Study;
FA = fluorescein angiography;
NAb = neutralizing antibody;
PDR = proliferative diabetic retinopathy;
PRP = panretinal photocoagulation;
SD-OCT = spectral domain-optical coherence tomography;
SOC = standard of care;
TAb = total binding antibody;
TP = transgene product;
VEGF = vascular endothelial growth factor

### 6.11.2 Inclusion Criteria

Participants must meet all the following criteria in order to be eligible for this study. All ocular criteria refer to the study eye:
1. Men or women 25-89 years of age with DR secondary to diabetes mellitus Type 1 or
2. Participants must have a hemoglobin A1c≤10% (as confirmed by laboratory assessments obtained at Screening Visit 2 or by a documented laboratory report dated within 60 days prior to Screening Visit 2).
2. Study eye with moderately-severe NPDR, severe NPDR, or mild PDR (ETDRS-DRSS levels 47, 53, or 61 using standard 4-widefield digital stereoscopic fundus photographs, as determined by the CRC) for which PRP or anti-VEGF injections can be safely deferred, in the opinion of the investigator, for at least 6 months after Screening Visit 2.
3. No evidence in the study eye of high-risk characteristics typically associated with vision loss, per the investigator, including the following:
   New vessels within 1-disc area of the optic nerve
   Vitreous or preretinal hemorrhage associated with less extensive new vessels at the optic disc, or with new vessels elsewhere that are half a disc area or more in size.
   No evidence in the study eye of anterior segment (eg, iris or angle) neovascularization on clinical examination.
4. Must have a negative or low (≤300) serum titer result for AAV8 NAbs.
5. Best-corrected visual acuity in the study eye of ≥69 ETDRS letters (approximate Snellen equivalent 20/40 or better); note: if both eyes are eligible, the study eye must be the participant's worse-seeing eye, as determined by the investigator, prior to enrollment.
6. Prior history of CI-DME in the study eye is acceptable if no intravitreal anti-VEGF or short-acting steroid injections have been given within the last 6 months, AND no more than 10 documented injections have been given in the 3 years prior to Screening Visit 2.
7. Sexually active male participants with female partners of childbearing potential must be willing to use condoms plus a medically accepted form of partner contraception from Screening Visit 2 until 24 weeks after vector administration.
8. Must be willing and able to comply with all study procedures and be available for the duration of the study.
9. Must be willing and able to provide written, signed informed consent.

### 6.11.3 Exclusion Criteria

Participants are excluded from the study if any of the following criteria apply:
1. Women of childbearing potential (ie, women who are not postmenopausal or surgically sterile) are excluded from this clinical study.
   Postmenopausal is defined to be documented 12 consecutive months without menses.
   Surgically sterile is defined as having bilateral tubal ligation/bilateral salpingectomy, bilateral tubal occlusive procedure, hysterectomy, or bilateral oophorectomy.
2. Presence of any active CI-DME, as determined by the investigator, on clinical examination or within the central subfield thickness (CST) of the study eye, as determined by SD-OCT evaluated by CRC, using the following threshold:
   Heidelberg Spectralis: CST greater than 320 m
3. Neovascularization in the study eye from a cause other than DR, per investigator.
4. Evidence in the study eye of optic nerve pallor on clinical examination, as determined by the investigator.
5. Any evidence or documented history of PRP or retinal laser in the study eye.
6. Ocular or periocular infection in the study eye that may interfere with the SCS procedure.
7. Any ocular condition in the study eye that could require surgical intervention within the 6 months after Screening Visit 2 (vitreous hemorrhage, cataract, retinal traction, epiretinal membrane, etc) or any condition in the study eye that may, in the opinion of the investigator, increase the risk to the participant, require either medical or surgical intervention during the study to prevent or treat vision loss, or interfere with the study procedures or assessments.
8. Active or history of retinal detachment in the study eye.
9. Presence of an implant in the study eye at Screening Visit 2 (excluding intraocular lens).
10. Participants who had a prior vitrectomy surgery.
11. Advanced glaucoma in the study eye, as defined by an IOP >23 mmHg, not controlled by 2 IOP-lowering medications, any invasive procedure to treat glaucoma (eg, shunt, tube, or MIGS devices; however, selective laser trabeculectomy and argon laser trabeculoplasty are permitted), or visual field loss encroaching on central fixation.
12. History of intraocular surgery in the study eye within 12 weeks prior to Screening Visit 2; yttrium aluminum garnet (YAG) capsulotomy is permitted if performed >10 weeks prior to Screening Visit 2.
13. History of intravitreal therapy in the study eye, including anti-VEGF therapy, within 6 months prior to Screening Visit 2, and documentation of more than 10 prior anti-VEGF or short-acting steroid intravitreal injections in the study eye within 36 months of Screening Visit 2.
14. Any prior intravitreal steroid injection in the study eye within 6 months prior to Screening Visit 2, administration in the study eye of Ozurdex® within 12 months prior to Screening Visit 2, or administration in the study eye of Iluvien® within 36 months prior to Screening Visit 2.
15. Any prior systemic anti-VEGF treatment within the 6 months prior to or plans to use systemic anti-VEGF therapy during the next 48 weeks after Screening Visit 2.
16. History of therapy known to have caused retinal toxicity, or concomitant therapy with any drug that may affect VA or with known retinal toxicity, eg, chloroquine or hydroxychloroquine.
17. Myocardial infarction, cerebrovascular accident, or transient ischemic attacks within the 6 months prior to Screening Visit 2.
18. Uncontrolled hypertension (systolic blood pressure [BP]>180 mmHg, diastolic BP >100 mmHg) despite maximal medical treatment; note that if BP is brought below 180/100 mmHg and stabilized by antihypertensive treatment, as determined by the investigator and/or primary care physician, the participant can be rescreened for eligibility.
19. A systemic condition that, in the opinion of the investigator, would preclude participation in the study (poor glycemic control, uncontrolled hypertension, etc).
20. Any concomitant treatment that, in the opinion of the investigator, may interfere with the ocular procedure or the healing process.
21. History of malignancy with or without therapy or hematologic malignancy that may compromise the immune system requiring chemotherapy and/or radiation in the 5 years prior to Screening Visit 2. Localized basal cell carcinoma will be permitted.
22. Has a serious, chronic, or unstable medical or psychological condition that, in the opinion of the investigator, may compromise the participant's safety or ability to complete all assessments and follow-up in the study.
23. Meets any one of the following exclusionary laboratory values at Screening Visit 2:
   Aspartate aminotransferase (AST) and/or alanine aminotransferase (ALT) >2.5×upper limit of normal (ULN).
   Total bilirubin >1.5×ULN, unless the participant has a previously known history of Gilbert's syndrome and a fractionated bilirubin that shows conjugated bilirubin <35% of total bilirubin.
   Prothrombin time >1.5×ULN, unless the participant is anticoagulated.
   Hemoglobin <10 g/dL for male participants and <9 g/dL for female participants.
   Platelets <100×10³ μL.
   Estimated glomerular filtration rate <30 mL/min/1.73 m2.
24. History of chronic renal failure requiring dialysis or kidney transplant.
25. Initiation of intensive insulin treatment (pump or multiple daily injections) within the 6 months prior to Screening Visit 2 or plans to do so within 48 weeks of Day 1.
26. Participation in any other gene therapy study, including Construct II, or receipt of any investigational product within 30 days prior to enrollment or 5 half-lives of the investigational product, whichever is longer, or any plans to use an investigational product within 6 months following enrollment.
27. Known hypersensitivity to ranibizumab or any of its components.

### 6.11.4 Study Intervention(s) Administered

Eligible participants will be assigned either to receive a single dose of Construct II (Dose 1 or Dose 2) in the study eye or be followed for observation only. Information regarding Construct II follows.

TABLE 18
Information regarding Construct II

| Arm Name | Construct II Dose 1 | Construct II Dose 2 |
|---|---|---|
| Type | Gene therapy (AAV8.CB7.CI.amd42.RBG) | |
| Dose Formulation | Solution | |
| Unit Dose Strength | 2.5 × 10¹² GC/mL | 2.5 × 10¹² GC/mL |
| Dosage Level(s) | 2.5 × 10¹¹ GC/eye delivered via | 5.0 × 10¹¹ GC/eye delivered via two |
| | a single 100 μL SCS injection | 100 μL SCS injections at the same |
| | (100 μL total volume) | visit (200 μL total volume) |

| | |
|---|---|
| Route of | Suprachoroidal space injection in the study eye using a microinjector. |
| Administration | |
| Physical | Construct II investigational product is supplied as a frozen, sterile, single-use |
| Description | solution of the AAV vector active ingredient (AAV8.CB7.CI.amd42.RBG) in |
| | a formulation buffer. The solution appears clear to opalescent, colorless, and |
| | free of visible particulates at room temperature. |
| Packaging and | Construct II will be supplied as a sterile, single-use solution in 2-mL Crystal |
| Labeling | Zenith ® vials sealed with latex-free rubber stoppers and aluminum flip-off |
| | seals. Each vial will be labeled as required per country regulatory requirements. |

### 6.11.5 Vector Shedding

Sampling of blood (serum), urine, and tears will be performed for Construct II participants for measurement of vector concentrations. Refer to the Investigator Laboratory Manual for additional information regarding the processing, handling, and shipping of the samples.

Shedding data collected in these biological fluids provide a shedding profile of Construct II in the target patient population and is used to estimate the potential of transmission to untreated individuals. Shedding will be measured using quantitative polymerase chain reaction.

### 6.12 Example 12: a Phase 2, Randomized, Dose-Escalation, Observation-Controlled Study to Evaluate the Efficacy, Safety, and Tolerability of Construct II Gene Therapy Delivered Via One or Two Suprachoroidal Space (SCS) Injections in Participants with Diabetic Retinopathy (DR) without Center Involved-Diabetic Macular Edema (CI-DME)

This example provides an overview of a phase 2a, dose assessment of Construct II gene therapy in participants with diabetic retinopathy (DR).

### 6.12.1 Objectives and Endpoints

TABLE 19
Objectives and Endpoints

| | |
|---|---|
| Objectives | Endpoints |

Primary

| | | |
|---|---|---|
| Efficacy | To evaluate the effect | Proportion of participants achieving a 2-step or |
| | of Construct II on DR | greater improvement in DR by ETDRS-DRSS on |
| | by the ETDRS-DRSS | 4-widefield digital stereoscopic fundus |
| | at Week 48 | photography at Week 48 |

Secondary

| | | |
|---|---|---|
| Efficacy | To evaluate the effect | Proportion of participants achieving a 2-step or |
| | of Construct II on DR | greater improvement in DR per ETDRS-DRSS on |
| | (ETDRS-DRSS) over time | 4-widefield digital stereoscopic fundus |
| | | photography at Week 4, Week 12, and Week 24, |
| | | and Week 36 |
| | | Proportion of participants achieving a 0-step (no |
| | | change), a 1-step or greater, or a 3-step or greater |
| | | improvement in DR per ETDRS-DRSS on 4- |
| | | widefield digital stereoscopic fundus photography |
| | | at Week 4, Week 12, Week 24, Week 36, and Week 48 |
| | | Proportion of participants with a 1-step or greater, |
| | | a 2-step or greater, or a 3-step or greater |
| | | worsening in DR per ETDRS-DRSS on |
| | | 4-widefield digital stereoscopic fundus |
| | | photography at Week 4, Week 12, Week 24, |
| | | Week 36, and Week 48 |
| | | Proportion of participants graded as Level 61 |
| | | (PDR) at baseline achieving regression to Level |
| | | 47 or 53 (NPDR) at Week 24, Week 36, and Week 48 |
| Safety/ | To assess the safety, | Incidences of overall and ocular AEs |
| Immunogenicity | tolerability, and | Immunogenicity measurements (AAV8: NAbs, |
| | immunogenicity of | TAbs, and ELISpot; Construct II TP: anti- |
| | Construct II | Construct II TP antibodies and ELISpot) over |
| | | 48 weeks |
| Safety/Tolerability | To evaluate the | Proportion of participants who experience ocular |
| | incidences of ocular | inflammation following SCS administration |
| | inflammation | |
| | following SCS | |
| | administration of | |
| | Construct II | |
| Safety/Efficacy | To evaluate the need | Proportion of participants requiring any additional |
| | for additional SOC | intervention for ocular diabetic complications to |
| | intervention due to | Week 48 |
| | ocular diabetic | Proportion of participants with any sight- |
| | complications | threatening ocular diabetic complications to Week 48 |
| | | Proportion of participants developing ocular |
| | | diabetic complications (eg, CI-DME or |
| | | neovascularization) requiring anti-VEGF |
| | | treatment per SOC through Week 48; for this |
| | | population, the following endpoints will be |
| | | evaluated: |
| | | Number of anti-VEGF injections received |
| | | Duration of time from study intervention (Day |
| | | 1) to first anti-VEGF administration per SOC |
| | | Proportion of participants developing ocular |
| | | diabetic complications (eg, neovascularization due |
| | | to DR) requiring PRP per SOC through Week 48; |
| | | for this population, the following endpoints will be |
| | | evaluated: |
| | | Duration of time from study intervention (Day |
| | | 1) to first PRP |
| | | Proportion of participants requiring more than 1 PRP |
| | | Proportion of participants developing ocular |
| | | diabetic complications (eg, retinal detachment) |
| | | requiring surgical intervention (pneumatic |
| | | retinopexy, cryopexy, or scleral buckle) per SOC; |
| | | for this population, the following endpoint will be |
| | | evaluated: |
| | | Duration of time from study intervention (Day |
| | | 1) to surgical intervention |
| Pharmacodynamics | To measure aqueous | Aqueous Construct II TP concentration at assessed |
| | and serum Construct II | time points |
| | TP concentrations | Serum Construct II TP concentration at assessed |
| | | time points |

Exploratory

| | | |
|---|---|---|
| Efficacy/Safety | To evaluate the effect | Proportion of participants with visual stability |
| | of Construct II on | (within 5 ETDRS letters or ±5 ETDRS letters) |
| | vision outcomes | from baseline to Week 48 |
| | (BCVA in all | Proportion of participants with vision gain or |
| | Construct II treated | vision loss >5 ETDRS letters from baseline to |
| | participants) | Week 48 |
| | To evaluate the effect | Proportion of participants with clinically |
| | of Construct II on | significant changes in visual field from baseline |
| | visual field in all | to Week 48, as determined by the investigator |
| | Construct II treated | |
| | participants | |
| | To evaluate the effect | Mean change from baseline in CST on SD-OCT at |
| | of Construct II on | Week 24 and Week 48 |
| | anatomic outcomes | Proportion of participants achieving ≤290 μm in |
| | assessed using SD- | CST on SD-OCT at Week 24 and Week 48 |
| | OCT in all Construct II | Proportion of participants with clinically |
| | treated participants | significant macular thickening in CST ≥30 μm |
| | | from baseline at Week 24 and Week 48, as |
| | | determined by the CRC |
| | | Mean change in macular volume and percent |
| | | reduction in macular volume at Week 48 relative |
| | | to baseline on SD-OCT, as determined by the CRC |
| | To assess evidence of | Proportion of participants graded as Level 61 at |
| | vessel regression on | baseline with evidence of vessel regression at |
| | FA for participants | Week 12, Week 24 and Week 48 based on FA, as |
| | with baseline PDR | determined by the CRC |
| | (Level 61) | |
| | To assess changes in | Proportion of participants graded as Level 61 at |
| | the area of leakage on | baseline with change in the area of leakage from |
| | FA for participants | baseline to Week 12, Week 24 and Week 48 based |
| | with baseline PDR | on FA, as determined by the CRC |
| | (Level 61) | |
| | To assess changes | Mean change from baseline in the area of retinal |
| | from baseline in the | nonperfusion at Week 12, Week 24 and Week 48 |
| | area of retinal | based on FA in all evaluable participants, as |
| | nonperfusion on Optos | determined by the CRC |
| | widefield FA in all | |
| | evaluable participants | |
| Efficacy/ | To evaluate the effect | Association of positive serum AAV8 NAb results |
| Immunogenicity | of positive serum | with aqueous humor and serum Construct II TP |
| | adeno-associated virus | concentrations |
| | serotype 8 (AAV8) | |
| | neutralizing antibody | |
| | (NAb) results on the | |
| | concentration of | |
| | Construct IITP in | |
| | aqueous humor and | |
| | serum | |

AAV8 = adeno-associated virus serotype 8;
AE = adverse event;
BCVA = best-corrected visual acuity;
CI-DME = center involved-diabetic macular edema;
CRC = central reading center;
CST = central subfield thickness;
DR = diabetic retinopathy;
DRSS = Diabetic Retinopathy Severity Scale;
ELISpot = enzyme-linked ImmunoSpot;
ETDRS = Early Treatment Diabetic Retinopathy Study;
FA = fluorescein angiography;
NAb = neutralizing antibody;
PDR = proliferative diabetic retinopathy;
PRP = panretinal photocoagulation;
SD-OCT = spectral domain-optical coherence tomography;
SOC = standard of care;
TAb = total binding antibody;
TP = transgene product;
VEGF = vascular endothelial growth factor

### 6.12.2 Study Design

In this phase 2, randomized (3:1), dose-escalation, observation-controlled study, approximately 100 participants with DR will be enrolled into 3 dose cohorts. Cohorts 1-5 will receive Construct II (n=15 each in Cohorts 1, 2, 4, and 5; and n=20 in Cohort 3) and approximately 20 total participants across Cohorts 1, 2, 4, and 5 will be evaluated as observational controls (n=5 per cohort). The control arm participants in Cohorts 1 and 2 will also serve as the control for Cohort 3, which will not be randomized.

All participants in Cohorts 1 and 2 will have negative or low serum titer results (≤300) for AAV8 NAbs and will be randomized to either receive Construct II (at a dose of 2.5×10¹¹ GC/eye [Cohort 1] or 5.0×10¹¹ GC/eye [Cohort 2]) or be evaluated as observational controls. All participants in Cohort 3 will have a serum titer result >300 for AAV8 NAbs and will receive Construct II at a dose of 5.0×10¹¹ GC/eye (ie, the same dose level given to participants in Cohort 2). The participants in both Cohorts 4 and 5 will receive a dose level of Construct II (1.0×10¹² GC/eye) that is higher than either of the doses tested in Cohorts 1-3. Enrollment into Cohorts 4 and 5 will be stratified by the central reading center (CRC)-confirmed Screening Visit 2 ETDRS-DRSS level. Eligible Cohort 4 participants will require an ETDRS-DRSS level 47 or 53. Cohort 5 participants require an ETDRS-DRSS level 61 or 65, with a minimum of 5 participants at each PDR level. Both Cohorts 4 and 5 will be randomized to either receive Construct II at dose level 3 (1.0×10¹² GC/eye), or be evaluated as observational controls.

Participants in the observation control arms (Cohorts 1, 2, 4, and 5) will be offered, if eligible, an opportunity to receive Construct II treatment after completion of the study.

### Control

Participants will be strongly encouraged to participate in a long-term follow-up (LTFU) study for continued safety evaluation. A separate informed consent for the follow-up study will be signed at that time.

For Cohorts 1-3, certain required screening test results (ie, AAV8 NAb and AAV8 total binding antibody (TAb) titers and ETDRS-DRSS score from 4-widefield fundus photography [bilateral]), formerly collected at Screening Visit 1 or obtained via the participant's enrollment in a separate NAb screening study, will be obtained exclusively through the participant's enrollment in the study. With this change, Screening Visits 1 and 2 in this study may be combined into a single screening visit and considered as Screening Visit 2. For a participant to be eligible for screening in the study, they must have an AAV8 NAb titer from the study and an ETDRS-DRSS score from the study within reasonable range of the inclusion criteria specified below. Fundus photography will be repeated in the study at Screening Visit 2 to ensure a CRC-determined, qualifying ETDRS-DRSS score is obtained. AAV8 NAbs will be measured at baseline (pretreatment) in Cohorts 4 and 5, but will not be used for screening purposes to determine eligibility and enrollment. Further, Cohorts 4 and 5 participants will not be required to enroll in a separate NAb screening study. Screening ETDRS-DRSS scores to determine study eligibility for Cohorts 4 and 5 will be obtained at Screening Visit 2. Participants who do not meet the entry criteria will be considered screen failures.

Participants receiving Construct II will have their study intervention administered at the study center during the Day 1 visit. Construct II will be given as a single injection by SCS delivery using the Clearside SCS Microinjector investigational device. The Treatment Period of the study begins at the time of Construct II administration. All investigators will be trained on the SCS procedure. A detailed description of the procedure can be found in the SCS Administration Manual.

All Cohort 4 and Cohort 5 participants randomized to Construct II will receive a protocol-mandated steroid regimen to be administered following SCS Construct II administration.

Participants receiving Construct II (all cohorts) will have 2 visits for postinjection safety (1 day postprocedure and 1 week postprocedure). Participants randomized to observation control or Construct II (Cohorts 1, 2, 4, and 5) will have their first postrandomization visit at Week 4, along with all participants enrolled to receive Construct II (Cohort 3). Following the Week 4 visit, all participants will have a Week 12 visit followed by visits every 12 weeks through Week 48. Starting on Day 2, all participants, regardless of treatment assignment, may receive approved anti-vascular endothelial growth factor (VEGF) supplemental therapy or other SOC therapy if they have ocular diabetic complications warranting intervention.

Enrollment will begin with the randomization of 2 sentinel participants in Cohort 1: one sentinel participant will be randomized to Construct II at dose level 1 and the other to observation control. The Construct II sentinel participant will be observed for safety during the 2-week postadministration period. Following this, the Sponsor's Internal Safety Committee (ISC) will review the 2-week postadministration safety data for the sentinel participant receiving Construct II and, if no safety review triggers (SRTs) are observed, the remaining participants within Cohort 1 will be randomized to either Construct II dose level 1 (n=14) or observation control (n=4).

Once all Construct II participants within Cohort 1 are enrolled, the Independent Data Monitoring Committee (IDMC) will review the available cumulative safety data from Cohort 1 (dose level 1) inclusive of the 2-week postadministration safety data from the last dosed Construct II Cohort 1 participant to determine if enrollment of dose level 2 may be initiated. During any safety review, the IDMC may recommend halting dosing, proceeding with the same or lower dose level, or proceeding to the next planned dose level. If the decision is made to dose escalate and upon full enrollment of Cohort 1, randomization of 2 sentinel participants in Cohort 2 will begin, again with one randomized to Construct II (this time at dose level 2) and the other to observation control. The ISC will review 2-week postadministration safety data from the sentinel participant receiving Construct II and, if there are no observed SRTs, the remaining participants within Cohort 2 will be randomized to either Construct II dose level 2 (n=14) or observation control (n=4). In addition, simultaneous enrollment of Cohort 3 (n=20) may also begin after the ISC approval from the Construct II sentinel participant, with all participants in the cohort receiving Construct II at dose level 2 (ie, the same dose level of Construct II evaluated in Cohort 2).

Once all Construct II participants within Cohort 2 are enrolled, the IDMC will review the available cumulative safety data inclusive of the 2-week postadministration safety data from the last dosed Construct II Cohort 2 participant to determine if enrollment of dose level 3 may be initiated. If the decision is made to dose escalate and upon full enrollment of Cohort 2, randomization of 2 sentinel participants in Cohort 4 or 5 will begin, again with one randomized to Construct II (this time at dose level 3), and the other to observation control. The ISC will review the 2-week postadministration safety data for the Construct II-treated sentinel participant in Cohort 4 (or Cohort 5) and, if there are no observed SRTs, the remaining participants within Cohorts 4 and 5 (n=38) may be enrolled in parallel.

The dose escalation plan is designed to ensure that all eligible participants assigned to receive Construct II having a negative or low serum titer result (≤300) for screening serum AAV8 NAbs will complete dosing of Construct II at a given dose level, followed by IDMC review, before escalation may occur to the next dose level of Construct II. Prior to any dose escalation, the IDMC will review available cumulative safety data, inclusive of the 2-week postadministration safety visit from the last dosed Construct II participant within the dose level having a negative or low serum titer result for screening serum AAV8 NAbs.

For participants in the Construct II treatment arms, immunogenicity to the vector (as assessed by anti-AAV8 antibodies [serum], anti-Construct II TP antibodies [serum], and enzyme-linked ImmunoSpot [ELISpot][whole blood]), and Construct II TP concentrations (aqueous humor and serum) will be assessed throughout the study.

Efficacy will be the primary focus over the 48-week study period (primary study period), as assessed through the ETDRS-DRSS score on 4-widefield digital stereoscopic fundus photography, visual acuity, and SD-OCT. Participants will be evaluated for safety through the assessment of adverse events (AEs), including serious adverse events (SAEs) and adverse events of special interest (AESIs), as well as assessments of clinical laboratory tests (chemistry, hematology, coagulation, urinalysis), vector shedding (tears, urine, and serum), and ocular examinations and imaging (BCVA, intraocular pressure [IOP], slit-lamp biomicroscopy, indirect ophthalmoscopy, ultra-widefield FA, ultra-widefield Optos fundus autofluorescence [FAF], ultra-widefield Optos color fundus photography [CFP], 4-widefield digital stereoscopic fundus photography, Humphrey visual field or microperimetry, and SD-OCT). Note that AEs will be collected at all study visits. Participants who show evidence of new retinal hypo/hyper pigmentation changes as compared with baseline will be monitored using SD-OCT scans.

Planned safety monitoring of the study participants will be conducted on an ongoing basis. The monitoring will include reviews conducted by the Medical Monitor and routine reviews conducted by the Sponsor's ISC. Separately, an IDMC will also be established and will meet on a periodic basis to independently review the clinical data.

### 6.12.3 Inclusion Criteria

Participants must meet all the following criteria in order to be eligible for this study.

All ocular criteria refer to the study eye:
1. Men or women 25-89 years of age with DR secondary to diabetes mellitus Type 1 or
2. Participants must have a hemoglobin A1c≤12% (as confirmed by laboratory assessments obtained at Screening Visit 2 or by a documented laboratory report dated within 60 days prior to Screening Visit 2).
   For Cohorts 1-3, study eye with moderately-severe NPDR, severe NPDR, or mild PDR (ETDRS-DRSS levels 47, 53, or 61 using standard 4-widefield digital stereoscopic fundus photographs, as determined by the CRC) for which PRP or anti-VEGF injections can be safely deferred, in the opinion of the investigator, for at least 6 months after Screening Visit 2.
   For Cohort 4, study eye with moderately-severe NPDR or severe NPDR (ETDRS-DRSS levels 47 or 53) or for Cohort 5, study eye with mild PDR or moderate PDR (ETDRS-DRSS levels 61 or 65) using standard 4-widefield digital stereoscopic fundus photographs, for which PRP or anti-VEGF injections can be safely deferred, in the opinion of the investigator, for at least 6 months after Screening Visit 2.
2. No evidence in the study eye of high-risk characteristics typically associated with vision loss, per the investigator, including the following:
   New vessels within 1-disc area of the optic nerve
   Vitreous or preretinal hemorrhage associated with less extensive new vessels at the optic disc, or with new vessels elsewhere that are half a disc area or more in size.
   No evidence in the study eye of anterior segment (eg, iris or angle) neovascularization on clinical examination.
3. Participants in Cohorts 1 and 2 must have a negative or low serum titer result (≤300) for AAV8 NAbs. Participants in Cohort 3 must have a serum titer result >300 for AAV8 NAbs. Participants in Cohorts 4 and 5 do not have an eligibility requirement based on screening AAV8 NAbs.
4. Best-corrected visual acuity in the study eye of ≥69 ETDRS letters (approximate Snellen equivalent 20/40 or better); note: if both eyes are eligible, the study eye must be the participant's worse-seeing eye, as determined by the investigator, prior to enrollment.
5. Prior history of CI-DME in the study eye is acceptable if no intravitreal anti-VEGF or short-acting steroid injections have been given within the last 6 months, AND no more than 10 documented injections have been given in the 3 years prior to Screening Visit 2.
6. Sexually active male participants with female partners of childbearing potential must be willing to use condoms plus a medically accepted form of partner contraception from Screening Visit 2 until 4 weeks after vector administration.
7. Must be willing and able to comply with all study procedures and be available for the duration of the study.
8. Must be willing and able to provide written, signed informed consent.

### 6.12.4 Exclusion Criteria

Participants are excluded from the study if any of the following criteria apply:
1. Women of childbearing potential (ie, women who are not postmenopausal or surgically sterile) are excluded from this clinical study.
   Postmenopausal is defined to be documented 12 consecutive months without menses.
   Surgically sterile is defined as having bilateral tubal ligation/bilateral salpingectomy, bilateral tubal occlusive procedure, hysterectomy, or bilateral oophorectomy.
2. Presence of any active CI-DME, as determined by the investigator, on clinical examination or within the central subfield thickness (CST) of the study eye, as determined by SD-OCT evaluated by CRC, using the following threshold:
   Heidelberg Spectralis: CST greater than 320 m
3. Neovascularization in the study eye from a cause other than DR, per investigator.
4. Evidence in the study eye of optic nerve pallor on clinical examination, as determined by the investigator.
5. Any evidence or documented history of PRP or retinal laser in the study eye.
6. Ocular or periocular infection in the study eye that may interfere with the SCS procedure.
7. Any ocular condition in the study eye that could require surgical intervention within the 6 months after Screening Visit 2 (vitreous hemorrhage, cataract, retinal traction, epiretinal membrane, etc) or any condition in the study eye that may, in the opinion of the investigator, increase the risk to the participant, require either medical or surgical intervention during the study to prevent or treat vision loss, or interfere with the study procedures or assessments.
8. Active or history of retinal detachment in the study eye.
9. Presence of an implant in the study eye at Screening Visit 2 (excluding intraocular lens).
10. Participants who had a prior vitrectomy surgery.
11. Cohorts 1-3: Advanced glaucoma in the study eye, as defined by an IOP >23 mmHg, not controlled by 2 IOP-lowering medications, any invasive procedure to treat glaucoma (eg, shunt, tube, or minimally invasive glaucoma surgery devices; however, selective laser trabeculectomy and argon laser trabeculoplasty are permitted), or visual field loss encroaching on central fixation. Cohorts 4-5: Active or history of glaucoma or ocular hypertension in the study eye, defined as IOP >21 mmHg.
12. History of intraocular surgery in the study eye within 12 weeks prior to Screening Visit 2; yttrium aluminum garnet (YAG) capsulotomy is permitted if performed >10 weeks prior to Screening Visit 2.
13. History of intravitreal therapy in the study eye, including anti-VEGF therapy, within 6 months prior to Screening Visit 2, and documentation of more than 10 prior anti-VEGF or short-acting steroid intravitreal injections in the study eye within 36 months of Screening Visit 2.
14. Any prior intravitreal steroid injection in the study eye within 6 months prior to Screening Visit 2, administration in the study eye of Ozurdex® within 12 months prior to Screening Visit 2, or administration in the study eye of Iluvien® within 36 months prior to Screening Visit 2.
15. Any prior systemic anti-VEGF treatment within the 6 months prior to or plans to use systemic anti-VEGF therapy during the next 48 weeks after Screening Visit 2.
16. History of therapy known to have caused retinal toxicity, or concomitant therapy with any drug that may affect VA or with known retinal toxicity, eg, chloroquine or hydroxychloroquine.
17. Myocardial infarction, cerebrovascular accident, or transient ischemic attacks within the 6 months prior to Screening Visit 2.
18. Uncontrolled hypertension (systolic blood pressure [BP]>180 mmHg, diastolic BP >100 mmHg) despite maximal medical treatment; note that if BP is brought below 180/100 mmHg and stabilized by antihypertensive treatment, as determined by the investigator and/or primary care physician, the participant can be rescreened for eligibility.
19. A systemic condition that, in the opinion of the investigator, would preclude participation in the study (poor glycemic control, uncontrolled hypertension, etc).
20. Any concomitant treatment that, in the opinion of the investigator, may interfere with the ocular procedure or the healing process.
21. History of malignancy with or without therapy or hematologic malignancy that may compromise the immune system requiring chemotherapy and/or radiation in the 5 years prior to Screening Visit 2. Localized basal cell carcinoma will be permitted.
22. Has a serious, chronic, or unstable medical or psychological condition that, in the opinion of the investigator, may compromise the participant's safety or ability to complete all assessments and follow-up in the study.
23. Any laboratory values at Screening Visit 2 that, in the opinion of the investigator and Medical Monitor, may compromise the participant's safety or ability to complete all assessments and follow-up in the study. In the event Screening Visit 2 test results are unavailable prior to randomization or enrollment, the investigator, with the approval of the Medical Monitor, may consider historical laboratory test results collected any time within the 6 months prior to Screening Visit 2, except for HbA1c testing for which a historical lab result is acceptable only with a documented laboratory report dated within 60 days prior to Screening Visit 2.
24. History of chronic renal failure requiring dialysis or kidney transplant.
25. Initiation of intensive insulin treatment (pump or multiple daily injections) within the 6 months prior to Screening Visit 2 or plans to do so within 48 weeks of Day 1.
26. Participation in any other gene therapy study, including Construct II, or receipt of any investigational product within 30 days prior to enrollment or 5 half-lives of the investigational product, whichever is longer, or any plans to use an investigational product within 6 months following enrollment.
27. Known hypersensitivity to ranibizumab or any of its components.

### 6.12.5 Study Intervention(s) Administered

Eligible participants will be assigned either to receive a single dose of Construct II (Dose 1, Dose 2, or Dose 3) in the study eye or be followed for observation only. Cohort 1 comprises patients having negative or low (<300) NAb serum titers for AAV8, and receives Dose 1 (2.5×10¹¹ GC/eye) in a single 100 μL SCS injection. Cohort 2 comprises patients having negative or low (<300) NAb serum titers for AAV8, and receives Dose 2 (5.0×10¹¹ GC/eye) in a single 100 μL SCS injection. Cohort 3 comprises patients having positive or higher (>300) NAb serum titers for AAV8, and receives Dose 2 (5.0×10¹¹ GC/eye) in a single 100 μL SCS injection. Cohort 4 comprises patients having an ETDRS-DRSS level 47 or 53, and receives Dose 3 (1.0×10¹² GC/eye) in a single 100 μL SCS injection, and also receives a steroid treatment regime described herein below. Cohort 5 comprises patients having an ETDRS-DRSS level 61 or 65, and receives Dose 3 (1.0×10¹² GC/eye) in a single 100 μL SCS injection, and also receives a steroid treatment regime described herein below. Screening NAb titers is not used to determine eligibility in Cohorts 4 and 5. Information regarding Construct II follows.

TABLE 20
Information regarding Construct II

| Arm | Construct II | Construct II | Construct | |
|---|---|---|---|---|
| Name | Dose 1 | Dose 2 | Dose 3 | DUREZOL ® |
| Type | Gene therapy (AAV8.CB7.CI.amd42.RBG) | | | Drug (topical |
| | | | | corticosteroid) |
| Dose | Solution | | | Ophthalmic |
| Formulation | | | | emulsion |
| Unit Dose | Cohort 1: | Cohorts 2 & 3: | Cohorts 4&5: | 0.5 mg/mL (0.05%) |
| Strength | 2.5 × 10¹² GC/mL | 3.0 × 10¹³ GC/mL | 3.0 × 10¹³ GC/mL | |
| | | (diluted to 5.0 × | (diluted to 1.0 × | |
| | | 10¹² GC/mL) | 10¹³ GC/mL) | |
| Dose | 2.5 × 10¹¹ GC/eye | 5.0 × 10¹¹ GC/eye | 1.0 × 10¹² GC/eye | 1 drop |
| Level(s) | delivered via a | delivered via a | delivered via a | (see below for |
| | single 100 μL SCS | single 100 μL SCS | single 100 uL SCS | dosing regimen) |
| | injection (100 μL | injection (100 μL | injection (100 uL | |
| | total volume) | total volume) | total volume) | |

| | | |
|---|---|---|
| Route of | Suprachoroidal space injection(s) in the study eye using the | Topical administration |
| Administration | Clearside SCS Microinjector ™ investigational device | of 1 drop into the |
| | | conjunctival sac of |
| | | the study eye |
| Physical | Construct II investigational product is supplied as a frozen, | DUREZOL (difluprednate |
| Description | sterile, single-use solution of the AAV vector active ingredient | ophthalmic emulsion) |
| | (AAV8.CB7.CI.amd42.RBG) in a formulation buffer. The | 0.05% is supplied as |
| | solution appears clear to opalescent, colorless, and free of | a sterile, preserved, |
| | visible particulates at room temperature. | aqueous topical |
| | | ophthalmic emulsion |
| Manufacturer | Advanced BioScience Laboratories, Inc | Alcon ®, a Novartis |
| | | Company |
| Packaging | Construct II will be supplied as a sterile, single-use solution | DUREZOL (difluprednate |
| and Labeling | in 2-mL Crystal Zenith ® vials sealed with latex-free rubber | ophthalmic emulsion) |
| | stoppers and aluminum flip-off seals. Each vial will be labeled | 0.05% is a sterile, |
| | as required per country regulatory requirements. | aqueous topical |
| | | ophthalmic emulsion |
| | | supplied in an opaque |
| | | plastic bottle with a |
| | | controlled drop tip |
| | | and a pink cap; 5 |
| | | mL in an 8 mL bottle |

### 6.12.6 Steroid Regime

Cohort 4 and Cohort 5 participants randomized to Construct II treatment will receive a protocol-mandated postprocedure steroid regimen, as follows.

Beginning the evening of SCS administration of Construct II (Day 1), participants will instill 1 drop of difluprednate ophthalmic emulsion 0.0500 (DUREZOL) in the study eye, then instill drops daily in the study eye starting on Day 2, gradually decreasing in a tapered regimen over a total of 7 weeks:
4 times daily for 4 weeks, followed by
3 times daily for 1 week, followed by
2 times daily for 1 week, followed by
1 time daily for 1 week.

For any ocular inflammatory events occurring during or after the prophylaxis period, the Sponsor Clinical Development Lead should be contacted for management/treatment recommendations.

### 6.12.7 Vector Shedding

Sampling of blood (serum), urine, and tears will be performed for Construct II participants for measurement of vector concentrations. Refer to the Investigator Laboratory Manual for additional information regarding the processing, handling, and shipping of the samples.

Shedding data collected in these biological fluids provide a shedding profile of Construct II in the target patient population and is used to estimate the potential of transmission to untreated individuals. Shedding will be measured using quantitative polymerase chain reaction.

### 6.12.8 Results

As of the data cutoff, suprachoroidal delivery of Construct II has been well tolerated across 15 patients dosed in Cohort 1. Two SAEs were reported, none of which were considered related to Construct II. For those dosed in Cohort 1, all common treatment emergent adverse events (TEAEs) through 6 months in the study eye included conjunctival hemorrhage and conjunctival hyperemia, and were not considered related to Construct II. One case of mild episcleritis was reported 2-weeks post-dosing and was resolved with topical administration of a corticosteroid.

Patients treated with Construct II continue to demonstrate stable (+0.3 letters for Cohort 1, as compared to −2.0 letters for observational control) Best Corrected Visual Acuity (BCVA) at 6 months. With a single suprachoroidal injection of Construct II, patients demonstrate clinically meaningful improvements in disease severity over time, in particular 33% of patients achieved a ≥2 step improvement in DRSS at 3 months, and 47% achieved a ≥2 step improvement in DRSS at 6 months.

### 6.13 Example 13: a Phase 2, Randomized, Dose-Escalation, Observation-Controlled Study to Evaluate the Efficacy, Safety, and Tolerability of Construct II Gene Therapy Delivered Via One or Two Suprachoroidal Space (SCS) Injections in Participants with Diabetic Retinopathy (DR) without Center Involved-Diabetic Macular Edema (CI-DME)

This example is an update of Example 12.

### 6.13.1 Objectives and Endpoints

TABLE 21
Objectives and Endpoints

| | |
|---|---|
| Objectives | Endpoints |

Primary

| | | |
|---|---|---|
| Efficacy | To evaluate the effect of | Proportion of participants achieving a 2-step or |
| | Construct II on DR by the | greater improvement in DR by ETDRS-DRSS on |
| | ETDRS-DRSS at | 4-widefield digital stereoscopic fundus |
| | Week 48 | photography at Week 48 |

Secondary

| | | |
|---|---|---|
| Efficacy | To evaluate the effect of | Proportion of participants achieving a 2-step or |
| | Construct II on DR | greater improvement in DR per ETDRS-DRSS |
| | (ETDRS-DRSS) over | on 4-widefield digital stereoscopic fundus |
| | time | photography at Week 4, Week 12, and Week 24, |
| | | and Week 36 |
| | | Proportion of participants achieving a 0-step (no |
| | | change), a 1-step or greater, or a 3-step or greater |
| | | improvement in DR per ETDRS-DRSS on 4-widefield |
| | | digital stereoscopic fundus photography at Week 4, |
| | | Week 12, Week 24, Week 36, and Week 48 |
| | | Proportion of participants with a 1-step or |
| | | greater, a 2-step or greater, or a 3-step or |
| | | greater worsening in DR per ETDRS-DRSS on |
| | | 4-widefield digital stereoscopic fundus |
| | | photography at Week 4, Week 12, Week 24, |
| | | Week 36, and Week 48 |
| | | Proportion of participants graded as Level 61 |
| | | (PDR) at baseline achieving regression to Level |
| | | 47 or 53 (NPDR) at Week 24, Week 36, and Week 48 |
| Safety/ | To assess the safety, | Incidences of overall and ocular AEs |
| Immunogenicity | tolerability, and | Immunogenicity measurements (AAV8: NAbs, |
| | immunogenicity of | TAbs, and ELISpot; Construct II TP: anti- |
| | Construct II | Construct II TP antibodies and ELISpot) over |
| | | 48 weeks |
| Safety/Tolerability | To evaluate the | Proportion of participants who experience ocular |
| | incidences of ocular | inflammation following SCS administration |
| | inflammation following | |
| | SCS administration of | |
| | Construct II | |
| Safety/Efficacy | To evaluate the need for | Proportion of participants requiring any |
| | additional SOC | additional intervention for ocular diabetic |
| | intervention due to ocular | complications to Week 48 |
| | diabetic complications | Proportion of participants with any sight- |
| | | threatening ocular diabetic complications to |
| | | Week 48 |
| | | Proportion of participants developing ocular |
| | | diabetic complications (eg, CI-DME or |
| | | neovascularization) requiring anti-VEGF |
| | | treatment per SOC through Week 48; for this |
| | | population, the following endpoints will be |
| | | evaluated: |
| | | Number of anti-VEGF injections received |
| | | Duration of time from study intervention (Day |
| | | 1) to first anti-VEGF administration per SOC |
| | | Proportion of participants developing ocular |
| | | diabetic complications (eg, neovascularization |
| | | due to DR) requiring PRP per SOC through |
| | | Week 48; for this population, the following |
| | | endpoints will be evaluated: |
| | | Duration of time from study intervention |
| | | (Day 1) to first PRP |
| | | Proportion of participants requiring more than 1 PRP |
| | | Proportion of participants developing ocular |
| | | diabetic complications (eg, retinal detachment) |
| | | requiring surgical intervention (pneumatic |
| | | retinopexy, cryopexy, or scleral buckle) per SOC; |
| | | for this population, the following endpoint will be |
| | | evaluated: |
| | | Duration of time from study intervention (Day |
| | | 1) to surgical intervention |
| Pharmacodynamics | To measure aqueous and | Aqueous Construct II TP concentration at |
| | serum Construct II TP | assessed time points |
| | concentrations | Serum Construct II TP concentration at assessed |
| | | time points |

Exploratory

| | | |
|---|---|---|
| Efficacy/Safety | To evaluate the effect of | Proportion of participants with visual stability |
| | Construct II on vision | (within 5 ETDRS letters or ±5 ETDRS letters) |
| | outcomes (BCVA in all | from baseline to Week 48 |
| | Construct II treated | Proportion of participants with vision gain or |
| | participants) | vision loss >5 ETDRS letters from baseline to |
| | | Week 48 |
| | To evaluate the effect of | Proportion of participants with clinically |
| | Construct II on visual | significant changes in visual field from baseline |
| | field in all Construct II | to Week 48, as determined by the investigator |
| | treated participants | |
| | To evaluate the effect of | Mean change from baseline in CST on SD-OCT |
| | Construct II on anatomic | at Week 24 and Week 48 |
| | outcomes assessed using | Proportion of participants achieving ≤290 μm in |
| | SD-OCT in all Construct | CST on SD-OCT at Week 24 and Week 48 |
| | II treated participants | Proportion of participants with clinically |
| | | significant macular thickening in CST ≥30 μm |
| | | from baseline at Week 24 and Week 48, as |
| | | determined by the CRC |
| | | Mean change in macular volume and percent |
| | | reduction in macular volume at Week 48 relative |
| | | to baseline on SD-OCT, as determined by the CRC |
| | To assess evidence of | Proportion of participants graded as Level 61 at |
| | vessel regression on FA | baseline with evidence of vessel regression at |
| | for participants with | Week 12, Week 24 and Week 48 based on FA, as |
| | baseline PDR (Level 61) | determined by the CRC |
| | To assess changes in the | Proportion of participants graded as Level 61 at |
| | area of leakage on FA for | baseline with change in the area of leakage from |
| | participants with baseline | baseline to Week 12, Week 24 and Week 48 |
| | PDR (Level 61) | based on FA, as determined by the CRC |
| | To assess changes from | Mean change from baseline in the area of retinal |
| | baseline in the area of | nonperfusion at Week 12, Week 24 and Week 48 |
| | retinal nonperfusion on | based on FA in all evaluable participants, as |
| | Optos widefield FA in all | determined by the CRC |
| | evaluable participants | |
| Efficacy/ | To evaluate the effect of | Association of positive serum AAV8 NAb results |
| Immunogenicity | positive serum adeno- | with aqueous humor and serum Construct II TP |
| | associated virus serotype | concentrations |
| | 8 (AAV8) neutralizing | |
| | antibody (NAb) results on | |
| | the concentration of | |
| | Construct IITP in aqueous | |
| | humor and serum | |

AAV8 = adeno-associated virus serotype 8;
AE = adverse event;
BCVA = best-corrected visual acuity;
CI-DME = center involved-diabetic macular edema;
CRC = central reading center;
CST = central subfield thickness;
DR = diabetic retinopathy;
DRSS = Diabetic Retinopathy Severity Scale;
ELISpot = enzyme-linked ImmunoSpot;
ETDRS = Early Treatment Diabetic Retinopathy Study;
FA = fluorescein angiography;
NAb = neutralizing antibody;
PDR = proliferative diabetic retinopathy;
PRP = panretinal photocoagulation;
SD-OCT = spectral domain-optical coherence tomography;
SOC = standard of care;
TAb = total binding antibody;
TP = transgene product;
VEGF = vascular endothelial growth factor

### 6.13.2 Study Design

In this phase 2, randomized (3:1), dose-escalation, observation-controlled study, approximately 100 participants with DR will be enrolled into 3 dose cohorts. Cohorts 1-5 will receive Construct II (n=15 each in Cohorts 1, 2, 4, and 5; and n=20 in Cohort 3) and approximately 20 total participants across Cohorts 1, 2, 4, and 5 will be evaluated as observational controls (n=5 per cohort). The control arm participants in Cohorts 1 and 2 will also serve as the control for Cohort 3, which will not be randomized.

All participants in Cohorts 1 and 2 will have negative or low serum titer results (≤300) for AAV8 NAbs and will be randomized to either receive Construct II (at a dose of 2.5×10¹¹ GC/eye [Cohort 1] or 5.0×10¹¹ GC/eye [Cohort 2]) or be evaluated as observational controls. All participants in Cohort 3 will have a serum titer result >300 for AAV8 NAbs and will receive Construct II at a dose of 5.0×10¹¹ GC/eye (ie, the same dose level given to participants in Cohort 2). The participants in both Cohorts 4 and 5 will receive a dose level of Construct II (1.0×10¹² GC/eye) that is higher than either of the doses tested in Cohorts 1-3. Enrollment into Cohorts 4 and 5 will be stratified by the central reading center (CRC)-confirmed Screening Visit 2 ETDRS-DRSS level. Eligible Cohort 4 participants will require an ETDRS-DRSS level 47 or 53. Cohort 5 participants require an ETDRS-DRSS level 61 or 65, with a minimum of 5 participants at each PDR level. Both Cohorts 4 and 5 will be randomized to either receive Construct II at dose level 3 (1.0×10¹² GC/eye), or be evaluated as observational controls.

Participants in the observation control arms (Cohorts 1, 2, 4, and 5) will be offered, if eligible, an opportunity to receive Construct II treatment after completion of the study.

### Control

Participants will be strongly encouraged to participate in a long-term follow-up (LTFU) study for continued safety evaluation. A separate informed consent for the follow-up study will be signed at that time.

For Cohorts 1-3, certain required screening test results (ie, AAV8 NAb and AAV8 total binding antibody (TAb) titers and ETDRS-DRSS score from 4-widefield fundus photography [bilateral]), formerly collected at Screening Visit 1 or obtained via the participant's enrollment in a separate NAb screening study, will be obtained exclusively through the participant's enrollment in the study. With this change, Screening Visits 1 and 2 in this study may be combined into a single screening visit and considered as Screening Visit 2. For a participant to be eligible for screening in the study, they must have an AAV8 NAb titer from the study and an ETDRS-DRSS score from the study within reasonable range of the inclusion criteria specified below. Fundus photography will be repeated in the study at Screening Visit 2 to ensure a CRC-determined, qualifying ETDRS-DRSS score is obtained. AAV8 NAbs will be measured at baseline (pretreatment) in Cohorts 4 and 5, but will not be used for screening purposes to determine eligibility and enrollment. Further, Cohorts 4 and 5 participants will not be required to enroll in a separate NAb screening study. Screening ETDRS-DRSS scores to determine study eligibility for Cohorts 4 and 5 will be obtained at Screening Visit 2. Participants who do not meet the entry criteria will be considered screen failures.

Participants receiving Construct II will have their study intervention administered at the study center during the Day 1 visit. Construct II will be given as a single injection by SCS delivery using the Clearside SCS Microinjector investigational device. The Treatment Period of the study begins at the time of Construct II administration. All investigators will be trained on the SCS procedure. A detailed description of the procedure can be found in the SCS Administration Manual.

All Cohort 4 and Cohort 5 participants randomized to Construct II will receive a protocol-mandated steroid regimen to be administered following SCS Construct II administration.

Participants receiving Construct II (all cohorts) will have 2 visits for postinjection safety (1 day postprocedure and 1 week postprocedure). Participants randomized to observation control or Construct II (Cohorts 1, 2, 4, and 5) will have their first postrandomization visit at Week 4, along with all participants enrolled to receive Construct II (Cohort 3). Following the Week 4 visit, all participants will have a Week 12 visit followed by visits every 12 weeks through Week 48. Starting on Day 2, all participants, regardless of treatment assignment, may receive approved anti-vascular endothelial growth factor (VEGF) supplemental therapy or other SOC therapy if they have ocular diabetic complications warranting intervention.

Enrollment will begin with the randomization of 2 sentinel participants in Cohort 1: one sentinel participant will be randomized to Construct II at dose level 1 and the other to observation control. The Construct II sentinel participant will be observed for safety during the 2-week postadministration period. Following this, the Sponsor's Internal Safety Committee (ISC) will review the 2-week postadministration safety data for the sentinel participant receiving Construct II and, if no safety review triggers (SRTs) are observed, the remaining participants within Cohort 1 will be randomized to either Construct II dose level 1 (n=14) or observation control (n=4).

Once all Construct II participants within Cohort 1 are enrolled, the Independent Data Monitoring Committee (IDMC) will review the available cumulative safety data from Cohort 1 (dose level 1) inclusive of the 2-week postadministration safety data from the last dosed Construct II Cohort 1 participant to determine if enrollment of dose level 2 may be initiated. During any safety review, the IDMC may recommend halting dosing, proceeding with the same or lower dose level, or proceeding to the next planned dose level. If the decision is made to dose escalate and upon full enrollment of Cohort 1, randomization of 2 sentinel participants in Cohort 2 will begin, again with one randomized to Construct II (this time at dose level 2) and the other to observation control. The ISC will review 2-week postadministration safety data from the sentinel participant receiving Construct II and, if there are no observed SRTs, the remaining participants within Cohort 2 will be randomized to either Construct II dose level 2 (n=14) or observation control (n=4). In addition, simultaneous enrollment of Cohort 3 (n=20) may also begin after the ISC approval from the Construct II sentinel participant, with all participants in the cohort receiving Construct II at dose level 2 (ie, the same dose level of Construct II evaluated in Cohort 2).

Once all Construct II participants within Cohort 2 are enrolled, the IDMC will review the available cumulative safety data inclusive of the 2-week postadministration safety data from the last dosed Construct II Cohort 2 participant to determine if enrollment of dose level 3 may be initiated. If the decision is made to dose escalate and upon full enrollment of Cohort 2, randomization of 2 sentinel participants in Cohort 4 or 5 will begin, again with one randomized to Construct II (this time at dose level 3), and the other to observation control. The ISC will review the 2-week postadministration safety data for the Construct II-treated sentinel participant in Cohort 4 (or Cohort 5) and, if there are no observed SRTs, the remaining participants within Cohorts 4 and 5 (n=38) may be enrolled in parallel.

The dose escalation plan is designed to ensure that all eligible participants assigned to receive Construct II having a negative or low serum titer result (≤300) for screening serum AAV8 NAbs will complete dosing of Construct II at a given dose level, followed by IDMC review, before escalation may occur to the next dose level of Construct II. Prior to any dose escalation, the IDMC will review available cumulative safety data, inclusive of the 2-week postadministration safety visit from the last dosed Construct II participant within the dose level having a negative or low serum titer result for screening serum AAV8 NAbs.

For participants in the Construct II treatment arms, immunogenicity to the vector (as assessed by anti-AAV8 antibodies [serum], anti-Construct II TP antibodies [serum], and enzyme-linked ImmunoSpot [ELISpot][whole blood]), and Construct II TP concentrations (aqueous humor and serum) will be assessed throughout the study.

Efficacy will be the primary focus over the 48-week study period (primary study period), as assessed through the ETDRS-DRSS score on 4-widefield digital stereoscopic fundus photography, visual acuity, and SD-OCT. Participants will be evaluated for safety through the assessment of adverse events (AEs), including serious adverse events (SAEs) and adverse events of special interest (AESIs), as well as assessments of clinical laboratory tests (chemistry, hematology, coagulation, urinalysis), vector shedding (tears, urine, and serum), and ocular examinations and imaging (BCVA, intraocular pressure [IOP], slit-lamp biomicroscopy, indirect ophthalmoscopy, ultra-widefield FA, ultra-widefield Optos fundus autofluorescence [FAF], ultra-widefield Optos color fundus photography [CFP], 4-widefield digital stereoscopic fundus photography, Humphrey visual field or microperimetry, and SD-OCT). Note that AEs will be collected at all study visits. Participants who show evidence of new retinal hypo/hyper pigmentation changes as compared with baseline will be monitored using SD-OCT scans.

Planned safety monitoring of the study participants will be conducted on an ongoing basis. The monitoring will include reviews conducted by the Medical Monitor and routine reviews conducted by the Sponsor's ISC. Separately, an IDMC will also be established and will meet on a periodic basis to independently review the clinical data.

### 6.13.3 Inclusion Criteria

Participants must meet all the following criteria in order to be eligible for this study.

All ocular criteria refer to the study eye:
1. Men or women 25-89 years of age with DR secondary to diabetes mellitus Type 1 or
2. Participants must have a hemoglobin A1c≤12% (as confirmed by laboratory assessments obtained at Screening Visit 2 or by a documented laboratory report dated within 60 days prior to Screening Visit 2).
   For Cohorts 1-3, study eye with moderately-severe NPDR, severe NPDR, or mild PDR (ETDRS-DRSS levels 47, 53, or 61 using standard 4-widefield digital stereoscopic fundus photographs, as determined by the CRC) for which PRP or anti-VEGF injections can be safely deferred, in the opinion of the investigator, for at least 6 months after Screening Visit
   For Cohort 4, study eye with moderately-severe NPDR or severe NPDR (ETDRS-DRSS levels 47 or 53) or for Cohort 5, study eye with mild PDR or moderate PDR (ETDRS-DRSS levels 61 or 65) using standard 4-widefield digital stereoscopic fundus photographs, for which PRP or anti-VEGF injections can be safely deferred, in the opinion of the investigator, for at least 6 months after Screening Visit 2.
2. No evidence in the study eye of high-risk characteristics typically associated with vision loss, per the investigator, including the following:
   New vessels within 1-disc area of the optic nerve
   Vitreous or preretinal hemorrhage associated with less extensive new vessels at the optic disc, or with new vessels elsewhere that are half a disc area or more in size.
   No evidence in the study eye of anterior segment (eg, iris or angle) neovascularization on clinical examination.
3. Participants in Cohorts 1 and 2 must have a negative or low serum titer result (≤300) for AAV8 NAbs. Participants in Cohort 3 must have a serum titer result >300 for AAV8 NAbs. Participants in Cohorts 4 and 5 do not have an eligibility requirement based on screening AAV8 NAbs.
4. Best-corrected visual acuity in the study eye of ≥69 ETDRS letters (approximate Snellen equivalent 20/40 or better); note: if both eyes are eligible, the study eye must be the participant's worse-seeing eye, as determined by the investigator, prior to enrollment.
5. Prior history of CI-DME in the study eye is acceptable if no intravitreal anti-VEGF or short-acting steroid injections have been given within the last 6 months, AND no more than 10 documented injections have been given in the 3 years prior to Screening Visit 2.
6. Sexually active male participants with female partners of childbearing potential must be willing to use condoms plus a medically accepted form of partner contraception from Screening Visit 2 until 4 weeks after vector administration.
7. Must be willing and able to comply with all study procedures and be available for the duration of the study.
8. Must be willing and able to provide written, signed informed consent.

### 6.13.4 Exclusion Criteria

Participants are excluded from the study if any of the following criteria apply:
1. Women of childbearing potential (ie, women who are not postmenopausal or surgically sterile) are excluded from this clinical study.
   Postmenopausal is defined to be documented 12 consecutive months without menses.
   Surgically sterile is defined as having bilateral tubal ligation/bilateral salpingectomy, bilateral tubal occlusive procedure, hysterectomy, or bilateral oophorectomy.
2. Presence of any active CI-DME, as determined by the investigator, on clinical examination or within the central subfield thickness (CST) of the study eye, as determined by SD-OCT evaluated by CRC, using the following threshold:
   Heidelberg Spectralis: CST greater than 320 m
3. Neovascularization in the study eye from a cause other than DR, per investigator.
4. Evidence in the study eye of optic nerve pallor on clinical examination, as determined by the investigator.
5. Any evidence or documented history of PRP or retinal laser in the study eye.
6. Ocular or periocular infection in the study eye that may interfere with the SCS procedure.
7. Any ocular condition in the study eye that could require surgical intervention within the 6 months after Screening Visit 2 (vitreous hemorrhage, cataract, retinal traction, epiretinal membrane, etc) or any condition in the study eye that may, in the opinion of the investigator, increase the risk to the participant, require either medical or surgical intervention during the study to prevent or treat vision loss, or interfere with the study procedures or assessments.
8. Active or history of retinal detachment in the study eye.
9. Presence of an implant in the study eye at Screening Visit 2 (excluding intraocular lens).
10. Participants who had a prior vitrectomy surgery.
11. Cohorts 1-3: Advanced glaucoma in the study eye, as defined by an IOP >23 mmHg, not controlled by 2 IOP-lowering medications, any invasive procedure to treat glaucoma (eg, shunt, tube, or minimally invasive glaucoma surgery devices; however, selective laser trabeculectomy and argon laser trabeculoplasty are permitted), or visual field loss encroaching on central fixation. Cohorts 4-5: Active or history of glaucoma or ocular hypertension in the study eye, defined as IOP >21 mmHg.
12. History of intraocular surgery in the study eye within 12 weeks prior to Screening Visit 2; yttrium aluminum garnet (YAG) capsulotomy is permitted if performed >10 weeks prior to Screening Visit 2.
13. History of intravitreal therapy in the study eye, including anti-VEGF therapy, within 6 months prior to Screening Visit 2, and documentation of more than 10 prior anti-VEGF or short-acting steroid intravitreal injections in the study eye within 36 months of Screening Visit 2.
14. Any prior intravitreal steroid injection in the study eye within 6 months prior to Screening Visit 2, administration in the study eye of Ozurdex® within 12 months prior to Screening Visit 2, or administration in the study eye of Iluvien® within 36 months prior to Screening Visit 2.
15. Any prior systemic anti-VEGF treatment within the 6 months prior to or plans to use systemic anti-VEGF therapy during the next 48 weeks after Screening Visit 2.
16. History of therapy known to have caused retinal toxicity, or concomitant therapy with any drug that may affect VA or with known retinal toxicity, eg, chloroquine or hydroxychloroquine.
17. Myocardial infarction, cerebrovascular accident, or transient ischemic attacks within the 6 months prior to Screening Visit 2.
18. Uncontrolled hypertension (systolic blood pressure [BP]>180 mmHg, diastolic BP >100 mmHg) despite maximal medical treatment; note that if BP is brought below 180/100 mmHg and stabilized by antihypertensive treatment, as determined by the investigator and/or primary care physician, the participant can be rescreened for eligibility.
19. A systemic condition that, in the opinion of the investigator, would preclude participation in the study (poor glycemic control, uncontrolled hypertension, etc).
20. Any concomitant treatment that, in the opinion of the investigator, may interfere with the ocular procedure or the healing process.
21. History of malignancy with or without therapy or hematologic malignancy that may compromise the immune system requiring chemotherapy and/or radiation in the 5 years prior to Screening Visit 2. Localized basal cell carcinoma will be permitted.
22. Has a serious, chronic, or unstable medical or psychological condition that, in the opinion of the investigator, may compromise the participant's safety or ability to complete all assessments and follow-up in the study.
23. Any laboratory values at Screening Visit 2 that, in the opinion of the investigator and Medical Monitor, may compromise the participant's safety or ability to complete all assessments and follow-up in the study. In the event Screening Visit 2 test results are unavailable prior to randomization or enrollment, the investigator, with the approval of the Medical Monitor, may consider historical laboratory test results collected any time within the 6 months prior to Screening Visit 2, except for HbA1c testing for which a historical lab result is acceptable only with a documented laboratory report dated within 60 days prior to Screening Visit 2.
24. History of chronic renal failure requiring dialysis or kidney transplant.
25. Initiation of intensive insulin treatment (pump or multiple daily injections) within the 6 months prior to Screening Visit 2 or plans to do so within 48 weeks of Day 1.
26. Participation in any other gene therapy study, including Construct II, or receipt of any investigational product within 30 days prior to enrollment or 5 half-lives of the investigational product, whichever is longer, or any plans to use an investigational product within 6 months following enrollment.
27. Known hypersensitivity to ranibizumab or any of its components.

### 6.13.5 Study Intervention(s) Administered

Eligible participants will be assigned either to receive a single dose of Construct II (Dose 1, Dose 2, or Dose 3) in the study eye or be followed for observation only. Cohort 1 comprises patients having negative or low (<300) NAb serum titers for AAV8, and receives Dose 1 (2.5×10¹¹ GC/eye) in a single 100 μL SCS injection. Cohort 2 comprises patients having negative or low (<300) NAb serum titers for AAV8, and receives Dose 2 (5.0×10¹¹ GC/eye) in a single 100 μL SCS injection. Cohort 3 comprises patients having positive or higher (>300) NAb serum titers for AAV8, and receives Dose 2 (5.0×10¹¹ GC/eye) in a single 100 μL SCS injection. Cohort 4 comprises patients having an ETDRS-DRSS level 47 or 53, and receives Dose 3 (1.0×10¹² GC/eye) in a single 100 μL SCS injection, and also receives a steroid treatment regime described herein below. Cohort 5 comprises patients having an ETDRS-DRSS level 61 or 65, and receives Dose 3 (1.0×10¹² GC/eye) in a single 100 μL SCS injection, and also receives a steroid treatment regime described herein below. Screening NAb titers is not used to determine eligibility in Cohorts 4 and 5. Information regarding Construct II follows.

TABLE 22
Information regarding Construct II

| Arm | Construct II | Construct II | Construct II | |
|---|---|---|---|---|
| Name | Dose 1 | Dose 2 | Dose 3 | DUREZOL ® |
| Type | Gene therapy (AAV8.CB7.CI.amd42.RBG) | | | Drug (topical |
| | | | | corticosteroid) |
| Dose | Solution | | | Ophthalmic |
| Formulation | | | | emulsion |
| Unit Dose | Cohort 1: | Cohorts 2 & 3: | Cohorts 4&5: | 0.5 mg/mL (0.05%) |
| Strength | 2.5 × 10¹² GC/mL | 3.0 × 10¹³ GC/mL | 3.0 × 10¹³ GC/mL | |
| | | (diluted to 5.0 × | (diluted to 1.0 × | |
| | | 10¹² GC/mL) | 10¹³ GC/mL) | |
| Dose | 2.5 × 10¹¹ GC/eye | 5.0 × 10¹¹ GC/eye | 1.0 × 10¹² GC/eye | 1 drop |
| Level(s) | delivered via a | delivered via a | delivered via a | (see below for |
| | single 100 μL SCS | single 100 μL SCS | single 100 μL SCS | dosing regimen) |
| | injection (100 μL | injection (100 μL | injection (100 μL | |
| | total volume) | total volume) | total volume) | |

| | | |
|---|---|---|
| Route of | Suprachoroidal space injection(s) in the study eye using the | Topical administration |
| Administration | Clearside SCS Microinjector ™ investigational device | of 1 drop into the |
| | | conjunctival sac of |
| | | the study eye |
| Physical | Construct II investigational product is supplied as a frozen, | DUREZOL (difluprednate |
| Description | sterile, single-use solution of the AAV vector active ingredient | ophthalmic emulsion) |
| | (AAV8.CB7.CI.amd42.RBG) in a formulation buffer. The | 0.05% is supplied as |
| | solution appears clear to opalescent, colorless, and free of | a sterile, preserved, |
| | visible particulates at room temperature. | aqueous topical |
| | | ophthalmic emulsion |
| Manufacturer | Advanced BioScience Laboratories, Inc | Alcon ®, a |
| | | Novartis Company |
| Packaging | Construct II will be supplied as a sterile, single-use solution | DUREZOL (difluprednate |
| and Labeling | in 2-mL Crystal Zenith ® vials sealed with latex-free rubber | ophthalmic emulsion) |
| | stoppers and aluminum flip-off seals. Each vial will be labeled | 0.05% is a sterile, |
| | as required per country regulatory requirements. | aqueous topical |
| | | ophthalmic emulsion |
| | | supplied in an opaque |
| | | plastic bottle with a |
| | | controlled drop tip |
| | | and a pink cap; 5 |
| | | mL in an 8 mL bottle |

### 6.13.6 Steroid Regime

Cohort 4 and Cohort 5 participants randomized to Construct II treatment will receive a protocol-mandated postprocedure steroid regimen, as follows.

Beginning the evening of SCS administration of Construct II (Day 1), participants will instill 1 drop of difluprednate ophthalmic emulsion 0.0500 (DUREZOL) in the study eye, then instill drops daily in the study eye starting on Day 2, gradually decreasing in a tapered regimen over a total of 7 weeks:
4 times daily for 4 weeks, followed by
3 times daily for 1 week, followed by
2 times daily for 1 week, followed by
1 time daily for 1 week.

For any ocular inflammatory events occurring during or after the prophylaxis period, the Sponsor Clinical Development Lead should be contacted for management/treatment recommendations.

### 6.13.7 Vector Shedding

Sampling of blood (serum), urine, and tears will be performed for Construct II participants for measurement of vector concentrations. Refer to the Investigator Laboratory Manual for additional information regarding the processing, handling, and shipping of the samples.

Shedding data collected in these biological fluids provide a shedding profile of Construct II in the target patient population and is used to estimate the potential of transmission to untreated individuals. Shedding will be measured using quantitative polymerase chain reaction.

### 6.13.8 Results

As of the data cutoff, suprachoroidal delivery of Construct II has been well-tolerated in Cohorts 1-3 (Dose 1: 2.5×10¹¹ GC/eye; n=15 and Dose 2: 5.0×10¹¹ GC/eye; n=35). Five SAEs were reported, none of which were considered related to Construct II. No cases of chorioretinal vasculitis or occlusion, or hypotony were observed. For those dosed in Cohorts 1-3, all common treatment emergent adverse events (TEAEs) through 6 months in the study eye included conjunctival hemorrhage and conjunctival hyperemia, and were not considered related to Construct II. Comparing the results from Cohorts 2 and 3, there was no meaningful difference in the occurrence of TEAEs based on baseline AAV8 Nab levels.

Patients treated with Construct II in Cohorts 1-3 (n=50) continued to demonstrate stable Best Corrected Visual Acuity (BCVA) at 6 months. With a single suprachoroidal injection of Construct II, patients demonstrated clinically meaningful improvements in disease severity over time. In particular, 60% of patients in Cohort 1 (n=15), 47% of patients in Cohort 2 (n=15), and 55% of patients in Cohort 3 (n=20) achieved a >1 step improvement in DRSS at 6 months, compared to only 20% in the control (n=10). Moreover, 40% of patients in Cohort 1 (n=15), 20% of patients in Cohort 2 (n=15), and 5% of patients in Cohort 3 (n=20) achieved a >2 step improvement in DRSS at 6 months, compared to only 10% in the control (n=10). No patient in Cohorts 1-3 worsened by ≥2 steps in DRSS at 6 months as compared to 20% in the control.

### 6.13.9 Interim Data

As shown in Table 23, in Cohorts 1-3 (Dose 1 (2.5×10¹¹ GC/eye) and Dose 2 (5.0×10¹¹ GC/eye)), a few cases of mild intraocular inflammation were observed, which were resolved with topical corticosteroids.

TABLE 23
Cohorts 1-3 Interim Safety Summary

Cohorts 1 to 3: Common

| Ocular TEAEs^{a} and | Cohort 1 | | Cohort 2 | | Cohort 3 | | | |
|---|---|---|---|---|---|---|---|---|
| Intraocular Inflammation | Dose 1 | | Dose 2 | | Dose 2 | | | |
| in the Study Eye through 6 | NAb− | | NAb− | | NAb+ | | Total | |
| Months | (N = 15) | | (N = 15) | | (N = 20) | | (N = 50) | |
| Conjunctival hyperemia | 4 | (26.7%) | 5 | (33.3%) | 4 | (20.0%) | 13 | (26.0%) |
| Conjunctival hemorrhage | 3 | (20.0%) | 2 | (13.3%) | 1 | (5.0%) | 6 | (12.0%) |
| Episcleritis^{b} | 1 | (6.7%) | 1 | (6.7%) | 4 | (20.0%) | 6 | (12.0%) |
| Intraocular Inflammation^{c} | 0 | (0.0%) | 3 | (20.0%) | 0 | (0.0%) | 3 | (6.0%) |

^{a}Common TEAEs include AEs for total group ≥10% with onset up to 6 m visit.
^{b}All cases were mild (grade 1) and are resolved or resolving on topical corticosteroids.
^{c}All cases were mild (range +0.5 to +1) and most presented 2-6 weeks post injection, predominantly as anterior cells on slit lamp examination. Resolved on topical corticosteroids.
SAE: Serious Adverse Event; TEAE: Treatment Emergent Adverse Event.

Construct II was reported to be well tolerated in patients from Cohorts 4 and 5 (Dose 3 (1.0×10¹² GC/eye)), with no drug-related serious adverse events. Time of post-administration follow up ranged from 12 weeks to six months. There were zero cases of intraocular inflammation in Cohorts 4 and 5 (see Table 24), where all patients received short-course prophylactic topical steroids.

TABLE 24
Dose Level 3 with Short-Course Prophylactic
Topical Steroids: Interim Safety

| | w/PPX 11-24 Weeks Follow-up | |
|---|---|---|
| Common Ocular TEAEs¹ | Cohort 4 & Cohort 5 | |
| in the Study Eye | Dose Level 3 (N = 29) | |
| Episcleritis² | 7 | (24.1%) |
| Conjunctival Hemorrhage | 5 | (17.2%) |
| Intraocular Pressure Increased³ | 3 | (10.3%) |
| Conjunctival Hyperemia | 1 | (3.4%) |
| Intraocular Inflammation | 0 | (0.0%) |

¹Includes AEs ≥10% of the total groups.
²All mild to moderate (grade 1 and 2), presented within 1 week to 24 weeks post injection and have resolved or are tapering off topical corticosteroids.
³Intraocular pressure increased and ocular hypertension have been combined into one group. All mild to moderate and all controlled.

### EQUIVALENTS

Although embodiments are described in detail with reference to specific embodiments thereof, it will be understood that variations which are functionally equivalent are within the scope of this disclosure. Indeed, various modifications of embodiments described herein in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A method of treating neovascular age-related macular degeneration (nAMD) in a subject in need thereof, wherein the method comprises administering an anti-hVEGF treatment and a steroid treatment; wherein
a. the anti-hVEGF treatment comprises administering a therapeutically effective amount of a recombinant viral vector comprising a nucleotide sequence encoding an anti-hVEGF antigen-binding fragment to an eye of the subject; and
b. the steroid treatment comprises administering a therapeutically effective amount of a steroid to the eye of the subject.

2. A method of treating neovascular age-related macular degeneration (nAMD) or diabetic retinopathy (DR) in a subject in need thereof, wherein the method comprises administering an anti-hVEGF treatment and a steroid treatment; wherein
a. the anti-hVEGF treatment comprises administering a therapeutically effective amount of a recombinant viral vector comprising a nucleotide sequence encoding an anti-hVEGF antigen-binding fragment to the suprachoroidal space of an eye of the subject; and
b. the steroid treatment comprises administering a therapeutically effective amount of a steroid to the eye of the subject.

3. A method of treating neovascular age-related macular degeneration (nAMID) or diabetic retinopathy (DR) in a subject in need thereof, wherein the method comprises administering an anti-hVEGF treatment and a steroid treatment; wherein
a. the anti-hVEGF treatment comprises administering a therapeutically effective amount of a recombinant viral vector comprising a nucleotide sequence encoding an anti-hVEGF antigen-binding fragment to an eye of the subject; and
b. the steroid treatment comprises administering a therapeutically effective amount of triamcinolone acetonide to the eye of the subject.

4. A method of treating neovascular age-related macular degeneration (nAMID) or diabetic retinopathy (DR) in a subject in need thereof, wherein the method comprises administering an anti-hVEGF treatment and a steroid treatment; wherein
a. the anti-hVEGF treatment comprises administering a therapeutically effective amount of a recombinant viral vector comprising a nucleotide sequence encoding an anti-hVEGF antigen-binding fragment to an eye of the subject; and
b. the steroid treatment comprises administering a therapeutically effective amount of difluprednate to the eye of the subject.

5. The method of any one of claims 2-4, wherein the method is a method of treating neovascular age-related macular degeneration (nAMD).

6. The method of any one of claims 2-4, wherein the method is a method of treating diabetic retinopathy (DR).

7. The method of any one of claims 1 or 3-6, wherein the recombinant viral vector is administered to the suprachoroidal space of the eye of the subject.

8. The method of any one of claims 1-7, wherein the recombinant viral vector is administered by injection into the suprachoroidal space of the eye using a suprachoroidal drug delivery device.

9. The method of claim 8, wherein the suprachoroidal drug delivery device is a microinjector.

10. The method of any one of claims 1 or 3-6, wherein the recombinant viral vector is administered to the subretinal space of the eye of the subject.

11. The method of claim 10, wherein the method does not comprise performing a vitrectomy on the eye of the subject.

12. The method of claim 10, wherein the subretinal administration comprises performing a vitrectomy on the eye of the subject.

13. The method of claim 12, wherein the vitrectomy is a partial vitrectomy.

14. The method of claim 10 or claim 11, wherein the recombinant viral vector is administered to the subretinal space via the suprachoroidal space of the eye of the subject.

15. The method of claim 14, wherein the recombinant viral vector is administered with a subretinal drug delivery device comprising a catheter that can be inserted and tunneled through the suprachoroidal space toward the posterior pole, where a small needle injects into the subretinal space.

16. The method of claim 15, wherein the anti-hVEGF treatment comprises inserting and tunneling the catheter of the subretinal drug delivery device through the suprachoroidal space to administer the recombinant viral vector.

17. The method of any one of claims 1-16, wherein the steroid treatment ameliorates or prevents intraocular inflammation.

18. The method of any one of claims 1-17, wherein the steroid treatment ameliorates or prevents intraocular inflammation associated with the dose of the recombinant viral vector, the number of suprachoroidal injections, and/or the location of suprachoroidal injections.

19. The method of any one of claims 1, 2, and 4-18, wherein the steroid is topically administered.

20. The method of any one of claims 1, 2, and 5-19, wherein the steroid is a corticosteroid.

21. The method of any one of claims 1, 2, and 5-20, wherein the steroid is triamcinolone acetonide.

22. The method of any one of claims 1, 2, and 5-20, wherein the steroid is difluprednate.

23. The method of any one of claims 1-3, 5-9, and 17-21, wherein
a. the anti-hVEGF treatment comprises administering a recombinant viral vector comprising a nucleotide sequence encoding an anti-hVEGF antigen-binding fragment to the suprachoroidal space of an eye of the subject; and
b. the steroid treatment comprises administering triamcinolone acetonide to the eye of the subject.

24. The method of any one of claims 3, 5-21, and 23, wherein the triamcinolone acetonide is administered after administering the recombinant viral vector.

25. The method of any one of claims 3, 5-21, and 23, wherein the triamcinolone acetonide is administered before administering the recombinant viral vector.

26. The method of any one of claims 3, 5-21, and 23-25, wherein the triamcinolone acetonide is administered to the eye of the subject within about 24 hours, about 20 hours, about 16 hours, about 12 hours, about 8 hours, about 4 hours, about 3 hours, about 2 hours, about 1 hour, about 50 minutes, about 40 minutes, about 30 minutes, about 20 minutes, about 10 minutes, about 9 minutes, about 8 minutes, about 7 minutes, about 6 minutes, about 5 minutes, about 4 minutes, about 3 minutes, about 2 minutes, or about 1 minute of administering the recombinant viral vector.

27. The method of any one of claims 3, 5-18, 20, 21, and 23-26, wherein the triamcinolone acetonide is administered by injection into the eye of the subject.

28. The method of claim 27, wherein the triamcinolone acetonide is administered by a single injection into the eye of the subject.

29. The method of any one of claims 3, 5-18, 20, 21, and 23-28, wherein the steroid treatment consists of a single injection of triamcinolone acetonide into the eye of the subject.

30. The method of any one of claims 3, 5-18, 20, 21, and 23-29, wherein the triamcinolone acetonide is administered in a different quadrant of the eye than is the recombinant viral vector.

31. The method of any one of claims 3, 5-18, 20, 21, and 23-30, wherein the triamcinolone acetonide is administered to the subtenon of the eye.

32. The method of any one of claims 3, 5-18, 20, 21, and 23-31, wherein the triamcinolone acetonide is administered at a dose of about 40 mg.

33. The method of any one of claims 3, 5-18, 20, 21, and 23-32, wherein the triamcinolone acetonide is administered in a volume of about 1 mL.

34. The method of any one of claims 1, 2, 4-9, 17-20, and 22, wherein
a. the anti-hVEGF treatment comprises administering a recombinant viral vector comprising a nucleotide sequence encoding an anti-hVEGF antigen-binding fragment to the suprachoroidal space of an eye of the subject; and
b. the steroid treatment comprises administering difluprednate to the eye of the subject.

35. The method of any one of claims 4-20, 22, and 34, wherein the difluprednate is administered daily to the eye of the subject.

36. The method of claim 35, wherein the steroid treatment comprises administering difluprednate four times daily.

37. The method of claim 36, wherein the difluprednate is administered four times daily for at least one week, at least two weeks, at least three weeks, or at least four weeks.

38. The method of claim 37, wherein the difluprednate is administered four times daily for about four weeks.

39. The method of any one of claims 35-38, wherein the steroid treatment comprises administering difluprednate three times daily.

40. The method of claim 39, wherein the difluprednate is administered three times daily for at least one day, at least two days, at least three days, at least four days, at least five days, at least six days, or at least one week.

41. The method of claim 40, wherein the difluprednate is administered three times daily for about one week.

42. The method of any one of claims 35-41, wherein the steroid treatment comprises administering difluprednate two times daily.

43. The method of claim 42, wherein the difluprednate is administered two times daily for at least one day, at least two days, at least three days, at least four days, at least five days, at least six days, or at least one week.

44. The method of claim 44, wherein the difluprednate is administered two times daily for about one week.

45. The method of any one of claims 35-44, wherein the steroid treatment comprises administering difluprednate one time daily.

46. The method of claim 45, wherein the difluprednate is administered one time daily for at least one day, at least two days, at least three days, at least four days, at least five days, at least six days, or at least one week.

47. The method of claim 46, wherein the difluprednate is administered one time daily for about one week.

48. The method of any one of claims 4-20, 22, and 34-47, wherein the difluprednate is administered to the eye of the subject for a period of at least one week, at least two weeks, at least three weeks, at least four weeks, at least five weeks, at least six weeks, or at least seven weeks.

49. The method of claim 48, wherein the difluprednate is administered to the eye of the subject for a period of about seven weeks.

50. The method of claim 49, wherein the steroid treatment comprises administering difluprednate once on the first day of the steroid treatment, followed by four times daily for about four weeks, followed by three times daily for about one week, followed by two times daily for about one week, followed by one time daily for about one week.

51. The method of claim 49, wherein the steroid treatment consists of administering difluprednate once on the first day of the steroid treatment, followed by four times daily for about four weeks, followed by three times daily for about one week, followed by two times daily for about one week, followed by one time daily for about one week.

52. The method of any one of claims 4-20, 22, and 34-51, wherein the difluprednate is administered in the form of a ophthalmic emulsion.

53. The method of claim 52, wherein the ophthalmic emulsion comprises 0.5 mg/mL (0.05%) difluprednate.

54. The method of claim 52 or claim 53, wherein each administration of difluprednate comprises instilling one drop of the ophthalmic emulsion in the eye of the subject.

55. The method of claim 52 or claim 53, wherein each administration of difluprednate consists of instilling one drop of the ophthalmic emulsion in the eye of the subject.

56. The method of any one of claims 4-20, 22, and 34-55, wherein difluprednate is first administered to the eye of the subject within about seven days, about six days, about five days, about four days, about three days, about two days, or about one day of administering the recombinant viral vector.

57. The method of claim 56, wherein difluprednate is first administered to the eye of the subject on the same day as the recombinant viral vector is administered.

58. The method of claim 56 or claim 57, wherein the first administration of difluprednate occurs after the first administration of the recombinant viral vector.

59. The method of any one of claims 1-58, wherein the anti-hVEGF antigen-binding fragment is a Fab, F(ab′)₂, or single chain variable fragment (scFv).

60. The method of any one of claims 1-59, wherein the anti-hVEGF antigen-binding fragment comprises a heavy chain comprising the amino acid sequence of SEQ ID NO: 2 or SEQ ID NO: 4, and a light chain comprising the amino acid sequence of SEQ ID NO: 1 or SEQ ID NO:3.

61. The method of any one of claims 1-60, wherein the anti-hVEGF antigen-binding fragment comprises (a) a heavy chain comprising heavy chain CDRs 1-3 of the amino acid sequence of SEQ ID NO: 2, and (b) a light chain comprising light chain CDRs 1-3 of the amino acid sequence of SEQ ID NO: 1.

62. The method of any one of claims 1-60, wherein the anti-hVEGF antigen-binding fragment comprises (a) a heavy chain comprising heavy chain CDRs 1-3 of the amino acid sequence of SEQ ID NO: 4, and (b) a light chain comprising light chain CDRs 1-3 of the amino acid sequence of SEQ ID NO: 3.

63. The method of any one of claims 1-62, wherein the anti-hVEGF antigen-binding fragment comprises light chain CDRs 1-3 of SEQ ID NOs: 14-16 or SEQ ID NOs: 14, 15 and 63 and heavy chain CDRs 1-3 of SEQ ID NOs: 17-19 or SEQ ID NOs: 20, 18, and 21.

64. The method of any one of claims 1-63, wherein administration of the recombinant viral vector delivers a therapeutically effective amount of the anti-hVEGF antigen-binding fragment to the retina of said human subject.

65. The method of claim 65, wherein the therapeutically effective amount of the anti-hVEGF antigen-binding fragment is produced by retinal cells of the subject.

66. The method of any one of claims 1-65, wherein the recombinant viral vector is an rAAV vector.

67. The method of any one of claims 1-66, wherein the recombinant viral vector is an rAAV8 vector.

68. The method of any one of claims 1-67, wherein the recombinant viral vector comprises an expression cassette encoding an anti-hVEGF antigen-binding fragment, wherein the expression cassette is flanked by AAV2 inverted terminal repeats (ITRs), and wherein the expression cassette comprises:
a. a CB7 promotor consisting of a chicken β-actin promoter and a CMV enhancer;
b. a chicken β-actin intron;
c. a nucleotide sequence encoding:
i. an IL-2 signal peptide;
ii. a heavy chain of the anti-hVEGF antigen-binding fragment comprising the amino acid sequence of SEQ ID NO: 2;
iii. a self-cleaving furin (F)/F2A linker;
iv. a second IL-2 signal peptide; and
v. a light chain of the anti-hVEGF antigen-binding fragment comprising the amino acid sequence of SEQ ID NO: 1; and
d. a rabbit β-globin poly A signal.

69. The method of any one of claims 1-68, wherein the recombinant viral vector comprises the nucleotide sequence of SEQ ID NO: 56.

70. The method of any one of claims 1-69, wherein the recombinant viral vector is administered at a dose about 2.5×10¹¹ genome copies per eye.

71. The method of any one of claims 1-69, wherein the recombinant viral vector is administered at a dose about 5.0×10¹¹ genome copies per eye.

72. The method of any one claims 1-69, wherein the recombinant viral vector is administered at a dose about 1.0×10¹² genome copies per eye.

73. The method of any one of claims 1-72, wherein the recombinant viral vector is administered by double suprachoroidal injections.

74. The method of any one of claims 1-72, wherein the recombinant viral vector is administered by a single suprachoroidal injection.

75. A kit for use in a method of treating neovascular age-related macular degeneration (nAMD) according to any one of claims 1-5 and 7-74 comprising
a. a recombinant viral vector comprising a nucleotide sequence encoding an anti-hVEGF antigen-binding fragment; and
b. a steroid.

76. A kit for use in a method of treating diabetic retinopathy (DR) according to any one of claims 2-4 and 6-74 comprising
a. a recombinant viral vector comprising a nucleotide sequence encoding an anti-hVEGF antigen-binding fragment; and
b. a steroid.

77. The kit of claim 75 or claim 76, wherein the recombinant viral vector is formulated to be suitable for administration to the suprachoroidal space of the eye of the subject.

78. The kit of claim 75 or claim 76, wherein the recombinant viral vector is formulated to be suitable for administration to the subretinal space of the eye of the subject.

79. The kit of any one of claims 75-78, wherein the steroid is triamcinolone acetonide.

80. The kit of any one of claims 75-78, wherein the steroid is difluprednate.

81. Use of a recombinant viral vector comprising a nucleotide sequence encoding an anti-hVEGF antigen-binding fragment and a steroid in the manufacture of a medicament for the treatment of neovascular age-related macular degeneration (nAMD) according to any one claims 1-5 and 7-74.

82. Use of a recombinant viral vector comprising a nucleotide sequence encoding an anti-hVEGF antigen-binding fragment and a steroid in the manufacture of a medicament for the treatment of diabetic retinopathy (DR) according to any one of claims 2-4 and 6-74.

83. The use of claim 81 or claim 82, wherein the recombinant viral vector is formulated to be suitable for administration to the suprachoroidal space of the eye of the subject.

84. The use of claim 81 or claim 82, wherein the recombinant viral vector is formulated to be suitable for administration to the subretinal space of the eye of the subject.

85. The use of any one of claims 81-84, wherein the steroid is triamcinolone acetonide.

86. The use of any one of claims 81-84, wherein the steroid is difluprednate.
